(19) United States
(12) Patent Application Publication
(10) Pub. No.: US 2025/0243472 A1
(43) Pub. Date: Jul. 31, 2025

(54) MODIFIED HELICASES

(71) Applicant: Oxford Nanopore Technologies PLC, Oxford (GB)

(72) Inventors: Mark John Bruce, Oxford (GB); Andrew John Heron, Oxford (GB); Ruth Moysey, Oxford (GB); Szabolcs Soeroes, Oxford (GB); Elizabeth Jayne Wallace, Oxford (GB); James White, Oxford (GB)

(73) Assignee: Oxford Nanopore Technologies PLC, Oxford (GB)

(21) Appl. No.: 19/050,384

(22) Filed: Feb. 11, 2025

Related U.S. Application Data
(63) Continuation of application No. 18/047,019, filed on Oct. 17, 2022, now Pat. No. 12,258,591.
Application No. 18/047,019 is a continuation of application No. 16/893,332, filed on Jun. 4, 2020, now Pat. No. 11,525,125.
Application No. 16/893,332 is a division of application No. 15/028,651, filed on Apr. 11, 2016, now Pat. No. 10,724,018.
Application No. 15/028,651 is a continuation-in-part of application No. PCT/GB2014/050175, filed on Jan. 22, 2014.

(30) Foreign Application Priority Data
Oct. 18, 2013 (GB) 1318464.3
Mar. 17, 2014 (GB) 1404718.7
Apr. 4, 2014 (GB) 1406151.9

(51) Int. Cl. C12N 9/14; C12N 9/90; C12Q 1/6869

(52) U.S. Cl. CPC C12N 9/14; C12N 9/90; C12Q 1/6869; C12Q 2565/631; C12Y 306/04012

(57) ABSTRACT

The invention relates to a new method of characterising a target polynucleotide. The method uses a pore and a Dda helicase. The helicase controls the movement of the target polynucleotide through the pore. The invention also relates to modified Dda helicases which can be used to control the movement of polynucleotides and are particularly useful for sequencing polynucleotides.

### RELATED APPLICATIONS

This application is a continuation of U.S. application Ser. No. 18/047,019, filed Oct. 17, 2022, which is a continuation of U.S. application Ser. No. 16/893,332, filed Jun. 4, 2020, which is a division of U.S. application Ser. No. 15/028,651, filed Apr. 11, 2016, which is a national stage filing under U.S.C. § 371 of PCT International Application PCT/GB2014/052736, which has an international filing date of Sep. 10, 2014; is a continuation-in-part of PCT International Application PCT/GB2014/050175, which has an international filing date of Jan. 22, 2014; and claims foreign priority benefits under 35 U.S.C. § 119(a)-(d) or 35 U.S.C. § 365(b) of British application number 1406151.9, filed Apr. 4, 2014, British application number 1404718.7, filed Mar. 17, 2014, and British application number 1318464.3, filed Oct. 18, 2013, the contents of which are herein incorporated by reference in their entireties.

### REFERENCE TO AN ELECTRONIC SEQUENCE LISTING

The content of the electronic sequence listing (0036670033US03-SEQ-FL.xml; Size: 233,325 bytes; and Date of Creation: Feb. 11, 2025) is herein incorporated by reference in its entirety.

### FIELD OF THE INVENTION

The invention relates to a new method of characterising a target polynucleotide. The method uses a pore and a DNA-dependent ATPase (Dda) helicase. The helicase controls the movement of the target polynucleotide through the pore. The invention also relates to modified Dda helicases which can be used to control the movement of polynucleotides and are particularly useful for sequencing polynucleotides.

### BACKGROUND OF THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the “strand sequencing” method, a single polynucleotide strand is passed through the pore and the identity of the nucleotides are derived. Strand sequencing can involve the use of a nucleotide handling protein, such as a helicase, to control the movement of the polynucleotide through the pore.

### SUMMARY OF THE INVENTION

The inventors have demonstrated that a Dda helicase can control the movement of a polynucleotide through a pore especially when a potential, such as a voltage, is applied. The helicase is capable of moving a target polynucleotide in a controlled and stepwise fashion against or with the field resulting from the applied voltage.

The inventors have also surprisingly identified specific Dda mutants which have an improved ability to control the movement of a polynucleotide through a pore. Such mutants typically comprise one or more modifications in (i) the tower domain and/or (ii) the pin domain and/or (iii) the 1A (RecA-like motor) domain.

Accordingly, the invention provides a Dda helicase in which at least one cysteine residue and/or at least one non-natural amino acid have been introduced into (i) the tower domain and/or (ii) the pin domain and/or (iii) the 1A (RecA-like motor) domain, wherein the helicase retains its ability to control the movement of a polynucleotide.

The invention also provides:
a Dda helicase in which at least one cysteine residue and/or at least one non-natural amino acid have been introduced into the hook domain and/or the 2A (RecA-like motor) domain, wherein the helicase retains its ability to control the movement of a polynucleotide;
a Dda helicase which is modified to reduce its surface negative charge, wherein the helicase retains its ability to control the movement of a polynucleotide;
a first polypeptide comprising the pin domain and the 1A (RecA-like motor) domain from a Dda helicase and not comprising any other domains from a Dda helicase, wherein at least one cysteine residue and/or at least one non-natural amino acid have been introduced into the pin domain and/or the 1A (RecA-like motor) domain;
a second polypeptide comprising the 2A (RecA-like motor) domain, tower domain and hook domain from a Dda helicase and not comprising any other domains from a Dda helicase, wherein at least one cysteine residue and/or at least one non-natural amino acid have been introduced into the tower domain;
a helicase comprising a first polypeptide of the invention covalently attached to a second polypeptide of the invention, wherein the helicase has the ability to control the movement of a polynucleotide;
a construct comprising a Dda helicase or a helicase of the invention and an additional polynucleotide binding moiety, wherein the helicase is attached to the polynucleotide binding moiety and the construct has the ability to control the movement of a polynucleotide;
a polynucleotide comprising a sequence which encodes a helicase of the invention, a polypeptide of the invention or a construct of the invention;
a vector which comprises a polynucleotide of the invention operably linked to a promoter;
a host cell comprising a vector of the invention;
a method of making a helicase of the invention, a polypeptide of the invention or a construct of the invention, which comprises expressing a polynucleotide of the invention, transfecting a cell with a vector of the invention or culturing a host cell of the invention; a method of controlling the movement of a polynucleotide, comprising contacting the polynucleotide with a Dda helicase or a construct of the invention and thereby controlling the movement of the polynucleotide;
a method of characterising a target polynucleotide, comprising (a) contacting the target polynucleotide with a transmembrane pore and a Dda helicase or a construct of the invention such that the helicase controls the movement of the target polynucleotide through the pore and (b) taking one or more measurements as the polynucleotide moves with respect to the pore wherein the measurements are indicative of one or more characteristics of the target polynucleotide and thereby characterising the target polynucleotide;
method of forming a sensor for characterising a target polynucleotide, comprising forming a complex between (a) a pore and (b) a Dda helicase or a construct of the invention and thereby forming a sensor for characterising the target polynucleotide;
sensor for characterising a target polynucleotide, comprising a complex between (a) a pore and (b) a Dda helicase or a construct of the invention;
use of a Dda helicase or a construct of the invention to control the movement of a target polynucleotide through a pore;
a kit for characterising a target polynucleotide comprising (a) a pore and (b) a Dda helicase or a construct of the invention;
an apparatus for characterising target polynucleotides in a sample, comprising (a) a plurality of pores and (b) a plurality of Dda helicases or a plurality of constructs of the invention; and
a series of two or more helicases attached to a polynucleotide, wherein at least one of the two or more helicases is a Dda helicase of the invention.

### DESCRIPTION OF THE FIGURES

FIG. 1 shows an example current trace (y-axis label=Current (pA, 20 to 120), x-axis label=Time (s, 3500 to 8000)) of when a helicase (T4 Dda—E94C/A360C (SEQ ID NO: 8 with mutations E94C/A360C)) controlled the translocation of the Lambda DNA construct (0.2 nM, SEQ ID NO: 60 attached by its 3′ end to four iSpC3 spacers which are attached to the 5′ end of SEQ ID NO: 61 which is attached at its 3′ end to SEQ ID NO: 62, the SEQ ID NO: 61 region of this construct is hybridised to SEQ ID NO: 63 (which has a 3′ cholesterol tether)) through a nanopore (MS(B1-G75S/G77S/L88N/Q126R)8 MspA (SEQ ID NO: 2 with mutations G75S/G77S/L88N/Q126R)).

FIGS. 2A-2B show zoomed in regions of the helicase-controlled DNA movement shown in the current trace in FIG. 1 (y-axis label=Current (pA, upper trace 20 to 80, lower trace 20 to 60), x-axis label=Time (s, upper trace 2995 to 3020, lower trace 8140 to 8170) upper and lower trace). FIG. 2A shows the beginning of the helicase-controlled DNA movement and FIG. 2B shows the end of the helicase controlled DNA movement.

FIGS. 3A-3B show a fluorescence assay for testing helicase binding to linear (FIG. 3A) or circular (FIG. 3B) single-stranded DNA. FIG. 3A shows a custom fluorescent substrate used to assay the ability of T4 Dda—E94C/A360C (SEQ ID NO: 8 with mutations E94C/A360C) helicase to bind to linear single-stranded DNA. The 44 nt single-stranded DNA substrate (1 nM final, SEQ ID NO: 64, labelled W) has a carboxyfluorescein (FAM) attached to the thymine base at position 37 in SEQ ID NO: 64 (circle labelled X). As the helicase (labelled Y) bound to the DNA substrate in buffered solution (25 mM potassium phosphate, 151.5 mM KCl, pH8.0, 10 mM MgCl₂), the fluorescence anisotropy (a property relating to the speed of tumbling of the DNA substrate in solution) increased. The lower the amount of helicase needed to affect an increase in anisotropy, the tighter the binding affinity between the DNA and helicase. In situation 1 with no enzyme bound the DNA substrate exhibited faster tumbling and low anisotropy, whereas, in situation 2 with enzyme bound to the DNA substrate it exhibited slower tumbling and high anisotropy (this was attributed to the mass increase upon binding of a large protein molecule to the DNA). The black bar labelled Z corresponds to increasing helicase concentration (the thicker the bar the higher the helicase concentration). FIG. 3B shows a custom fluorescent substrate used to assay the ability of T4 Dda—E94C/A360C (SEQ ID NO: 8 with mutations E94C/A360C) helicase to bind to circular single-stranded DNA. The 75 nt circular single-stranded DNA substrate (1 nM final, SEQ ID NO: 65, labelled V) had a carboxyfluorescein (FAM) attached to one of the thymine bases in SEQ ID NO: 65 (circle labelled X). As the helicase (labelled Y) bound to the oligonucleotide in buffered solution (25 mM potassium phosphate, 151.5 mM KCl, pH8.0, 10 mM MgCl₂), the fluorescence anisotropy (a property relating to the rate of tumbling of the oligonucleotide in solution) increased. The lower the amount of helicase needed to affect an increase in anisotropy, the tighter the binding affinity between the DNA and helicase. In situation 1 with no enzyme bound the DNA substrate exhibited faster tumbling and low anisotropy, whereas, in situation 2 with enzyme bound to the DNA substrate it exhibited slower tumbling and high anisotropy (this was attributed to the mass increase upon binding of a large protein molecule to the DNA). The black bar labelled Z corresponds to increasing helicase concentration (the thicker the bar the higher the helicase concentration).

FIG. 4 shows the change in anisotropy of the linear and circular single-stranded DNA oligonucleotides (SEQ ID NO: 64 or 65) with increasing amounts of T4 Dda—E94C/A360C (SEQ ID NO: 8 with mutations E94C/A360C) (y-axis label=Anisotropy (blank subtracted, 50 to 200), x-axis label=Concentration T4 Dda (nM, 0.01 to 1000)) at the end of a 60 min incubation period. The data with black circles corresponded to the linear ssDNA construct. The data with the empty squares corresponded to the circular ssDNA construct.

FIG. 5 shows the equilibrium dissociation constants (K_{d}) for T4 Dda—E94C/A360C (SEQ ID NO: 8 with mutations E94C/A360C) binding to linear or circular single-stranded DNA after a 60 minute incubation. The graph was obtained through fitting one phase dissociation binding curves through the data shown in FIG. 4 using Graphpad Prism software (y-axis label=dissociation constant Kd (nM, 0 to 12), x-axis label=Ref. Number, where Ref. Number 1 corresponded to the linear single-stranded DNA oligonucleotide and Ref. Number 2 corresponded to the circular single-stranded DNA oligonucleotide).

FIG. 6 shows an example current trace (y-axis label=Current (pA, upper trace 50 to 200, lower trace 55 to 75), x-axis label=Time (s, upper trace 11420 to 11620, lower trace 11524 to 11527)) of when a helicase (TrwC Cba (SEQ ID NO: 66)) controlled the translocation of DNA (0.2 nM, SEQ ID NO: 67 attached by its 3′ end to four iSpC3 spacers which are attached to the 5′ end of SEQ ID NO: 61 which is attached at its 3′ end to four 5-nitroindoles the last of which is attached to the 5′ end of SEQ ID NO: 68, in addition SEQ ID NO: 63 is hybridised to SEQ ID NO: 61) through a nanopore (MS(B1-G75S/G77S/L88N/Q126R)8 MspA (SEQ ID NO: 2 with mutations G75S/G77S/L88N/Q126R)). The upper trace shows two helicase controlled DNA movements and the lower trace shows a zoomed in region labelled X in the upper level. As the helicase moved the DNA through the nanopore the current levels detected have been labelled a to k. When TrwC Cba controlled translocation through the nanopore, the DNA stepped back and therefore levels corresponding to b, c, h and i were observed several times.

FIG. 7 shows an example current trace (y-axis label=Current (pA, upper trace 50 to 250, lower trace 55 to 75), x-axis label=Time (s, upper trace 300 to 700, lower trace 572 to 577)) of when a helicase (T4 Dda E94C/A360C (SEQ ID NO: 8 with mutations E94C/A360C)) controlled the translocation of DNA (0.2 nM, SEQ ID NO: 67 attached by its 3′ end to four iSpC3 spacers which are attached to the 5′ end of SEQ ID NO: 61 which is attached at its 3′ end to four 5-nitroindoles the last of which is attached to the 5′ end of SEQ ID NO: 68, in addition SEQ ID NO: 63 is hybridised to SEQ ID NO: 61) through a nanopore (MS(B1-G75S/G77S/L88N/Q126R)8 MspA (SEQ ID NO: 2 with mutations G75S/G77S/L88N/Q126R)). The upper trace shows three helicase controlled DNA movements and the lower trace shows a zoomed in region labelled X in the upper level. As the helicase moved the DNA through the nanopore the current levels detected have been labelled a to k. When T4 Dda E94C/A360C (SEQ ID NO: 8 with mutations E94C/A360C) controlled translocation through the nanopore, the DNA did not step back and therefore single current levels corresponding to levels a to i were observed.

FIG. 8 shows a diagram of the lambda DNA construct used in Examples 1 and 4. SEQ ID NO: 60 (labelled A) is attached at its 3′ end to four iSpC3 spacers (labelled B). The four iSpC3 spacers are attached to the 5′ end of SEQ ID NO: 61 (labelled C). SEQ ID NO: 61 is attached to four iSpC3 spacers (labelled D) which are attached to SEQ ID NO: 62 (labelled E) at its 5′ end. SEQ ID NO: 61 is hybridised to SEQ ID NO: 63 (labelled F, which has a 3′ cholesterol tether). Two separate sections of labelled region E are highlighted as region 1 (shown as a solid grey line) and region 2 (shown as a dotted grey line) in the figure and are referred to in Example 4.

FIG. 9 shows example current traces (both traces have the following axes labels y-axis label=Current (pA), x-axis label=Time (s)) of when a helicase (T4 Dda—E94C/A360C/C109A/C136A (SEQ ID NO: 8 with mutations E94C/A360C/C109A/C136A and then (ΔM1)G1G2)) controlled the translocation of DNA (0.1 nM, SEQ ID NO: 67 attached by its 3′ end to four iSpC3 spacers which are attached to the 5′ end of SEQ ID NO: 61 which is attached at its 3′ end to four 5-nitroindoles spacers which are attached to the 5′ end of SEQ ID NO: 69, the SEQ ID NO: 61 region of this construct is hybridised to SEQ ID NO: 63 (which has a 3′ cholesterol tether)) through an MspA nanopore. Both traces showed multiple helicase controlled DNA movements.

FIG. 10 shows example current traces (both traces have the following axes labels y-axis label=Current (pA), x-axis label=Time (s)) of when a helicase (T4 Dda—E94C/A360C/C114A/C171A/C421D (SEQ ID NO: 8 with mutations E94C/A360C/C114A/C171A/C421D and then (ΔM1)G1G2)) controlled the translocation of DNA (0.1 nM, SEQ ID NO: 67 attached by its 3′ end to four iSpC3 spacers which are attached to the 5′ end of SEQ ID NO: 61 which is attached at its 3′ end to four 5-nitroindoles spacers which are attached to the 5′ end of SEQ ID NO: 69, the SEQ ID NO: 61 region of this construct is hybridised to SEQ ID NO: 63 (which has a 3′ cholesterol tether)) through an MspA nanopore. Both traces showed multiple helicase controlled DNA movements.

FIG. 11 shows how the helicase controlled DNA movement speed for the mutant T4 Dda—E94C/A360C varied during the course of a 6 hour 5 minute experimental run (y-axis label=events per second, x-axis label=time (hours)). The bars in the graph labelled with a star (*) corresponded to helicase controlled movement speed of region 2 of the lambda DNA construct (shown in FIG. 8) passing through the nanopore and those without a star corresponded to the helicase controlled movement speed of region 1 of the lambda DNA construct (shown in FIG. 8) passing through the nanopore. Events per second was used in the examples as a measure of the speed of translocation of DNA movement through the nanopore.

FIG. 12 shows how the helicase controlled DNA movement speed for the mutant T4 Dda—E94C/A360C/C114A/C171A/C421D varied during the course of a six hour five minute experimental run (y-axis label=events per second, x-axis label=time (hours)). The bars in the graph labelled with a star (*) corresponded to helicase controlled movement speed of region 2 of the lambda DNA construct (shown in FIG. 8) passing through the nanopore and those without a star corresponded to the helicase controlled movement speed of region 1 of the lambda DNA construct (shown in FIG. 8) passing through the nanopore. Events per second was used in the examples as a measure of the speed of translocation of DNA movement through the nanopore.

FIG. 13 shows how the helicase controlled DNA movement speed for the mutant T4 Dda—E94C/A360C/C109A/C136A varied during the course of a six hour five minute experimental run (y-axis label=events per second, x-axis label=time (hours)). The bars in the graph labelled with a star (*) corresponded to helicase controlled movement speed of region 2 of the lambda DNA construct (shown in FIG. 8) passing through the nanopore and those without a star corresponded to the helicase controlled movement speed of region 1 of the lambda DNA construct (shown in FIG. 8) passing through the nanopore. Events per second was used in the examples as a measure of the speed of translocation of DNA movement through the nanopore.

FIG. 14 shows a diagram of the DNA construct used in Example 5. Label A corresponds to 25iSpC3 spacers which are attached at the 3′ end to SEQ ID NO: 70 (labelled B). Label B is attached at its 3′ end to four iSp18 spacers (labelled C). The four iSp18 spacers are attached to the 5′ end of SEQ ID NO: 61 (labelled D). SEQ ID NO: 61 is attached to four 5-nitroindoles (labelled E) which are attached to SEQ ID NO: 71 (labelled F) at its 5′ end. SEQ ID NO: 61 is hybridised to SEQ ID NO: 63 (labelled G). SEQ ID NO: 63 has six iSp18 spacers, two thymines and a 3′ cholesterol TEG attached at its 3′ end.

FIG. 15 shows an example current trace (y-axis label=Current (pA, 10 to 120), x-axis label=Time (s, 210.5 to 287)) of when a helicase (T4 Dda—E94C/C109A/C136A/A360C/W378A (SEQ ID NO: 8 with mutations E94C/C109A/C136A/A360C/W378A)) controlled the translocation of DNA construct Z (shown in FIG. 8) through an MspA nanopore.

FIGS. 16A-16C show zoomed in regions of the helicase-controlled DNA movement shown in the current trace in FIG. 15 (y-axis label=Current (pA, upper trace 20 to 95, middle trace 28.3 to 72.7 and lower trace 20 to 95), x-axis label=Time (s, upper trace 211.3 to 214.4, middle trace 212.9 to 213.7 and lower trace 283.2 to 286.2). FIG. 16A shows the beginning of the helicase-controlled DNA movement, FIG. 16B shows a zoomed in region of trace A and FIG. 16C shows the end of the helicase controlled DNA movement.

FIG. 17 shows DNA construct X which was used in Example 6. Section a of DNA construct X corresponds to 25 iSpC3 spacers, which are attached to the 5′ end of SEQ ID NO: 70 (labelled b). Section b is the region of construct X to which the helicase enzymes T4 Dda—E94C/A360C or T4 Dda—E94C/C109A/C136A/A360C (depending on the experiment) bound (labelled c). The length of section b corresponded to the footprint (binding region) of two enzymes e.g. it was long enough to allow two enzymes to bind to this region. Section d corresponds to four iSp18 spacers. Section e corresponds to SEQ ID NO: 61. Section f corresponds to four 5′-nitroindoles. Section g corresponds to SEQ ID NO: 72 (this section of the strand was referred to as region 3 of DNA construct X). Section h (shown by black dots) corresponds to four iSpC3 spacers, which are attached to the 5′ end of SEQ ID NO: 73 (labelled i which was referred to as region 4 of DNA construct X). Section j corresponds to SEQ ID NO: 74 and section k corresponds to SEQ ID NO: 75 which is attached to a 5′ cholesterol TEG. It was possible to distinguish between regions 3 and 4 as they translocated through a nanopore as they produced different characteristics. Furthermore, the section h spacers (four iSpC3 spacers) produced a current spike in the current trace which aided identification of the transition from region 3 to region 4.

FIGS. 18A-18B show example plots of when the helicase T4 Dda—E94C/A360C (SEQ ID NO: 24 with mutations E94C/A360C) controlled the translocation of DNA construct X (see FIG. 17 for details) through an MspA nanopore. The x-axis corresponds to the movement index and the y-axis corresponds to the current (pA). For each DNA strand which moved through the pore the current was measured as a function of time. The moving DNA resulted in stepwise changes in the measured current levels. The observed current levels were fitted to obtain a mean current for each step, and assigned an incrementing movement index point. The mean current against movement index therefore closely approximated the original current signal, and was used to characterise the translocated DNA. Plots in FIGS. 18A and 18B each showed a single DNA strand moving through the nanopore under the control of helicases, the labelled regions 3 and 4 corresponded to the translocation of region 3 and 4 of DNA construct X (see FIG. 17). Trace A shows the movement index observed when construct X was translocated through the pore under the control of a single T4 Dda—E94C/A360C helicase. Trace B shows the movement index observed when construct X was translocated through the pore under the control of two T4 Dda—E94C/A360C helicases. As region 3 and region 4 were approximately the same length, the movement index observed for each region would have been expected to have had approximately the same number of points in the movement index. Plot A shows a significantly reduced number of points in the movement index for region 4 when compared to region 3, therefore, less information was derived from region 4 than region 3. However, plot B (where the movement of construct X was controlled by two T4 Dda—E94C/A360C helicases) showed many more points in the movement index of region 4, which indicated that approximately the same amount of information was derived from region 4 as region 3. Using two helicases to control the movement of construct X provided improved movement as more information was derived from region 4 than when a single helicase controlled the movement.

FIGS. 19A-19B show example plots of when the helicase T4 Dda—E94C/C109A/C136A/A360C (SEQ ID NO: 24 with mutations E94C/C109A/C136A/A360C) controlled the translocation of DNA construct X (see FIG. 17 for details) through an MspA nanopore. The x-axis corresponds to the movement index (see FIGS. 18A-18B's figure legend for description of movement index) and the y-axis corresponds to the current (pA). Plots A and B each showed a single DNA strand moving through the nanopore under the control of helicases, the labelled regions 3 and 4 corresponded to the translocation of region 3 and 4 of DNA construct X (see FIG. 17). Trace A shows the movement index observed when construct X was translocated through the pore under the control of a single T4 Dda—E94C/C109A/C136A/A360C. Trace B shows the movement index observed when construct X was translocated through the pore under the control of two T4 Dda—E94C/C109A/C136A/A360C helicases. As region 3 and region 4 were approximately the same length, the movement index observed for each region would have been expected to have had approximately the same number of points in the movement index. Plot A shows a significantly reduced number of points in the movement index for region 4 when compared to region 3, therefore, less information was derived from region 4 than region 3. However, plot B (where the movement of construct X was controlled by two T4 Dda—E94C/C109A/C136A/A360C helicases) showed approximately the same number of points in both sections of the movement index, and therefore approximately the same amount of information was derived from region 4 as region 3. Using two helicases to control the movement of construct X provided improved movement as more information was derived from region 4 than when a single helicase controlled the movement.

FIG. 20 shows DNA construct Z which was used in Example 7 and 8. Section m of DNA construct Z corresponds to 40 iSpC3 spacers, which are attached to the 5′ end of SEQ ID NO: 76 (labelled n). Section n is a region of construct Z to which the helicase enzyme T4 Dda—E94C/C109A/C136A/A360C or T4 Dda—E94C/C109A/C136A/A360C/W378A bound. The length of section n corresponded to the footprint (binding region) of one enzyme e.g. it was long enough to allow one enzyme to bind to this region. The sections labelled d correspond to four iSp18 spacers. Section o corresponds to SEQ ID NO: 77, part of this section was a region of construct Z to which the helicase enzyme T4 Dda—E94C/C109A/C136A/A360C/W378A bound. Section p corresponds to SEQ ID NO: 78 (part of this section of the strand was referred to as region 5 of DNA construct Z). Section h (shown by black dots) corresponds to four iSpC3 spacers, which are attached to the 5′ end of SEQ ID NO: 79 (labelled q). Section r corresponds to the complementary sequence of SEQ ID NO: 78 (labelled r, which was referred to as region 6 of DNA construct Z). Section s corresponds to SEQ ID NO: 74. Section k corresponds to SEQ ID NO: 75 which is attached to a 5′ cholesterol TEG (labelled 1). Section t corresponds to SEQ ID NO: 80. It was possible to distinguish between regions 5 and 6 as they translocated through a nanopore as they produced different characteristics. Furthermore, the section h spacers (four iSpC3 spacers) produced a current spike in the current trace which aided identification of the transition from region 5 to region 6.

FIGS. 21A-21B show example plots of when either the helicase T4 Dda—E94C/C109A/C136A/A360C (FIG. 21A, SEQ ID NO: 24 with mutations E94C/C109A/C136A/A360C) or the helicases T4 Dda—E94C/C109A/C136A/A360C and T4 Dda—E94C/C109A/C136A/A360C/W378A (FIG. 21B) controlled the translocation of DNA construct Z (FIG. 20) through an MspA nanopore. The x-axis corresponds to the movement index and the y-axis corresponds to the current (pA). For each DNA strand which moved through the pore the current was measured as a function of time. The moving DNA resulted in stepwise changes in the measured current levels. The observed current levels were fitted to obtain a mean current for each step, and assigned an incrementing movement index point. The mean current against movement index therefore closely approximated the original current signal, and was used to characterise the translocated DNA. Plots in FIGS. 21A-21B each showed a single DNA strand moving through the nanopore under the control of helicases, the labelled regions 5 and 6 corresponded to the translocation of region 5 and 6 of DNA construct Z (see FIG. 20). Trace A shows the movement index observed when construct Z was translocated through the pore under the control of a single T4 Dda—E94C/C109A/C136A/A360C helicase. Trace B shows the movement index observed when construct Z was translocated through the pore under the control of both T4 Dda—E94C/C109A/C136A/A360C and T4 Dda—E94C/C109A/C136A/A360C/W378A. As region 5 and region 6 were approximately the same length, the movement index observed for each region would have been expected to have had approximately the same number of points in the movement index. Plot A shows a significantly reduced number of points in the movement index for region 6 when compared to region 5, therefore, less information was derived from region 6 than region 5. However, plot B (where the movement of construct Z was controlled by both T4 Dda—E94C/C109A/C136A/A360C and T4 Dda—E94C/C109A/C136A/A360C/W378A) showed many more points in the movement index of region 6, which indicated that approximately the same amount of information was derived from region 6 as region 5. Using two different helicases to control the movement of construct Z provided improved movement as more information was derived from region 6 than when a single helicase controlled the movement.

FIGS. 22A-22B show example plots of when either the single helicase T4 Dda—E94C/C109A/C136A/A360C/W378A FIG. 22A, SEQ ID NO: 24 with mutations E94C/C109A/C136A/A360C/W378A) or two T4 Dda—E94C/C109A/C136A/A360C/W378A helicases (FIG. 22B) were used to controlled the translocation of DNA construct Z (FIG. 20) through an MspA nanopore. The x-axis corresponds to the movement index and the y-axis corresponds to the current (pA). For each DNA strand which moved through the pore the current was measured as a function of time. The moving DNA resulted in stepwise changes in the measured current levels. The observed current levels were fitted to obtain a mean current for each step, and assigned an incrementing movement index point. The mean current against movement index therefore closely approximated the original current signal, and was used to characterise the translocated DNA. Plots in FIGS. 22A-22B showed a single DNA strand moving through the nanopore under the control of either one or two a helicases, the labelled regions 5 and 6 corresponded to the translocation of region 5 and 6 of DNA construct Z (see FIG. 20). Trace A shows the movement index observed when construct Z was translocated through the pore under the control of a single T4 Dda—E94C/C109A/C136A/A360C/W378A helicase. Trace B shows the movement index observed when construct Z was translocated through the pore under the control of two T4 Dda—E94C/C109A/C136A/A360C/W378A helicases. As region 5 and 6 were approximately the same length, the movement index observed for each region would have been expected to have had approximately the same number of points in the movement index. Plot A shows a significantly reduced number of points in the movement index for region 6 when compared to region 5, therefore, less information was derived from region 6 than region 5. However, plot B (where the movement of construct Z was controlled by two T4 Dda—E94C/C109A/C136A/A360C/W378A helicases) showed many more points in the movement index of region 6, which indicated that approximately the same amount of information was derived from region 6 as region 5. Therefore, using two helicases to control the movement of construct Z provided improved movement as more information was derived from region 6 than when a single helicase controlled the movement.

FIGS. 23A-23L show an alignment of the preferred Dda helicases of the invention. Top to bottom and left to right the sequences in FIGS. 23A-23H correspond to SEQ ID NOs: 8-23, and 8-23. Top to bottom and left to right the sequences in FIGS. 23I-23L correspond to SEQ ID NOs: 8-23.

### DESCRIPTION OF THE SEQUENCE LISTING

SEQ ID NO: 1 shows the codon optimised polynucleotide sequence encoding the MS-B1 mutant MspA monomer. This mutant lacks the signal sequence and includes the following mutations: D90N, D91N, D93N, D118R, D134R and E139K.

SEQ ID NO: 2 shows the amino acid sequence of the mature form of the MS-B1 mutant of the MspA monomer. This mutant lacks the signal sequence and includes the following mutations: D90N, D91N, D93N, D118R, D134R and E139K.

SEQ ID NO: 3 shows the polynucleotide sequence encoding one monomer of ca-hemolysin-E111N/K147N ((α-HL-NN; Stoddart et al., PNAS, 2009; 106(19): 7702-7707).

SEQ ID NO: 4 shows the amino acid sequence of one monomer of α-HL-NN.

SEQ ID NOs: 5 to 7 show the amino acid sequences of MspB, C and D.

SEQ ID NOs: 8 to 23 show the amino acid sequences of the Dda helicases shown in Tables 1 and 2.

SEQ ID NO: 24 shows the amino acid sequence of a preferred HhH domain.

SEQ ID NO: 25 shows the amino acid sequence of the ssb from the bacteriophage RB69, which is encoded by the gp32 gene.

SEQ ID NO: 26 shows the amino acid sequence of the ssb from the bacteriophage T7, which is encoded by the gp2.5 gene.

SEQ ID NO: 27 shows the amino acid sequence of the UL42 processivity factor from Herpes virus 1.

SEQ ID NO: 28 shows the amino acid sequence of subunit 1 of PCNA.

SEQ ID NO: 29 shows the amino acid sequence of subunit 2 of PCNA.

SEQ ID NO: 30 shows the amino acid sequence of subunit 3 of PCNA.

SEQ ID NO: 31 shows the amino acid sequence of Phi29 DNA polymerase.

SEQ ID NO: 32 shows the amino acid sequence (from 1 to 319) of the UL42 processivity factor from the Herpes virus 1.

SEQ ID NO: 33 shows the amino acid sequence of the ssb from the bacteriophage RB69, i.e.

SEQ ID NO: 25, with its C terminus deleted (gp32RB69CD).

SEQ ID NO: 34 shows the amino acid sequence (from 1 to 210) of the ssb from the bacteriophage T7 (gp2.5T7-R211Del). The full length protein is shown in SEQ ID NO: 96.

SEQ ID NO: 35 shows the amino acid sequence of the 5^{th} domain of Hel308 Hla.

SEQ ID NO: 36 shows the amino acid sequence of the 5^{th} domain of Hel308 Hvo.

SEQ ID NO: 37 shows the amino acid sequence of the (HhH)2 domain.

SEQ ID NO: 38 shows the amino acid sequence of the (HhH)2-(HhH)2 domain.

SEQ ID NO: 39 shows the amino acid sequence of the human mitochondrial SSB (HsmtSSB).

SEQ ID NO: 40 shows the amino acid sequence of the p5 protein from Phi29 DNA polymerase.

SEQ ID NO: 41 shows the amino acid sequence of the wild-type SSB from E. coli.

SEQ ID NO: 42 shows the amino acid sequence of the ssb from the bacteriophage T4, which is encoded by the gp32 gene.

SEQ ID NO: 43 shows the amino acid sequence of EcoSSB-CterAla.

SEQ ID NO: 44 shows the amino acid sequence of EcoSSB-CterNGGN.

SEQ ID NO: 45 shows the amino acid sequence of EcoSSB-Q152del.

SEQ ID NO: 46 shows the amino acid sequence of EcoSSB-G117del.

SEQ ID NO: 47 shows the amino acid sequence of Topoisomerase V Mka (Methanopyrus Kandleri).

SEQ ID NO: 48 shows the amino acid sequence of domains H-L of Topoisomerase V Mka (Methanopyrus Kandleri).

SEQ ID NO: 49 shows the amino acid sequence of Mutant S (Escherichia coli).

SEQ ID NO: 50 shows the amino acid sequence of Sso7d (Sufolobus solfataricus).

SEQ ID NO: 51 shows the amino acid sequence of Sso10b1 (Sulfolobus solfataricus P2).

SEQ ID NO: 52 shows the amino acid sequence of Sso10b2 (Sulfolobus solfataricus P2).

SEQ ID NO: 53 shows the amino acid sequence of Tryptophan repressor (Escherichia coli).

SEQ ID NO: 54 shows the amino acid sequence of Lambda repressor (Enterobacteria phage lambda).

SEQ ID NO: 55 shows the amino acid sequence of Cren7 (Histone crenarchaea Cren7 Sso).

SEQ ID NO: 56 shows the amino acid sequence of human histone (Homo sapiens).

SEQ ID NO: 57 shows the amino acid sequence of dsbA (Enterobacteria phage T4).

SEQ ID NO: 58 shows the amino acid sequence of Rad51 (Homo sapiens).

SEQ ID NO: 59 shows the amino acid sequence of PCNA sliding clamp (Citromicrobium bathyomarinum JL354).

SEQ ID NO: 60 shows a polynucleotide sequence used in Example 1. SEQ ID NO: 60 is attached by its 3′ end to four iSpC3 spacers which are attached to the 5′ end of SEQ ID NO: 61.

SEQ ID NO: 61 shows a polynucleotide sequence used in Example 1, 3, 4 and 6.

SEQ ID NO: 62 shows a polynucleotide sequence used in Example 1. SEQ ID NO: 62 is attached by its 5′ end to three iSpC3 spacers which are attached to the 3′ end of SEQ D NO: 61.

SEQ ID NO: 63 shows a polynucleotide sequence used in Example 1 which at the 3′ end of the sequence has six iSp18 spacers attached to two thymine residues and a 3′ cholesterol TEG.

SEQ ID NO: 64 shows a polynucleotide sequence used in Example 2. The sequence has a carboxyfluorescein (FAM) attached to the thymine at position 37 in the sequence.

SEQ ID NO: 65 shows a circular polynucleotide sequence used in Example 2. The sequence has a carboxyfluorescein (FAM) attached to one thymine in the sequence.

SEQ ID NO: 66 shows the amino acid sequence for the Trwc Cba helicase.

SEQ ID NO: 67 shows a polynucleotide sequence used in Example 3 and 4.

SEQ ID NO: 68 shows a polynucleotide sequence used in Example 3. SEQ ID NO: 68 is attached by its 5′ end to four 5-nitroindoles which are attached to the 3′ end of SEQ ID NO: 61.

SEQ ID NO: 69 shows a polynucleotide sequence used in Example 4.

SEQ ID NO: 70 shows a polynucleotide sequence used in Example 5 and 6.

SEQ ID NO: 71 shows a polynucleotide sequence used in Example 5.

SEQ ID NO: 72 shows a polynucleotide sequence used in Example 6.

SEQ ID NO: 73 shows a polynucleotide sequence used in Example 6.

SEQ ID NO: 74 shows a polynucleotide sequence used in Example 6, 7 and 8.

SEQ ID NO: 75 shows a polynucleotide sequence used in Example 6, 7 and 8.

SEQ ID NO: 76 shows a polynucleotide sequence used in Example 7 and 8.

SEQ ID NO: 77 shows a polynucleotide sequence used in Example 7 and 8.

SEQ ID NO: 78 shows a polynucleotide sequence used in Example 7 and 8.

SEQ ID NO: 79 shows a polynucleotide sequence used in Example 7 and 8.

SEQ ID NO: 80 shows a polynucleotide sequence used in Example 7 and 8.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a helicase” includes “helicases”, reference to “a modification” includes two or more such modifications, reference to “a transmembrane protein pore” includes two or more such pores, and the like.

All publications, patents and patent applications cited herein, whether supra or infra, are hereby incorporated by reference in their entirety.

### Modified Dda Helicases

The present invention provides a modified Dda helicase. The one or more specific modifications are discussed in more detail below. The modification(s) allows the modified helicase to remain bound to the polynucleotide for longer. The modified helicase retains its ability to control the movement of a polynucleotide. In other words, the modified helicase is still capable of controlling the movement of a polynucleotide. The extent to which the helicase can control the movement of a polynucleotide is typically altered by the modifications as discussed in more detail below.

The Dda helicase of the invention is modified. The modified helicase is typically modified compared with the corresponding wild-type helicase or natural helicase. The helicase of the invention is artificial or non-natural.

The ability of a helicase to bind to and unbind from a polynucleotide can be determined using any method known in the art. Suitable binding/unbinding assays include, but are not limited to, native polyacrylamide gel electrophoresis (PAGE), fluorescence anisotropy, calorimetry and Surface plasmon resonance (SPR, such as Biacore™). The ability of a helicase to unbind from a polynucleotide can of course be determined by measuring the time for which the helicase can control the movement of a polynucleotide. This may also be determined using any method known in the art. The ability of a helicase to control the movement of a polynucleotide is typically assayed in a nanopore system, such as the ones described below. The ability of a helicase to control the movement of a polynucleotide can be determined as described in the Examples.

A modified helicase of the invention is a useful tool for controlling the movement of a polynucleotide during Strand Sequencing. The Dda helicase can control the movement of DNA in at least two active modes of operation (when the helicase is provided with all the necessary components to facilitate movement e.g. ATP and Mg²⁺) and one inactive mode of operation (when the helicase is not provided with the necessary components to facilitate movement). When provided with all the necessary components to facilitate movement the Dda helicase moves along the DNA in the 5′-3′ direction, but the orientation of the DNA in the nanopore (dependent on which end of the DNA is captured) means that the enzyme can be used to either move the DNA out of the nanopore against the applied field, or move the DNA into the nanopore with the applied field. When the 3′ end of the DNA is captured the helicase works against the direction of the field applied by the voltage, pulling the threaded DNA out of the nanopore and into the cis chamber. However, when the DNA is captured 5′-down in the nanopore, the helicase works with the direction of the field applied by the voltage, pushing the threaded DNA into the nanopore and into the trans chamber. When the Dda helicase is not provided with the necessary components to facilitate movement it can bind to the DNA and act as a brake slowing the movement of the DNA when it is pulled into the pore by the applied field. In the inactive mode it does not matter whether the DNA is captured either 3′ or 5′ down, it is the applied field which pulls the DNA into the nanopore towards the trans side with the enzyme acting as a brake. When in the inactive mode the movement control of the DNA by the helicase can be described in a number of ways including ratcheting, sliding and braking.

A problem which occurs in sequencing polynucleotides, particularly those of 500 nucleotides or more, is that the molecular motor which is controlling the movement of the polynucleotide may disengage from the polynucleotide. This allows the polynucleotide to be pulled through the pore rapidly and in an uncontrolled manner in the direction of the applied field. A modified helicase of the invention is less likely to unbind or disengage from the polynucleotide being sequenced. The modified helicase can provide increased read lengths of the polynucleotide as they control the movement of the polynucleotide through a nanopore. The ability to move an entire polynucleotide through a nanopore under the control of a modified helicase of the invention allows characteristics of the polynucleotide, such as its sequence, to be estimated with improved accuracy and speed over known methods. This becomes more important as strand lengths increase and molecular motors are required with improved processivity. A modified helicase of the invention is particularly effective in controlling the movement of target polynucleotides of 500 nucleotides or more, for example 1000 nucleotides, 5000, 10000, 20000, 50000, 100000 or more.

In addition, using a modified helicase in accordance with the invention means that a lower concentration of helicase may be used. For instance, in Example 3, 1 nM of a modified helicase of the invention is used. In contrast, in Example 3, 1 μM of TrwC Cba, which is not a modified Dda helicase of the invention, is used.

A modified helicase of the invention is also a useful tool for isothermal polymerase chain reaction (PCR). In such methods, the strands of double stranded DNA are typically first separated by a helicase of the invention and coated by single stranded DNA (ssDNA)-binding proteins. In the second step, two sequence specific primers typically hybridise to each border of the DNA template. DNA polymerases may then be used to extend the primers annealed to the templates to produce a double stranded DNA and the two newly synthesized DNA products may then be used as substrates by the helicases of the invention, entering the next round of the reaction. Thus, a simultaneous chain reaction develops, resulting in exponential amplification of the selected target sequence.

The modified helicase has the ability to control the movement of a polynucleotide. The ability of a helicase to control the movement of a polynucleotide can be assayed using any method known in the art. For instance, the helicase may be contacted with a polynucleotide and the position of the polynucleotide may be determined using standard methods. The ability of a modified helicase to control the movement of a polynucleotide is typically assayed in a nanopore system, such as the ones described below and, in particular, as described in the Examples.

A modified helicase of the invention may be isolated, substantially isolated, purified or substantially purified. A helicase is isolated or purified if it is completely free of any other components, such as lipids, polynucleotides, pore monomers or other proteins. A helicase is substantially isolated if it is mixed with carriers or diluents which will not interfere with its intended use. For instance, a helicase is substantially isolated or substantially purified if it is present in a form that comprises less than 10%, less than 5%, less than 2% or less than 1% of other components, such as lipids, polynucleotides, pore monomers or other proteins.

Any Dda helicase may be modified in accordance with the invention. Preferred Dda helicases are discussed below.

Dda helicases typically comprises the following five domains: 1A (RecA-like motor) domain, 2A (RecA-like motor) domain, tower domain, pin domain and hook domain (Xiaoping He et al., 2012, Structure; 20: 1189-1200). The domains may be identified using protein modelling, x-ray diffraction measurement of the protein in a crystalline state (Rupp B (2009). Biomolecular Crystallography: Principles, Practice and Application to Structural Biology. New York: Garland Science.), nuclear magnetic resonance (NMR) spectroscopy of the protein in solution (Mark Rance; Cavanagh, John; Wayne J. Fairbrother; Arthur W. Hunt III; Skelton, NNicholas J. (2007). Protein NMR spectroscopy: principles and practice (2nd ed.). Boston: Academic Press.) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33: 307-69). Structural information of proteins determined by above mentioned methods are publicly available from the protein bank (PDB) database.

Protein modelling exploits the fact that protein structures are more conserved than protein sequences amongst homologues. Hence, producing atomic resolution models of proteins is dependent upon the identification of one or more protein structures that are likely to resemble the structure of the query sequence. In order to assess whether a suitable protein structure exists to use as a “template” to build a protein model, a search is performed on the protein data bank (PDB) database. A protein structure is considered a suitable template if it shares a reasonable level of sequence identity with the query sequence. If such a template exists, then the template sequence is “aligned” with the query sequence, i.e. residues in the query sequence are mapped onto the template residues. The sequence alignment and template structure are then used to produce a structural model of the query sequence. Hence, the quality of a protein model is dependent upon the quality of the sequence alignment and the template structure.

Modifications in the Tower Domain and/or Pin Domain and/or 1A Domain

In one embodiment, the Dda helicase of the invention is one in which at least one cysteine residue (i.e. one or more cysteine residues) and/or at least one non-natural amino acid (i.e. one or more non-natural amino acids) have been introduced into (i) the tower domain and/or (ii) the pin domain and/or the (iii) 1A (RecA-like motor) domain, wherein the helicase retains its ability to control the movement of a polynucleotide. At least one cysteine residue and/or at least one non-natural amino acid may be introduced into the tower domain, the pin domain, the 1A domain, the tower domain and the pin domain, the tower domain and the 1A domain or the tower domain, the pin domain and the 1A domain.

The Dda helicase of the invention is preferably one in which at least one cysteine residue and/or at least one non-natural amino acid have been introduced into each of (i) the tower domain and (ii) the pin domain and/or the 1A (RecA-like motor) domain, i.e. into the tower domain and the pin domain, the tower domain and the 1A domain or the tower domain, the pin domain and the 1A domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are/is preferably introduced by substitution. Methods for doing this are known in the art.

These modifications do not prevent the helicase from binding to a polynucleotide. These modifications decrease the ability of the polynucleotide to unbind or disengage from the helicase. In other words, the one or more modifications increase the processivity of the Dda helicase by preventing dissociation from the polynucleotide strand. The thermal stability of the enzyme is typically also increased by the one or more modifications giving it an improved structural stability that is beneficial in Strand Sequencing.

A non-natural amino acid is an amino that is not naturally found in a Dda helicase. The non-natural amino acid is preferably not histidine, alanine, isoleucine, arginine, leucine, asparagine, lysine, aspartic acid, methionine, cysteine, phenylalanine, glutamic acid, threonine, glutamine, tryptophan, glycine, valine, proline, serine or tyrosine. The non-natural amino acid is more preferably not any of the twenty amino acids in the previous sentence or selenocysteine

Preferred non-natural amino acids for use in the invention include, but are not limited, to 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O-2-Propyn-1-yl-L-tyrosine, 4-(Dihydroxyboryl)-L-phenylalanine, 4-[(Ethylsulfanyl)carbonyl]-L-phenylalanine, (2S)-2-amino-3-{4-[(propan-2-ylsulfanyl)carbonyl]phenyl}propanoic acid, (2S)-2-amino-3-{4-[(2-amino-3-sulfanylpropanoyl)amino]phenyl}propanoic acid, O-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, O-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2,2′-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, 0-(4,5-Dimethoxy-2-nitrobenzyl)-L-serine, (2S)-2-amino-6-({[(2-nitrobenzyl)oxy]carbonyl}amino)hexanoic acid, O-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-1-naphthyl]sulfonylamino]propanoic acid, (2S)-2-amino-4-(7-hydroxy-2-oxo-2H-chromen-4-yl)butanoic acid, (2S)-3-[(6-acetylnaphthalen-2-yl)amino]-2-aminopropanoic acid, 4-(Carboxymethyl)phenylalanine, 3-Nitro-L-tyrosine, O-Sulfo-L-tyrosine, (2R)-6-Acetamido-2-ammoniohexanoate, 1-Methylhistidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N⁶-{[(2R,3R)-3-Methyl-3,4-dihydro-2H-pyrrol-2-yl]carbonyl}-L-lysine, N⁶-[(Benzyloxy)carbonyl]lysine, (2S)-2-amino-6-[(cyclopentylcarbonyl)amino]hexanoic acid, N⁶-[(Cyclopentyloxy)carbonyl]-L-lysine, (2S)-2-amino-6-{[(2R)-tetrahydrofuran-2-ylcarbonyl]amino}hexanoic acid, (2S)-2-amino-8-[(2R,3S)-3-ethynyltetrahydrofuran-2-yl]-8-oxooctanoic acid, N⁶-(tert-Butoxycarbonyl)-L-lysine, (2S)-2-Hydroxy-6-({[(2-methyl-2-propanyl)oxy]carbonyl}amino)hexanoic acid, N⁶-[(Allyloxy)carbonyl]lysine, (2S)-2-amino-6-({[(2-azidobenzyl)oxy]carbonyl}amino)hexanoic acid, N⁶-L-Prolyl-L-lysine, (2S)-2-amino-6-{[(prop-2-yn-1-yloxy)carbonyl]amino}hexanoic acid and N⁶-[(2-Azidoethoxy)carbonyl]-L-lysine. The most preferred non-natural amino acid is 4-azido-L-phenylalanine (Faz).

Table 1 below summarises the preferred Dda helicases which may be modified in accordance with the invention.

| | | | | | Sequence | Number of | |
|---|---|---|---|---|---|---|---|
| Dda Homologue | | | | | Identity | D/E vs. K/R | |
| (SEQ ID NO:) | | Habitat | Uniprot | Length | to 1993/% | amino acids | # C |
| Rma-DSM | Rhodothermus marinus | Mild halophile, | D0MKQ2 | 678 | 21 | −84/+85 | 2 |
| (SEQ ID | | moderate | | | | | |
| NO: 9) | | thermophile >65° C. | | | | | |
| Csp | Cyanothece sp. | Marine bacterium | B1X365 | 496 | 24 | −76/+76 | 5 |
| (SEQ ID | (strain ATCC 51142) | | | | | | |
| NO: 10) | | | | | | | |
| Sru | Salinibacter ruber | Extremely halophilic, | Q2S429 | 421 | 26 | −78/+54 | 3 |
| (SEQ ID | | 35-45° C. | | | | | |
| NO: 11) | | | | | | | |
| Sgo | Sulfurimonas | Habitat: | B6BJ43 | 500 | 27 | −72/+64 | 2 |
| (SEQ ID | gotlandica GD1 | hydrothermal vents, | | | | | |
| NO: 12) | | coastal sediments | | | | | |
| Vph12B8 | Vibrio phage | Host found in | M4MBC3 | 450 | 27 | −62/+47 | 6 |
| (SEQ ID | henriette 12B8 | saltwater, | | | | | |
| NO: 13) | | stomach bug | | | | | |
| Vph | Vibrio phage | Host found in | I6XGX8 | 421 | 39 | −55/+45 | 5 |
| (SEQ ID | phi-pp2 | saltwater, | | | | | |
| NO: 14) | | stomach bug | | | | | |
| Aph65 | Aeromonas phage | Host found in | E5DRP6 | 434 | 40 | −57/+48 | 4 |
| (SEQ ID | 65 | fresh/brackish | | | | | |
| NO: 15) | | water, stomach bug | | | | | |
| AphCC2 | Aeromonas phage | Host found in | I6XH64 | 420 | 41 | −53/+44 | 4 |
| (SEQ ID | CC2 | fresh/brackish | | | | | |
| NO: 16) | | water, stomach bug | | | | | |
| Cph | Cronobacter phage | Host member of | K4FBD0 | 443 | 42 | −59/+57 | 4 |
| (SEQ ID | vB CsaM GAP161 | enterobacteriaceae | | | | | |
| NO: 17) | | | | | | | |
| Kph | Klebsiella phage | Host member of | D5JF67 | 442 | 44 | −59/+58 | 5 |
| (SEQ ID | KP15 | enterobacteriaceae | | | | | |
| NO: 18) | | | | | | | |
| SphlME13 | Stenotrophomonas | Host found in soil | J7HXT5 | 438 | 51 | −58/+59 | 7 |
| (SEQ ID | phage IME13 | | | | | | |
| NO: 19) | | | | | | | |
| AphAc42 | Acinetobacter phage | Host found in soil | E5EYE6 | 442 | 59 | −53/+49 | 9 |
| (SEQ ID | Ac42 | | | | | | |
| NO: 20) | | | | | | | |
| SphSP18 | Shigella phage | Host member of | E3SFA5 | 442 | 59 | −55/+55 | 9 |
| (SEQ ID | SP18 | enterobacteriaceae | | | | | |
| NO: 21) | | | | | | | |
| Yph | Yersinia phage | Host member of | I7J3V8 | 439 | 64 | −52/+52 | 7 |
| (SEQ ID | phiR1-RT | enterobacteriaceae | | | | | |
| NO: 22) | | | | | | | |
| SphS16 | Salmonella phage | Host member of | M1EA88 | 441 | 72 | −56/+55 | 5 |
| (SEQ ID | S16 | enterobacteriaceae | | | | | |
| NO: 23) | | | | | | | |
| 1993 | Enterobateria phage | Host member of | P32270 | 439 | 100 | −57/+58 | 5 |
| (SEQ ID | T4 | enterobacteriaceae | | | | | |
| NO: 8) | | | | | | | |

Table 2 below (which is separated in two parts) identifies the residues making up each domain in each Dda homologue (SEQ ID NOs: 8 to 23).

| | SEQ | | |
|---|---|---|---|
| | ID | | |
| Homologue | NO | 1A | 2A |
| Dda-Rma- | 9 | M1-I84 + R113-Y211 | R212-E294 + G422-S678 |
| DSM | | | |
| Dda-Csp | 10 | M1-L147 + S166-V240 | R241-N327 + A449-G496 |
| Dda-Sru | 11 | M1-L90 + E108-H173 | R174-D260 + A371-V421 |
| Dda-Sgo | 12 | M1-L115 + N136-V205 | R206-K293 + I408-L500 |
| Dda- | 13 | M1-L96 + F114-V194 | R195-D287 + V394-Q450 |
| Vph12B8 | | | |
| Dda-Vph | 14 | M1-L77 + V96-V166 | R167-T249 + L372-N421 |
| Dda-Aph65 | 15 | M1-M81 + L99-M171 | R172-T254 + L381-K434 |
| Dda- | 16 | M1-M68 + M86-M158 | R159-T241 + L367-K420 |
| AphCC2 | | | |
| Dda-Cph | 17 | M1-L87 + A108-M181 | R182-T262 + L393-V443 |
| Dda-Kph | 18 | M1-L87 + A108-M181 | R182-T262 + L392-V442 |
| Dda- | 19 | M1-L85 + T103-K176 | R177-N257 + L387-V438 |
| SphIME13 | | | |
| Dda- | 20 | M1-L91 + V109-M183 | R184-T265 + L393-I442 |
| AphAc42 | | | |
| Dda- | 21 | M1-L87 + M105-M179 | R180-T261 + L393-V442 |
| SphSP18 | | | |
| Dda-Yph | 22 | M1-L86 + V104-K178 | R179-T260 + L390-I439 |
| Dda-SphS16 | 23 | M1-L86 + V104-M178 | R179-T260 + L391-V441 |
| Dda-1993 | 8 | M1-L85 + V103-K177 | R178-T259 + L390-V439 |

| | SEQ | | | |
|---|---|---|---|---|
| Homologue | ID | tower | pin | hook |
| Dda-Rma- | 9 | G295-N309 + F316-Y421 | Y85-L112 | A310-L315 |
| DSM | | | | |
| Dda-Csp | 10 | V328-P342 + N360-Y448 | K148-N165 | V343-L359 |
| Dda-Sru | 11 | A261-T275 + T285-Y370 | G91-E107 | W276-L284 |
| Dda-Sgo | 12 | G294-I307 + T314-Y407 | G116-T135 | R308-Y313 |
| Dda- | 13 | V288-E301 + N307-N393 | G97-P113 | M302- |
| Vph12B8 | | | | W306 |
| Dda-Vph | 14 | S250-P264 + E278-S371 | K78-E95 | V265-I277 |
| Dda-Aph65 | 15 | K255-P269 + T284-S380 | K82-K98 | V270-F283 |
| Dda-AphCC2 | 16 | D242-P256 + T271-S366 | K69-K85 | V257-F270 |
| Dda-Cph | 17 | T263-P277 + N295-P392 | K88-K107 | L278-Y294 |
| Dda-Kph | 18 | D263-P277 + N295-A391 | K88-K107 | L278-Y294 |
| Dda- | 19 | A258-P272 + N290-P386 | K86-G102 | L273-F289 |
| SpHIME13 | | | | |
| Dda- | 20 | L266-P280 + N298-A392 | K92-D108 | L281-F297 |
| AphAc42 | | | | |
| Dda- | 21 | D262-P276 + N294-A392 | K88-E104 | H277-F293 |
| SphSP18 | | | | |
| Dda-Yph | 22 | D261-P275 + N293-A389 | K87-E103 | L276-F292 |
| Dda-SphS16 | 23 | E261-P275 + T293-A390 | K87-E103 | L276-F292 |
| Dda-1993 | 8 | D260-P274 + N292-A389 | K86-E102 | L275-F291 |

The helicase of the invention preferably comprises a variant of SEQ ID NO: 8 in which at least one cysteine residue and/or at least one non-natural amino acid have been introduced into (i) the tower domain (residues D260-P274 and N292-A389) and/or (ii) the pin domain (residues K86-E102) and/or the (iii) 1A domain (residues M1-L85 and V103-K177). The at least one cysteine residue and/or at least one non-natural amino acid are preferably introduced into residues N292-A389 of the tower domain.

The helicase of the invention preferably comprises a variant of SEQ ID NO: 9 in which at least one cysteine residue and/or at least one non-natural amino acid have been introduced into (i) the tower domain (residues G295-N309 and F316-Y421) and/or (ii) the pin domain (residues Y85-L112) and/or the (iii) 1A domain (residues M1-I84 and R113-Y211). The at least one cysteine residue and/or at least one non-natural amino acid are preferably introduced into residues F316-Y421 of the tower domain.

The helicase of the invention preferably comprises a variant of SEQ ID NO: 10 in which at least one cysteine residue and/or at least one non-natural amino acid have been introduced into (i) the tower domain (residues V328-P342 and N360-Y448) and/or (ii) the pin domain (residues K148-N165) and/or the (iii) 1A domain (residues M1-L147 and 5166-V240). The at least one cysteine residue and/or at least one non-natural amino acid are preferably introduced into residues N360-Y448 of the tower domain.

The helicase of the invention preferably comprises a variant of SEQ ID NO: 11 in which at least one cysteine residue and/or at least one non-natural amino acid have been introduced into (i) the tower domain (residues A261-T275 and T285-Y370) and/or (ii) the pin domain (residues G91-E107) and/or the (iii) 1A domain (residues M1-L90 and E108-H173). The at least one cysteine residue and/or at least one non-natural amino acid are preferably introduced into residues T285-Y370 of the tower domain.

The helicase of the invention preferably comprises a variant of SEQ ID NO: 12 in which at least one cysteine residue and/or at least one non-natural amino acid have been introduced into (i) the tower domain (residues G294-I307 and T314-Y407) and/or (ii) the pin domain (residues G116-T135) and/or the (iii) 1A domain (residues M1-L115 and N136-V205). The at least one cysteine residue and/or at least one non-natural amino acid are preferably introduced into residues T314-Y407 of the tower domain.

The helicase of the invention preferably comprises a variant of SEQ ID NO: 13 in which at least one cysteine residue and/or at least one non-natural amino acid have been introduced into (i) the tower domain (residues V288-E301 and N307-N393) and/or (ii) the pin domain (residues G97-P113) and/or the (iii) 1A domain (residues M1-L96 and F114-V194). The at least one cysteine residue and/or at least one non-natural amino acid are preferably introduced into residues N307-N393 of the tower domain.

The helicase of the invention preferably comprises a variant of SEQ ID NO: 14 in which at least one cysteine residue and/or at least one non-natural amino acid have been introduced into (i) the tower domain (residues 5250-P264 and E278-S371) and/or (ii) the pin domain (residues K78-E95) and/or the (iii) 1A domain (residues M1-L77 and V96-V166). The at least one cysteine residue and/or at least one non-natural amino acid are preferably introduced into residues E278-S371 of the tower domain.

The helicase of the invention preferably comprises a variant of SEQ ID NO: 15 in which at least one cysteine residue and/or at least one non-natural amino acid have been introduced into (i) the tower domain (residues K255-P269 and T284-S380) and/or (ii) the pin domain (residues K82-K98) and/or the (iii) 1A domain (residues M1-M81 and L99-M171). The at least one cysteine residue and/or at least one non-natural amino acid are preferably introduced into residues T284-S380 of the tower domain.

The helicase of the invention preferably comprises a variant of SEQ ID NO: 16 in which at least one cysteine residue and/or at least one non-natural amino acid have been introduced into (i) the tower domain (residues D242-P256 and T271-S366) and/or (ii) the pin domain (residues K69-K85) and/or the (iii) 1A domain (residues M1-M68 and M86-M158). The at least one cysteine residue and/or at least one non-natural amino acid are preferably introduced into residues T271-S366 of the tower domain.

The helicase of the invention preferably comprises a variant of SEQ ID NO: 17 in which at least one cysteine residue and/or at least one non-natural amino acid have been introduced into (i) the tower domain (residues T263-P277 and N295-P392) and/or (ii) the pin domain (residues K88-K107) and/or the (iii) 1A domain (residues M1-L87 and A108-M181). The at least one cysteine residue and/or at least one non-natural amino acid are preferably introduced into residues N295-P392 of the tower domain.

The helicase of the invention preferably comprises a variant of SEQ ID NO: 18 in which at least one cysteine residue and/or at least one non-natural amino acid have been introduced into (i) the tower domain (residues D263-P277 and N295-A391) and/or (ii) the pin domain (residues K88-K107) and/or the (iii) 1A domain (residues M1-L87 and A108-M181). The at least one cysteine residue and/or at least one non-natural amino acid are preferably introduced into residues N295-A391 of the tower domain.

The helicase of the invention preferably comprises a variant of SEQ ID NO: 19 in which at least one cysteine residue and/or at least one non-natural amino acid have been introduced into (i) the tower domain (residues A258-P272 and N290-P386) and/or (ii) the pin domain (residues K86-G102) and/or the (iii) 1A domain (residues M1-L85 and T103-K176). The at least one cysteine residue and/or at least one non-natural amino acid are preferably introduced into residues N290-P386 of the tower domain.

The helicase of the invention preferably comprises a variant of SEQ ID NO: 20 in which at least one cysteine residue and/or at least one non-natural amino acid have been introduced into (i) the tower domain (residues L266-P280 and N298-A392) and/or (ii) the pin domain (residues K92-D108) and/or the (iii) 1A domain (residues M1-L91 and V109-M183). The at least one cysteine residue and/or at least one non-natural amino acid are preferably introduced into residues N298-A392 of the tower domain.

The helicase of the invention preferably comprises a variant of SEQ ID NO: 21 in which at least one cysteine residue and/or at least one non-natural amino acid have been introduced into (i) the tower domain (residues D262-P276 and N294-A392) and/or (ii) the pin domain (residues K88-E104) and/or the (iii) 1A domain (residues M1-L87 and M105-M179). The at least one cysteine residue and/or at least one non-natural amino acid are preferably introduced into residues N294-A392 of the tower domain.

The helicase of the invention preferably comprises a variant of SEQ ID NO: 22 in which at least one cysteine residue and/or at least one non-natural amino acid have been introduced into (i) the tower domain (residues D261-P275 and N293-A389) and/or (ii) the pin domain (residues K87-E103) and/or the (iii) 1A domain (residues M1-L86 and V104-K178). The at least one cysteine residue and/or at least one non-natural amino acid are preferably introduced into residues N293-A389 of the tower domain.

The helicase of the invention preferably comprises a variant of SEQ ID NO: 23 in which at least one cysteine residue and/or at least one non-natural amino acid have been introduced into (i) the tower domain (residues E261-P275 and T293-A390) and/or (ii) the pin domain (residues K87-E103) and/or the (iii) 1A domain (residues M1-L86 and V104-M178). The at least one cysteine residue and/or at least one non-natural amino acid are preferably introduced into residues T293-A390 of the tower domain.

The helicase of the invention preferably comprises a variant of any one of SEQ ID NOs: 8 to 23 in which at least one cysteine residue and/or at least one non-natural amino acid have been introduced into each of (i) the tower domain and (ii) the pin domain and/or the 1A domain. The helicase of the invention more preferably comprises a variant of any one of SEQ ID NOs: 8 to 23 in which at least one cysteine residue and/or at least one non-natural amino acid have been introduced into each of (i) the tower domain, (ii) the pin domain and (iii) the 1A domain. Any number and combination of cysteine residues and non-natural amino acids may be introduced as discussed above.

The helicase of the invention preferably comprises a variant of SEQ ID NO: 8 which comprises (or only comprises) (i) E94C and/or A360C; (ii) E93C and/or K358C; (iii) E93C and/or A360C; (iv) E93C and/or E361C; (v) E93C and/or K364C; (vi) E94C and/or L354C; (vii) E94C and/or K358C; (viii) E93C and/or L354C; (ix) E94C and/or E361C; (x) E94C and/or K364C; (xi) L97C and/or L354C; (xii) L97C and/or K358C; (xiii) L97C and/or A360C; (xiv) L97C and/or E361C; (xv) L97C and/or K364C; (xvi) K123C and/or L354C; (xvii) K123C and/or K358C; (xviii) K123C and/or A360C; (xix) K123C and/or E361C; (xx) K123C and/or K364C; (xxi) N155C and/or L354C; (xxii) N155C and/or K358C; (xxiii) N155C and/or A360C; (xxiv) N155C and/or E361C; (xxv) N155C and/or K364C; (xxvi) any of (i) to (xxv) and G357C; (xxvii) any of (i) to (xxv) and Q1000C; (xxviii) any of (i) to (xxv) and I127C; (xxix) any of (i) to (xxv) and Q100C and I127C; (xxx) E94C and/or F377C; (xxxi) N95C; (xxxii) T91C; (xxxiii) Y92L, E94Y, Y350N, A360C and Y363N; (xxxiv) E94Y and A360C; (xxxv) A360C; (xxxvi) Y92L, E94C, Y350N, A360Y and Y363N; (xxxvii) Y92L, E94C and A360Y; (xxxviii) E94C and/or A360C and F276A; (xxxix) E94C and/or L356C; (xl) E93C and/or E356C; (xli) E93C and/or G357C; (xlii) E93C and/or A360C; (xliii) N95C and/or W378C; (xliv) T91C and/or S382C; (xlv) T91C and/or W378C; (xlvi) E93C and/or N353C; (xlvii) E93C and/or S382C; (xlviii) E93C and/or K381C; (xlix) E93C and/or D379C; (1) E93C and/or S375C; (li) E93C and/or W378C; (lii) E93C and/or W374C; (liii) E94C and/or N353C; (liv) E94C and/or S382C; (lv) E94C and/or K381C; (lvi) E94C and/or D379C; (lvii) E94C and/or S375C; (lviii) E94C and/or W378C; (lix) E94C and/or W374C; (lx) E94C and A360Y; (lxi) E94C, G357C and A360C or (lxii) T2C, E94C and A360C. In any one of (i) to (lxii), and/or is preferably and.

The helicase of the invention preferably comprises a variant of any one of SEQ ID NOs: 9 to 23 which comprises a cysteine residue at the positions which correspond to those in SEQ ID NO: 8 as defined in any of (i) to (lxii). Positions in any one of SEQ ID NOs: 9 to 23 which correspond to those in SEQ ID NO: 8 can be identified using the alignment of SEQ ID NOs: 8 to 23 below. The helicase of the invention preferably comprises a variant of SEQ ID NO: 11 which comprises (or only comprises) (a) D99C and/or L341C, (b) Q98C and/or L341C or (d) Q98C and/or A340C. The helicase of the invention preferably comprises a variant of SEQ ID NO: 15 which comprises (or only comprises) D90C and/or A349C. The helicase of the invention preferably comprises a variant of SEQ ID NO: 21 which comprises (or only comprises) D96C and/or A362C.

The helicase of the invention preferably comprises a variant of any one of SEQ ID NOs: 8 to 23 as defined in any one of (i) to (lxii) in which Faz is introduced at one or more of the specific positions instead of cysteine. Faz may be introduced at each specific position instead of cysteine. The helicase of the invention preferably comprises a variant of SEQ ID NO: 8 which comprises (or only comprises) (i) E94Faz and/or A360C; (ii) E94C and/or A360Faz; (iii) E94Faz and/or A360Faz; (iv) Y92L, E94Y, Y350N, A360Faz and Y363N; (v) A360Faz; (vi) E94Y and A360Faz; (vii) Y92L, E94Faz, Y350N, A360Y and Y363N; (viii) Y92L, E94Faz and A360Y; (ix) E94Faz and A360Y; and (x) E94C, G357Faz and A360C.

The helicase of the invention preferably further comprises one or more single amino acid deletions from the pin domain. Any number of single amino acid deletions may be made, such as 1, 2, 3, 4, 5, 6, 7, 8, 9, 10 or more. The helicase more preferably comprises a variant of SEQ ID NO: 8 which comprises deletion of E93, deletion of E95 or deletion of E93 and E95. The helicase more preferably comprises a variant of SEQ ID NO: 8 which comprises (or only comprises) (a) E94C, deletion of N95 and A360C; (b) deletion of E93, deletion of E94, deletion of N95 and A360C; (c) deletion of E93, E94C, deletion of N95 and A360C or (d) E93C, deletion of N95 and A360C. The helicase of the invention preferably comprises a variant of any one of SEQ ID NOs: 9 to 23 which comprises deletion of the position corresponding to E93 in SEQ ID NO: 8, deletion of the position corresponding to E95 in SEQ ID NO: 8 or deletion of the positions corresponding to E93 and E95 in SEQ ID NO: 8.

The helicase of the invention preferably further comprises one or more single amino acid deletions from the hook domain. Any number of single amino acid deletions may be made, such as 1, 2, 3, 4, 5, 6, 7, 8, 9, 10 or more. The helicase more preferably comprises a variant of SEQ ID NO: 8 which comprises deletion of any number of positions T278 to S287. The helicase more preferably comprises a variant of SEQ ID NO: 8 which comprises (a) E94C, deletion of Y279 to K284 and A360C, (b) E94C, deletion of T278, Y279, V286 and S287 and A360C, (c) E94C, deletion of I281 and K284 and replacement with a single G and A360C, (d) E94C, deletion of K280 and P2845 and replacement with a single G and A360C, or (e) deletion of Y279 to K284, E94C, F276A and A230C. The helicase of the invention preferably comprises a variant of any one of SEQ ID NOs: 9 to 23 which comprises deletion of any number of the positions corresponding to 278 to 287 in SEQ ID NO: 8.

The helicase of the invention preferably further comprises one or more single amino acid deletions from the pin domain and one or more single amino acid deletions from the hook domain.

The helicase of the invention is preferably one in which at least one cysteine residue and/or at least one non-natural amino acid have been further introduced into the hook domain and/or the 2A (RecA-like) domain. Any number and combination of cysteine residues and non-natural amino acids may be introduced as discussed above for the tower, pin and 1A domains.

The helicase of the invention preferably comprises a variant of SEQ ID NO: 8 in which at least one cysteine residue and/or at least one non-natural amino acid have further been introduced into the hook domain (residues L275-F291) and/or the 2A (RecA-like) domain (residues R178-T259 and L390-V439).

The helicase of the invention preferably comprises a variant of SEQ ID NO: 9 in which at least one cysteine residue and/or at least one non-natural amino acid have further been introduced into the hook domain (residues A310-L315) and/or the 2A (RecA-like) domain (residues R212-E294 and G422-S678).

The helicase of the invention preferably comprises a variant of SEQ ID NO: 10 in which at least one cysteine residue and/or at least one non-natural amino acid have further been introduced into the hook domain (residues V343-L359) and/or the 2A (RecA-like) domain (residues R241-N327 and A449-G496).

The helicase of the invention preferably comprises a variant of SEQ ID NO: 11 in which at least one cysteine residue and/or at least one non-natural amino acid have further been introduced into the hook domain (residues W276-L284) and/or the 2A (RecA-like) domain (residues R174-D260 and A371-V421).

The helicase of the invention preferably comprises a variant of SEQ ID NO: 12 in which at least one cysteine residue and/or at least one non-natural amino acid have further been introduced into the hook domain (residues R308-Y313) and/or the 2A (RecA-like) domain (residues R206-K293 and I408-L500).

The helicase of the invention preferably comprises a variant of SEQ ID NO: 13 in which at least one cysteine residue and/or at least one non-natural amino acid have further been introduced into the hook domain (residues M302-W306) and/or the 2A (RecA-like) domain (residues R195-D287 and V394-Q450).

The helicase of the invention preferably comprises a variant of SEQ ID NO: 14 in which at least one cysteine residue and/or at least one non-natural amino acid have further been introduced into the hook domain (residues V265-I277) and/or the 2A (RecA-like) domain (residues R167-T249 and L372-N421).

The helicase of the invention preferably comprises a variant of SEQ ID NO: 15 in which at least one cysteine residue and/or at least one non-natural amino acid have further been introduced into the hook domain (residues V270-F283) and/or the 2A (RecA-like) domain (residues R172-T254 and L381-K434).

The helicase of the invention preferably comprises a variant of SEQ ID NO: 16 in which at least one cysteine residue and/or at least one non-natural amino acid have further been introduced into the hook domain (residues V257-F270) and/or the 2A (RecA-like) domain (residues R159-T241 and L367-K420).

The helicase of the invention preferably comprises a variant of SEQ ID NO: 17 in which at least one cysteine residue and/or at least one non-natural amino acid have further been introduced into the hook domain (residues L278-Y294) and/or the 2A (RecA-like) domain (residues R182-T262 and L393-V443).

The helicase of the invention preferably comprises a variant of SEQ ID NO: 18 in which at least one cysteine residue and/or at least one non-natural amino acid have further been introduced into the hook domain (residues L278-Y294) and/or the 2A (RecA-like) domain (residues R182-T262 and L392-V442).

The helicase of the invention preferably comprises a variant of SEQ ID NO: 19 in which at least one cysteine residue and/or at least one non-natural amino acid have further been introduced into the hook domain (residues L273-F289) and/or the 2A (RecA-like) domain (residues R177-N257 and L387-V438).

The helicase of the invention preferably comprises a variant of SEQ ID NO: 20 in which at least one cysteine residue and/or at least one non-natural amino acid have further been introduced into the hook domain (residues L281-F297) and/or the 2A (RecA-like) domain (residues R184-T265 and L393-I442).

The helicase of the invention preferably comprises a variant of SEQ ID NO: 21 in which at least one cysteine residue and/or at least one non-natural amino acid have further been introduced into the hook domain (residues H277-F293) and/or the 2A (RecA-like) domain (residues R180-T261 and L393-V442).

The helicase of the invention preferably comprises a variant of SEQ ID NO: 22 in which at least one cysteine residue and/or at least one non-natural amino acid have further been introduced into the hook domain (residues L276-F292) and/or the 2A (RecA-like) domain (residues R179-T260 and L390-I439).

The helicase of the invention preferably comprises a variant of SEQ ID NO: 23 in which at least one cysteine residue and/or at least one non-natural amino acid have further been introduced into the hook domain (residues L276-F292) and/or the 2A (RecA-like) domain (residues R179-T260 and L391-V441).

The helicase of the invention preferably comprises a variant of SEQ ID NO: 8 which comprises one or more of (i) I181C; (ii) Y279C; (iii) I281C; and (iv) E288C. The helicase may comprise any combination of (i) to (iv), such as (i); (ii); (iii); (iv); (i) and (ii); (i) and (iii); (i) and (iv); (ii) and (iii); (ii) and (iv); (iii) and (iv); or (i), (ii), (iii) and (iv). The helicase more preferably comprises a variant of SEQ ID NO: 8 which comprises (or only comprises) (a) E94C, I281C and A360C or (b) E94C, I281C, G357C and A360C. The helicase of the invention preferably comprises a variant of any one of SEQ ID NOs: 9 to 23 which comprises a cysteine residue at one or more of the position(s) which correspond to those in SEQ ID NO: 8 as defined in (i) to (iv), (a) and (b). The helicase may comprise any of these variants in which Faz is introduced at one or more of the specific positions (or each specific position) instead of cysteine.

The helicase of the invention is further modified to reduce its surface negative charge. Surface residues can be identified in the same way as the Dda domains disclosed above. Surface negative charges are typically surface negatively-charged amino acids, such as aspartic acid (D) and glutamic acid (E).

The helicase is preferably modified to neutralise one or more surface negative charges by substituting one or more negatively charged amino acids with one or more positively charged amino acids, uncharged amino acids, non-polar amino acids and/or aromatic amino acids or by introducing one or more positively charged amino acids, preferably adjacent to one or more negatively charged amino acids. Suitable positively charged amino acids include, but are not limited to, histidine (H), lysine (K) and arginine (R). Uncharged amino acids have no net charge. Suitable uncharged amino acids include, but are not limited to, cysteine (C), serine (S), threonine (T), methionine (M), asparagine (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y).

Preferred substitutions include, but are not limited to, substitution of E with R, substitution of E with K, substitution of E with N, substitution of D with K and substitution of D with R.

The helicase of the invention preferably comprises a variant of SEQ ID NO: 8 and the one or more negatively charged amino acids are one or more of D5, E8, E23, E47, D167, E172, D202, D212 and E273. Any number of these amino acids may be neutralised, such as 1, 2, 3, 4, 5, 6, 7 or 8 of them. Any combination may be neutralised. The helicase of the invention preferably comprises a variant of any one of SEQ ID NOs: 9 to 23 and the one or more negatively charged amino acids correspond to one or more of D5, E8, E23, E47, D167, E172, D202, D212 and E273 in SEQ ID NO: 8. Amino acids in SEQ ID NOs: 9 to 23 which correspond to D5, E8, E23, E47, D167, E172, D202, D212 and E273 in SEQ ID NO: 8 can be determined using the alignment below. The helicase of the invention preferably comprises a variant of SEQ ID NO: 8 which comprises (or only comprises) (a) E94C, E273G and A360C or (b) E94C, E273G, N292G and A360C.

The helicase of the invention is preferably further modified by the removal of one or more native cysteine residues. Any number of native cysteine residues may be removed. The number of cysteine residues in each of SEQ ID NOs: 9 to 23 is shown in Table 1 (as #C). The one or more cysteine residues are preferably removed by substitution. The one or more cysteine residues are preferably substituted with alanine (A), serine (S) or valine (V). The helicase of the invention preferably comprises a variant of SEQ ID NO: 8 and the one or more native cysteine residues are one or more of C109, C114, C136, C171 and C412. Any number and combination of these cysteine residues may be removed. For instance, the variant of SEQ ID NO: 8 may comprise {C109}; {C114}; {C136}; {C171}; {C412}; {C109 and C114}; {C109 and C136}; {C109 and C171}; {C109 and C412}; {C114 and C136}; {C114 and C171}; {C114 and C412}; {C136 and C171}; {C136 and C412}; {C171 and C412}; {C109, C114 and C136}; {C109, C114 and C171}; {C109, C114 and C412}; {C109, C136 and C171}; {C109, C136 and C412}; {C109, C171 and C412}; {C114, C136 and C171}; {C114, C136 and C412}; {C114, C171 and C412}; {C136, C171 and C412}; {C109, C114, C136 and C171}; {C109, C114, C136 and C412}; {C109, C114, C171 and C412}; {C109, C136, C171 and C412}; {C114, C136, C171 and C412}; or {C109, C114, C136, C171 and C412}.

The helicase of the invention is preferably one in which at least one cysteine residue (i.e. one or more cysteine residues) and/or at least one non-natural amino acid (i.e. one or more non-natural amino acids) have been introduced into the tower domain only. Suitable modifications are discussed above.

The helicase of the invention preferably comprises a variant of SEQ ID NO: 8 comprising (or comprising only) the following mutations:
E93C and K364C;
E94C and K364C;
E94C and A360C;
L97C and E361C;
L97C and E361C and C412A;
K123C and E361C;
K123C, E361C and C412A;
N155C and K358C;
N155C, K358C and C412A;
N155C and L354C;
N155C, L354C and C412A;
deltaE93, E94C, deltaN95 and A360C;
E94C, deltaN95 and A360C;
E94C, Q100C, I127C and A360C;
L354C;
G357C;
E94C, G357C and A360C;
E94C, Y279C and A360C;
E94C, I281C and A360C;
E94C, Y279Faz and A360C;
Y279C and G357C;
I281C and G357C;
E94C, Y279C, G357C and A360C;
E94C, I281C, G357C and A360C;
E8R, E47K, E94C, D202K and A360C;
D5K, E23N, E94C, D167K, E172R, D212R and A360C;
D5K, E8R, E23N, E47K, E94C, D167K, E172R, D202K, D212R and A360C;
E94C, C114A, C171A, A360C and C412D;
E94C, C114A, C171A, A360C and C412S;
E94C, C109A, C136A and A360C;
E94C, C109A, C114A, C136A, C171A, A360C and C412S;
E94C, C109V, C114V, C171A, A360C and C412S;
C109A, C114A, C136A, G153C, C171A, E361C and C412A;
C109A, C114A, C136A, G153C, C171A, E361C and C412D;
C109A, C114A, C136A, G153C, C171A, E361C and C412S;
C109A, C114A, C136A, G153C, C171A, K358C and C412A;
C109A, C114A, C136A, G153C, C171A, K358C and C412D
C109A, C114A, C136A, G153C, C171A, K358C and C412S;
C109A, C114A, C136A, N155C, C171A, K358C and C412A;
C109A, C114A, C136A, N155C, C171A, K358C and C412D;
C109A, C114A, C136A, N155C, C171A, K358C and C412S;
C109A, C114A, C136A, N155C, C171A, L354C and C412A;
C109A, C114A, C136A, N155C, C171A, L354C and C412D;
C109A, C114A, C136A, N155C, C171A, L354C and C412S;
C109A, C114A, K123C, C136A, C171A, E361C and C412A;
C109A, C114A, K123C, C136A, C171A, E361C and C412D;
C109A, C114A, K123C, C136A, C171A, E361C and C412S;
C109A, C114A, K123C, C136A, C171A, K358C and C412A;
C109A, C114A, K123C, C136A, C171A, K358C and C412D;
C109A, C114A, K123C, C136A, C171A, K358C and C412S;
C109A, C114A, C136A, G153C, C171A, E361C and C412A;
E94C, C109A, C114A, C136A, C171A, A360C and C412D;
E94C, C109A, C114V, C136A, C171A, A360C and C412D;
E94C, C109V, C114A, C136A, C171A, A360C and C412D;
L97C, C109A, C114A, C136A, C171A, E361C and C412A;
L97C, C109A, C114A, C136A, C171A, E361C and C412D; or
L97C, C109A, C114A, C136A, C171A, E361C and C412S.
   Modifications in the Hook Domain and/or 2A Domain

In one embodiment, the Dda helicase of the invention is one in which at least one cysteine residue and/or at least one non-natural amino acid have been introduced into the hook domain and/or the 2A (RecA-like motor) domain, wherein the helicase retains its ability to control the movement of a polynucleotide. At least one cysteine residue and/or at least one non-natural amino acid is preferably introduced into the hook domain and the 2A (RecA-like motor) domain.

Any number of cysteine residues and/or non-natural amino acids may be introduced into each domain. For instance, 1, 2, 3, 4, 5, 6, 7, 8, 9, 10 or more cysteine residues may be introduced and/or 1, 2, 3, 4, 5, 6, 7, 8, 9, 10 or more non-natural amino acids may be introduced. Only one or more cysteine residues may be introduced. Only one or more non-natural amino acids may be introduced. A combination of one or more cysteine residues and one or more non-natural amino acids may be introduced.

The at least one cysteine residue and/or at least one non-natural amino acid are preferably introduced by substitution. Methods for doing this are known in the art. Suitable modifications of the hook domain and/or the 2A (RecA-like motor) domain are discussed above.

The helicase of the invention is preferably a variant of SEQ ID NO: 8 comprising (or comprising only) (a) Y279C, I181C, E288C, Y279C and I181C, (b) Y279C and E288C, (c) I181C and E288C or (d) Y279C, I181C and E288C. The helicase of the invention preferably comprises a variant of any one of SEQ ID NOs: 9 to 23 which comprises a mutation at one or more of the position(s) which correspond to those in SEQ ID NO: 8 as defined in (a) to (d).

### Surface Modification

In one embodiment, the Dda helicase is modified to reduce its surface negative charge, wherein the helicase retains its ability to control the movement of a polynucleotide. Suitable modifications are discussed above. Any number of surface negative charges may be neutralised.

The helicase of the invention preferably comprises a variant of SEQ ID NO: 8 comprising (or comprising only) the following mutations:
E273G;
E8R, E47K and D202K;
D5K, E23N, D167K, E172R and D212R;
D5K, E8R, E23N, E47K, D167K, E172R, D202K and D212R.

### Other Modified Helicases

In one embodiment, the Dda helicase of the invention comprises a variant of SEQ ID NO: 8 comprising (or comprising only):
A360K;
Y92L and/or A360Y;
Y92L, Y350N and Y363N;
Y92L and/or Y363N; or
Y92L.

### Other Modifications

In addition to the specific mutations disclosed above, a variant of SEQ ID NO: 8 may comprise (or may only comprise) one or more of the following mutations:

K38A;
H64N;
H64K;
H64Q;
H64S;
H64W;
T80K;
T80N;
H82A;
H82F;
H82Q;
H82R;
H82W;
H82Y;
S83K;
S83N;
S83T;
N88H;
N88Q;
P89A;
P89F;
P89S;
P89T;
P89W;
P89Y;
T91F;
T91N;
T91Q;
T91W;
V96E;
V96F;
V96L
V96Q;
V96R;
V96W;
V96Y;
F98A
F98L;
F98V;
F98W;
F98Y;
V150A;
V150F;
V150I;
V150K;
V150L;
V150S;
V150T;
V150W;
V150Y;
F240W;
F240Y;
N242K;
P274G;
F276A;
F276I;
F276M;
F276V;
F276W;
F276Y;
V286F;
V286W;
V286Y;
S287F;
S287W;
S287Y;
F291G;
N292F;
N292G;
N292P;
N292Y;
N293F;
N293K;
N293Q;
N293Y;
G294Y;
G294F;
K364A;
W378A;
T394K;
T394N;
H396Q;
H396S;
H396W;
Y415F;
Y415K;
Y415M; or
Y415W.

The helicase of the invention preferably comprises a variant of SEQ ID NO: 8 which comprises (or only comprises):

K38A, E94C and A360C;
H64K; E94C and A360C;
H64N; E94C and A360C;
H64Q; E94C and A360C;
H64S; E94C and A360C;
H64W, E94C and A360C;
T80K, E94C and A360C;
T80K, S83K, E94C, N242K,
N293K and A360C;
T80K, S83K, E94C, N242K,
N293K, A360C and T394K;
T80K, S83K, E94C, N293K and
A360C;
T80K, S83K, E94C, A360C and
T394K;
T80K, S83K, E94C, A360C and
T394N;
T80K, E94C, N242K and
A360C;
T80K, E94C, N242K, N293K
and A360C;
T80K, E94C, N293K and
A360C;
T80N, E94C and A360C;
H82A, E94C and A360C;
H82A, P89A, E94C, F98A and
A360C;
H82F, E94C and A360C;
H82Q, E94C, A360C;
H82R, E94C and A360C;
H82W, E94C and A360C;
H82W, P89W, E94C, F98W and
A360C;
H82Y, E94C and A360C;
S83K, E94C and A360C;
S83K, T80K, E94C, A360C and
T394K;
S83N, E94C and A360C;
S83T, E94C and A360C;
N88H, E94C and A360C;
N88Q, E94C and A360C;
P89A, E94C and A360C;
P89A, F98W, E94C and A360C;
P89A, E94C, F98Y and A360C;
P89A, E94C, F98A and A360C;
P89F, E94C and A360C;
P89S, E94C and A360C;
P89T, E94C and A360C;
P89W, E94C, F98W and A360C;
P89Y, E94C and A360C;
T91F, E94C and A360C;
T91N, E94C and A360C;
T91Q, E94C and A360C;
T91W, E94C and A360C;
E94C, V96E and A360C;
E94C, V96F and A360C;
E94C, V96L and A360C;
E94C, V96Q and A360C;
E94C, V96R and A360C;
E94C, V96W and A360C;
E94C, V96Y and A360C;
E94C, F98A and A360C;
E94C, F98L and A360C;
E94C, F98V and A360C;
E94C, F98Y and A360C;
E94C; F98W and A360C;
E94C, V150A and A360C;
E94C, V150F and A360C;
E94C, V150I and A360C;
E94C, V150K and A360C;
E94C, V150L and A360C;
E94C, V150S and A360C;
E94C, V150T and A360C;
E94C, V150W and A360C;
E94C, V150Y and A360C;
E94C, F240Y and A360C;
E94C, F240W and A360C;
E94C, N242K and A360C;
E94C, N242K, N293K and
A360C;
E94C, P274G and A360C;
E94C, L275G and A360C
E94C, F276A and A360C;
E94C, F276I and A360C;
E94C, F276M and A360C;
E94C, F276V and A360C;
E94C, F276W and A360C;
E94C, F276Y and A360C;
E94C, V286F and A360C;
E94C, V286W and A360C;
E94C, V286Y and A360C;
E94C, S287F and A360C;
E94C, S287W and A360C;
E94C, S287Y and A360C;
E94C, F291G and A360C;
E94C, N292F and A360C;
E94C, N292G and A360C;
E94C, N292P and A360C;
E94C, N292Y and A360C;
E94C, N293F and A360C;
E94C, N293K and A360C;
E94C, N293Q and A360C;
E94C, N293Y and A360C;
E94C, G294F and A360C;
E94C, G294Y and A360C;
E94C, A36C and K364A;
E94C, A360C, W378A;
E94C, A360C and T394K;
E94C, A360C and H396Q;
E94C, A360C and H396S;
E94C, A360C and H396W;
E94C, A360C and Y415F;
E94C, A360C and Y415K;
E94C, A360C and Y415M; or
E94C, A360C and Y415W.

The helicase of the invention preferably comprises a variant of SEQ ID NO: 8 which comprises (or only comprises) (a) E94C/A360C/W378A, (b) E94C/A360C/W378A W378A and then (ΔM1)G1G2 (i.e. deletion of M1 and then addition G1 and G2), (c) E94C/A360C/C109A/C136A/W378A or (d) E94C/A360C/C109A/C136A/W378A and then (ΔM1)G1G2 (i.e. deletion of M1 and then addition G1 and G2).

### Variants

A variant of a Dda helicase is an enzyme that has an amino acid sequence which varies from that of the wild-type helicase and which retains polynucleotide binding activity. In particular, a variant of any one of SEQ ID NOs: 8 to 23 is an enzyme that has an amino acid sequence which varies from that of any one of SEQ ID NOs: 8 to 23 and which retains polynucleotide binding activity. Polynucleotide binding activity can be determined using methods known in the art. Suitable methods include, but are not limited to, fluorescence anisotropy, tryptophan fluorescence and electrophoretic mobility shift assay (EMSA). For instance, the ability of a variant to bind a single stranded polynucleotide can be determined as described in the Examples.

The variant retains helicase activity. This can be measured in various ways. For instance, the ability of the variant to translocate along a polynucleotide can be measured using electrophysiology, a fluorescence assay or ATP hydrolysis.

The variant may include modifications that facilitate handling of the polynucleotide encoding the helicase and/or facilitate its activity at high salt concentrations and/or room temperature.

Over the entire length of the amino acid sequence of any one of SEQ ID NOs: 8 to 23, a variant will preferably be at least 20% homologous to that sequence based on amino acid identity. More preferably, the variant polypeptide may be at least 30%, at least 40%, at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of any one of SEQ ID NOs: 8 to 23 over the entire sequence. There may be at least 70%, for example at least 80%, at least 85%, at least 90% or at least 95%, amino acid identity over a stretch of 100 or more, for example 150, 200, 300, 400 or 500 or more, contiguous amino acids (“hard homology”). Homology is determined as described below. The variant may differ from the wild-type sequence in any of the ways discussed below with reference to SEQ ID NOs: 2 and 4. In particular, in addition to the specific modifications discussed above, the variant of any one of SEQ ID NOs: 8 to 23 may comprise one or more substitutions, one or more deletions and/or one or more additions as discussed below.

Preferred variants of any one of SEQ ID NOs: 8 to 23 have a non-natural amino acid, such as Faz, at the amino- (N-) terminus and/or carboxy (C-) terminus. Preferred variants of any one of SEQ ID NOs: 8 to 23 have a cysteine residue at the amino- (N-) terminus and/or carboxy (C-) terminus. Preferred variants of any one of SEQ ID NOs: 8 to 23 have a cysteine residue at the amino- (N-) terminus and a non-natural amino acid, such as Faz, at the carboxy (C-) terminus or vice versa.

Preferred variants of SEQ ID NO: 8 contain one or more of, such as all of, the following modifications E54G, D151E, 1196N and G357A.

The most preferred variants of any one of SEQ ID NOs: 8 to 23 have (in addition to the modifications of the invention) the N-terminal methionine (M) deleted and replaced with two glycine residues (GG). In the examples this is shown as (ΔM1)G1G2. For instance, preferred variants of SEQ ID NO: 8 comprise (or only comprise):
E94C, A360C and then (ΔM1)G1G2; and
E94C, C109A, C136A, A360C and then (ΔM1)G1G2.

### Dda Helicase Fragments

The invention also provides fragments of Dda helicases which may be used to produce a helicase of the invention. In a first embodiment, the polypeptide comprises the pin domain and the 1A (RecA-like motor) domain from a Dda helicase and does not comprise any other domains from a Dda helicase, wherein at least one cysteine residue and/or at least one non-natural amino acid have been introduced into the pin domain and/or the 1A (RecA-like motor) domain. Preferred helicases from which the domains may be derived include any of SEQ ID NOs: 8 to 23. The relevant domains of these helicases are defined in Table 2 above. The pin domain and/or the 1A domain may be modified in any of the ways discussed above for the helicases of the invention. In particular, the polypeptide may comprise any of the variants of the pin domains and the 1A domains defined above and any of the pin domain and/or 1A domain mutations defined above.

In a second embodiment, the polypeptide comprises the 2A (RecA-like motor) domain, tower domain and hook domain from a Dda helicase and does not comprise any other domains from a Dda helicase, wherein at least one cysteine residue and/or at least one non-natural amino acid have been introduced into the tower domain. Preferred helicases from which the domains may be derived include any of SEQ ID NOs: 8 to 23. The relevant domains of these helicases are defined in Table 2 above. The tower domain may be modified in any of the ways discussed above for the helicases of the invention. In particular, the polypeptide may comprise any of the variants of the tower defined above and any of the tower mutations defined above.

In addition to the specific modifications discussed above, a polypeptide of the invention may comprise one or more substitutions, one or more deletions and/or one or more additions as discussed below with reference to SEQ ID NOs: 2 and 4.

The invention also provides a helicase comprising a polypeptide of the first embodiment covalently attached to a polypeptide of the second embodiment, wherein the helicase has the ability to control the movement of a polynucleotide. The ability of the helicase to control the movement of a polynucleotide may be determined as discussed above.

### No Connection

In one preferred embodiment, none of the introduced cysteines and/or non-natural amino acids in a modified Dda helicase of the invention are connected to one another.

Connecting Two or More of the Introduced Cysteines and/or Non-Natural Amino Acids

In another preferred embodiment, two or more of the introduced cysteines and/or non-natural amino acids in a modified Dda helicase of the invention are connected to one another. This typically reduces the ability of the helicase of the invention to unbind from a polynucleotide.

Any number and combination of two or more of the introduced cysteines and/or non-natural amino acids may be connected to one another. For instance, 3, 4, 5, 6, 7, 8 or more cysteines and/or non-natural amino acids may be connected to one another. One or more cysteines may be connected to one or more cysteines. One or more cysteines may be connected to one or more non-natural amino acids, such as Faz. One or more non-natural amino acids, such as Faz, may be connected to one or more non-natural amino acids, such as Faz.

The two or more cysteines and/or non-natural amino acids may be connected in any way. The connection can be transient, for example non-covalent. Even transient connection will reduce unbinding of the polynucleotide from the helicase.

The two or more cysteines and/or non-natural amino acids are preferably connected by affinity molecules. Suitable affinity molecules are known in the art. The affinity molecules are preferably (a) complementary polynucleotides (International Application No. PCT/GB10/000132 (published as WO 2010/086602), (b) an antibody or a fragment thereof and the complementary epitope (Biochemistry 6th Ed, W.H. Freeman and co (2007) pp 953-954), (c) peptide zippers (O'Shea et al., Science 254 (5031): 539-544), (d) capable of interacting by 3-sheet augmentation (Remaut and Waksman Trends Biochem. Sci. (2006) 31 436-444), (e) capable of hydrogen bonding, pi-stacking or forming a salt bridge, (f) rotaxanes (Xiang Ma and He Tian Chem. Soc. Rev., 2010, 39, 70-80), (g) an aptamer and the complementary protein (James, W. in Encyclopedia of Analytical Chemistry, R. A. Meyers (Ed.) pp. 4848-4871 John Wiley & Sons Ltd, Chichester, 2000) or (h) half-chelators (Hammerstein et al. J Biol Chem. 2011 Apr. 22; 286(16): 14324-14334). For (e), hydrogen bonding occurs between a proton bound to an electronegative atom and another electronegative atom. Pi-stacking requires two aromatic rings that can stack together where the planes of the rings are parallel. Salt bridges are between groups that can delocalize their electrons over several atoms, e. g. between aspartate and arginine.

The two or more parts may be transiently connected by a hexa-his tag or Ni-NTA.

The two or more cysteines and/or non-natural amino acids are preferably permanently connected. In the context of the invention, a connection is permanent if is not broken while the helicase is used or cannot be broken without intervention on the part of the user, such as using reduction to open —S—S— bonds.

The two or more cysteines and/or non-natural amino acids are preferably covalently-attached. The two or more cysteines and/or non-natural amino acids may be covalently attached using any method known in the art.

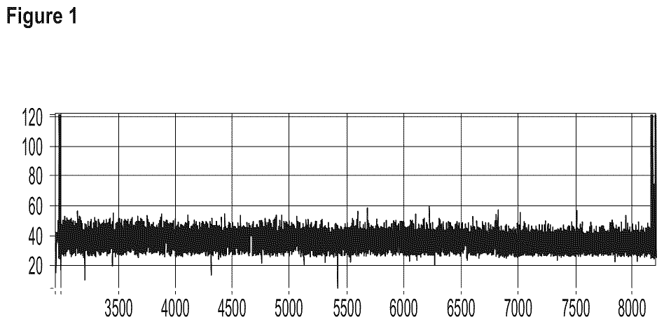

The two or more cysteines and/or non-natural amino acids may be covalently attached via their naturally occurring amino acids, such as cysteines, threonines, serines, aspartates, asparagines, glutamates and glutamines. Naturally occurring amino acids may be modified to facilitate attachment. For instance, the naturally occurring amino acids may be modified by acylation, phosphorylation, glycosylation or farnesylation. Other suitable modifications are known in the art. Modifications to naturally occurring amino acids may be post-translation modifications. The two or more cysteines and/or non-natural amino acids may be attached via amino acids that have been introduced into their sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), any one of the amino acids numbered 1-71 included in FIG. 1 of Liu C. C. and Schultz P. G., Annu. Rev. Biochem., 2010, 79, 413-444 or any one of the amino acids listed below. The introduced amino acids may be modified as discussed above.

In a preferred embodiment, the two or more cysteines and/or non-natural amino acids are connected using linkers. Linker molecules are discussed in more detail below. One suitable method of connection is cysteine linkage. This is discussed in more detail below. The two or more cysteines and/or non-natural amino acids are preferably connected using one or more, such as two or three, linkers. The one or more linkers may be designed to reduce the size of, or close, the opening as discussed above. If one or more linkers are being used to close the opening as discussed above, at least a part of the one or more linkers is preferably oriented such that it is not parallel to the polynucleotide when it is bound by the helicase. More preferably, all of the linkers are oriented in this manner. If one or more linkers are being used to close the opening as discussed above, at least a part of the one or more linkers preferably crosses the opening in an orientation that is not parallel to the polynucleotide when it bound by the helicase. More preferably, all of the linkers cross the opening in this manner. In these embodiments, at least a part of the one or more linkers may be perpendicular to the polynucleotide. Such orientations effectively close the opening such that the polynucleotide cannot unbind from the helicase through the opening.

Each linker may have two or more functional ends, such as two, three or four functional ends. Suitable configurations of ends in linkers are well known in the art.

One or more ends of the one or more linkers are preferably covalently attached to the helicase. If one end is covalently attached, the one or more linkers may transiently connect the two or more cysteines and/or non-natural amino acids as discussed above. If both or all ends are covalently attached, the one or more linkers permanently connect the two or more cysteines and/or non-natural amino acids.

The one or more linkers are preferably amino acid sequences and/or chemical crosslinkers.

Suitable amino acid linkers, such as peptide linkers, are known in the art. The length, flexibility and hydrophilicity of the amino acid or peptide linker are typically designed such that it reduces the size of the opening, but does not to disturb the functions of the helicase. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline. The amino acid sequence of a linker preferably comprises a polynucleotide binding moiety. Such moieties and the advantages associated with their use are discussed below.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to dithiothreitol (DTT).

Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyllamine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S—S-PEG3-biotin, DBCO-S—S-PEG3-biotin, DBCO-S—S-PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). The most preferred crosslinker is maleimide-propyl-SRDFWRS-(1,2-diaminoethane)-propyl-maleimide.

The one or more linkers may be cleavable. This is discussed in more detail below.

The two or more cysteines and/or non-natural amino acids may be connected using two different linkers that are specific for each other. One of the linkers is attached to one part and the other is attached to another part. The linkers should react to form a modified helicase of the invention. The two or more cysteines and/or non-natural amino acids may be connected using the hybridization linkers described in International Application No. PCT/GB10/000132 (published as WO 2010/086602). In particular, the two or more cysteines and/or non-natural amino acids may be connected using two or more linkers each comprising a hybridizable region and a group capable of forming a covalent bond. The hybridizable regions in the linkers hybridize and link the two or more cysteines and/or non-natural amino acids. The linked cysteines and/or non-natural amino acids are then coupled via the formation of covalent bonds between the groups. Any of the specific linkers disclosed in International Application No. PCT/GB10/000132 (published as WO 2010/086602) may be used in accordance with the invention.

The two or more cysteines and/or non-natural amino acids may be modified and then attached using a chemical crosslinker that is specific for the two modifications. Any of the crosslinkers discussed above may be used.

The linkers may be labeled. Suitable labels include, but are not limited to, fluorescent molecules (such as Cy3 or AlexaFluor®555), radioisotopes, e.g. ¹²⁵I, ³⁵S, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting two or more cysteines is via cysteine linkage. This can be mediated by a bi-functional chemical crosslinker or by an amino acid linker with a terminal presented cysteine residue.

The length, reactivity, specificity, rigidity and solubility of any bi-functional linker may be designed to ensure that the size of the opening is reduced sufficiently and the function of the helicase is retained. Suitable linkers include bismaleimide crosslinkers, such as 1,4-bis(maleimido)butane (BMB) or bis(maleimido)hexane. One drawback of bi-functional linkers is the requirement of the helicase to contain no further surface accessible cysteine residues if attachment at specific sites is preferred, as binding of the bi-functional linker to surface accessible cysteine residues may be difficult to control and may affect substrate binding or activity. If the helicase does contain several accessible cysteine residues, modification of the helicase may be required to remove them while ensuring the modifications do not affect the folding or activity of the helicase. This is discussed in International Application No. PCT/GB10/000133 (published as WO 2010/086603). The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S⁻ group. The reactivity of cysteine residues may be protected by thiol protective groups such as 5,5′-dithiobis-(2-nitrobenzoic acid) (dTNB). These may be reacted with one or more cysteine residues of the helicase before a linker is attached. Selective deprotection of surface accessible cysteines may be possible using reducing reagents immobilized on beads (for example immobilized tris(2-carboxyethyl) phosphine, TCEP). Cysteine linkage is discussed in more detail below.

Another preferred method of attachment via Faz linkage. This can be mediated by a bi-functional chemical linker or by a polypeptide linker with a terminal presented Faz residue.

### Other Modified Helicases of the Invention

The invention also provides a Dda helicase which has been modified to increase the attraction between (i) the tower domain and (ii) the pin domain and/or the 1A domain. Any known chemical modifications can be made in accordance with the invention.

In particular, the invention provides a Dda helicase in which at least one charged amino acid has been introduced into (i) the tower domain and/or (ii) the pin domain and/or (iii) the 1A (RecA-like motor) domain, wherein the helicase retains its ability to control the movement of a polynucleotide. The ability of the helicase to control the movement of a polynucleotide may be measured as discussed above. The invention preferably provides a Dda helicase in which at least one charged amino acid has been introduced into (i) the tower domain and (ii) the pin domain and/or the 1A domain.

The at least one charged amino acid may be negatively charged or positively charged. The at least one charged amino acid is preferably oppositely charged to any amino acid(s) with which it interacts in the helicase. For instance, at least one positively charged amino acid may be introduced into the tower domain at a position which interacts with a negatively charged amino acid in the pin domain. The at least one charged amino acid is typically introduced at a position which is not charged in the wild-type (i.e. unmodified) helicase. The at least one charged amino acid may be used to replace at least one oppositely charged amino acid in the helicase. For instance, a positively charged amino acid may be used to replace a negatively charged amino acid.

Suitable charged amino acids are discussed above. The at least one charged amino acid may be natural, such as arginine (R), histidine (H), lysine (K), aspartic acid (D) or glutamic acid (D). Alternatively, the at least one charged amino acid may be artificial or non-natural. Any number of charged amino acids may be introduced into each domain. For instance, 1, 2, 3, 4, 5, 6, 7, 8, 9, 10 or more charged amino acids may be introduced into each domain.

The helicase preferably comprises a variant of SEQ ID NO: 8 which comprises a positively charged amino acid at one or more of the following positions: (i) 93; (ii) 354; (iii) 360; (iv) 361; (v) 94; (vi) 97; (vii) 155; (viii) 357; (ix) 100; and (x) 127. The helicase preferably comprises a variant of SEQ ID NO: 8 which comprises a negatively charged amino acid at one or more of the following positions: (i) 354; (ii) 358; (iii) 360; (iv) 364; (v) 97; (vi) 123; (vii) 155; (viii); 357; (ix) 100; and (x) 127. The helicase preferably comprises a variant of any one of SEQ ID NOs: 9 to 23 which comprises a positively charged amino acid or negatively charged amino acid at the positions which correspond to those in SEQ ID NO: 8 as defined in any of (i) to (x). Positions in any one of SEQ ID NOs: 9 to 23 which correspond to those in SEQ ID NO: 8 can be identified using the alignment of SEQ ID NOs: 8 to 23 below.

The helicase preferably comprises a variant of SEQ ID NO: 8 which is modified by the introduction of at least one charged amino acid such that it comprises oppositely charged amino acid at the following positions: (i) 93 and 354; (ii) 93 and 358; (iii) 93 and 360; (iv) 93 and 361; (v) 93 and 364; (vi) 94 and 354; (vii) 94 and 358; (viii) 94 and 360; (ix) 94 and 361; (x) 94 and 364; (xi) 97 and 354; (xii) 97 and 358; (xiii) 97 and 360; (xiv) 97 and 361; (xv) 97 and 364; (xvi) 123 and 354; (xvii) 123 and 358; (xviii) 123 and 360; (xix) 123 and 361; (xx) 123 and 364; (xxi) 155 and 354; (xxii) 155 and 358; (xxiii) 155 and 360; (xxiv) 155 and 361; (xxv) 155 and 364. The helicase of the invention preferably comprises a variant of any one of SEQ ID NOs: 9 to 23 which comprises oppositely charged amino acids at the positions which correspond to those in SEQ ID NO: 8 as defined in any of (i) to (xxv).

The invention also provides a Dda helicase in which (i) at least one charged amino acid has been introduced into the tower domain and (ii) at least one oppositely charged amino acid has been introduced into the pin domain and/or the 1A (RecA-like motor) domain, wherein the helicase retains its ability to control the movement of a polynucleotide. The at least one charged amino acid may be negatively charged and the at least one oppositely charged amino acid may be positively charged or vice versa. Suitable charged amino acids are discussed above. Any number of charged amino acids and any number of oppositely charged amino acids may be introduced. For instance, 1, 2, 3, 4, 5, 6, 7, 8, 9, 10 or more charged amino acids may be introduced and/or 1, 2, 3, 4, 5, 6, 7, 8, 9, 10 or more oppositely charged amino acids may be introduced.

The charged amino acids are typically introduced at positions which are not charged in the wild-type helicase. One or both of the charged amino acids may be used to replace charged amino acids in the helicase. For instance, a positively charged amino acid may be used to replace a negatively charged amino acid. The charged amino acids may be introduced at any of the positions in the (i) tower domain and (ii) pin domain and/or 1A domain discussed above. The oppositely charged amino acids are typically introduced such that they will interact in the resulting helicase. The helicase preferably comprises a variant of SEQ ID NO: 8 in which oppositely charged amino acids have been introduced at the following positions: (i) 97 and 354; (ii) 97 and 360; (iii) 155 and 354; or (iv) 155 and 360. The helicase of the invention preferably comprises a variant of any one of SEQ ID NOs: 9 to 23 which comprises oppositely charged amino acids at the positions which correspond to those in SEQ ID NO: 8 as defined in any of (i) to (iv).

### Construct

The invention also provides a construct comprising a Dda helicase or a modified Dda helicase of the invention and an additional polynucleotide binding moiety, wherein the helicase is attached to the polynucleotide binding moiety and the construct has the ability to control the movement of a polynucleotide. The construct is artificial or non-natural.

A construct of the invention is a useful tool for controlling the movement of a polynucleotide during Strand Sequencing. A construct of the invention is even less likely than a modified helicase of the invention to disengage from the polynucleotide being sequenced. The construct can provide even greater read lengths of the polynucleotide as it controls the translocation of the polynucleotide through a nanopore.

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

The construct has the ability to control the movement of a polynucleotide. This can be determined as discussed above.

A construct of the invention may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

The Dda helicase may be any Dda helicase. Preferred Dda helicases include, but are not limited to, any one of SEQ ID NOs: 8 to 23 and variants thereof. Variants are defined above. Variants are preferably at least 20% homologous to any one of SEQ ID NOs: 8 to 23 based on amino acid identity. The Dda helicase in the construct does not have to comprise the specific modification(s) discussed above with reference to the modified Dda helicases of the invention (i.e. does not have to be modified in accordance with the invention). For instance, the construct may comprise a Dda helicase which comprises the sequence shown in any one of SEQ ID NOs: 8 to 23 or a variant thereof, wherein:
no cysteine residues and no non-natural amino acids have been introduced into the tower domain, the pin domain and the 1A (RecA-like motor) domain of the variant;
the variant does not comprise one or more single amino acid deletions from the pin domain;
no cysteine residues and no non-natural amino acids have been introduced into the hook domain and the 2A (RecA-like) domain;
the variant is not modified to reduce its surface negative charge;
the variant is not modified by the removal of one or more native cysteine residues;
no cysteine residues and no non-natural amino acids have been introduced into the tower domain only; or
no charged amino acids have introduced into the tower domain, the pin domain and the 1A domain of the variant.

The helicase is preferably a modified Dda helicase of the invention. Any of the helicases of the invention may be present in a construct of the invention.

The helicase is preferably covalently attached to the additional polynucleotide binding moiety. The helicase may be attached to the moiety at more than one, such as two or three, points.

The helicase can be covalently attached to the moiety using any method known in the art. Suitable methods are discussed above with reference to connecting the two or more parts.

The helicase and moiety may be produced separately and then attached together. The two components may be attached in any configuration. For instance, they may be attached via their terminal (i.e. amino or carboxy terminal) amino acids. Suitable configurations include, but are not limited to, the amino terminus of the moiety being attached to the carboxy terminus of the helicase and vice versa. Alternatively, the two components may be attached via amino acids within their sequences. For instance, the moiety may be attached to one or more amino acids in a loop region of the helicase. In a preferred embodiment, terminal amino acids of the moiety are attached to one or more amino acids in the loop region of a helicase.

In a preferred embodiment, the helicase is chemically attached to the moiety, for instance via one or more linker molecules as discussed above. In another preferred embodiment, the helicase is genetically fused to the moiety. A helicase is genetically fused to a moiety if the whole construct is expressed from a single polynucleotide sequence. The coding sequences of the helicase and moiety may be combined in any way to form a single polynucleotide sequence encoding the construct. Genetic fusion of a pore to a nucleic acid binding protein is discussed in International Application No. PCT/GB09/001679 (published as WO 2010/004265).

The helicase and moiety may be genetically fused in any configuration. The helicase and moiety may be fused via their terminal amino acids. For instance, the amino terminus of the moiety may be fused to the carboxy terminus of the helicase and vice versa. The amino acid sequence of the moiety is preferably added in frame into the amino acid sequence of the helicase. In other words, the moiety is preferably inserted within the sequence of the helicase. In such embodiments, the helicase and moiety are typically attached at two points, i.e. via the amino and carboxy terminal amino acids of the moiety. If the moiety is inserted within the sequence of the helicase, it is preferred that the amino and carboxy terminal amino acids of the moiety are in close proximity and are each attached to adjacent amino acids in the sequence of the helicase or variant thereof. In a preferred embodiment, the moiety is inserted into a loop region of the helicase.

The helicase may be attached directly to the moiety. The helicase is preferably attached to the moiety using one or more, such as two or three, linkers as discussed above. The one or more linkers may be designed to constrain the mobility of the moiety. The helicase and/or the moiety may be modified to facilitate attachment of the one or more linker as discussed above.

Cleavable linkers can be used as an aid to separation of constructs from non-attached components and can be used to further control the synthesis reaction. For example, a hetero-bifunctional linker may react with the helicase, but not the moiety. If the free end of the linker can be used to bind the helicase protein to a surface, the unreacted helicases from the first reaction can be removed from the mixture. Subsequently, the linker can be cleaved to expose a group that reacts with the moiety. In addition, by following this sequence of linkage reactions, conditions may be optimised first for the reaction to the helicase, then for the reaction to the moiety after cleavage of the linker. The second reaction would also be much more directed towards the correct site of reaction with the moiety because the linker would be confined to the region to which it is already attached.

The helicase may be covalently attached to the bifunctional crosslinker before the helicase/crosslinker complex is covalently attached to the moiety. Alternatively, the moiety may be covalently attached to the bifunctional crosslinker before the bifunctional crosslinker/moiety complex is attached to the helicase. The helicase and moiety may be covalently attached to the chemical crosslinker at the same time.

Preferred methods of attaching the helicase to the moiety are cysteine linkage and Faz linkage as described above. In a preferred embodiment, a reactive cysteine is presented on a peptide linker that is genetically attached to the moiety. This means that additional modifications will not necessarily be needed to remove other accessible cysteine residues from the moiety.

Cross-linkage of helicases or moieties to themselves may be prevented by keeping the concentration of linker in a vast excess of the helicase and/or moiety. Alternatively, a “lock and key” arrangement may be used in which two linkers are used. Only one end of each linker may react together to form a longer linker and the other ends of the linker each react with a different part of the construct (i.e. helicase or moiety). This is discussed in more detail below.

The site of attachment is selected such that, when the construct is contacted with a polynucleotide, both the helicase and the moiety can bind to the polynucleotide and control its movement.

Attachment can be facilitated using the polynucleotide binding activities of the helicase and the moiety. For instance, complementary polynucleotides can be used to bring the helicase and moiety together as they hybridize. The helicase can be bound to one polynucleotide and the moiety can be bound to the complementary polynucleotide. The two polynucleotides can then be allowed to hybridise to each other. This will bring the helicase into close contact with the moiety, making the linking reaction more efficient. This is especially helpful for attaching two or more helicases in the correct orientation for controlling movement of a target polynucleotide. An example of complementary polynucleotides that may be used are shown below.

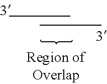

For helicase-Phi29 constructs the DNA below could be used.

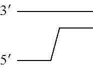

Tags can be added to the construct to make purification of the construct easier. These tags can then be chemically or enzymatically cleaved off, if their removal is necessary. Fluorophores or chromophores can also be included, and these could also be cleavable.

A simple way to purify the construct is to include a different purification tag on each protein (i.e. the helicase and the moiety), such as a hexa-His-tag and a Strep-Tag®. If the two proteins are different from one another, this method is particularly useful. The use of two tags enables only the species with both tags to be purified easily.

If the two proteins do not have two different tags, other methods may be used. For instance, proteins with free surface cysteines or proteins with linkers attached that have not reacted to form a construct could be removed, for instance using an iodoacetamide resin for maleimide linkers.

Constructs of the invention can also be purified from unreacted proteins on the basis of a different DNA processivity property. In particular, a construct of the invention can be purified from unreacted proteins on the basis of an increased affinity for a polynucleotide, a reduced likelihood of disengaging from a polynucleotide once bound and/or an increased read length of a polynucleotide as it controls the translocation of the polynucleotide through a nanopore

A targeted construct that binds to a specific polynucleotide sequence can also be designed. As discussed in more detail below, the polynucleotide binding moiety may bind to a specific polynucleotide sequence and thereby target the helicase portion of the construct to the specific sequence.

### Polynucleotide Binding Moiety

The constructs of the invention comprise a polynucleotide binding moiety. A polynucleotide binding moiety is a polypeptide that is capable of binding to a polynucleotide. The moiety is preferably capable of specific binding to a defined polynucleotide sequence. In other words, the moiety preferably binds to a specific polynucleotide sequence, but displays at least 10 fold less binding to different sequences or more preferably at least 100 fold less binding to different sequences or most preferably at least 1000 fold less binding to different sequences. The different sequence may be a random sequence. In some embodiments, the moiety binds to a specific polynucleotide sequence, but binding to different sequences cannot be measured. Moieties that bind to specific sequences can be used to design constructs that are targeted to such sequences.

The moiety typically interacts with and modifies at least one property of a polynucleotide. The moiety may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the target polynucleotide can be oxidized or methylated. One or more nucleotides in the target polynucleotide may be damaged. For instance, the polynucleotide may comprise a pyrimidine dimer. Such dimers are typically associated with damage by ultraviolet light and are the primary cause of skin melanomas. One or more nucleotides in the target polynucleotide may be modified, for instance with a label or a tag. Suitable labels are described above. The target polynucleotide may comprise one or more spacers.

A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), guanosine monophosphate (GMP), thymidine monophosphate (TMP), uridine monophosphate (UMP), cytidine monophosphate (CMP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP) and deoxycytidine monophosphate (dCMP). The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

It is preferred that the tertiary structure of the moiety is known. Knowledge of the three dimensional structure of the moiety allows modifications to be made to the moiety to facilitate its function in the construct of the invention.

The moiety may be any size and have any structure. For instance, the moiety may be an oligomer, such as a dimer or trimer. The moiety is preferably a small, globular polypeptide formed from one monomer. Such moieties are easy to handle and are less likely to interfere with the ability of the helicase to control the movement of the polynucleotide, particularly if fused to or inserted into the sequence of the helicase.

The amino and carboxy terminii of the moiety are preferably in close proximity. The amino and carboxy terminii of the moiety are more preferably presented on same face of the moiety. Such embodiments facilitate insertion of the moiety into the sequence of the helicase. For instance, if the amino and carboxy terminii of the moiety are in close proximity, each can be attached by genetic fusion to adjacent amino acids in the sequence of the helicase.

It is also preferred that the location and function of the active site of the moiety is known. This prevents modifications being made to the active site that abolish the activity of the moiety. It also allows the moiety to be attached to the helicase so that the moiety binds to the polynucleotide and controls its movement. Knowledge of the way in which a moiety may bind to and orient polynucleotides also allows an effective construct to be designed.

The constructs of the invention are useful in Strand Sequencing. The moiety preferably binds the polynucleotide in a buffer background which is compatible with Strand Sequencing and the discrimination of the nucleotides. The moiety preferably has at least residual activity in a salt concentration well above the normal physiological level, such as from 100 mM to 2M. The moiety is more preferably modified to increase its activity at high salt concentrations. The moiety may also be modified to improve its processivity, stability and shelf life.

Suitable modifications can be determined from the characterisation of polynucleotide binding moieties from extremophiles such as halophilic, moderately halophilic bacteria, thermophilic and moderately thermophilic organisms, as well as directed evolution approaches to altering the salt tolerance, stability and temperature dependence of mesophilic or thermophilic exonucleases.

The polynucleotide binding moiety preferably comprises one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

The helix-hairpin-helix (HhH) domains are polypeptide motifs that bind DNA in a sequence non-specific manner. They have been shown to confer salt stability and processivity when fused to polymerases, as well as increasing their thermal stability. Suitable domains include domain H (residues 696-751) and domain HI (residues 696-802) from Topoisomerase V from Methanopyrus kandleri (SEQ ID NO: 47). As discussed below, the polynucleotide binding moiety may be domains H-L of SEQ ID NO: 47 as shown in SEQ ID NO: 48. Topoisomerase V from Methanopyrus kandleri is an example of a double-stranded binding protein as discussed below.

The HhH domain preferably comprises the sequence shown in SEQ ID NO: 24 or 37 or 38 or a variant thereof. This domain increases the processivity and the salt tolerance of a helicase when used in a construct of the invention. A variant of SEQ ID NO: 24 or 37 or 38 is a protein that has an amino acid sequence which varies from that of SEQ ID NO: 24 or 37 or 38 and which retains polynucleotide binding activity. This can be measured as described above. A variant typically has at least 50% homology to SEQ ID NO: 24 or 37 or 38 based on amino acid identity over its entire sequence (or any of the % homologies discussed above in relation to helicases) and retains polynucleotide binding activity. A variant may differ from SEQ ID NO: 24 or 37 or 38 in any of the ways discussed above in relation to helicases or below in relation to pores. A variant preferably comprises one or more substituted cysteine residues and/or one or more substituted Faz residues to facilitate attachment to the helicase as discussed above.

SSBs bind single stranded DNA with high affinity in a sequence non-specific manner. They exist in all domains of life in a variety of forms and bind DNA either as monomers or multimers. Using amino acid sequence alignment and logarithms (such as Hidden Markov models) SSBs can be classified according to their sequence homology. The Pfam family, PF00436, includes proteins that all show sequence similarity to known SSBs. This group of SSBs can then be further classified according to the Structural Classification of Proteins (SCOP). SSBs fall into the following lineage: Class; All beta proteins, Fold; OB-fold, Superfamily: Nucleic acid-binding proteins, Family; Single strand DNA-binding domain, SSB. Within this family SSBs can be classified according to subfamilies, with several type species often characterised within each subfamily.

The SSB may be from a eukaryote, such as from humans, mice, rats, fungi, protozoa or plants, from a prokaryote, such as bacteria and archaea, or from a virus.

Eukariotic SSBs are known as replication protein A (RPAs). In most cases, they are hetero-trimers formed of different size units. Some of the larger units (e.g. RPA70 of Saccharomyces cerevisiae) are stable and bind ssDNA in monomeric form.

Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). The SSBs from archaeal genomes are considered to be related with eukaryotic RPAs. Few of them, such as the SSB encoded by the crenarchaeote Sulfolobus solfataricus, are homo-tetramers. The SSBs from most other species are closer related to the replication proteins from eukaryotes and are referred to as RPAs. In some of these species they have been shown to be monomeric (Methanococcus jannaschii and Methanothermobacter thermoautotrophicum). Still, other species of Archaea, including Archaeoglobus fulgidus and Methanococcoides burtonii, appear to each contain two open reading frames with sequence similarity to RPAs. There is no evidence at protein level and no published data regarding their DNA binding capabilities or oligomeric state. However, the presence of two oligonucleotide/oligosaccharide (OB) folds in each of these genes (three OB folds in the case of one of the M. burtonii ORFs) suggests that they also bind single stranded DNA.

Viral SSBs bind DNA as monomers. This, as well as their relatively small size renders them amenable to genetic fusion to other proteins, for instance via a flexible peptide linker. Alternatively, the SSBs can be expressed separately and attached to other proteins by chemical methods (e.g. cysteines, unnatural amino-acids). This is discussed in more detail below.

The SSB is preferably either (i) an SSB comprising a carboxy-terminal (C-terminal) region which does not have a net negative charge or (ii) a modified SSB comprising one or more modifications in its C-terminal region which decreases the net negative charge of the C-terminal region. Such SSBs do not block the transmembrane pore and therefore allow characterization of the target polynucleotide.

Examples of SSBs comprising a C-terminal region which does not have a net negative charge include, but are not limited to, the human mitochondrial SSB (HsmtSSB; SEQ ID NO: 39, the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 40) or a variant of any of those proteins. A variant is a protein that has an amino acid sequence which varies from that of the wild-type protein and which retains single stranded polynucleotide binding activity. Polynucleotide binding activity can be determined using methods known in the art (and as described above). For instance, the ability of a variant to bind a single stranded polynucleotide can be determined as described in the Examples.

A variant of SEQ ID NO: 39 or 40 typically has at least 50% homology to SEQ ID NO: 39 or 40 based on amino acid identity over its entire sequence (or any of the % homologies discussed above in relation to helicases) and retains single stranded polynucleotide binding activity. A variant may differ from SEQ ID NO: 39 or 40 in any of the ways discussed above in relation to helicases. In particular, a variant may have one or more conservative substitutions as shown in Tables 5 and 6.

Examples of SSBs which require one or more modifications in their C-terminal region to decrease the net negative charge include, but are not limited to, the SSB of E. coli (EcoSSB; SEQ ID NO: 41, the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDC13 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 42), the SSB from RB69 (gp32; SEQ ID NO: 25), the SSB from T7 (gp2.5; SEQ ID NO: 26) or a variant of any of these proteins. Hence, the SSB used in the method of the invention may be derived from any of these proteins.

In addition to the one or more modifications in the C-terminal region, the SSB used in the method may include additional modifications which are outside the C-terminal region or do not decrease the net negative charge of the C-terminal region. In other words, the SSB used in the method of the invention is derived from a variant of a wild-type protein. A variant is a protein that has an amino acid sequence which varies from that of the wild-type protein and which retains single stranded polynucleotide binding activity. Polynucleotide binding activity can be determined as discussed above.

The SSB used in the invention may be derived from a variant of SEQ ID NO: 25, 26, 41 or 42. In other words, a variant of SEQ ID NO: 25, 26, 41 or 42 may be used as the starting point for the SSB used in the invention, but the SSB actually used further includes one or more modifications in its C-terminal region which decreases the net negative charge of the C-terminal region. A variant of SEQ ID NO: 25, 26, 41 or 42 typically has at least 50% homology to SEQ ID NO: 25, 26, 41 or 42 based on amino acid identity over its entire sequence (or any of the % homologies discussed above in relation to helicases) and retains single stranded polynucleotide binding activity. A variant may differ from SEQ ID NO: 25, 26, 41 or 42 in any of the ways discussed above in relation to helicases. In particular, a variant may have one or more conservative substitutions as shown in Tables 5 and 6.

It is straightforward to identify the C-terminal region of the SSB in accordance with normal protein N to C nomenclature. The C-terminal region of the SSB is preferably about the last third of the SSB at the C-terminal end, such as the last third of the SSB at the C-terminal end. The C-terminal region of the SSB is more preferably about the last quarter, fifth or eighth of the SSB at the C-terminal end, such as the last quarter, fifth or eighth of the SSB at the C-terminal end. The last third, quarter, fifth or eighth of the SSB may be measured in terms of numbers of amino acids or in terms of actual length of the primary structure of the SSB protein. The length of the various amino acids in the N to C direction are known in the art.

The C-terminal region is preferably from about the last 10 to about the last 60 amino acids of the C-terminal end of the SSB. The C-terminal region is more preferably about the last 15, about the last 20, about the last 25, about the last 30, about the last 35, about the last 40, about the last 45, about the last 50 or about the last 55 amino acids of the C-terminal end of the SSB.

The C-terminal region typically comprises a glycine and/or proline rich region. This proline/glycine rich region gives the C-terminal region flexibility and can be used to identify the C-terminal region.

Suitable modifications for decreasing the net negative charge are disclosed in International Application No. PCT/GB2013/051924 (published as WO 2014/013259). The SSB may be any of the SSBs disclosed in this International application.

The modified SSB most preferably comprises a sequence selected from those shown in SEQ ID NOs: 33, 34, 43 to 46.

Double-stranded binding proteins bind double stranded DNA with high affinity. Suitable double-stranded binding proteins include, but are not limited to Mutator S (MutS; NCBI Reference Sequence: NP_417213.1; SEQ ID NO: 49), Sso7d (Sufolobus solfataricus P2; NCBI Reference Sequence: NP_343889.1; SEQ ID NO: 50; Nucleic Acids Research, 2004, Vol 32, No. 3, 1197-1207), Sso10b1 (NCBI Reference Sequence: NP_342446.1; SEQ ID NO: 51), Sso10b2 (NCBI Reference Sequence: NP_342448.1; SEQ ID NO: 52), Tryptophan repressor (Trp repressor; NCBI Reference Sequence: NP_291006.1; SEQ ID NO: 53), Lambda repressor (NCBI Reference Sequence: NP_040628.1; SEQ ID NO: 54), Cren7 (NCBI Reference Sequence: NP_342459.1; SEQ ID NO: 55), major histone classes H1/H5, H2A, H2B, H3 and H4 (NCBI Reference Sequence: NP_066403.2, SEQ ID NO: 56), dsbA (NCBI Reference Sequence: NP_049858.1; SEQ ID NO: 57), Rad51 (NCBI Reference Sequence: NP_002866.2; SEQ ID NO: 58), sliding clamps and Topoisomerase V Mka (SEQ ID NO: 47) or a variant of any of these proteins. A variant of SEQ ID NO: 47, 49, 50, 51, 52, 53, 54, 55, 56, 57 or 58 typically has at least 50% homology to SEQ ID NO: 47, 49, 50, 51, 52, 53, 54, 55, 56, 57 or 58 based on amino acid identity over its entire sequence (or any of the % homologies discussed above in relation to helicases) and retains single stranded polynucleotide binding activity. A variant may differ from SEQ ID NO: 47, 49, 50, 51, 52, 53, 54, 55, 56, 57 or 58 in any of the ways discussed above in relation to helicases. In particular, a variant may have one or more conservative substitutions as shown in Tables 5 and 6. Most polymerases achieve processivity by interacting with sliding clamps. In general, these are multimeric proteins (homo-dimers or homo-trimers) that encircle dsDNA. These sliding clamps require accessory proteins (clamp loaders) to assemble them around the DNA helix in an ATP-dependent process. They also do not contact DNA directly, acting as a topological tether. As sliding clamps interact with their cognate polymerases in a specific manner via a polymerase domain, this fragment could be fused to the helicase in order to incite recruitment of helicases onto the sliding clamp. This interaction could be further stabilized by the generation of a covalent bond (introduction of cysteines or unnatural amino-acids).

Related to DNA sliding clamps, processivity factors are viral proteins that anchor their cognate polymerases to DNA, leading to a dramatic increase in the length of the fragments generated. They can be monomeric (as is the case for UL42 from Herpes simplex virus 1) or multimeric (UL44 from Cytomegalovirus is a dimer), they do not form closed rings around the DNA strand and they contact DNA directly. UL42 has been shown to increase processivity without reducing the rate of its corresponding polymerase, suggesting that it interacts with DNA in a different mode to SSBs. The UL42 preferably comprises the sequence shown in SEQ ID NO: 27 or SEQ ID NO: 32 or a variant thereof. A variant of SEQ ID NO: 27 or 32 is a protein that has an amino acid sequence which varies from that of SEQ ID NO: 27 or 32 and which retains polynucleotide binding activity. This can be measured as described above. A variant typically has at least 50% homology to SEQ ID NO: 27 or 32 based on amino acid identity over its entire sequence (or any of the % homologies discussed above in relation to helicases) and retains polynucleotide binding activity. A variant may differ from SEQ ID NO: 27 or SEQ ID NO: 32 in any of the ways discussed above in relation to helicases or below in relation to pores. A variant preferably comprises one or more substituted cysteine residues and/or one or more substituted Faz residues to facilitate attachment to the helicase as discussed above.

Attaching UL42 to a helicase could be done via genetic fusion or chemical attachment (cysteines, unnatural amino-acids). As the polymerase polypeptide that binds UL42 is visible in the crystal structure, these 35 amino acids (residues 1200-1235) could be fused onto the C-terminus of the helicase and the natural affinity between this polypeptide and the processivity factor used to form a complex. The interaction could be stabilized by introducing a covalent interaction (cysteines or unnatural amino-acids). One option is to utilize a natural UL42 cysteine (C300) that is located close to the polypeptide interaction site and introduce a point mutation into the polymerase polypeptide (e.g. L1234C).

A reported method of increasing polymerase processivity is by exploiting the interaction between E. coli thioredoxin (Trx) and the thioredoxin binding domain (TBD) of bacteriophage T7 DNA polymerase (residues 258-333). The binding of Trx to TBD causes the polypeptide to change conformation to one that binds DNA. TBD is believed to clamp down onto a DNA strand and limit the polymerase off-rate, thus increasing processivity. Chimeric polymerases have been made by transferring TBD onto a non-processive polymerase, resulting in 1000 fold increase in polymerised fragment length. There were no attempts to attach TBD to any other class of proteins, but a covalent link between TBD and Trx was engineered and can be used to stabilise the interaction.

Some helicases use accessory proteins in-vivo to achieve processivity (e.g. cisA from phage Φx174 and geneII protein from phage M13 for E. coli Rep helicase). Some of these proteins have been shown to interact with more than one helicase (e.g. MutL acts on both UvrD and Rep, though not to the same extent). These proteins have intrinsic DNA binding capabilities, some of them recognizing a specific DNA sequence. The ability of some of these accessory proteins to covalently attach themselves to a specific DNA sequence could also be used to create a set starting point for the helicase activity.

The proteins that protect the ends of chromosomes bind to telomeric ssDNA sequences in a highly specific manner. This ability could either be exploited as is or by using point mutations to abolish the sequence specificity.

Small DNA binding motifs (such as helix-turn-helix) recognize specific DNA sequences. In the case of the bacteriophage 434 repressor, a 62 residue fragment was engineered and shown to retain DNA binding abilities and specificity.

An abundant motif in eukaryotic proteins, zinc fingers consist of around 30 amino-acids that bind DNA in a specific manner. Typically each zinc finger recognizes only three DNA bases, but multiple fingers can be linked to obtain recognition of a longer sequence.

Proliferating cell nuclear antigens (PCNAs) form a very tight clamp (doughnut) which slides up and down the dsDNA or ssDNA. The PCNA from crenarchaeota is unique in being a hetero-trimer so it is possible to functionalise one subunit and retain activity. Its subunits are shown in SEQ ID NOs: 28, 29 and 30. The PCNA is preferably a trimer comprising the sequences shown in SEQ ID NOs: 28, 29 and 30 or variants thereof. PCNA sliding clamp (NCBI Reference Sequence: ZP_06863050.1; SEQ ID NO: 59) forms a dimer. The PCNA is preferably a dimer comprising SEQ ID NO: 59 or a variant thereof. A variant is a protein that has an amino acid sequence which varies from that of SEQ ID NO: 28, 29, 30 or 59 and which retains polynucleotide binding activity. This can be measured as described above. A variant is typically a trimer comprising sequences that have at least 50% homology to SEQ ID NOs: 28, 29 and 30 or a dimer comprising sequences that have at least 50% homology to SEQ ID NO: 59 based on amino acid identity over each entire sequence (or any of the % homologies discussed above in relation to helicases) and which retains polynucleotide binding activity. A variant may comprise sequences which differ from SEQ ID NO: 28, 29, 30 or 59 in any of the ways discussed above in relation to helicases or below in relation to pores. A variant preferably comprises one or more substituted cysteine residues and/or one or more substituted Faz residues to facilitate attachment to the helicase as discussed above. In a preferred embodiment, subunits 1 and 2 of the PCNA from crenarchaeota (i.e. SEQ ID NOs: 28 and 29 or variants thereof) are attached, such as genetically fused, and the resulting protein is attached to a helicase to form a construct of the invention. During use of the construct, subunit 3 (i.e. SEQ ID NO: 30 or a variant thereof) may be added to complete the PCNA clamp (or doughnut) once the construct has bound the polynucleotide. In a preferred embodiment, one monomer of the PCNA sliding clamp (i.e. SEQ ID NO: 59 or a variant thereof) is attached, such as genetically fused, to a helicase to form a construct of the invention. During use of the construct, the second monomer (i.e. SEQ ID NO: 59 or a variant thereof) may be added to complete the PCNA clamp (or doughnut) once the construct has bound the polynucleotide.

The polynucleotide binding motif may be selected from any of those shown in Table 3 below.

TABLE 3
Suitable polynucleotide binding motifs

| No. | Name | Class | Organism | Structure | Sequence | Functional form | MW (Da) | Notes |
|---|---|---|---|---|---|---|---|---|
| 1 | SSBEco | ssb | Escherichia coli | 1QVC, | P0AGE0 | homo-tetramer | 18975 | |
| | | | | 1EYG | | | | |
| 2 | SSBBhe | ssb | Bartonella | 3LGJ, | Q6G302 | homo-tetramer | 16737 | structure only |
| | | | henselae | 3PGZ | | | | |
| 3 | SSBCbu | ssb | Coxiella burnetii | 3TQY | Q83EP4 | homo-tetramer | 17437 | structure only |
| 4 | SSBTma | ssb | Thermathoga | 1Z9F | Q9WZ73 | homo-dimer | 16298 | small, |
| | | | maritima | | | | | thermostable, |
| | | | | | | | | salt independent |
| | | | | | | | | DNA binding |
| 5 | SSBHpy | ssb | Helicobacter | 2VW9 | O25841 | homo-tetramer | 20143 | |
| | | | pylori | | | | | |
| 6 | SSBDra | ssb | Deinococcus | 1SE8 | Q9RY51 | homo-dimer | 32722 | |
| | | | radiodurans | | | | | |
| 7 | SSBTaq | ssb | Thermus | 2FXQ | Q9KH06 | homo-dimer | 30026 | |
| | | | aquaticus | | | | | |
| 8 | SSBMsm | ssb | Mycobacterium | 3A5U, | Q9AFI5 | homo-tetramer | 17401 | tetramer more |
| | | | smegmatis | 1X3E | | | | stable than |
| | | | | | | | | E. coli, binding |
| | | | | | | | | less salt |
| | | | | | | | | dependent |
| 9 | SSBSso | ssb/RPA | Sulfolobus | 1O7I | Q97W73 | homo-tetramer | 16138 | similarities with |
| | | | solfataricus | | | | | RPA |
| 10 | SSBMHsmt | ssb | Homo sapiens | 3ULL | Q04837 | homo-tetramer | 17260 | |
| 11 | SSBMle | ssb | Mycobacterium | 3AFP | P46390 | homo-tetramer | 17701 | |
| | | | leprae | | | | | |
| 12 | gp32T4 | ssb | Bacteriohage T4 | 1GPC | P03695 | monomer | 33506 | Homo-dimer in |
| | | | | | | | | the absence of |
| | | | | | | | | DNA, monomer |
| | | | | | | | | when binding |
| | | | | | | | | DNA. |
| 13 | gp32RB69 | ssb | Bacteriophage | 2A1K | Q7Y265 | monomer | 33118 | |
| | | | RB69 | | | | | |
| 14 | gp2.5T7 | ssb | Bacteriohage T7 | 1JE5 | P03696 | monomer | 25694 | |
| 15 | UL42 | processivity | Herpes virus 1 | 1DML | P10226 | monomer | 51159 | binds ssDNA |
| | | factor | | | | | | dsDNA, |
| | | | | | | | | structure shows |
| | | | | | | | | link with |
| | | | | | | | | polymerase |
| 16 | UL44 | processivity | Herpes virus 5 | 1YYP | P16790 | homo-dimer | 46233 | forms C shaped |
| | | factor | (cytomegalovirus) | | | | | clamp on DNA |
| 17 | pf8 | processivity | KSHV | 3I2M | Q77ZG5 | homo-dimer | 42378 | |
| | | factor | | | | | | |
| 18 | RPAMja | RPA | Methanococcus | 3DM3 | Q58559 | monomer | 73842 | contains 4 OB |
| | | | jannaschii | | | | | folds. Structure |
| | | | | | | | | of fragment |
| 19 | RPAMma | RPA | Methanococcus | 3E0E, | Q6LYF9 | monomer | 71388 | Core domain |
| | | | maripaludis | 2K5V | | | | structure |
| 20 | RPAMth | RPA | Methanothermobacter | | | monomer | 120000 | Shown to |
| | | | thermoautotrophicus | | | | | interact directly |
| | | | | | | | | with Hel308. |
| | | | | | | | | Sequence from |
| | | | | | | | | paper. |
| 21 | RPA70Sce | RPA | Saccharomyces | 1YNX | P22336 | hetero-trimer | 70348 | unit has two OB |
| | | | cerevisiae | | | | | folds and binds |
| | | | | | | | | DNA |
| 22 | RPAMbu1 | RPA | Methanococcoides | | Q12V72 | ? | 41227 | three OB folds |
| | | | burtonii | | | | | identified |
| 23 | RPAMbu2 | RPA | Methanococcoides | | Q12W96 | ? | 47082 | two OB folds |
| | | | burtonii | | | | | identified |
| 24 | RPA70Hsa | RPA | Homo sapiens | 1JMC | P27694 | hetero-trimer | 68138 | |
| 25 | RPA14Hsa | RPA | Homo sapiens | 3KDF | P35244 | hetero-trimer | 13569 | in complex with |
| | | | | | | | | RPA32 |
| 26 | gp45T4 | sliding | Bacteriophage | 1CZD | P04525 | homo-trimer | 24858 | ring shape |
| | | clamp | T4 | | | | | threads DNA |
| 27 | BetaEco | sliding | E. coli | 3BEP | P0A988 | homo-dimer | 40587 | ring shape |
| | | clamp | | | | | | threads DNA, |
| | | | | | | | | may bind ssDNA |
| | | | | | | | | in pocket |
| 28 | PCNASce | sliding | Saccharomyces | 1PLQ, | P15873 | homo-dimer | 28916 | ring shape |
| | | clamp | cerevisiae | 3K4X | | | | threads DNA |
| 29 | PCNATko | sliding | Thermococcus | 3LX1 | Q5JF32 | homo-dimer | 28239 | |
| | | clamp | kodakaraensis | | | | | |
| 30 | PCNAHvo | sliding | Haloferax | 3IFV | D0VWY8 | homo-dimer | 26672 | |
| | | clamp | volcanii | | | | | |
| 31 | PCNAPfu | sliding | Pyrococcus | 1GE8 | O73947 | homo-dimer | 28005 | |
| | | clamp | furiosus | | | | | |
| 32 | PCNAMbu | sliding | Methanococcoides | | Q12U18 | homo-dimer | 27121 | Inferred from |
| | | clamp | burtonii | | | | | homology |
| 33 | BetaMtu | sliding | Mycobacterium | 3P16 | Q50790 | homo-dimer | 42113 | |
| | | clamp | tuberculosis | | | | | |
| 34 | BetaTma | sliding | Thermotoga | 1VPK | Q9WYA0 | homo-dimer | 40948 | |
| | | clamp | maritima | | | | | |
| 35 | BetaSpy | sliding | Streptococcus | 2AVT | Q9EVR1 | homo-dimer | 41867 | |
| | | clamp | pyrogenes | | | | | |
| 36 | gp45RB69 | sliding | Bacteriophage | 1B77 | O80164 | homo-trimer | 25111 | Structure shows |
| | | clamp | RB69 | | | | | interaction with |
| | | | | | | | | polypeptide from |
| | | | | | | | | polymerase |
| 37 | p55Hsa | DNA | Homo sapiens | 2G4C, | Q9UHN | monomer | 54911 | interacts with |
| | | binding | (mitochondrial) | 3IKL, | | | | specific |
| | | protein | | 3IKM | | | | polymerase |
| | | | | | | | | domain |
| 38 | p55Dme | DNA | Drosophylla | | Q9VJV8 | monomer | 41027 | associates with |
| | | binding | melanogaster | | | | | polymerase |
| | | protein | | | | | | Gamma |
| | | | | | | | | conferring salt |
| | | | | | | | | tolerance, |
| | | | | | | | | processivity and |
| | | | | | | | | increased activity |
| 39 | p55Xla | DNA | Xenopus | | Q9W6G7 | monomer | 52283 | |
| | | binding | laevis | | | | | |
| | | protein | | | | | | |
| 40 | RepDSau | replication | Staphylococcus | | P08115 | homo-dimer | 37874 | increases |
| | | initiation | aureus | | | | | processivity of |
| | | protein | | | | | | PcrA, covalently |
| | | | | | | | | and specifically |
| | | | | | | | | links DNA |
| 41 | G2P | replication | Enterobacteria | | P69546 | monomer | 46168 | increases |
| | | initiation | phage 1 | | | | | processivity of |
| | | protein | | | | | | Rep, covalently |
| | | | | | | | | and specifically |
| | | | | | | | | links DNA |
| 42 | MutLEco | mismatch | Escherichia coli | 1BKN, | P23367 | homo-dimer | 67924 | increases |
| | | repair | | 1B62, | | | | processivity of |
| | | protein | | 1B63 | | | | UvrD (and Rep) |
| 43 | KuMtu | DNA | Mycobacterium | | O05866 | homo-dimer | 30904 | increases |
| | | repair | tuberculosis | | | | | processivity of |
| | | protein | | | | | | UvrD1. Structure |
| | | | | | | | | available for |
| | | | | | | | | human Ku |
| 44 | OnTEBP | telomere | Oxytricha nova- | 1OTC | P29549 | hetero-dimer | 56082 | Specific biding |
| | | binding | Alpha | | | | | to 3′ end |
| | | protein | | | | | | T4G4T4G4. |
| | | | | | | | | Alpha subunit |
| | | | | | | | | may be enough |
| | | | Oxytricha nova- | | P16458 | | 41446 | |
| | | | Beta | | | | | |
| 45 | EcrTEBP | telomere | Euplotes crassus | | Q06183 | monomer | 53360 | Homolog to |
| | | binding | | | | | | OnTEBP with no |
| | | protein | | | | | | Beta subunit in |
| | | | | | | | | genome |
| 46 | TteTEBP | telomere | Tetrachymena | | Q23FB9 | hetero-dimer | 53073 | Homolog to |
| | | binding | termophila Alpha | | | | | OnTEBP-Alpha |
| | | protein | | | | | | |
| | | | Tetrachymena | | Q23FH0 | | 54757 | May be homolog |
| | | | termophila Beta | | | | | to OnTEBP Beta |
| 47 | pot1Spo | telomere | Schizosaccharomyces | | O13988 | monomer | 64111 | related to TEBP |
| | | binding | pombe | | | | | |
| | | proteins | | | | | | |
| 48 | Cdc13pSce | telomere | Saccharomyces | | C7GSV7 | monomer | 104936 | specific binding |
| | | binding | cerevisiae | | | | | to telomeric |
| | | proteins | | | | | | DNA |
| 49 | C1 | repressor | Bacteriophage | | P16117 | homo-dimer | 10426 | binds DNA |
| | | | 434 | | | | | specifically as |
| | | | | | | | | homo-dimer |
| 50 | LexA | repressor | Escherichia coli | 1LEB | P0A7C2 | homo-dimer | 22358 | binds DNA |
| | | | | | | | | specifically as |
| | | | | | | | | homo-dimer |

The polynucleotide binding moiety is preferably derived from a polynucleotide binding enzyme. A polynucleotide binding enzyme is a polypeptide that is capable of binding to a polynucleotide and interacting with and modifying at least one property of the polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide binding moiety does not need to display enzymatic activity as long as it is capable of binding the polynucleotide and controlling its movement. For instance, the moiety may be derived from an enzyme that has been modified to remove its enzymatic activity or may be used under conditions which prevent it from acting as an enzyme.

The polynucleotide binding moiety is preferably derived from a nucleolytic enzyme. The enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are exonucleases, polymerases, helicases and topoisomerases, such as gyrases. Suitable exonucleases include, but are not limited to, exonuclease I from E. coli, exonuclease III enzyme from E. coli, RecJ from T. thermophilus and bacteriophage lambda exonuclease, TatD exonuclease and variants thereof.

The polymerase is preferably a member of any of the Moiety Classification (EC) groups 2.7.7.6, 2.7.7.7, 2.7.7.19, 2.7.7.48 and 2.7.7.49. The polymerase is preferably a DNA-dependent DNA polymerase, an RNA-dependent DNA polymerase, a DNA-dependent RNA polymerase or an RNA-dependent RNA polymerase. The polymerase may be PyroPhage® 3173 DNA Polymerase (which is commercially available from Lucigen® Corporation), SD Polymerase (commercially available from Bioron®) or variants thereof. The polynucleotide binding moiety is preferably derived from Phi29 DNA polymerase (SEQ ID NO: 31). The moiety may comprise the sequence shown in SEQ ID NO: 101 or a variant thereof. A variant of SEQ ID NO: 31 is an enzyme that has an amino acid sequence which varies from that of SEQ ID NO: 31 and which retains polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NO: 31, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 31 over the entire sequence. There may be at least 80%, for example at least 85%, 90% or 95%, amino acid identity over a stretch of 200 or more, for example 230, 250, 270 or 280 or more, contiguous amino acids (“hard homology”). Homology is determined as described below. The variant may differ from the wild-type sequence in any of the ways discussed below with reference to SEQ ID NOs: 2 and 4.

The helicase may be any of those discussed above. Helicase dimers and multimers are discussed in detail below. The polynucleotide binding moiety may be a polynucleotide binding domain derived from a helicase. For instance, the polynucleotide binding moiety preferably comprises the sequence shown in SEQ ID NOs: 35 or 36 or a variant thereof. A variant of SEQ ID NOs: 35 or 36 is a protein that has an amino acid sequence which varies from that of SEQ ID NOs: 35 or 36 and which retains polynucleotide binding activity. This can be measured as described above. The variant may include modifications that facilitate binding of the polynucleotide and/or facilitate its activity at high salt concentrations and/or room temperature.

Over the entire length of the amino acid sequence of SEQ ID NOs: 35 or 36, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NOs: 35 or 36 over the entire sequence. There may be at least 80%, for example at least 85%, 90% or 95%, amino acid identity over a stretch of 40 or more, for example 50, 60, 70 or 80 or more, contiguous amino acids (“hard homology”). Homology is determined as described below. The variant may differ from the wild-type sequence in any of the ways discussed below with reference to SEQ ID NOs: 2 and 4.

The topoisomerase is preferably a member of any of the Moiety Classification (EC) groups 5.99.1.2 and 5.99.1.3.

The polynucleotide binding moiety may be any of the enzymes discussed above.

The moiety may be labelled with a revealing label. The label may be any of those described above.

The moiety may be isolated from any moiety-producing organism, such as E. coli, T. thermophilus or bacteriophage, or made synthetically or by recombinant means. For example, the moiety may be synthesized by in vitro translation and transcription as described below. The moiety may be produced in large scale following purification as described below.

### Helicase Oligomers

As will be clear from the discussion above, the polynucleotide binding moiety is preferably derived from a helicase. For instance, it may be a polynucleotide domain from a helicase. The moiety more preferably comprises one or more helicases. The helicases may be any of those discussed above with reference to the constructs of the invention, including the helicases of the invention and helicases which are not modified in accordance with the invention. In such embodiments, the constructs of the invention of course comprise two or more helicases attached together. At least one of the helicases is preferably modified in accordance with the invention. The constructs may comprise two, three, four, five or more helicases. In other words, the constructs of the invention may comprise a helicase dimer, a helicase trimer, a helicase tetramer, a helicase pentamer and the like.

The two or more helicases can be attached together in any orientation. Identical or similar helicases may be attached via the same amino acid position or spatially proximate amino acid positions in each helicase. This is termed the “head-to-head” formation. Alternatively, identical or similar helicases may be attached via positions on opposite or different sides of each helicase. This is termed the “head-to-tail” formation. Helicase trimers comprising three identical or similar helicases may comprise both the head-to-head and head-to-tail formations.

The two or more helicases may be different from one another (i.e. the construct is a hetero-dimer, -trimer, -tetramer or -pentamer etc.). For instance, the constructs of the invention may comprise (a) one or more helicases of the invention and one or more helicases which are not modified in accordance with the invention; (b) two or more different helicases of the invention; or (c) two or more helicases which are not modified in accordance with the invention. The construct may comprise two different variants of the same Dda helicase. For instance, the construct may comprise two variants of one of the helicases discussed above with one or more cysteine residues or Faz residues introduced at different positions in each variant. In this instance, the helicases can be in a head-to-tail formation.

Hetero-dimers can be formed in two possible ways. The first involves the use of a homo-bifunctional linker as discussed above. One of the helicase variants can be modified with a large excess of linker in such a way that one linker is attached to one molecule of the protein. This linker modified variant can then be purified away from unmodified proteins, possible homo-dimers and unreacted linkers to react with the other helicase variant. The resulting dimer can then be purified away from other species.

The second involves the use of hetero-bifunctional linkers. For example, one of the helicase variants can be modified with a first PEG linker containing maleimide or iodoacetamide functional group at one end and a cyclooctyne functional group (DIBO) at the other end. An example of this is shown below:

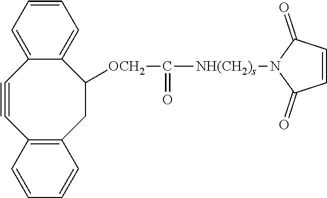

The second helicase variant can be modified with a second PEG linker containing maleimide or iodoacetamide functional group at one end and an azide functional group at the other end. An example is show below:

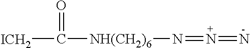

The two helicase variants with two different linkers can then be purified and clicked together (using copper free click chemistry) to make a dimer. Copper free click chemistry has been used in these applications because of its desirable properties. For example, it is fast, clean and not poisonous towards proteins. However, other suitable bio-orthogonal chemistries include, but are not limited to, Staudinger chemistry, hydrazine or hydrazide/aldehyde or ketone reagents (HyNic+4FB chemistry, including all Solulink™ reagents), Diels-Alder reagent pairs and boronic acid/salicyhydroxamate reagents.

These two ways of linking two different variants of the same helicase are also valid for any of the constructs discussed above in which the helicase and the moiety are different from one another, such as dimers of two different helicases and a helicase-polymerase dimer.

Similar methodology may also be used for linking different Faz variants. One Faz variant can be modified with a large excess of linker in such a way that one linker is attached to one molecule of the protein. This linker modified Faz variant can then be purified away from unmodified proteins, possible homo-dimers and unreacted linkers to react with the second Faz variant. The resulting dimer can then be purified away from other species.

Hetero-dimers can also be made by linking cysteine variants and Faz variants of the same helicase or different helicases. Hetero-bifunctional PEG linkers with maleimide or iodoacetamide functionalities at one end and DBCO functionality at the other end can be used in this combination of mutants. An example of such a linker is shown below (DBCO-PEG4-maleimide):

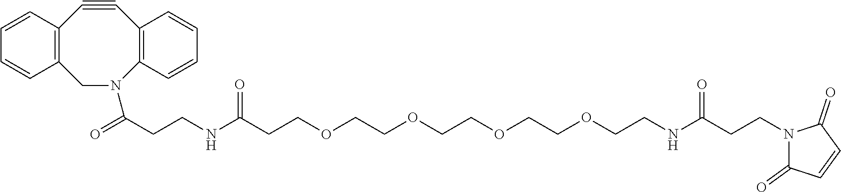 [SMILES: O=C(CCOCCOCCOCCOCCNC(=O)CCN1C(=O)C=CC1=O)NCCC(=O)N1Cc2ccccc2C#Cc2ccccc21]

The length of the linker can be varied by changing the number of PEG units between the two functional groups.

Helicase hetero-trimers can comprise three different types of helicases. The same is true for oligomers comprising more than three helicases. The two or more helicases within a construct may be different variants of the same helicase, such as different variants of any one of SEQ ID NOs: 8 to 23. The different variants may be modified at different positions to facilitate attachment via the different positions. The hetero-trimers may therefore be in a head-to-tail and head-to-head formation.

The two or more helicases in the constructs of the invention may be the same as one another (i.e. the construct is a homo-dimer, -trimer, -tetramer or -pentamer etc.) In such embodiments, the helicases are preferably attached using the same position in each helicase. The helicases are therefore attached head-to-head. The helicases may be linked using a cysteine residue or a Faz residue that has been substituted into the helicases at the same position. Cysteine residues in identical helicase variants can be linked using a homo-bifunctional linker containing thiol reactive groups such as maleimide or iodoacetamide. These functional groups can be at the end of a polyethyleneglycol (PEG) chain as in the following example:

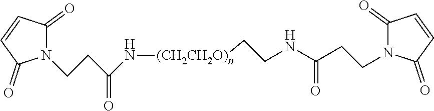

The length of the linker can be varied to suit the required applications. For example, n can be 2, 3, 4, 8, 11, 12, 16 or more. PEG linkers are suitable because they have favourable properties such as water solubility. Other non PEG linkers can also be used in cysteine linkage.

By using similar approaches, identical Faz variants can also be made into homo-dimers. Homo-bifunctional linkers with DIBO functional groups can be used to link two molecules of the same Faz variant to make homo-dimers using Cu²⁺, free click chemistry. An example of a linker is given below:

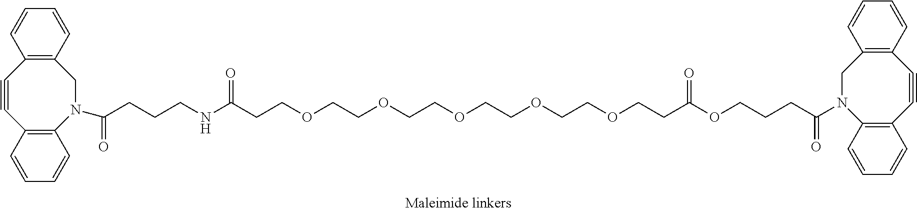 [SMILES: O=C(CCOCCOCCOCCOCCOCCC(=O)OCCCC(=O)N1Cc2ccccc2C#Cc2ccccc21)NCCCC(=O)N1Cc2ccccc2C#Cc2ccccc21]

The length of the PEG linker can vary to include 2, 4, 8, 12, 16 or more PEG units. Such linkers can also be made to incorporate a florescent tag to ease quantifications. Such fluorescence tags can also be incorporated into Maleimide linkers.

Homo-dimers or longer homo-oligomers may also be prepared in the head-to-tail formation if two or more cysteine residues or non-natural amino acids are introduced in the helicase in accordance with the invention and different cysteines or non-natural amino acids in the different helicase monomers are attached together. For instance, homo-oligomers may be formed from variants of SEQ ID NO: 8 comprising Y279C and G357C and the C at 279 in one monomer may be attached to the C at 357 in another monomer. Similarly, homo-oligomers may be formed from variants of SEQ ID NO: 8 comprising I281C and G357C and the C at 281 in one monomer may be attached to the C at 357 in another monomer. The same is true when Faz is introduced at these positions instead of C. Such C and Faz mutants allow series or trains of helicases to be created.

### Polynucleotide Sequences

The invention provides a polynucleotide comprising a sequence which encodes a helicase of the invention, a polypeptide of the invention or a construct of the invention. The polynucleotide may consist of such a sequence. The polynucleotide may be any of those discussed above.

Any of the proteins described herein may be expressed using methods known in the art. Polynucleotide sequences may be isolated and replicated using standard methods in the art. Chromosomal DNA may be extracted from a helicase producing organism, such as Methanococcoides burtonii, and/or a SSB producing organism, such as E. coli. The gene encoding the sequence of interest may be amplified using PCR involving specific primers. The amplified sequences may then be incorporated into a recombinant replicable vector such as a cloning vector. The vector may be used to replicate the polynucleotide in a compatible host cell. Thus polynucleotide sequences may be made by introducing a polynucleotide encoding the sequence of interest into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into a suitable expression vector. In an expression vector, the polynucleotide sequence is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence will be chosen to be compatible with the expression vector used to transform the cell. The host cell is typically bacterial and preferably E. coli. Any cell with a λ DE3 lysogen, for example Rosetta2(DE3)pLys, C41 (DE3), BL21 (DE3), JM109 (DE3), B834 (DE3), TUNER, Origami and Origami B, can express a vector comprising the T7 promoter.

### Series

The invention also provides a series of two or more helicases attached (or bound) to a polynucleotide, wherein at least one of the two or more helicases is a Dda helicase of the invention. The series may comprise any number of helicases such as 2, 3, 4, 5, 6, 7, 8, 9, 10 or more helicases. Any number of the helicases may be Dda helicases of the invention. All of the two or more helicases are preferably Dda helicases of the invention. The one or more Dda helicases of the invention may be any of those discussed above.

The two or more helicases may be the same helicase or may be different helicases. For instance, if the series comprises two or more Dda helicases of the invention, the Dda helicases of the invention may be the same or may be different.

The series may comprise any number and any combination of Dda helicases of the invention. The series of two or more helicases preferably comprises at least two Dda helicases of the invention. The series may comprise two or more Dda helicases each of which comprises a variant of SEQ ID NO: 8 comprising (or comprising only) (i) E94C/A360C, (ii) E94C/A360C and then (ΔM1)G1G2 (i.e. deletion of M1 and then addition G1 and G2), (iii) E94C/A360C/C109A/C136A, (iv) E94C/A360C/C109A/C136A and then (ΔM1)G1G2 (i.e. deletion of M1 and then addition G1 and G2), (v) E94C/A360C/W378A, (vi) E94C/A360C/W378A and then (ΔM1)G1G2 (i.e. deletion of M1 and then addition G1 and G2), (vii) E94C/A360C/C109A/C136A/W378A or (viii) E94C/A360C/C109A/C136A/W378A and then (ΔM1)G1G2 (i.e. deletion of M1 and then addition G1 and G2). One Dda helicase of the invention in the series preferably comprises a variant of SEQ ID NO: 8 comprising (or comprising only) one of (i) to (iv) and another Dda helicase of the invention in the series preferably comprises a variant of SEQ ID NO: 8 comprising (or comprising only) one of (v) to (viii).

In addition to one or more Dda helicases of the invention, the series may comprise one or more helicases which are not part of the invention. The one or more helicases may be or be derived from a Hel308 helicase, a RecD helicase, such as TraI helicase or a TrwC helicase, a XPD helicase or a Dda helicase. The one or more helicases may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013/098561); PCT/GB2013/051925 (published as WO 2014/013260); PCT/GB2013/051924 (published as WO 2014/013259) and PCT/GB2013/051928 (published as WO 2014/013262); and in UK Application No. 1318464.3 filed on 18 Oct. 2013. In particular, the one or more helicases are preferably modified to reduce the size of an opening in the polynucleotide binding domain through which in at least one conformational state the polynucleotide can unbind from the helicase. This is disclosed in WO 2014/013260.

The two or more helicases in the series may be separate from one another. The two or more helicases in the series may be brought together by a transmembrane pore as the polynucleotide moves through the pore. The two or more helicases in the series may contact one another.

The two or more helicases are preferably not attached to one another except via the polynucleotide. The two or more helicases are preferably not covalently attached to one another.

The two or more helicases may be attached or covalently attached to one another. The helicases may be attached in any order and using any method. A series of attached helicases may be called a train.

Polynucleotides to which the series of the invention may be attached/bound are discussed in more detail below.

### Methods of the Invention

The invention provides a method of controlling the movement of a target polynucleotide. The method comprises contacting the target polynucleotide with a Dda helicase, a modified helicase of the invention or a construct of the invention and thereby controlling the movement of the polynucleotide. The method is preferably carried out with a potential applied across the pore. As discussed in more detail below, the applied potential typically results in the formation of a complex between the pore and the helicase or construct. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11; 129(27):8650-5.

The invention also provides a method of characterising a target polynucleotide. The method comprises (a) contacting the target polynucleotide with a transmembrane pore and a Dda helicase, a modified helicase of the invention or a construct of the invention such that the helicase or construct controls the movement of the target polynucleotide through the pore. The method also comprises (b) taking one or more measurements as the polynucleotide moves with respect to the pore wherein the measurements are indicative of one or more characteristics of the target polynucleotide and thereby characterising the target polynucleotide.

In all of the methods of the invention, the helicase may be any of those discussed above with reference to the constructs of the invention, including the modified Dda helicases of the invention and Dda helicases which are not modified in accordance with the invention.

Any number of Dda helicases of the invention may be used in these methods. For instance, 1, 2, 3, 4, 5, 6, 7, 8, 9, 10 or more helicases may be used. If two or more Dda helicases of the invention are used, they may be the same or different. Suitable numbers and combinations are discussed above with reference to the series of the invention. These equally apply to the methods of the invention.

If two or more helicases are used, they may be attached to one another. The two or more helicases may be covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259) and PCT/GB2013/051928 (published as WO 2014/013262); and in UK Application No. 1318464.3 filed on 18 Oct. 2013.

If two or more helicases are used, they are preferably not attached to one another except via the polynucleotide. The two or more helicases are more preferably not covalently attached to one another.

Steps (a) and (b) are preferably carried out with a potential applied across the pore as discussed above. In some instances, the current passing through the pore as the polynucleotide moves with respect to the pore is used to determine the sequence of the target polynucleotide. This is Strand Sequencing.

The method of the invention is for characterising a target polynucleotide. A polynucleotide is defined above.

The whole or only part of the target polynucleotide may be characterised using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, sea water or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well in the membrane along which or into which hydrated ions may flow.

Any transmembrane pore may be used in the invention. The pore may be biological or artificial. Suitable pores include, but are not limited to, protein pores, polynucleotide pores and solid state pores.

Any membrane may be used in accordance with the invention. Suitable membranes are well-known in the art. The membrane is preferably an amphiphilic layer. An amphiphilic layer is a layer formed from amphiphilic molecules, such as phospholipids, which have both at least one hydrophilic portion and at least one lipophilic or hydrophobic portion. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer.

The amphiphilic layer may be a monolayer or a bilayer. The amphiphilic layer is typically a planar lipid bilayer or a supported bilayer.

The amphiphilic layer is typically a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Examples. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in International Application No. PCT/US2008/010637 (published as WO 2009/035647).

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial lipid bilayer. The layer may comprise other transmembrane and/or intramembrane proteins as well as other molecules in addition to the pore. Suitable apparatus and conditions are discussed below. The method of the invention is typically carried out in vitro.

The polynucleotide may be coupled to the membrane. This may be done using any known method. If the membrane is an amphiphilic layer, such as a lipid bilayer (as discussed in detail above), the polynucleotide is preferably coupled to the membrane via a polypeptide present in the membrane or a hydrophobic anchor present in the membrane. The hydrophobic anchor is preferably a lipid, fatty acid, sterol, carbon nanotube or amino acid.

The polynucleotide may be coupled directly to the membrane. The polynucleotide is preferably coupled to the membrane via a linker. Preferred linkers include, but are not limited to, polymers, such as polynucleotides, polyethylene glycols (PEGs) and polypeptides. If a polynucleotide is coupled directly to the membrane, then some data will be lost as the characterising run cannot continue to the end of the polynucleotide due to the distance between the membrane and the helicase. If a linker is used, then the polynucleotide can be processed to completion. If a linker is used, the linker may be attached to the polynucleotide at any position. The linker is typically attached to the polynucleotide at the tail polymer.

The coupling may be stable or transient. For certain applications, the transient nature of the coupling is preferred. If a stable coupling molecule were attached directly to either the 5′ or 3′ end of a polynucleotide, then some data will be lost as the characterising run cannot continue to the end of the polynucleotide due to the distance between the membrane and the helicase's active site. If the coupling is transient, then when the coupled end randomly becomes free of the membrane, then the polynucleotide can be processed to completion. Chemical groups that form stable or transient links with the membrane are discussed in more detail below. The polynucleotide may be transiently coupled to an amphiphilic layer, such as a lipid bilayer using cholesterol or a fatty acyl chain. Any fatty acyl chain having a length of from 6 to 30 carbon atoms, such as hexadecanoic acid, may be used.

In preferred embodiments, the polynucleotide is coupled to an amphiphilic layer. Coupling of polynucleotides to synthetic lipid bilayers has been carried out previously with various different tethering strategies. These are summarised in Table 4 below.

TABLE 4

| Attach- | | |
|---|---|---|
| ment | Type of | |
| group | coupling | Reference |
| Thiol | Stable | Yoshina-Ishii, C. and S. G. Boxer (2003). “Arrays |
| | | of mobile tethered vesicles on supported lipid |
| | | bilayers.” J Am Chem Soc 125(13): 3696-7. |
| Biotin | Stable | Nikolov, V., R. Lipowsky, et al. (2007). “Behavior |
| | | of giant vesicles with anchored DNA molecules.” |
| | | Biophys J 92(12): 4356-68 |
| Choles- | Transient | Pfeiffer, I. and F. Hook (2004). “Bivalent |
| terol | | cholesterol-based coupling of oligonucletides to |
| | | lipid membrane assemblies.” J Am Chem Soc |
| | | 126(33): 10224-5 |
| Lipid | Stable | van Lengerich, B., R. J. Rawle, et al. “Covalent |
| | | attachment of lipid vesicles to a fluid-supported |
| | | bilayer allows observation of DNA-mediated |
| | | vesicle interactions.” Langmuir 26(11): 8666-72 |

Polynucleotides may be functionalized using a modified phosphoramidite in the synthesis reaction, which is easily compatible for the addition of reactive groups, such as thiol, cholesterol, lipid and biotin groups. These different attachment chemistries give a suite of attachment options for polynucleotides. Each different modification group tethers the polynucleotide in a slightly different way and coupling is not always permanent so giving different dwell times for the polynucleotide to the membrane. The advantages of transient coupling are discussed above.

Coupling of polynucleotides can also be achieved by a number of other means provided that a reactive group can be added to the polynucleotide. The addition of reactive groups to either end of DNA has been reported previously. A thiol group can be added to the 5′ of ssDNA using polynucleotide kinase and ATPTS (Grant, G. P. and P. Z. Qin (2007). “A facile method for attaching nitroxide spin labels at the 5′ terminus of nucleic acids.” Nucleic Acids Res 35(10): e77). A more diverse selection of chemical groups, such as biotin, thiols and fluorophores, can be added using terminal transferase to incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169(2): 376-82).

Alternatively, the reactive group could be considered to be the addition of a short piece of DNA complementary to one already coupled to the membrane, so that attachment can be achieved via hybridisation. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89(20): 9823-5). Alternatively either ssDNA or dsDNA could be ligated to native dsDNA and then the two strands separated by thermal or chemical denaturation. To native dsDNA, it is possible to add either a piece of ssDNA to one or both of the ends of the duplex, or dsDNA to one or both ends. Then, when the duplex is melted, each single strand will have either a 5′ or 3′ modification if ssDNA was used for ligation or a modification at the 5′ end, the 3′ end or both if dsDNA was used for ligation. If the polynucleotide is a synthetic strand, the coupling chemistry can be incorporated during the chemical synthesis of the polynucleotide. For instance, the polynucleotide can be synthesized using a primer with a reactive group attached to it.

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. By using an antisense primer that has a reactive group, such as a cholesterol, thiol, biotin or lipid, each copy of the amplified target DNA will contain a reactive group for coupling.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as at least 6, at least 7, at least 8 or at least 9 subunits. The pore is preferably made up of 6, 7, 8 or 9 subunits. The pore is preferably a hexameric, heptameric, octameric or nonameric pore. The pore may be a homo-oligomer or a hetero-oligomer.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from Msp or from α-hemolysin (α-HL).

The transmembrane protein pore is preferably derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp typically comprises the sequence shown in SEQ ID NO: 2 or a variant thereof. SEQ ID NO: 2 is the MS-(B1)8 mutant of the MspA monomer. It includes the following mutations: D90N, D91N, D93N, D118R, D134R and E139K. A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (ncbi.nlm.nih.gov).

SEQ ID NO: 2 is the MS-(B1)8 mutant of the MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB: A138P. The variant may comprise one or more of the following substitutions present in MspC: A96G, N102E and A138P. The variant may comprise one or more of the following mutations present in MspD: Deletion of G1, L2V, E5Q, L8V, D13G, W21A, D22E, K47T, I49H, I68V, D91G, A96Q, N102D, S103T, V104I, S136K and G141A. The variant may comprise combinations of one or more of the mutations and substitutions from Msp B, C and D. The variant preferably comprises the mutation L88N. A variant of SEQ ID NO: 2 has the mutation L88N in addition to all the mutations of MS-(B1)8 and is called MS-(B2)8. The pore used in the invention is preferably MS-(B2)8. The further preferred variant comprises the mutations G75S/G77S/L88N/Q126R. The variant of SEQ ID NO: 2 has the mutations G75S/G77S/L88N/Q126R in addition to all the mutations of MS-(B1)8 and is called MS-(B2C)8. The pore used in the invention is preferably MS-(B2)8 or MS-(B2C)8.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 5 below. Where amino acids have similar polarity, this can also be determined by reference to the hydropathy scale for amino acid side chains in Table 6.

TABLE 5
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, charged (+) |
| Ile | aliphatic, hydrophobic, neutral |
| Lys | polar, hydrophilic, charged(+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE 6
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may include fragments of SEQ ID NO: 2. Such fragments retain pore forming activity. Fragments may be at least 50, 100, 150 or 200 amino acids in length. Such fragments may be used to produce the pores. A fragment preferably comprises the pore forming domain of SEQ ID NO: 2. Fragments must include one of residues 88, 90, 91, 105, 118 and 134 of SEQ ID NO: 2. Typically, fragments include all of residues 88, 90, 91, 105, 118 and 134 of SEQ ID NO: 2.

One or more amino acids may be alternatively or additionally added to the polypeptides described above. An extension may be provided at the amino terminal or carboxy terminal of the amino acid sequence of SEQ ID NO: 2 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 2 and which retains its ability to form a pore. A variant typically contains the regions of SEQ ID NO: 2 that are responsible for pore formation. The pore forming ability of Msp, which contains a β-barrel, is provided by β-sheets in each subunit. A variant of SEQ ID NO: 2 typically comprises the regions in SEQ ID NO: 2 that form β-sheets. One or more modifications can be made to the regions of SEQ ID NO: 2 that form β-sheets as long as the resulting variant retains its ability to form a pore. A variant of SEQ ID NO: 2 preferably includes one or more modifications, such as substitutions, additions or deletions, within its α-helices and/or loop regions.

The monomers derived from Msp may be modified to assist their identification or purification, for example by the addition of histidine residues (a hist tag), aspartic acid residues (an asp tag), a streptavidin tag or a flag tag, or by the addition of a signal sequence to promote their secretion from a cell where the polypeptide does not naturally contain such a sequence. An alternative to introducing a genetic tag is to chemically react a tag onto a native or engineered position on the pore. An example of this would be to react a gel-shift reagent to a cysteine engineered on the outside of the pore. This has been demonstrated as a method for separating hemolysin hetero-oligomers (Chem Biol. 1997 July; 4(7):497-505).

The monomer derived from Msp may be labelled with a revealing label. The revealing label may be any suitable label which allows the pore to be detected. Suitable labels are described above.

The monomer derived from Msp may also be produced using D-amino acids. For instance, the monomer derived from Msp may comprise a mixture of L-amino acids and D-amino acids. This is conventional in the art for producing such proteins or peptides.

The monomer derived from Msp contains one or more specific modifications to facilitate nucleotide discrimination. The monomer derived from Msp may also contain other non-specific modifications as long as they do not interfere with pore formation. A number of non-specific side chain modifications are known in the art and may be made to the side chains of the monomer derived from Msp. Such modifications include, for example, reductive alkylation of amino acids by reaction with an aldehyde followed by reduction with NaBH₄, amidination with methylacetimidate or acylation with acetic anhydride.

The monomer derived from Msp can be produced using standard methods known in the art. The monomer derived from Msp may be made synthetically or by recombinant means. For example, the pore may be synthesized by in vitro translation and transcription (IVTT). Suitable methods for producing pores are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

The transmembrane protein pore is also preferably derived from α-hemolysin (α-HL). The wild type α-HL pore is formed of seven identical monomers or subunits (i.e. it is heptameric). The sequence of one monomer or subunit of α-hemolysin-NN is shown in SEQ ID NO: 4. The transmembrane protein pore preferably comprises seven monomers each comprising the sequence shown in SEQ ID NO: 4 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 4 form loop regions. Residues 113 and 147 of SEQ ID NO: 4 form part of a constriction of the barrel or channel of α-HL.

In such embodiments, a pore comprising seven proteins or monomers each comprising the sequence shown in SEQ ID NO: 4 or a variant thereof are preferably used in the method of the invention. The seven proteins may be the same (homo-heptamer) or different (hetero-heptamer).

A variant of SEQ ID NO: 4 is a protein that has an amino acid sequence which varies from that of SEQ ID NO: 4 and which retains its pore forming ability. The ability of a variant to form a pore can be assayed using any method known in the art. For instance, the variant may be inserted into an amphiphilic layer, such as a lipid bilayer, along with other appropriate subunits and its ability to oligomerise to form a pore may be determined. Methods are known in the art for inserting subunits into amphiphilic layers, such as lipid bilayers. Suitable methods are discussed above.

The variant may include modifications that facilitate covalent attachment to or interaction with the helicase or construct. The variant preferably comprises one or more reactive cysteine residues that facilitate attachment to the helicase or construct. For instance, the variant may include a cysteine at one or more of positions 8, 9, 17, 18, 19, 44, 45, 50, 51, 237, 239 and 287 and/or on the amino or carboxy terminus of SEQ ID NO: 4. Preferred variants comprise a substitution of the residue at position 8, 9, 17, 237, 239 and 287 of SEQ ID NO: 4 with cysteine (A8C, T9C, N17C, K237C, S239C or E287C). The variant is preferably any one of the variants described in International Application No. PCT/GB09/001690 (published as WO 2010/004273), PCT/GB09/001679 (published as WO 2010/004265) or PCT/GB10/000133 (published as WO 2010/086603).

The variant may also include modifications that facilitate any interaction with nucleotides.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 4, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 4 over the entire sequence. There may be at least 80%, for example at least 85%, 90% or 95%, amino acid identity over a stretch of 200 or more, for example 230, 250, 270 or 280 or more, contiguous amino acids (“hard homology”). Homology can be determined as discussed above.

Amino acid substitutions may be made to the amino acid sequence of SEQ ID NO: 4 in addition to those discussed above, for example up to 1, 2, 3, 4, 5, 10, 20 or 30 substitutions. Conservative substitutions may be made as discussed above.

One or more amino acid residues of the amino acid sequence of SEQ ID NO: 4 may additionally be deleted from the polypeptides described above. Up to 1, 2, 3, 4, 5, 10, 20 or 30 residues may be deleted, or more.

Variants may be fragments of SEQ ID NO: 4. Such fragments retain pore-forming activity. Fragments may be at least 50, 100, 200 or 250 amino acids in length. A fragment preferably comprises the pore-forming domain of SEQ ID NO: 4. Fragments typically include residues 119, 121, 135. 113 and 139 of SEQ ID NO: 4.

One or more amino acids may be alternatively or additionally added to the polypeptides described above. An extension may be provided at the amino terminus or carboxy terminus of the amino acid sequence of SEQ ID NO: 4 or a variant or fragment thereof. The extension may be quite short, for example from 1 to 10 amino acids in length. Alternatively, the extension may be longer, for example up to 50 or 100 amino acids. A carrier protein may be fused to a pore or variant.

As discussed above, a variant of SEQ ID NO: 4 is a subunit that has an amino acid sequence which varies from that of SEQ ID NO: 4 and which retains its ability to form a pore. A variant typically contains the regions of SEQ ID NO: 4 that are responsible for pore formation. The pore forming ability of α-HL, which contains a β-barrel, is provided by β-strands in each subunit. A variant of SEQ ID NO: 4 typically comprises the regions in SEQ ID NO: 4 that form β-strands. The amino acids of SEQ ID NO: 4 that form β-strands are discussed above. One or more modifications can be made to the regions of SEQ ID NO: 4 that form β-strands as long as the resulting variant retains its ability to form a pore. Specific modifications that can be made to the β-strand regions of SEQ ID NO: 4 are discussed above.

A variant of SEQ ID NO: 4 preferably includes one or more modifications, such as substitutions, additions or deletions, within its α-helices and/or loop regions. Amino acids that form α-helices and loops are discussed above.

The variant may be modified to assist its identification or purification as discussed above.

Pores derived from α-HL can be made as discussed above with reference to pores derived from Msp.

In some embodiments, the transmembrane protein pore is chemically modified. The pore can be chemically modified in any way and at any site. The transmembrane protein pore is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The transmembrane protein pore may be chemically modified by the attachment of any molecule. For instance, the pore may be chemically modified by attachment of a dye or a fluorophore.

Any number of the monomers in the pore may be chemically modified. One or more, such as 2, 3, 4, 5, 6, 7, 8, 9 or 10, of the monomers is preferably chemically modified as discussed above.

The reactivity of cysteine residues may be enhanced by modification of the adjacent residues. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the pore before a linker is attached.

The molecule (with which the pore is chemically modified) may be attached directly to the pore or attached via a linker as disclosed in International Application Nos. PCT/GB09/001690 (published as WO 2010/004273), PCT/GB09/001679 (published as WO 2010/004265) or PCT/GB10/000133 (published as WO 2010/086603).

The helicase or construct may be covalently attached to the pore. The helicase or construct is preferably not covalently attached to the pore. The application of a voltage to the pore and helicase or construct typically results in the formation of a sensor that is capable of sequencing target polynucleotides. This is discussed in more detail below.

Any of the proteins described herein, i.e. the helicases, the transmembrane protein pores or constructs, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the helicase, pore or construct. An example of this would be to react a gel-shift reagent to a cysteine engineered on the outside of the pore. This has been demonstrated as a method for separating hemolysin hetero-oligomers (Chem Biol. 1997 July; 4(7):497-505).

The helicase, pore or construct may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Proteins may be made synthetically or by recombinant means. For example, the helicase, pore or construct may be synthesized by in vitro translation and transcription (IVTT). The amino acid sequence of the helicase, pore or construct may be modified to include non-naturally occurring amino acids or to increase the stability of the protein. When a protein is produced by synthetic means, such amino acids may be introduced during production. The helicase, pore or construct may also be altered following either synthetic or recombinant production.

The helicase, pore or construct may also be produced using D-amino acids. For instance, the pore or construct may comprise a mixture of L-amino acids and D-amino acids. This is conventional in the art for producing such proteins or peptides.

The helicase, pore or construct may also contain other non-specific modifications as long as they do not interfere with pore formation or helicase or construct function. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

The helicase, pore and construct can be produced using standard methods known in the art. Polynucleotide sequences encoding a helicase, pore or construct may be derived and replicated using standard methods in the art. Polynucleotide sequences encoding a helicase, pore or construct may be expressed in a bacterial host cell using standard techniques in the art. The helicase, pore and/or construct may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The helicase, pore and/or construct may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

The method of the invention involves measuring one or more characteristics of the target polynucleotide. The method may involve measuring two, three, four or five or more characteristics of the target polynucleotide. The one or more characteristics are preferably selected from (i) the length of the target polynucleotide, (ii) the identity of the target polynucleotide, (iii) the sequence of the target polynucleotide, (iv) the secondary structure of the target polynucleotide and (v) whether or not the target polynucleotide is modified. Any combination of (i) to (v) may be measured in accordance with the invention.

For (i), the length of the polynucleotide may be measured for example by determining the number of interactions between the target polynucleotide and the pore or the duration of interaction between the target polynucleotide and the pore.

For (ii), the identity of the polynucleotide may be measured in a number of ways. The identity of the polynucleotide may be measured in conjunction with measurement of the sequence of the target polynucleotide or without measurement of the sequence of the target polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the target polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106(19):7702-7, Lieberman K R et al, J Am Chem Soc. 2010; 132(50):17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the target polynucleotide is modified by methylation, by oxidation, by damage, with one or more proteins or with one or more labels, tags or spacers. Specific modifications will result in specific interactions with the pore which can be measured using the methods described below. For instance, methylcytosine may be distinguished from cytosine on the basis of the current flowing through the pore during its interaction with each nucleotide.

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and International Application WO-2000/28312. Alternatively, electrical measurements may be made using a multi-channel system, for example as described in International Application WO-2009/077734 and International Application WO-2011/067559.

In a preferred embodiment, the method comprises:
(a) contacting the target polynucleotide with a transmembrane pore and a helicase of the invention or a construct of the invention such that the target polynucleotide moves through the pore and the helicase or construct controls the movement of the target polynucleotide through the pore; and
(b) measuring the current passing through the pore as the polynucleotide moves with respect to the pore wherein the current is indicative of one or more characteristics of the target polynucleotide and thereby characterising the target polynucleotide.

The methods may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The methods may be carried out using the apparatus described in International Application No. PCT/GB08/000562 (WO 2008/102120).

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Examples. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. Hel308, XPD, RecD and TraI helicases surprisingly work under high salt concentrations. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffer include, but are not limited to, HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

The method may be carried out in the presence of free nucleotides or free nucleotide analogues and/or an enzyme cofactor that facilitates the action of the helicase or construct. The method may also be carried out in the absence of free nucleotides or free nucleotide analogues and in the absence of an enzyme cofactor. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the helicase or construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The target polynucleotide may be contacted with the helicase or construct and the pore in any order. In is preferred that, when the target polynucleotide is contacted with the helicase or construct and the pore, the target polynucleotide firstly forms a complex with the helicase or construct. When the voltage is applied across the pore, the target polynucleotide/helicase or construct complex then forms a complex with the pore and controls the movement of the polynucleotide through the pore.

### Other Methods

The invention also provides a method of forming a sensor for characterising a target polynucleotide. The method comprises forming a complex between a pore and a Dda helicase, a helicase of the invention or a construct of the invention. The helicase may be any of those discussed above with reference to the constructs of the invention, including the helicases of the invention and helicases which are not modified in accordance with the invention. Any number and combination of Dda helicases of the invention discussed above with reference to the series and methods of the invention may be used.

The complex may be formed by contacting the pore and the helicase or construct in the presence of the target polynucleotide and then applying a potential across the pore. The applied potential may be a chemical potential or a voltage potential as described above. Alternatively, the complex may be formed by covalently attaching the pore to the helicase or construct. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target polynucleotide. The method preferably comprises forming a complex between a pore derived from Msp and a helicase of the invention or a construct of the invention. Any of the embodiments discussed above with reference to the methods of the invention equally apply to this method. The invention also provides a sensor produced using the method of the invention.

### Kits

The present invention also provides a kit for characterising a target polynucleotide. The kit comprises (a) a pore and (b) a Dda helicase, a helicase of the invention of the invention or a construct of the invention. Any of the embodiments discussed above with reference to the method of the invention equally apply to the kits. The helicase may be any of those discussed above with reference to the constructs of the invention, including the helicases of the invention and helicases which are not modified in accordance with the invention. The kit may comprise any number and combination of Dda helicases of the invention discussed above with reference to the series and methods of the invention.

The kit may further comprise the components of a membrane, such as the phospholipids needed to form an amphiphilic layer, such as a lipid bilayer.

The kit of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

### Apparatus

The invention also provides an apparatus for characterising a target polynucleotide. The apparatus comprises a plurality of pores and a plurality of Dda helicases, a plurality of helicases of the invention or a plurality of constructs of the invention. The apparatus preferably further comprises instructions for carrying out the method of the invention. The apparatus may be any conventional apparatus for polynucleotide analysis, such as an array or a chip. Any of the embodiments discussed above with reference to the methods of the invention are equally applicable to the apparatus of the invention. The helicase may be any of those discussed above with reference to the constructs of the invention, including the helicases of the invention and helicases which are not modified in accordance with the invention. The apparatus may comprise any number and combination of Dda helicases of the invention discussed above with reference to the series and methods of the invention.

The apparatus is preferably set up to carry out the method of the invention.

The apparatus preferably comprises:
a sensor device that is capable of supporting the plurality of pores and being operable to perform polynucleotide characterisation using the pores and helicases or constructs; and
at least one port for delivery of the material for performing the characterisation.

Alternatively, the apparatus preferably comprises:
a sensor device that is capable of supporting the plurality of pores and being operable to perform polynucleotide characterisation using the pores and helicases or constructs; and
at least one reservoir for holding material for performing the characterisation.

The apparatus more preferably comprises:
a sensor device that is capable of supporting the membrane and plurality of pores and being operable to perform polynucleotide characterising using the pores and helicases or constructs;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device.
   The apparatus may be any of those described in International Application No. PCT/GB08/004127 (published as WO 2009/077734), PCT/GB10/000789 (published as WO 2010/122293), International Application No. PCT/GB10/002206 (published as WO 2011/067559) or International Application No. PCT/US99/25679 (published as WO 00/28312).

### Methods of Producing Helicases of the Invention

The invention also provides methods of producing a modified helicase of the invention. The method comprises providing a Dda helicase and modifying the helicase to form a modified helicase of the invention.

The method preferably further comprises determining whether or not the helicase is capable of controlling the movement of a polynucleotide. Assays for doing this are described above. If the movement of a polynucleotide can be controlled, the helicase has been modified correctly and a helicase of the invention has been produced. If the movement of a polynucleotide cannot be controlled, a helicase of the invention has not been produced.

### Methods of Producing Constructs of the Invention

The invention also provides a method of producing a construct of the invention. The method comprises attaching, preferably covalently attaching, a Dda helicase or a helicase of the invention to an additional polynucleotide binding moiety. Any of the helicases and moieties discussed above can be used in the methods. The site of and method of covalent attachment are selected as discussed above.

The method preferably further comprises determining whether or not the construct is capable of controlling the movement of a polynucleotide. Assays for doing this are described above. If the movement of a polynucleotide can be controlled, the helicase and moiety have been attached correctly and a construct of the invention has been produced. If the movement of a polynucleotide cannot be controlled, a construct of the invention has not been produced.

The following Examples illustrate the invention.

### EXAMPLES

### Example 1

This example describes how a T4 Dda—E94C/A360C (SEQ ID NO: 8 with mutations E94C/A360C and then (ΔM1)G1G2) controlled the movement of intact DNA strands through a single MspA nanopore (MS(B1-G75S/G77S/L88N/Q126R)8 MspA (MspA-B2C) (SEQ ID NO: 2 with mutations G75S/G77S/L88N/Q126R).

### Materials and Methods

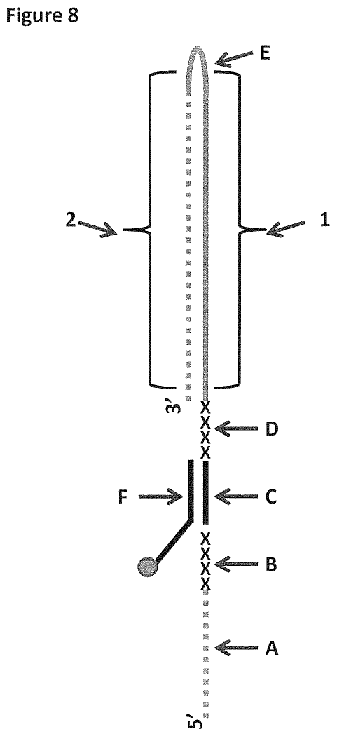

Prior to setting up the experiment, the Lambda DNA construct (SEQ ID NO: 60 attached by its 3′ end to four iSpC3 spacers which are attached to the 5′ end of SEQ ID NO: 61 which is attached at its 3′ end to four iSPC3 spacers which are attached to the 5′ end of SEQ ID NO: 62, the SEQ ID NO: 61 region of this construct is hybridised to SEQ ID NO: 63 (which has a 3′ cholesterol tether) see FIG. 8 for a diagram of the construct) and T4 Dda—E94C/A360C were pre-incubated together for 15 minutes at 23° C. in buffer (20 mM CAPS, pH 10.0, 500 mM NaCl, 5% Glycerol, 2 mM DTT).

Electrical measurements were acquired at 20° C. (by placing the experimental system on a cooler plate) from single MspA nanopores (MspA-B2C) inserted in block co-polymer in buffer (600 mM KCl, 25 mM potassium phosphate, 75 mM Potassium Ferrocyanide (II), 25 mM Potassium ferricyanide (III), pH 8). After achieving a single pore inserted in the block co-polymer, then buffer (1 mL, 600 mM KCl, 25 mM potassium phosphate, 75 mM Potassium Ferrocyanide (II), 25 mM Potassium ferricyanide (III), pH 8) was flowed through the system to remove any excess MspA nanopores (MspA-B2C) and finally experimental buffer was flowed into the system (2 mL 960 mM KCl, 25 mM potassium phosphate, 3 mM Potassium Ferrocyanide (II), 1 mM Potassium ferricyanide (III), pH 8). MgCl₂ (10 mM final concentration) and ATP (1 mM final concentration) were mixed together with buffer (960 mM KCl, 25 mM potassium phosphate, 3 mM Potassium Ferrocyanide (II), 1 mM Potassium ferricyanide (III), pH 8) and then added to the Lambda DNA construct (0.2 nM final concentration), T4 Dda—E94C/A360C (10 nM final concentration) buffer (20 mM CAPS, pH 10.0, 500 mM NaCl, 5% Glycerol, 2 mM DTT) pre-mix. The pre-mix was then added to the single nanopore experimental system. Experiments were carried out for four hours following a potential flip process (+100 mV for 2 s, then 0 V for 2 s, then −120 mV for 14500s applied at the cis side) and helicase-controlled DNA movement was monitored.

### Results and Discussion

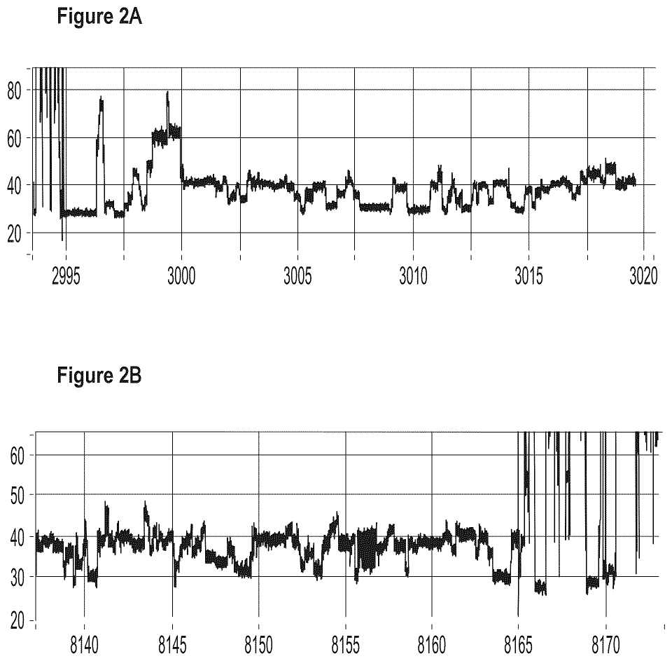

Helicase controlled DNA movement was observed for the Lambda DNA construct, an example of a helicase-controlled DNA movement is shown in FIG. 1. The helicase-controlled DNA movement was 5170 seconds long and corresponded to the translocation of approximately 30 kB of the lambda construct through the nanopore. FIG. 2 shows zoomed in regions of the beginning (a) and end (b) of the helicase-controlled DNA movement.

### Example 2

This example describes how a T4 Dda—E94C/A360C exhibited tight binding to both linear (SEQ ID NO: 64) and circular (SEQ ID NO: 65) single-stranded DNA. The tight binding of the enzyme was measured using a fluorescence anisotropy-based assay.

### Materials and Methods

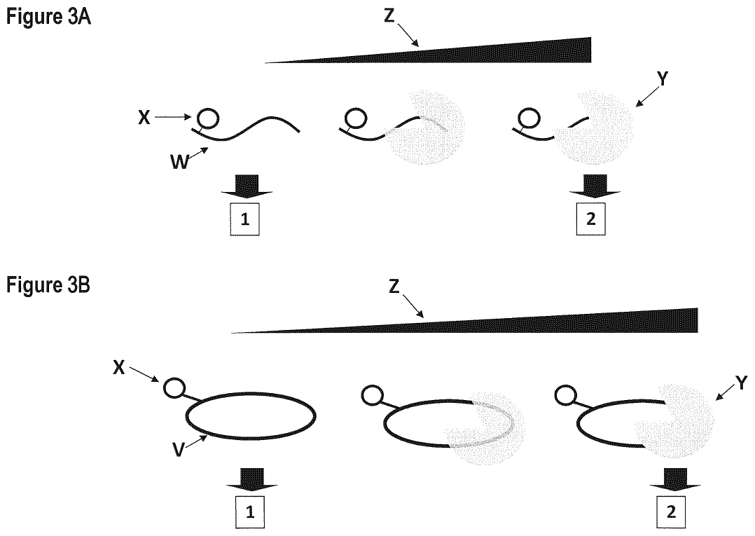

Two custom fluorescent substrates were used to assess the ability of T4 Dda—E94C/A360C helicase to bind to linear (SEQ ID NO: 64) and circular (SEQ ID NO: 65) single-stranded DNA. The 44 nt linear single-stranded DNA substrate (1 nM final, SEQ ID NO: 64) had a carboxyfluorescein (FAM) attached to the thymine base at position 37 in SEQ ID NO: 64. The 75 nt circular single-stranded DNA substrate (1 nM final, SEQ ID NO: 65) had a carboxyfluorescein (FAM) attached to a thymine base in SEQ ID NO: 65. As the helicase bound to either fluorescent substrate in a buffered solution (25 mM potassium phosphate, 151.5 mM KCl, pH8.0, 10 mM MgCl₂), the fluorescence anisotropy (a property relating to the speed of tumbling of the DNA substrate in solution) increased. The lower the amount of helicase needed to effect an increase in anisotropy, the tighter the binding affinity between the DNA and helicase (FIGS. 3A-3B).

T4 Dda—E94C/A360C was buffer exchanged into the binding buffer (25 mM potassium phosphate, 151.5 mM KCl, pH8.0, 10 mM MgCl₂) and then serially diluted over a concentration range of 0.02 nM to 750 nM. Each sample concentration was then mixed with linear or circular single-stranded DNA (1 nM of SEQ ID NO: 64 or 65) giving a final concentration range of T4 Dda—E94C/A360C of 0.01 nM to 375 nM and the fluorescence anisotropy assessed over the course of 60 min at 25° C.

### Results and Discussion

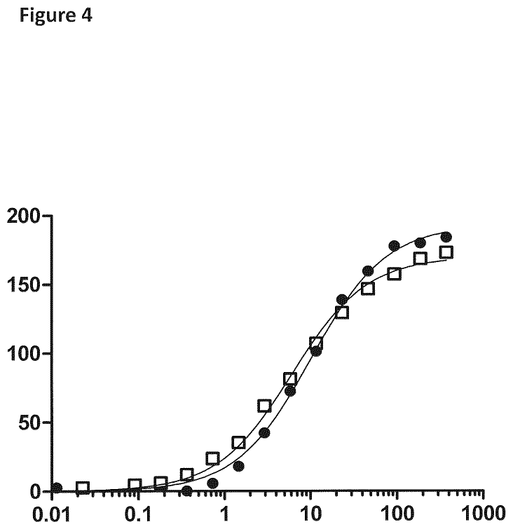

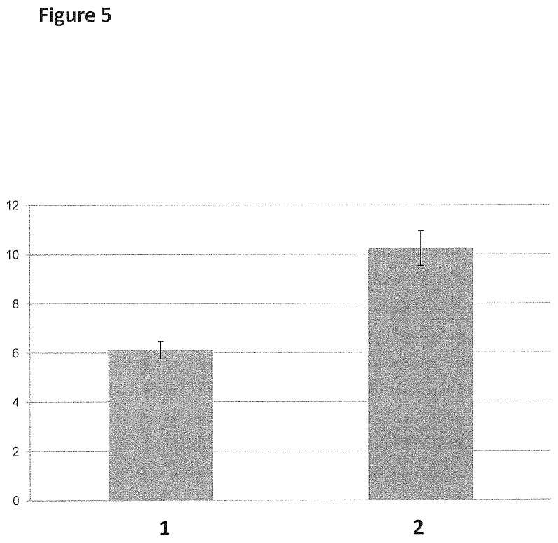

FIGS. 4 and 5 show the fluorescence binding assay data collected for the linear and circular single-stranded DNA binding experiments. FIG. 4 shows the change in anisotropy of the linear and circular single-stranded DNA oligonucleotides (SEQ ID NO: 64 or 65) with increasing amounts of T4 Dda—E94C/A360C at the end of a 60 minute incubation period. FIG. 5 shows the equilibrium dissociation constants (K_{d}) for T4 Dda—E94C/A360C binding to linear or circular single-stranded DNA after a 60 minute incubation, obtained through fitting one phase dissociation binding curves through the data shown in FIG. 4 using Graphpad Prism software (y-axis label=dissociation constant Kd (nM), x-axis label=Ref. Number, where Ref. Number 1 corresponded to the linear single-stranded DNA oligonucleotide and Ref. Number 2 corresponded to the circular single-stranded DNA oligonucleotide).

The T4 Dda—E94C/A360C helicase was found to exhibit tight binding affinity (sub 15 nM binding affinity) to both circular and linear single-stranded DNA (see FIGS. 4 and 5).

### Example 3

This example compared the helicase-controlled DNA movement of T4 Dda—E94C/A360C with that of TrwC Cba (SEQ ID NO: 66). Both helicases move along the polynucleotide in a 5′ to 3′ direction. When the 5′end of the polynucleotide (the end away from which the helicases move) is captured by the pore, the helicases work with the direction of the field resulting from the applied potential and move the threaded polynucleotide into the pore and into the trans chamber. T4 Dda was observed to control the translocation of DNA through the nanopore smoothly without the DNA stepping back (i.e. towards its 3′end relative the pore), whereas TrwC Cba resulted in stepping back of the DNA between states as it controlled translocation of the DNA. In this Example, stepping back involves the DNA moving backwards relative to the pore (i.e. towards its 5′ and away from it 3′ end in this Example). This phenomenon was called slipping in UK Application Nos. 1318464.3 and 1404718.7.

### Materials and Methods

Prior to setting up the experiments, the DNA strand (3 uL of 20 nM, SEQ ID NO: 67 attached by its 3′ end to four iSpC3 spacers which are attached to the 5′ end of SEQ ID NO: 61 which is attached at its 3′ end to four 5-nitroindoles the last of which is attached to the 5′ end of SEQ ID NO: 68, in addition SEQ ID NO: 63 is hybridised to SEQ ID NO: 61) and TrwC Cba (SEQ ID NO: 66, 22.5 uL of 13.3 μM) were pre-incubated together for over an hour at room temperature in buffer (50 mM CAPS, pH 10.0, 100 mM NaCl). In a separate tube, 3 μL of MgCl2 (1 M) and 3 μL of dTTP (100 mM) were mixed with 260 μL of buffer (960 mM KCl, 3 mM potassium ferrocyanide (II), 1 mM potassium ferricyanide (III) and 25 mM potassium phosphate pH 8.0). After the hour pre-incubation, the DNA enzyme mix was added to MgCl2/dTTP mix giving final concentrations of reagents as follows—DNA strand (0.2 nM), TrwC Cba (SEQ ID NO: 66, 1 μM), MgCl2 (10 mM), dTTP (1 mM) in buffer (960 mM KCl, 3 mM potassium ferrocyanide (II), 1 mM potassium ferricyanide (III) and 25 mM potassium phosphate pH 8.0). Prior to setting up the experiments, the DNA strand (0.2 uL of 300 nM, SEQ ID NO: 67 attached by its 3′ end to four iSpC3 spacers which are attached to the 5′ end of SEQ ID NO: 61 which is attached at its 3′ end to four 5-nitroindoles the last of which is attached to the 5′ end of SEQ ID NO: 68, in addition SEQ ID NO: 63 is hybridised to SEQ ID NO: 61) and T4 Dda—E94C/A360C (0.1 uL of 3300 nM) were pre-incubated together for 15 minutes at room temperature. In a separate tube, MgCl2 (3 uL of 1M) and ATP (3 uL of 100 mM) were mixed with 294 μL of buffer (960 mM KCl, 3 mM potassium ferrocyanide (II), 1 mM potassium ferricyanide (III) and 25 mM potassium phosphate, pH 8.0). After the 15 minute pre-incubation, the DNA enzyme mix was added to MgCl2/ATP mix giving final concentrations of reagents as follows—DNA strand (0.2 nM), T4 Dda—E94C/A360C (1 nM), MgCl2 (10 mM), ATP (1 mM) in buffer (960 mM KCl, 3 mM potassium ferrocyanide (II), 1 mM potassium ferricyanide (III) and 25 mM potassium phosphate pH 8.0).

Electrical measurements were acquired at 20° C. (by placing the experimental system on a cooler plate) from single MspA nanopores (MspA-B2C) inserted in block co-polymer in buffer (600 mM KCl, 25 mM potassium phosphate, 75 mM Potassium Ferrocyanide (II), 25 mM Potassium ferricyanide (III), pH 8). After achieving a single pore inserted in the block co-polymer, then buffer (3 mL, 960 mM KCl, 25 mM potassium phosphate, 3 mM Potassium Ferrocyanide (II), 1 mM Potassium ferricyanide (III), pH 8) was flowed through the system to remove any excess MspA nanopores (MspA-B2C). Either the TrwC Cba (SEQ ID NO: 66) or the T4 Dda E94C/A360C pre-mix was then added to the single nanopore experimental system. Each experiment was carried out for 6 hours at a holding potential of −120 mV) and helicase-controlled DNA movement was monitored.

### Results and Discussion

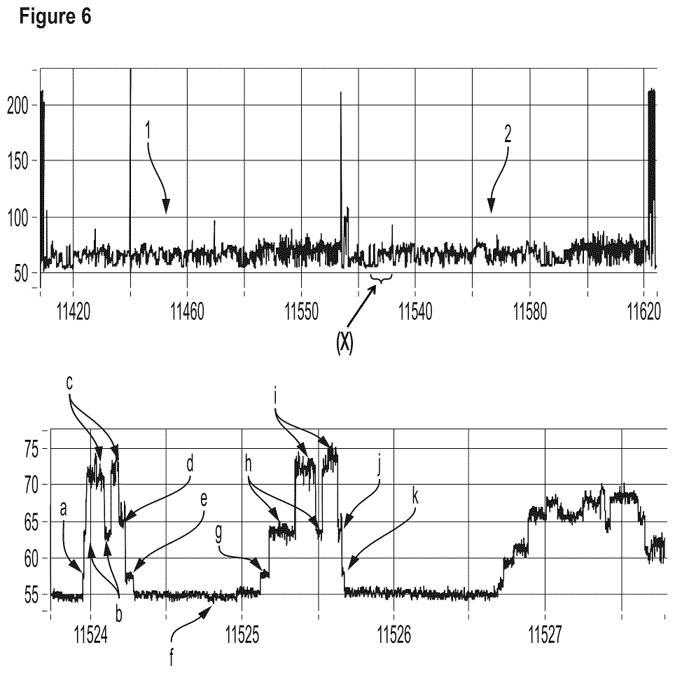

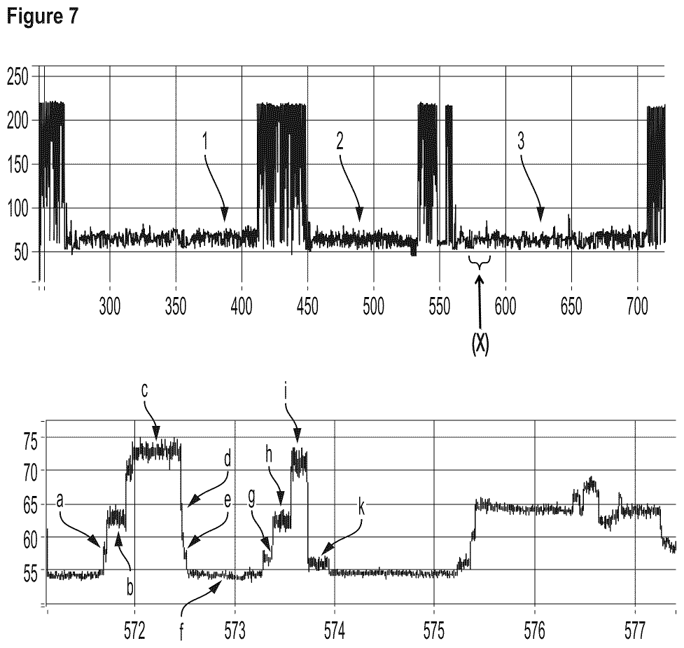

FIGS. 6 and 7 show helicase controlled DNA movements for the TrwC Cba (SEQ ID NO: 66) and T4 Dda E94C/A360C respectively. The upper trace of FIG. 6 shows two TrwC Cba (SEQ ID NO: 66) helicase controlled DNA movements (labelled 1 and 2) and the lower section shows zoomed in region X. The upper trace of FIG. 7 shows three T4 Dda E94C/A360C helicase controlled DNA movements (labelled 1, 2 and 3) and the lower section shows zoomed in region X. The Trwc Cba helicase controlled the movement of the DNA strand through the nanopore and the current changed as the DNA translocated. In the lower trace a number of current levels were labelled a to k which corresponded to consecutive current levels produced when the section of the DNA strand translocated through the pore. It was clear from zoomed in region X in FIG. 6 that the DNA stepped back so that levels corresponding to b, c, h and i were observed several times. Whereas, FIG. 7 lower trace shows that the T4 Dda E94C/A360C helicase controlled the movement of DNA through a nanopore such that stepping back was not observed and a single current level which corresponded to consecutive current levels a to k was observed. It was advantageous to have an enzyme which did not allow stepping back of the DNA strand as this meant it was much easier to map the changes in current to the sequence of the DNA strand when the enzyme moved in one direction along the strand. This made T4 Dda E94C/A360C an improved enzyme for DNA translocation when compared to TrwC Cba (SEQ ID NO: 66).

### Example 4

This example describes how T4 Dda—E94C/A360C, T4 Dda—E94C/A360C/C109A/C136A (SEQ ID NO: 8 with mutations E94C/A360C/C109A/C136A and then (ΔM1)G1G2) and T4 Dda—E94C/A360C/C114A/C171A/C421D (SEQ ID NO: 8 with mutations E94C/A360C/C114A/C171A/C421D and then (ΔM1)G1G2) controlled the movement of intact DNA strands through a single MspA nanopore. The helicase controlled movement speed of both region 1 and region 2 of the lambda DNA construct (shown in FIG. 8) was observed to decrease overtime for T4 Dda—E94C/A360C and T4 Dda—E94C/A360C/C114A/C171A/C421D. However, T4 Dda—E94C/A360C/C109A/C136A exhibited improved helicase controlled DNA movement in comparison as the speed of movement remained high and fairly constant during the entire experimental run.

### Materials and Methods

Prior to setting up the experiment, the DNA construct X (5.2 μL, 25 nM, SEQ ID NO: 67 attached by its 3′ end to four iSpC3 spacers which are attached to the 5′ end of SEQ ID NO: 61 which is attached at its 3′ end to four 5-nitroindoles spacers which are attached to the 5′ end of SEQ ID NO: 69, the SEQ ID NO: 61 region of this construct is hybridised to SEQ ID NO: 63 (which has a 3′ cholesterol tether) this is a similar construct as shown in FIG. 8 except the region labelled A corresponds to SEQ ID NO: 67 and the region labelled E corresponds to SEQ ID NO: 69) in buffer (in 50 mM NaCl, 10 mM Tris pH7.5) was pre-incubated for 5 minutes at ambient temperature with either T4 Dda—E94C/A360C, T4 Dda—E94C/A360C/C109A/C136A or T4 Dda—E94C/A360C/C114A/C171A/C421D in buffer (5.2 μL, 250 nM in 253 mM KCl, 50 mM potassium phosphate pH 8.0 2 mM EDTA). TMAD (2.6 μL, 500 μM) was then added to the DNA/enzyme pre-mix and incubated for a further 5 minutes. Finally, buffer (1241.5 μL, 25 mM potassium phosphate, 150 mM potassium ferrocyanide (II) and 150 mM potassium ferricyanide (III), pH 8.0) MgCl₂ (13 μL, 1M) and ATP (32.5 μL, 100 mM) were added to the pre-mix.

Electrical measurements were acquired from single MspA nanopores inserted in block co-polymer in buffer (25 mM potassium phosphate, 150 mM potassium ferrocyanide (II), 150 mM potassium ferricyanide (III)) at a peltier temperature of 28° C. After achieving a single pore inserted in the block co-polymer, then buffer (2 mL, 25 mM potassium phosphate pH 8.0, 150 mM potassium ferrocyanide (II) and 150 mM potassium ferricyanide (III)) was flowed through the system to remove any excess MspA nanopores. The enzyme (either T4 Dda—E94C/A360C, T4 Dda—E94C/A360C/C109A/C136A or T4 Dda—E94C/A360C/C114A/C171A/C421D (1 nM final concentration)), DNA (0.1 nM final concentration), fuel (MgCl2 10 nM final concentration, ATP 2.5 mM final concentration) pre-mix was then added to the single nanopore experimental system. Each experiment was carried out for 6 hours at a holding potential of 120 mV with potential flicks every hour with an applied potential of −120 mV and helicase-controlled DNA movement was monitored.

### Results and Discussion

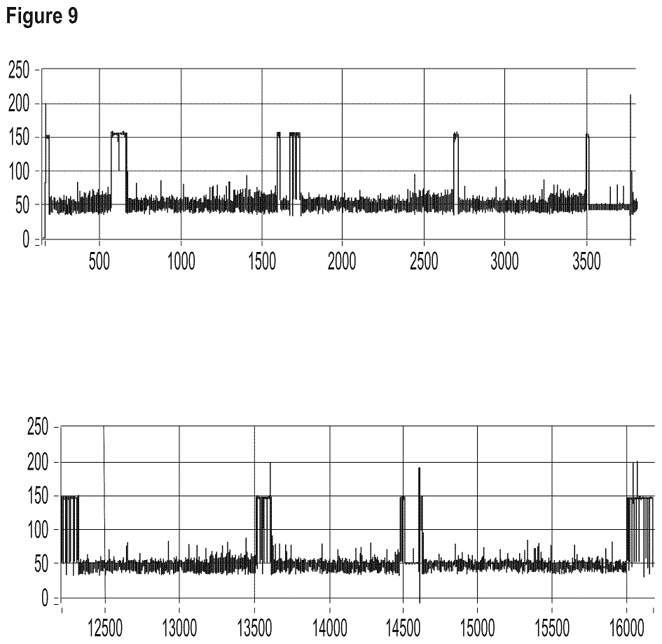

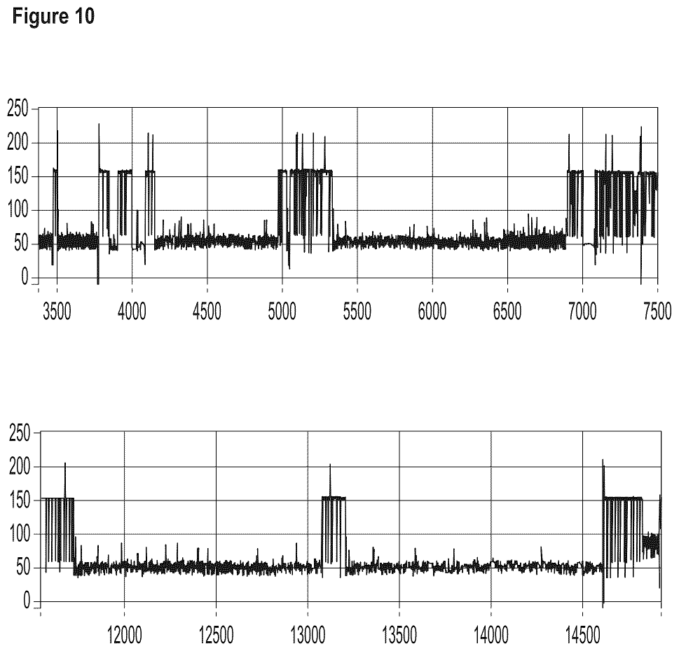

Helicase controlled DNA movement was observed for DNA construct X, with all mutant helicases investigated (T4 Dda—E94C/A360C, T4 Dda—E94C/A360C/C109A/C136A or T4 Dda—E94C/A360C/C114A/C171A/C421D). Examples of T4 Dda—E94C/A360C/C109A/C136A and T4 Dda—E94C/A360C/C114A/C171A/C421D helicase-controlled DNA movements are shown in FIGS. 9 and 10 respectively.

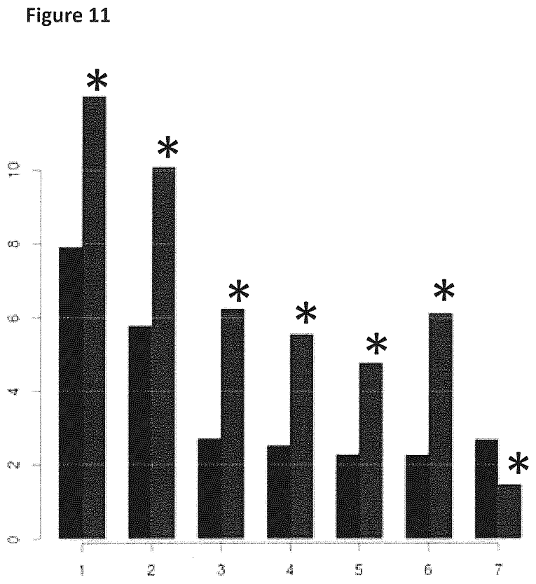

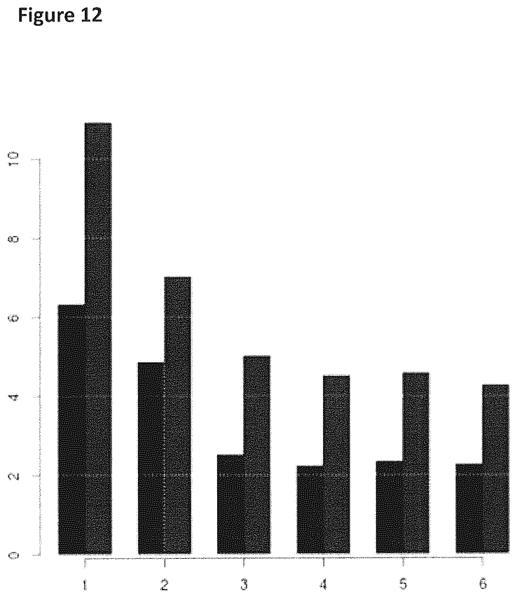

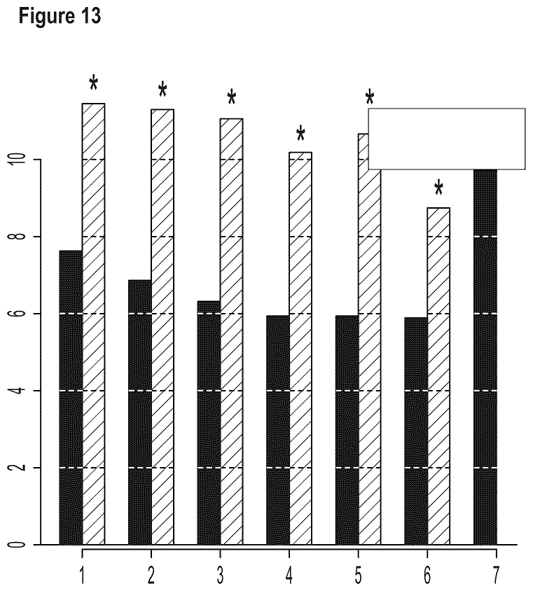

The helicase controlled DNA movement speed was monitored through both region 1 and the region 2 of the lambda DNA construct X. For T4 Dda—E94C/A360C and T4 Dda—E94C/A360C/C114A/C171A/C421D the number of helicase controlled DNA movements per second was found to gradually decrease over the seven hour run time for both region 1 and 2 (See FIG. 11 for T4 Dda—E94C/A360C and FIG. 12 for T4 Dda—E94C/A360C/C114A/C171A/C421D). However, the T4 Dda—E94C/A360C/C109A/C136A mutant helicase observed only a slight decrease in the number of helicase controlled DNA movements per second over the 7 hour experimental run for both region 1 and region 2 (see FIG. 13). The T4 Dda—E94C/A360C/C109A/C136A mutant therefore showed improved helicase controlled DNA movement as the speed of movement remained high and fairly constant during the entire experimental run. This allowed increased throughput in comparison to the T4 Dda—E94C/A360C which exhibited a gradual reduction in speed over time.

### Example 5

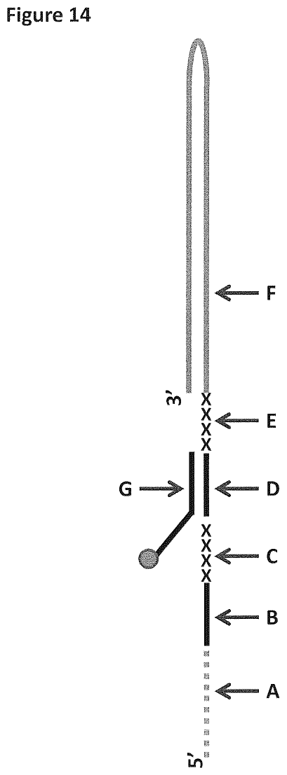

This example describes how a T4 Dda—E94C/C109A/C136A/A360C/W378A (SEQ ID NO: 8 with mutations E94C/C109A/C136A/A360C/W378A and then (ΔM1)G1G2) helicase can control the movement of intact DNA construct Z strands (shown in FIG. 14) through a single MspA nanopore.

### Materials and Methods

Prior to setting up the experiment, the DNA construct Z (see FIG. 8 for a diagram of the construct and sequences, 1.2 μL) and T4 Dda—E94C/C109A/C136A/A360C/W378A (2.84 μL) were pre-incubated together for 5 minutes at 23° C. in buffer (151 mM KCl, 25 mM potassium phosphate pH 8, 1 mM EDTA, 5% Glycerol). TMAD (500 μM, 0.92 μL) was added to the DNA enzyme mix and incubated at 23° C. for a further five minutes. Finally, buffer (282 μL of 500 mM KCl, 25 mM potassium phosphate pH 8), ATP (final concentration of 2 mM) and MgCL2 (final concentration 2 mM) were added to the mixture.

Electrical measurements were acquired as described in Example 1 using MspA nanopores inserted in block co-polymer in buffer (500 mM KCl, 25 mM potassium phosphate, pH 8). The pre-mix was added to the single nanopore experimental system and the experiment run at a holding potential of −120 mV for 6 hours (with potential flips to +60 mV for 2 seconds) and helicase-controlled DNA movement monitored.

### Results and Discussion

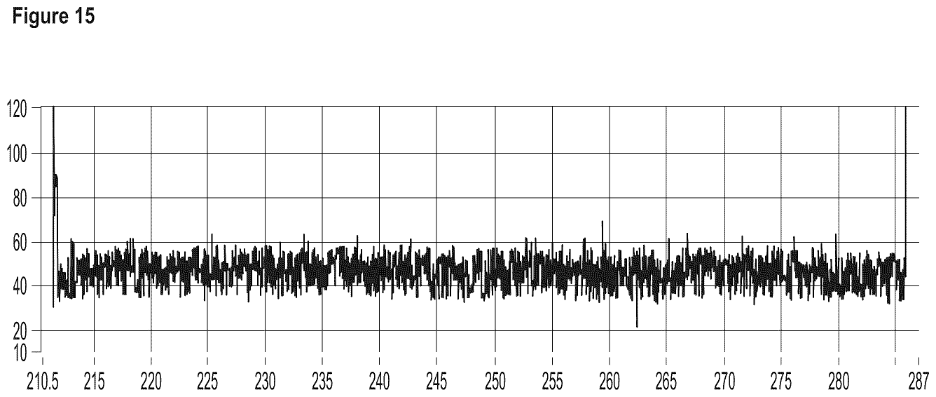

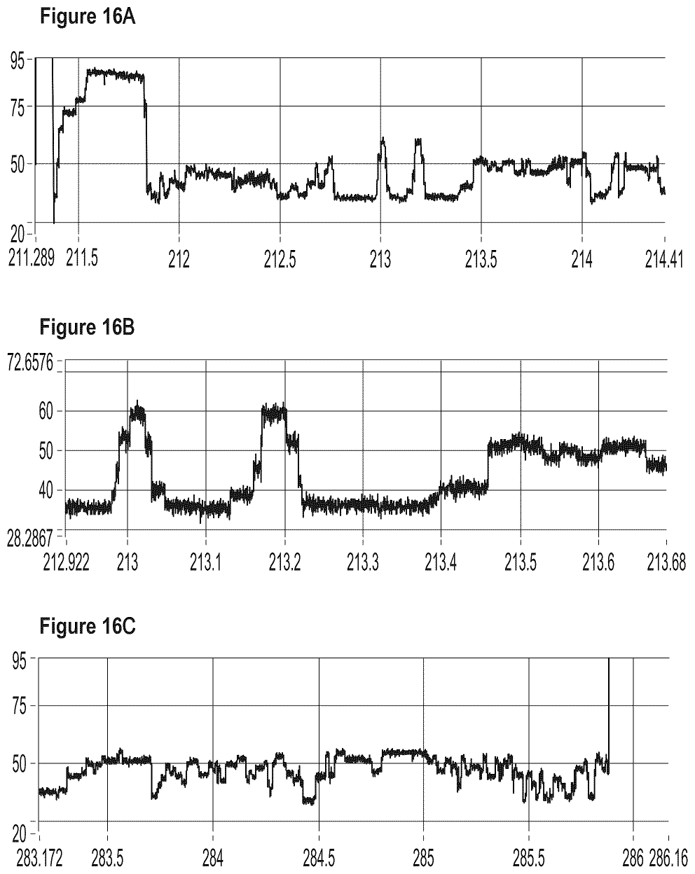

Helicase controlled DNA movement was observed for DNA construct Z, an example of a helicase-controlled DNA movement is shown in FIG. 15. FIGS. 16A-16C show the beginning of the helicase-controlled DNA movement in trace (A), shows a zoomed in region of trace A in trace (B) and shows the end of the helicase controlled DNA movement in trace (C).

### Example 6

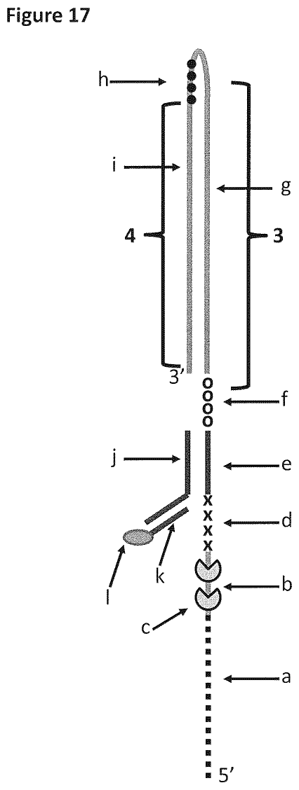

This example compared the use of a single T4 Dda—E94C/A360C or Ta Dda—E94C/C109A/C136A/A360C to two T4 Dda—E94C/A360C (SEQ ID NO: 24 with mutations E94C/A360C) or two T4 Dda—E94C/C109A/C136A/A360C (SEQ ID NO: 24 with mutations E94C/C109A/C136A/A360C) helicases in order to control the movement of DNA construct X (shown in FIG. 17) through an MspA nanopore. When two helicases were used to control the movement of the construct through the nanopore then improved movement was observed in comparison to when the movement was controlled by a single helicase.

### Materials and Methods

Prior to setting up the experiment, DNA construct X (see FIG. 17 for diagram and sequences used in construct X, final concentration added to the nanopore system 0.1 nM) was pre-incubated at room temperature for five minutes with T4 Dda—E94C/A360C (final concentration added to the nanopore system 1 nM, SEQ ID NO: 24 with mutations E94C/A360C) or T4 Dda—E94C/C109A/C136A/A360C (final concentration added to the nanopore system 1 nM, SEQ ID NO: 24 with mutations E94C/C109A/C136A/A360C, which was provided in buffer (253 mM KCl, 50 mM potassium phosphate, pH 8.0, 2 mM EDTA)). After five minutes, TMAD (1 μM final concentration added to the nanopore system) was added to the pre-mix and the mixture incubated for a further 5 minutes. Finally, MgCl2 (2 mM final premix concentration), ATP (2 mM final premix concentration) and buffer (25 mM potassium phosphate and 500 mM KCl pH 8.0) were added to the pre-mix.

Electrical measurements were acquired from single MspA nanopores inserted in block co-polymer in buffer (25 mM potassium phosphate, 150 mM potassium ferrocyanide (II), 150 mM potassium ferricyanide (III), pH 8.0). After achieving a single pore inserted in the block co-polymer, then buffer (2 mL, 25 mM potassium phosphate pH 8.0, 150 mM potassium ferrocyanide (II) and 150 mM potassium ferricyanide (III)) was flowed through the system to remove any excess MspA nanopores. The enzyme (T4 Dda—E94C/A360C or T4 Dda—E94C/C109A/C136A/A360C, 1 nM final concentration), DNA construct X (0.1 nM final concentration), fuel (MgCl2 2 mM final concentration, ATP 2 mM final concentration) pre-mix (300 μL total) was then flowed into the single nanopore experimental system and the experiment run at a holding potential of 120 mV for 6 hours and helicase-controlled DNA movement monitored.

### Results

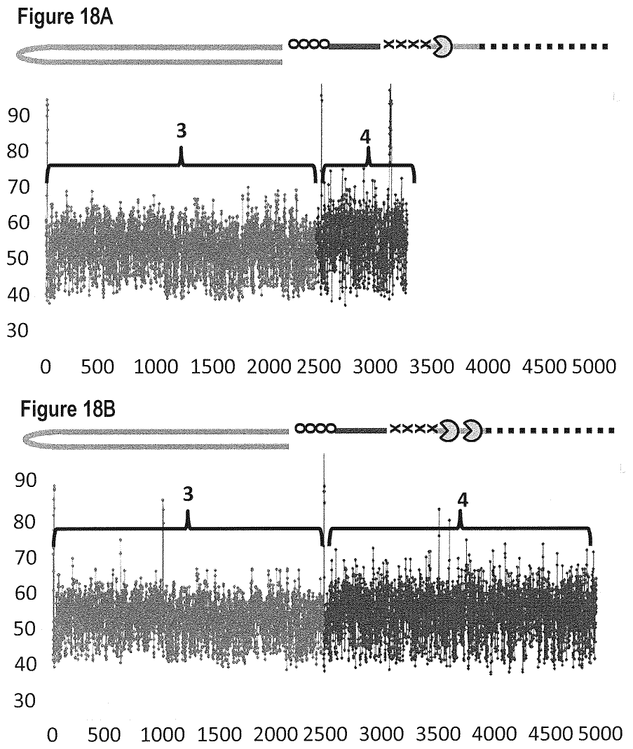

Helicase controlled DNA movement was observed for DNA construct X (FIG. 17) using T4 Dda—E94C/A360C and T4 Dda—E94C/C109A/C136A/A360C (see FIGS. 18A and 18B respectively). When a single enzyme was bound to DNA construct X (movement index shown in FIG. 18A), then helicase controlled DNA movement through the nanopore was observed for regions 3 and 4 (see FIGS. 18A-18B). Region 3 moved through the pore in a controlled manner in which it was possible to observe a movement index (see FIGS. 18A-18B's figure legend for description of movement index) for the region which was plotted in FIG. 18A. However, when region 4 translocated through the nanopore, the movement index plotted in FIG. 18A showed many less points than that produced for region 3. As region 3 and 4 were approximately the same length, the movement index observed for each region would have been expected to have had approximately the same number of points. This meant that the movement control of region 4 provided by a single enzyme (T4 Dda—E94C/A360C) resulted in less points and therefore less information was obtained for region 4 in comparison to region 3. Less information was obtained owing to the enzyme movement not being as consistent when region 4 was translocated through the nanopore (e.g. the DNA slipped forward along sections of region 4) that meant sections of DNA sequence were missed.

In this Example, the helicases move along the polynucleotide in a 5′ to 3′ direction. When the 5′end of the polynucleotide (the end away from which the helicases move) is captured by the pore, the helicases work with the direction of the field resulting from the applied potential and move the threaded polynucleotide into the pore and into the trans chamber. In this Example, slipping forward involves the DNA moving forwards relative to the pore (i.e. towards its 3′ and away from it 5′ end in this Example) at least 4 consecutive nucleotides and typically more than 10 consecutive nucleotides. Slipping forward may involve movement forward of 100 consecutive nucleotides or more and this may happen more than once in each strand. This phenomenon was called skipping and slipping in UK Application Nos. 1406151.9.

FIG. 18B shows the movement index produced when the movement of DNA construct X (regions 3 and 4) was controlled using a “series” of enzymes, in this case two T4 Dda—E94C/A360C enzymes. The movement index of region 3 of DNA construct X was similar to that observed for the single enzyme. However, when region 4 translocated through the nanopore under the control of two enzymes then the DNA movement index was significantly different from that observed when a single T4 Dda—E94C/A360C helicase controlled the movement. A similar movement index was observed for region 4 as for region 3 when the movement was controlled using two T4 Dda—E94C/A360C enzymes, with both regions having approximately the same number of points. This illustrated that improved helicase-controlled DNA movement was observed when two T4 Dda—E94C/A360C enzymes in a “series” were used to control movement. This was because a similar amount of information was obtained for region 4 as region 3, whereas movement controlled using a single enzyme resulted in less information for region 4 than region 3. More information was obtained because the series of helicases resulted in more consistent movement of the DNA (e.g. slower movement or less slipping forward of the DNA region labelled 4). This meant that a series of T4 Dda—E94C/A360C enzymes could be used to improve sequencing of a strand of DNA.

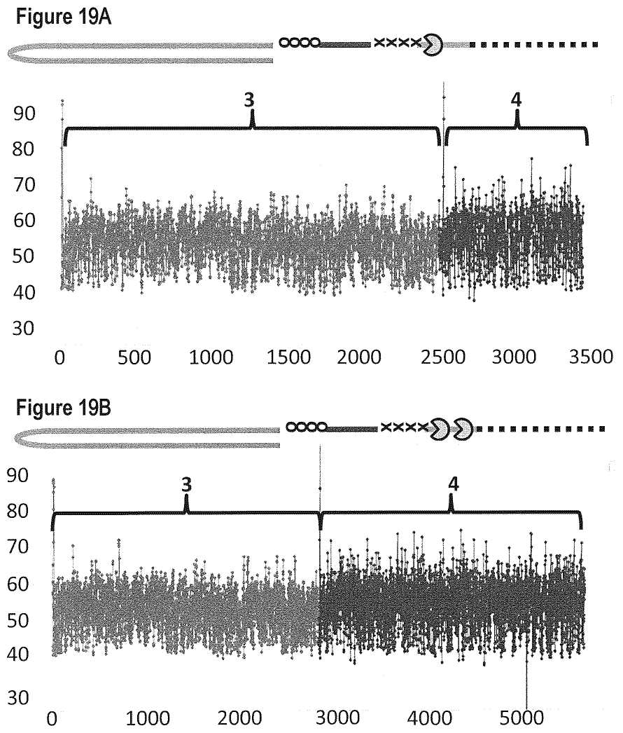

The same experiment was carried out using the helicase T4 Dda—E94C/C109A/C136A/A360C to control the movement of DNA construct X through the nanopore. FIG. 19A shows the movement index for construct X when movement was controlled by a single T4 Dda—E94C/C109A/C136A/A360C enzyme and FIG. 19B shows the movement index when the movement was controlled by two T4 Dda—E94C/C109A/C136A/A360C helicases. As was observed for T4 Dda—E94C/A360C, a series of two T4 Dda—E94C/C109A/C136A/A360C helicases resulted in more points being observed in the movement index when the movement of region 2 of the DNA was controlled by two enzymes, which indicated improved movement of this region (slower movement or less slipping forward). This meant that a series of T4 Dda—E94C/C109A/C136A/A360C enzymes could be used to improve sequencing of a strand of DNA. DNA construct X, shown and described in FIG. 17, has a section labelled b onto which two enzymes could bind. Control experiments where the length of section b was only sufficient to allow one enzyme to bind (10-12 T binding sites) were carried out for both T4 Dda—E94C/A360C and T4 Dda—E94C/C109A/C136A/A360C. In the control experiments, when region 4 translocated through the nanopore no strands with improved movement were detected when only a single enzyme bound to the construct and controlled the movement of the strand through the nanopore. In comparison, in the experiments above where two enzymes could have bound to the DNA, although we observed some strands with poor movement because only a single enzyme bound, it was also possible to identify strands with improved movement indexes which corresponded to DNA translocation controlled by two enzymes, rather than just one.

### Example 7

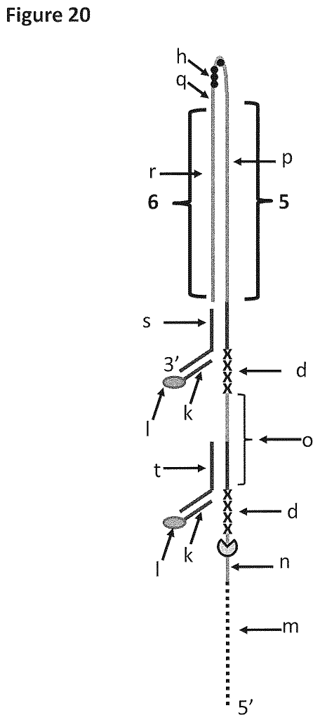

This example compared the use of a single T4 Dda—E94C/C109A/C136A/A360C or both T4 Dda—E94C/C109A/C136A/A360C and T4 Dda—E94C/C109A/C136A/A360C/W378A (SEQ ID NO: 24 with mutations E94C/C109A/C136A/A360C/W378A) in order to control the movement of DNA construct Z (shown in FIG. 20) through an MspA nanopore. T4 Dda—E94C/C109A/C136A/A360C and T4 Dda—E94C/C109A/C136A/A360C/W378A are both active helicases which moved along the DNA when provided with appropriate fuel. When these two different helicases were used to control the movement of the construct through the nanopore then improved movement was observed in comparison to when the movement was controlled by a single helicase (T4 Dda—E94C/C109A/C136A/A360C).

### Materials and Methods

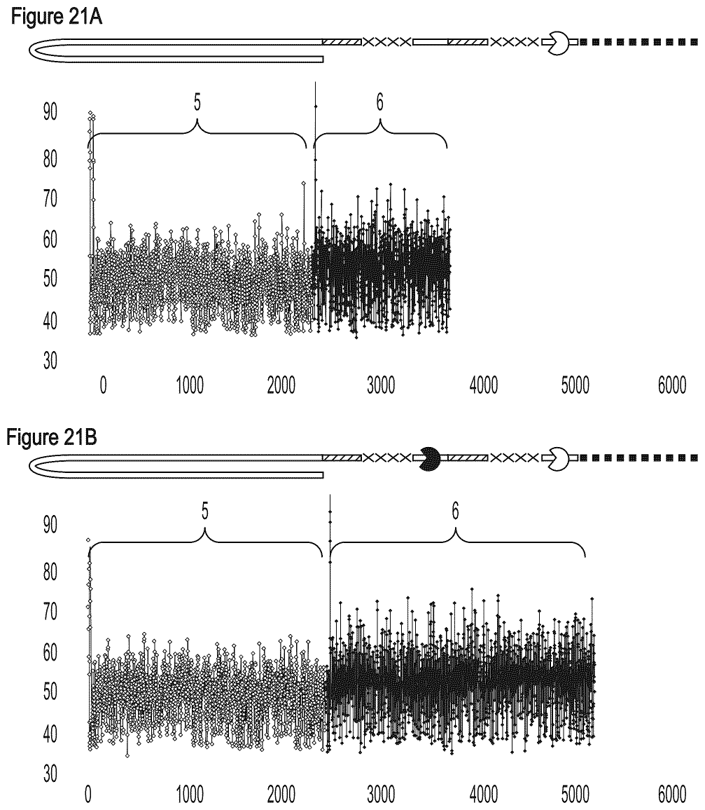

The DNA construct Z (final concentration added to the nanopore system 0.1 nM) which either had both enzymes pre-bound (see FIG. 21B data) or only T4 Dda—E94C/C109A/C136A/A360C pre-bound (control experiment, see FIG. 21A data) was added to buffer (final concentrations added to the nanopore system were 500 mM KCl, 25 mM potassium phosphate pH 8.0), ATP (final concentration added to the nanopore system 2 mM) and MgCL2 (final concentration added to the nanopore system 2 mM). This was the pre-mix which was then added to the nanopore system (total volume 150 μL).

Electrical measurements were acquired from single MspA nanopores inserted in block co-polymer in buffer (25 mM potassium phosphate, 75 mM potassium ferrocyanide (II), 25 mM potassium ferricyanide (III), 600 mM KCl, pH 8.0). After achieving a single pore inserted in the block co-polymer, then buffer (2 mL, 25 mM potassium phosphate, 75 mM potassium ferrocyanide (II), 25 mM potassium ferricyanide (III), 600 mM KCl, pH 8.0) was flowed through the system to remove any excess MspA nanopores. The enzyme pre-bound to construct Z (either a single T4 Dda—E94C/C109A/C136A/A360C (control) or T4 Dda—E94C/C109A/C136A/A360C and T4 Dda—E94C/C109A/C136A/A360C/W378A), fuel (MgCl2 and ATP) pre-mix (150 μL total) was then flowed into the single nanopore experimental system and the experiment run at a holding potential of −120 mV for 6 hours (with potential flips to +60 mV for 2 seconds) and helicase-controlled DNA movement monitored.

### Results

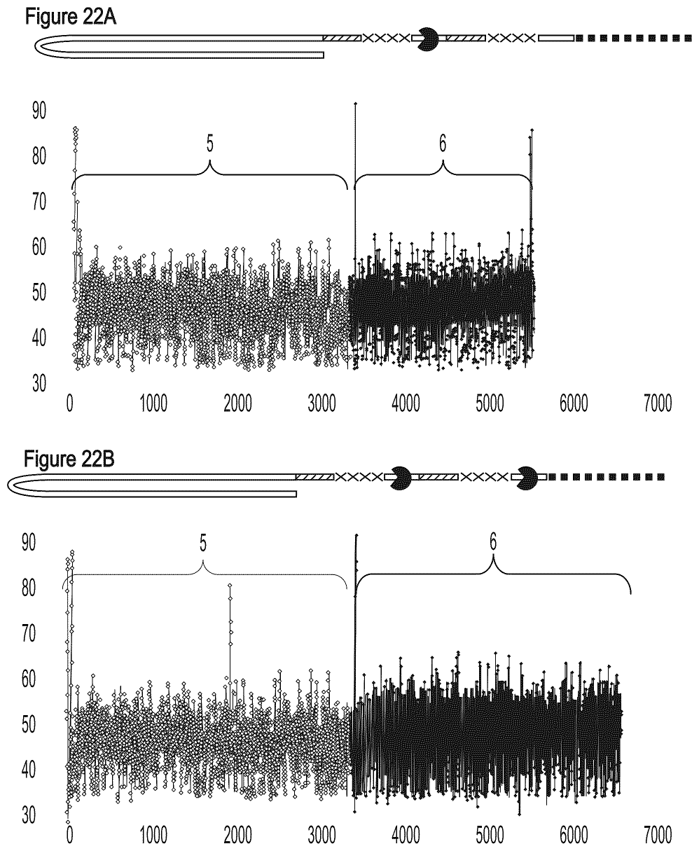

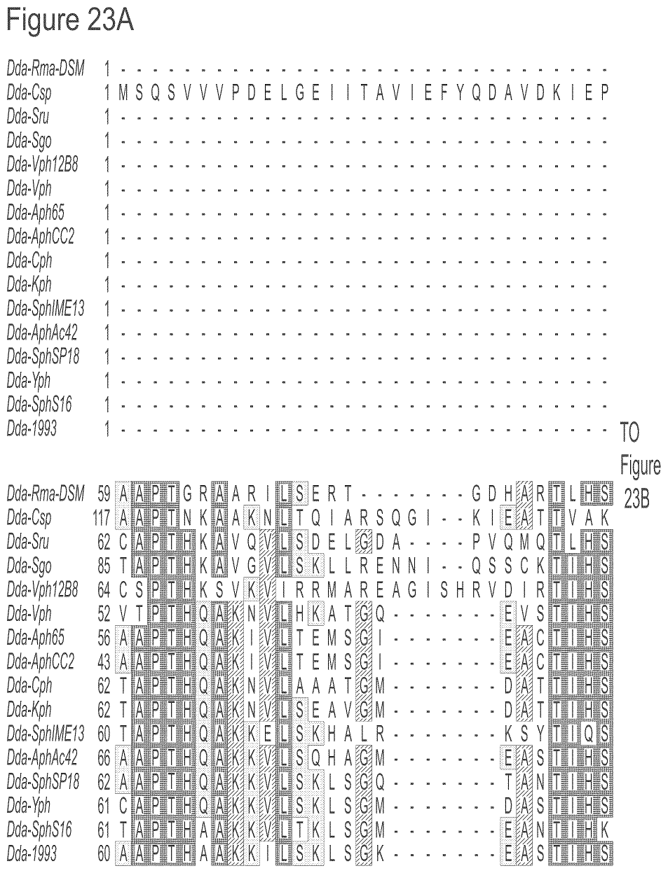

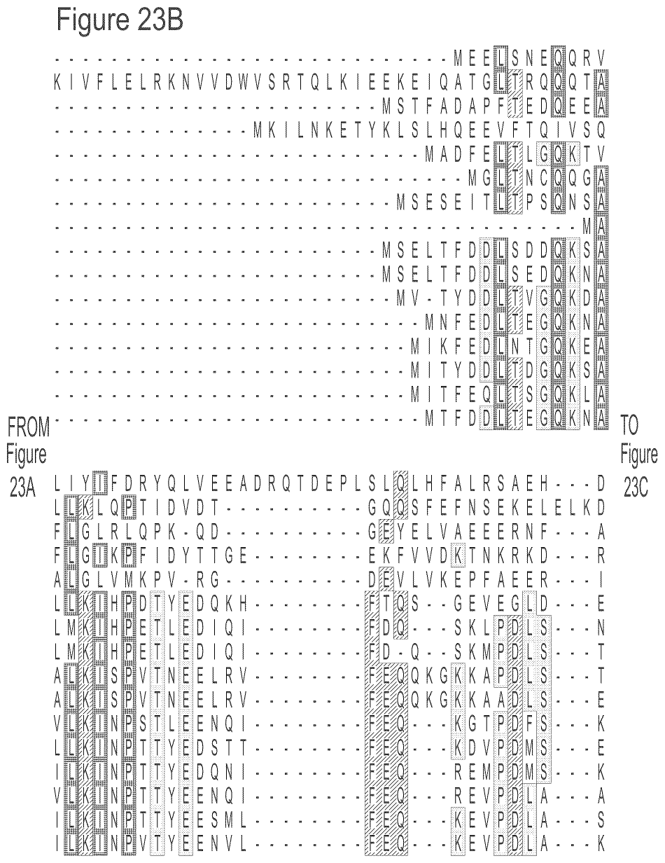

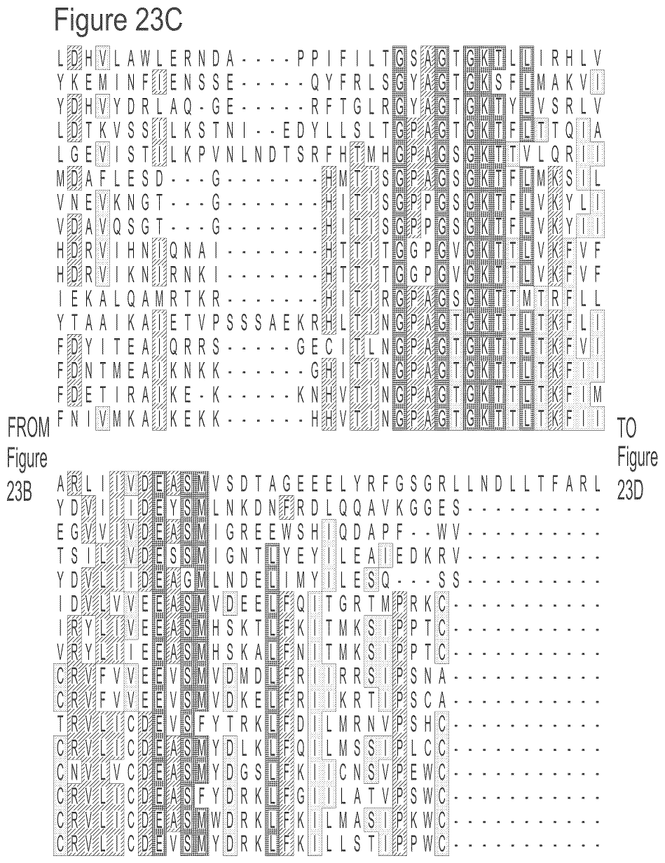

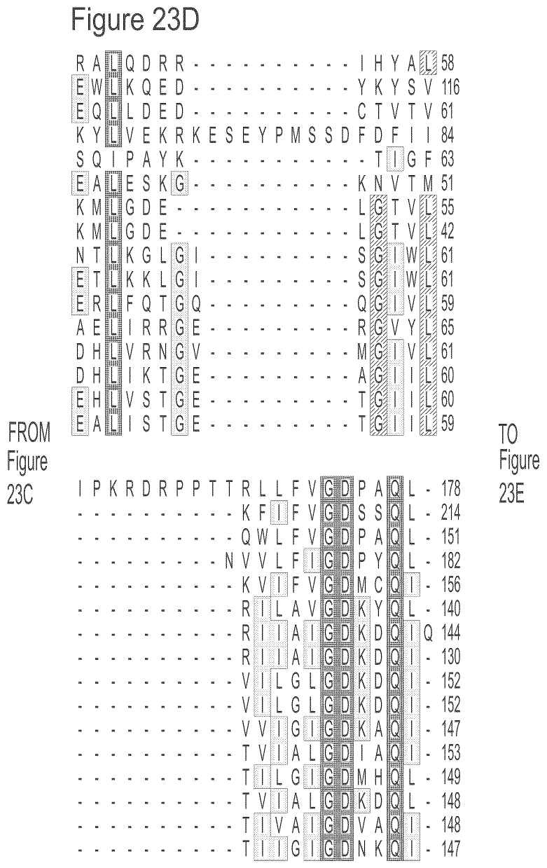

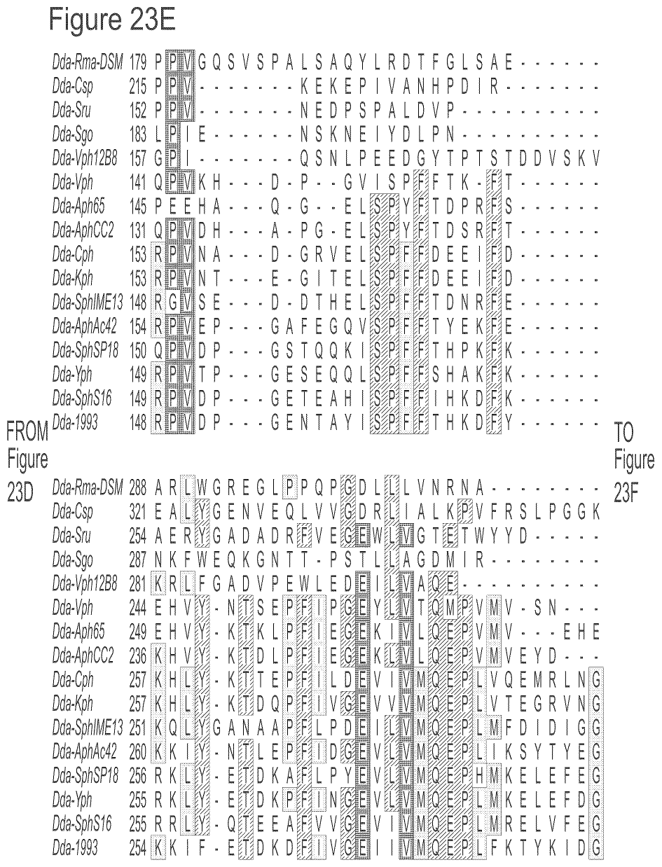

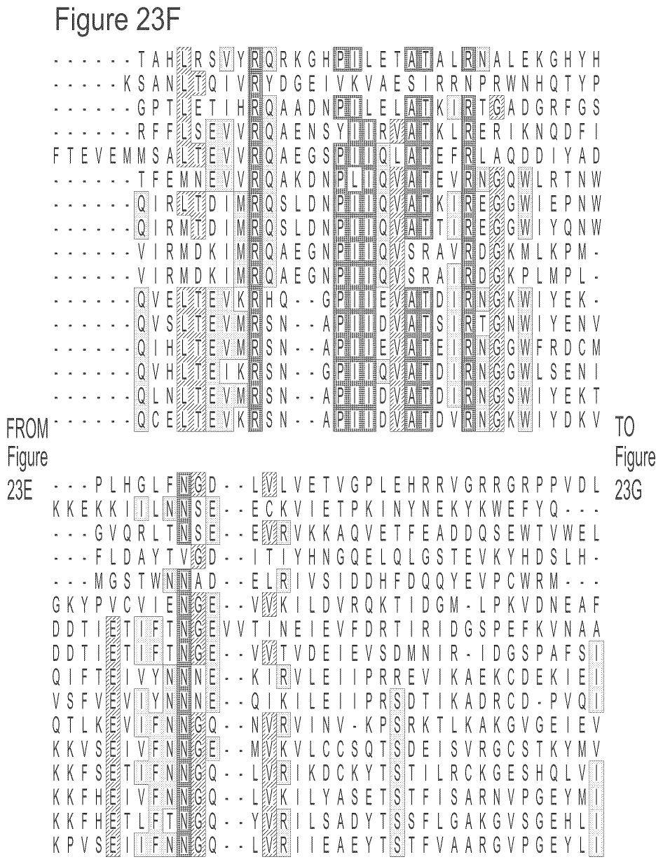

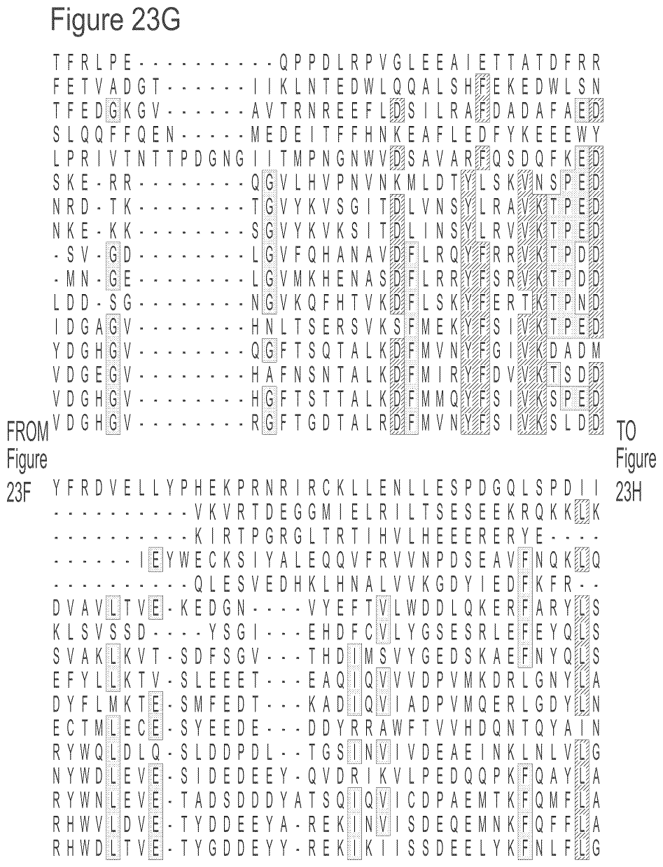

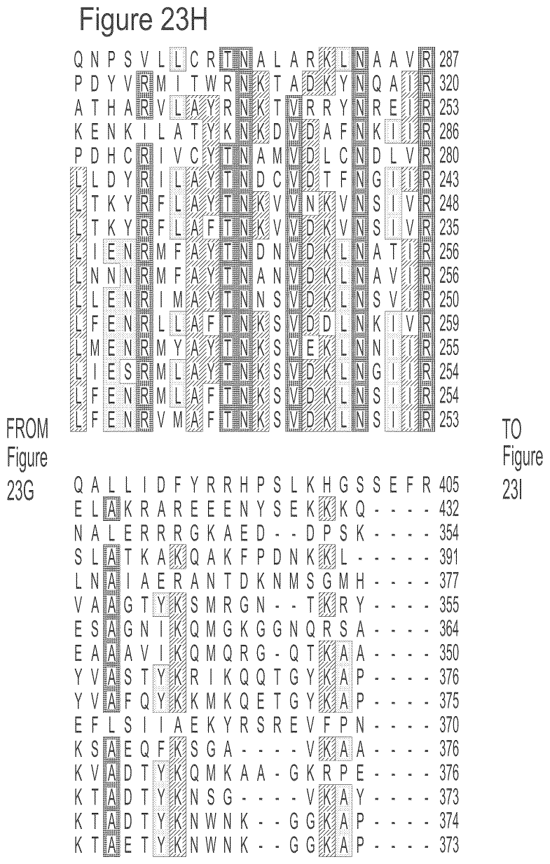

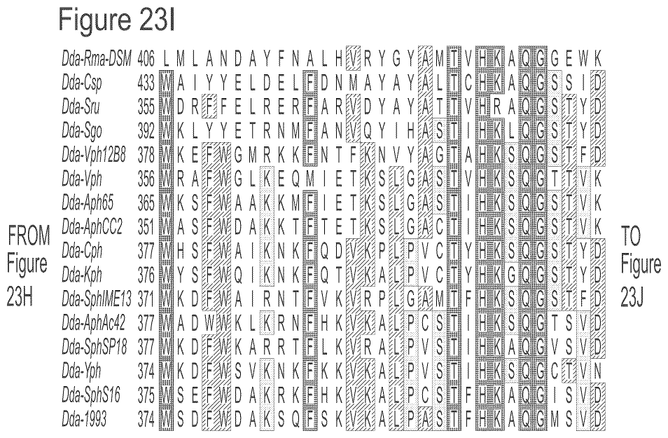

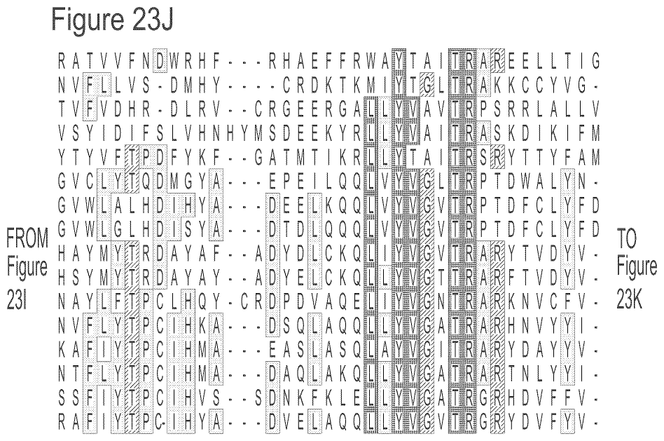

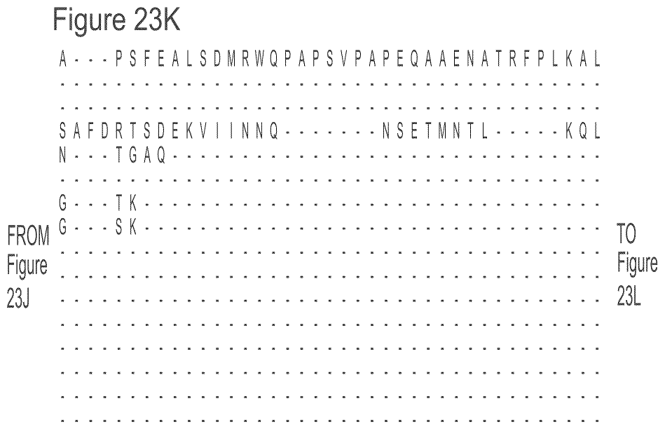

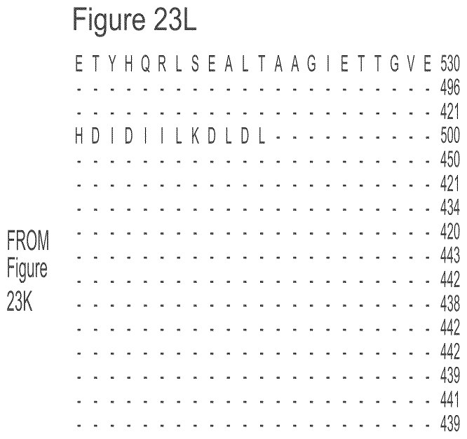

Helicase controlled DNA movements corresponding to controlled translocation by T4 Dda—E94C/C109A/C136A/A360C only (control experiment, FIG. 21A) or both T4 Dda—E94C/C109A/C136A/A360C and T4 Dda—E94C/C109A/C136A/A360C/W378A (FIG. 22B) were observed. The trace shown in FIG. 21A showed an example movement index plot when only the helicase T4 Dda—E94C/C109A/C136A/A360C controlled the translocation of DNA construct Z (see FIG. 20) through an MspA nanopore. When region 5 translocated through the nanopore, it was possible to observe the movement index for region 5. However, this figure showed that the movement index for region 6 had less points than for region 5 which indicated that less information was obtained for this region of DNA construct Y when it translocated through the nanopore. This resulted in DNA movement that was less consistent (e.g. more slipping forward of the DNA region labelled 6) and sections of DNA sequence were missed.

FIG. 21B shows the movement index when T4 Dda—E94C/C109A/C136A/A360C and T4 Dda—E94C/C109A/C136A/A360C/W378A controlled the translocation of DNA construct Z (see FIG. 20) through an MspA nanopore. When region 5 translocated through the nanopore under the control of T4 Dda—E94C/C109A/C136A/A360C and T4 Dda—E94C/C109A/C136A/A360C/W378A, it was possible to observe a movement index. Moreover, when region 6 translocated through the nanopore, the movement was again controlled by both T4 Dda—E94C/C109A/C136A/A360C and T4 Dda—E94C/C109A/C136A/A360C/W378A. When region 6 translocated through the nanopore under the control of the two enzymes (T4 Dda—E94C/C109A/C136A/A360C and T4 Dda—E94C/C109A/C136A/A360C/W378A) then the DNA movement was significantly different from that observed when a single T4 Dda—E94C/C109A/C136A/A360C helicase controlled the movement of this region (see FIG. 21A section 6). This figure showed that the movement index for region 6, when the helicase movement was controlled using T4 Dda—E94C/C109A/C136A/A360C and T4 Dda—E94C/C109A/C136A/A360C/W378A, had many more points than for region 6 when the helicase movement was controlled by the single enzyme T4 Dda—E94C/C109A/C136A/A360C which indicated that more information was obtained for this region of DNA construct Z when it translocated through the nanopore under the control of two different enzymes and that the DNA movement was more consistent (e.g. slower movement or less slipping forward of the DNA region labelled 6). This meant that the combination of T4 Dda—E94C/C109A/C136A/A360C and T4 Dda—E94C/C109A/C136A/A360C/W378A enzymes were used to improve sequencing of a strand of DNA.

### Example 8

This example compared the use of either a single T4 Dda—E94C/C109A/C136A/A360C/W378A or two T4 Dda—E94C/C109A/C136A/A360C/W378A helicases (SEQ ID NO: 24 with mutations E94C/C109A/C136A/A360C/W378A) in order to control the movement of DNA construct Z (shown in FIG. 20) through an MspA nanopore. T4 Dda—E94C/C109A/C136A/A360C/W378A is an active helicase which moved along the DNA when provided with appropriate fuel. When two helicases (T4 Dda—E94C/C109A/C136A/A360C/W378A) were used to control the movement of the construct through the nanopore then improved movement was observed in comparison to when the movement was controlled by a single helicase (T4 Dda—E94C/C109A/C136A/A360C/W378A).

### Materials and Methods

The DNA construct Z (final concentration added to the nanopore system 0.1 nM) which either had two T4 Dda—E94C/C109A/C136A/A360C/W378A helicases pre-bound (see FIG. 21B data) or a single T4 Dda—E94C/C109A/C136A/A360C/W378A pre-bound (control experiment, see FIG. 21A data) was added to buffer (final concentrations added to the nanopore system were 500 mM KCl, 25 mM potassium phosphate pH 8.0), ATP (final concentration added to the nanopore system 2 mM) and MgCL2 (final concentration added to the nanopore system 2 mM). This was the pre-mix which was then added to the nanopore system (total volume 150 μL).

Electrical measurements were acquired from single MspA nanopores as described in Example 7 above, except either the DNA construct Z with a single T4 Dda—E94C/C109A/C136A/A360C/W378A pre-bound (as a control experiment) or two T4 Dda—E94C/C109A/C136A/A360C/W378A helicases pre-bound were added to the nanopore system.

### Results

Helicase controlled DNA movements corresponding to controlled translocation by T4 Dda—E94C/C109A/C136A/A360C/W378A only (control experiment, FIG. 22A) or two T4 Dda—E94C/C109A/C136A/A360C/W378A helicases (FIG. 22B) were observed. The trace shown in FIG. 22A showed an example movement index plot when only a single helicase T4 Dda—E94C/C109A/C136A/A360C/W378A controlled the translocation of DNA construct Z (see FIG. 20) through an MspA nanopore. When region 5 translocated through the nanopore, it was possible to observe the movement index for region 5. However, this figure showed that the movement index for region 6 had less points than for region 5 which indicated that less information was obtained for this region of DNA construct Z when it translocated through the nanopore. This resulted in DNA movement that was less consistent (e.g. more slipping forward of the DNA region labelled 6) and sections of DNA sequence were missed.

FIG. 22B shows the movement index when two T4 Dda—E94C/C109A/C136A/A360C/W378A helicases controlled the translocation of DNA construct Z (see FIG. 20) through an MspA nanopore. When region 5 translocated through the nanopore under the control of two T4 Dda—E94C/C109A/C136A/A360C/W378A helicases, it was possible to observe a movement index. Moreover, when region 6 translocated through the nanopore, the movement was again controlled by twoT4 Dda—E94C/C109A/C136A/A360C/W378A helicases. When region 6 translocated through the nanopore under the control of the two enzymes (two T4 Dda—E94C/C109A/C136A/A360C/W378A helicases) then the DNA movement was significantly different from that observed when a single T4 Dda—E94C/C109A/C136A/A360C/W378A helicase controlled the movement of region 6 (see FIG. 22A section 6). This figure showed that the movement index for region 6, when the helicase movement was controlled using two T4 Dda-E94C/C109A/C136A/A360C/W378A helicases, had many more points than when the helicase movement was controlled by the single enzyme T4 Dda—E94C/C109A/C136A/A360C/W378A which indicated that more information was obtained for this region of DNA construct Z when it translocated through the nanopore under the control of two enzymes than was observed for the region 6 of construct Z under the control of a single T4 Dda—E94C/C109A/C136A/A360C/W378A helicase. Furthermore, the DNA movement which was observed when DNA translocation was controlled by two T4 Dda—E94C/C109A/C136A/A360C/W378A helicases was also more consistent (e.g. slower movement or less slipping forward of the DNA region labelled 8). This meant that the use of two T4 Dda—E94C/C109A/C136A/A360C/W378A enzymes resulted in improved sequencing of a strand of DNA.

1.-59. (canceled)

60. An apparatus for characterising target polynucleotides in a sample, comprising:
a plurality of pores; and
a plurality of DNA-dependent ATPase (Dda) helicases in which at least one cysteine residue and/or at least one non-natural amino acid have been introduced into (i) tower domains, and/or (ii) pin domains, and/or (iii) a1A (RecA-like motor) domains of the plurality of Dda helicases;
wherein the plurality of Dda helicases retain their ability to control the movement of a polynucleotide; and
wherein the plurality of Dda helicases comprises:
(a) a variant of SEQ ID NO: 8 in which at least one cysteine residue and/or at least one non-natural amino acid have been introduced into (i) the tower domain (residues D260-P274 and N292-A389) and/or (ii) the pin domain (residues K86-E102) and/or the (iii) 1A domain (residues M1-L85 and V103-K177);
(b) a variant of SEQ ID NO: 9 in which at least one cysteine residue and/or at least one non-natural amino acid have been introduced into (i) the tower domain (residues G295-N309 and F316-Y421) and/or (ii) the pin domain (residues Y85-L112) and/or the (iii) 1A domain (residues M1-I84 and R113-Y211);
(c) a variant of SEQ ID NO: 10 in which at least one cysteine residue and/or at least one non-natural amino acid have been introduced into (i) the tower domain (residues V328-P342 and N360-Y448) and/or (ii) the pin domain (residues K148-N165) and/or the (iii) 1A domain (residues M1-L147 and S166-V240);
(d) a variant of SEQ ID NO: 11 in which at least one cysteine residue and/or at least one non-natural amino acid have been introduced into (i) the tower domain (residues A261-T275 and T285-Y370) and/or (ii) the pin domain (residues G91-E107) and/or the (iii) 1A domain (residues M1-L90 and E108-H173);
(e) a variant of SEQ ID NO: 12 in which at least one cysteine residue and/or at least one non-natural amino acid have been introduced into (i) the tower domain (residues G294-1307 and T314-Y407) and/or (ii) the pin domain (residues G116-T135) and/or the (iii) 1A domain (residues M1-L115 and N136-V205);
(f) a variant of SEQ ID NO: 13 in which at least one cysteine residue and/or at least one non-natural amino acid have been introduced into (i) the tower domain (residues V288-E301 and N307-N393) and/or (ii) the pin domain (residues G97-P113) and/or the (iii) 1A domain (residues M1-L96 and F114-V194);
(g) a variant of SEQ ID NO: 14 in which at least one cysteine residue and/or at least one non-natural amino acid have been introduced into (i) the tower domain (residues 5250-P264 and E278-5371) and/or (ii) the pin domain (residues K78-E95) and/or the (iii) 1A domain (residues M1-L77 and V96-V166);
(h) a variant of SEQ ID NO: 15 in which at least one cysteine residue and/or at least one non-natural amino acid have been introduced into (i) the tower domain (residues K255-P269 and T284-5380) and/or (ii) the pin domain (residues K82-K98) and/or the (iii) 1A domain (residues M1-M81 and L99-M171);
(i) a variant of SEQ ID NO: 16 in which at least one cysteine residue and/or at least one non-natural amino acid have been introduced into (i) the tower domain (residues D242-P256 and T271-S366) and/or (ii) the pin domain (residues K69-K85) and/or the (iii) 1A domain (residues M1-M68 and M86-M158);
(j) a variant of SEQ ID NO: 17 in which at least one cysteine residue and/or at least one non-natural amino acid have been introduced into (i) the tower domain (residues T263-P277 and N295-P392) and/or (ii) the pin domain (residues K88-K107) and/or the (iii) 1A domain (residues M1-L87 and A108-M181);
(k) a variant of SEQ ID NO: 18 in which at least one cysteine residue and/or at least one non-natural amino acid have been introduced into (i) the tower domain (residues D263-P277 and N295-A391) and/or (ii) the pin domain (residues K88-K107) and/or the (iii) 1A domain (residues M1-L87 and A108-M181);
(l) a variant of SEQ ID NO: 19 in which at least one cysteine residue and/or at least one non-natural amino acid have been introduced into (i) the tower domain (residues A258-P272 and N290-P386) and/or (ii) the pin domain (residues K86-G102) and/or the (iii) 1A domain (residues M1-L85 and T103-K176);
(m) a variant of SEQ ID NO: 20 in which at least one cysteine residue and/or at least one non-natural amino acid have been introduced into (i) the tower domain (residues L266-P280 and N298-A392) and/or (ii) the pin domain (residues K92-D108) and/or the (iii) 1A domain (residues M1-L91 and V109-M183);
(n) a variant of SEQ ID NO: 21 in which at least one cysteine residue and/or at least one non-natural amino acid have been introduced into (i) the tower domain (residues D262-P276 and N294-A392) and/or (ii) the pin domain (residues K88-E104) and/or the (iii) 1A domain (residues M1-L87 and M105-M179);
(o) a variant of SEQ ID NO: 22 in which at least one cysteine residue and/or at least one non-natural amino acid have been introduced into (i) the tower domain (residues D261-P275 and N293-A389) and/or (ii) the pin domain (residues K87-E103) and/or the (iii) 1A domain (residues M1-L86 and V104-K178); or
(p) a variant of SEQ ID NO: 23 in which at least one cysteine residue and/or at least one non-natural amino acid have been introduced into (i) the tower domain (residues E261-P275 and T293-A390) and/or (ii) the pin domain (residues K87-E103) and/or the (iii) 1A domain (residues M1-L86 and V104-M178).

61. The apparatus according to claim 60, wherein the apparatus comprises:
a sensor device that is configured to support the plurality of pores and operable to perform polynucleotide characterisation using the plurality of pores and the plurality of Dda helicases; and
at least one port for delivery of material for performing the characterisation.

62. The apparatus according to claim 60, wherein the apparatus comprises:
a sensor device that is configured to support the plurality of pores and operable to perform polynucleotide characterisation using the plurality of pores and the plurality of Dda helicases; and
at least one reservoir for holding material for performing the characterisation.

63. The apparatus according to claim 62, wherein the apparatus comprises:
a sensor device that is configured to support the plurality of pores and operable to perform polynucleotide characterisation using the plurality of pores and the plurality of Dda helicases;
at least one reservoir for holding material for performing the characterisation;
a fluidics system configured to controllably supply material from the at least one reservoir to the sensor device; and
one or more containers for receiving respective samples, the fluidics system being configured to supply the samples selectively from the one or more containers to the sensor device.

64. A method of producing a DNA-dependent ATPase (Dda) helicase, the method comprising:
(a) providing a Dda helicase; and
(b) modifying the helicase to introduce at least one cysteine residue and/or at least one non-natural amino acid into (i) a tower domain, and/or (ii) a pin domain, and/or (iii) a1A (RecA-like motor) domain of the Dda helicase;
wherein the Dda helicase retains its ability to control the movement of a polynucleotide.

65.-69. (canceled)

70. The apparatus of claim 60, wherein:
(A) the plurality of Dda helicases further comprise:
(i) one or more single amino acid deletions from pin domains and/or hook domains of the plurality of Dda helicases;
(ii) one or more single amino acid deletions from pin domains and/or hook domains of the plurality of Dda helicases, and a variant of SEQ ID NO: 8 which comprises deletion of E93 and/or deletion of E95; or
(iii) one or more single amino acid deletions from pin domains and/or hook domains of the plurality of Dda helicases, and a variant of any one of SEQ ID NOs: 9 to 23 which comprises deletion of the position corresponding to E93 in SEQ ID NO: 8 and/or deletion of the position corresponding to E95 in SEQ ID NO: 8; or
(B) at least one cysteine residue and/or at least one non-natural amino acid have been further introduced into hook domains and/or 2A (RecA-like) domains of the plurality of Dda helicases; or
(C) the plurality of Dda helicases comprises:
(a) a variant of SEQ ID NO: 8 in which at least one cysteine residue and/or at least one non-natural amino acid have further been introduced into the hook domain (residues L275-F291) and/or the 2A (RecA-like) domain (residues R178-T259 and L390-V439);
(b) a variant of SEQ ID NO: 9 in which at least one cysteine residue and/or at least one non-natural amino acid have further been introduced into the hook domain (residues A310-L315) and/or the 2A (RecA-like) domain (residues R212-E294 and G422-S678);
(c) a variant of SEQ ID NO: 10 in which at least one cysteine residue and/or at least one non-natural amino acid have further been introduced into the hook domain (residues V343-L359) and/or the 2A (RecA-like) domain (residues R241-N327 and A449-G496);
(d) a variant of SEQ ID NO: 11 in which at least one cysteine residue and/or at least one non-natural amino acid have further been introduced into the hook domain (residues W276-L284) and/or the 2A (RecA-like) domain (residues R174-D260 and A371-V421);
(e) a variant of SEQ ID NO: 12 in which at least one cysteine residue and/or at least one non-natural amino acid have further been introduced into the hook domain (residues R308-Y313) and/or the 2A (RecA-like) domain (residues R206-K293 and I408-L500);
(f) a variant of SEQ ID NO: 13 in which at least one cysteine residue and/or at least one non-natural amino acid have further been introduced into the hook domain (residues M302-W306) and/or the 2A (RecA-like) domain (residues R195-D287 and V394-Q450);
(g) a variant of SEQ ID NO: 14 in which at least one cysteine residue and/or at least one non-natural amino acid have further been introduced into the hook domain (residues V265-1277) and/or the 2A (RecA-like) domain (residues R167-T249 and L372-N421);
(h) a variant of SEQ ID NO: 15 in which at least one cysteine residue and/or at least one non-natural amino acid have further been introduced into the hook domain (residues V270-F283) and/or the 2A (RecA-like) domain (residues R172-T254 and L381-K434);
(i) a variant of SEQ ID NO: 16 in which at least one cysteine residue and/or at least one non-natural amino acid have further been introduced into the hook domain (residues V257-F270) and/or the 2A (RecA-like) domain (residues R159-T241 and L367-K420);
(j) a variant of SEQ ID NO: 17 in which at least one cysteine residue and/or at least one non-natural amino acid have further been introduced into the hook domain (residues L278-Y294) and/or the 2A (RecA-like) domain (residues R182-T262 and L393-V443);
(k) a variant of SEQ ID NO: 18 in which at least one cysteine residue and/or at least one non-natural amino acid have further been introduced into the hook domain (residues L278-Y294) and/or the 2A (RecA-like) domain (residues R182-T262 and L392-V442);
(l) a variant of SEQ ID NO: 19 in which at least one cysteine residue and/or at least one non-natural amino acid have further been introduced into the hook domain (residues L273-F289) and/or the 2A (RecA-like) domain (residues R177-N257 and L387-V438);
(m) a variant of SEQ ID NO: 20 in which at least one cysteine residue and/or at least one non-natural amino acid have further been introduced into the hook domain (residues L281-F297) and/or the 2A (RecA-like) domain (residues R184-T265 and L393-1442);
(n) a variant of SEQ ID NO: 21 in which at least one cysteine residue and/or at least one non-natural amino acid have further been introduced into the hook domain (residues H277-F293) and/or the 2A (RecA-like) domain (residues R180-T261 and L393-V442);
(o) a variant of SEQ ID NO: 22 in which at least one cysteine residue and/or at least one non-natural amino acid have further been introduced into the hook domain (residues L276-F292) and/or the 2A (RecA-like) domain (residues R179-T260 and L390-1439); or
(p) a variant of SEQ ID NO: 23 in which at least one cysteine residue and/or at least one non-natural amino acid have further been introduced into the hook domain (residues L276-F292) and/or the 2A (RecA-like) domain (residues R179-T260 and L391-V441); or
(D) the plurality of Dda helicases are further modified to reduce their surface negative charge; or
(E) the plurality of Dda helicases are further modified by the removal of one or more native cysteine residues; or
(F) at least one cysteine residue and/or at least one non-natural amino acid have been introduced into only tower domains of the plurality of Dda helicases, wherein the plurality of Dda helicases retain their ability to control the movement of a polynucleotide; or
(G) the at least one non-natural amino acids is selected from 4-Azido-L-phenylalanine (Faz), 4-Acetyl-L-phenylalanine, 3-Acetyl-L-phenylalanine, 4-Acetoacetyl-L-phenylalanine, O-Allyl-L-tyrosine, 3-(Phenylselanyl)-L-alanine, O-2-Propyn-1-yl-L-tyrosine, 4-(Dihydroxyboryl)-L-phenylalanine, 4-[(Ethylsulfanyl)carbonyl]-L-phenylalanine, (2S)-2-amino-3-{4-[(propan-2-ylsulfanyl)carbonyl]phenyl propanoic acid, (2S)-2-amino-3-{4-[(2-amino-3-sulfanylpropanoyl)amino]phenyl}propanoic acid, 0-Methyl-L-tyrosine, 4-Amino-L-phenylalanine, 4-Cyano-L-phenylalanine, 3-Cyano-L-phenylalanine, 4-Fluoro-L-phenylalanine, 4-Iodo-L-phenylalanine, 4-Bromo-L-phenylalanine, 0-(Trifluoromethyl)tyrosine, 4-Nitro-L-phenylalanine, 3-Hydroxy-L-tyrosine, 3-Amino-L-tyrosine, 3-Iodo-L-tyrosine, 4-Isopropyl-L-phenylalanine, 3-(2-Naphthyl)-L-alanine, 4-Phenyl-L-phenylalanine, (2S)-2-amino-3-(naphthalen-2-ylamino)propanoic acid, 6-(Methylsulfanyl)norleucine, 6-Oxo-L-lysine, D-tyrosine, (2R)-2-Hydroxy-3-(4-hydroxyphenyl)propanoic acid, (2R)-2-Ammoniooctanoate3-(2,2′-Bipyridin-5-yl)-D-alanine, 2-amino-3-(8-hydroxy-3-quinolyl)propanoic acid, 4-Benzoyl-L-phenylalanine, S-(2-Nitrobenzyl)cysteine, (2R)-2-amino-3-[(2-nitrobenzyl)sulfanyl]propanoic acid, (2S)-2-amino-3-[(2-nitrobenzyl)oxy]propanoic acid, 0-(4,5-Dimethoxy-2-nitrobenzyl)-L-serine, (2S)-2-amino-6-({[(2-nitrobenzyl)oxy]carbonyl amino)hexanoic acid, 0-(2-Nitrobenzyl)-L-tyrosine, 2-Nitrophenylalanine, 4-[(E)-Phenyldiazenyl]-L-phenylalanine, 4-[3-(Trifluoromethyl)-3H-diaziren-3-yl]-D-phenylalanine, 2-amino-3-[[5-(dimethylamino)-I-naphthyl]sulfonylaminolpropanoic acid, (2S)-2-amino-4-(7-hydroxy-2-oxo-2H-chromen-4-yl)butanoic acid, (2S)-3-[(6-acetylnaphthalen-2-yl)aminol-2-aminopropanoic acid, 4-(Carboxymethyl)phenylalanine, 3-Nitro-L-tyrosine, O-Sulfo-L-tyrosine, (2R)-6-Acetamido-2-ammoniohexanoate, I-Methyl histidine, 2-Aminononanoic acid, 2-Aminodecanoic acid, L-Homocysteine, 5-Sulfanylnorvaline, 6-Sulfanyl-L-norleucine, 5-(Methylsulfanyl)-L-norvaline, N.sup.6-{[(2R,3R)-3-Methyl-3,4-dihydro-2H-pyrrol-2-yl]carbonyl}-L-lysine, N.sup.6-[(Benzyloxy)carbonyl]lysine, (2S)-2-amino-6-[(cyclopentylcarbonyl)amino]hexanoic acid, N.sup.6-[(Cyclopentyloxy)carbonyl]-L-lysine, (2S)-2-amino-6-[(2R)-tetrahydrofuran-2-ylcarbonyl]amino}hexanoic acid, (2S)-2-amino-8-[(2R,3S)-3-ethynyltetrahydrofuran-2-yl]-8-oxooctanoic acid, N.sup.6-(tert-Butoxycarbonyl)-L-lysine, (2S)-2-Hydroxy-6-({[(2-methyl-2-propanyl)oxy]carbonyl}amino)hexanoic acid, N.sup.6-[(Allyloxy)carbonyl]lysine, (2S)-2-amino-6-({[(2-azidobenzyl)oxy]carbonyl}amino)hexanoic acid, N.sup.6-L-Prolyl-L-lysine, (2S)-2-amino-6-{[(prop-2-yn-1-yloxy)carbonyl]amino}hexanoic acid and N.sup.6-[(2-Azidoethoxy)carbonyl]-L-lysine.

71. The apparatus of claim 60, wherein the plurality of Dda helicases comprises:
(a) a variant of SEQ ID NO: 8 which comprises:
(i) E94C and/or A360C; (ii) E93C and/or K358C; (iii) E93C and/or A360C; (iv) E93C and/or E361C; (v) E93C and/or K364C; (vi) E94C and/or L354C; (vii) E94C and/or K358C; (viii) E93C and/or L354C; (ix) E94C and/or E361C; (x) E94C and/or K364C; (xi) L97C and/or L354C; (xii) L97C and/or K358C; (xiii) L97C and/or A360C; (xiv) L97C and/or E361C; (xv) L97C and/or K364C; (xvi) K123C and/or L354C; (xvii) K123C and/or K358C; (xviii) K123C and/or A360C; (xix) K123C and/or E361C; (xx) K123C and/or K364C; (xxi) N155C and/or L354C; (xxii) N155C and/or K358C; (xxiii) N155C and/or A360C; (xxiv) N155C and/or E361C; (xxv) N155C and/or K364C; (xxvi) any of (i) to (xxv) and G357C; (xxvii) any of (i) to (xxv) and Q100C; (xxviii) any of (i) to (xxv) and I127C; (xxix) any of (i) to (xxv) and Q100C and I127C; (xxx) E94C and/or F377C; (xxxi) N95C; (xxxii) T91C; (xxxiii) Y92L, E94Y, Y350N, A360C and Y363N; (xxxiv) E94Y and A360C; (xxxv) A360C; (xxxvi) Y92L, E94C, Y350N, A360Y and Y363N; (xxxvii) Y92L, E94C and A360Y; (xxxviii) E94C and/or A360C and F276A; (xxxix) E94C and/or L356C; (xl) E93C and/or E356C; (xli) E93C and/or G357C; (xlii) E93C and/or A360C; (xliii) N95C and/or W378C; (xliv) T91C and/or S382C; (xlv) T91C and/or W378C; (xlvi) E93C and/or N353C; (xlvii) E93C and/or S382C; (xlviii) E93C and/or K381C; (xlix) E93C and/or D379C; (1) E93C and/or S375C; (li) E93C and/or W378C; (lii) E93C and/or W374C; (liii) E94C and/or N353C; (liv) E94C and/or S382C; (lv) E94C and/or K381C; (lvi) E94C and/or D379C; (lvii) E94C and/or S375C; (lviii) E94C and/or W378C; (lix) E94C and/or W374C; (lx) E94C and A360Y; (lxi) E94C, G357C and A360C or (lxii) T2C, E94C and A360C; or
(b) a variant of any one of SEQ ID NOs: 9 to 23 which comprises a cysteine residue at the positions which correspond to those in SEQ ID NO: 8 in any of (i) to (xxix); or
(c) a variant of SEQ ID NO:8 which comprises any of (i) to (xxix), wherein 4-Azido-L-phenylalanine (Faz) is introduced at one or more of the specific positions instead of cysteine, or
(d) a variant of any one of SEQ ID NOs: 9 to 23 which comprises a 4-Azido-L-phenylalanine (Faz) residue at the positions which correspond to those in SEQ ID NO: 8 in any of ci) to (xxix).

72. The apparatus according to claim 71, wherein the plurality of Dda helicases comprises:
(a) a variant of SEQ ID NO: 8 which comprises one or more of (i) I181C; (ii) Y279C; (iii) I281C; and (iv) E288C; or
(b) a variant of any one of SEQ ID NOs: 9 to 23 which comprises a cysteine residue at one or more of the position(s) which correspond to those in SEQ ID NO: 8 as defined in (i) to (iv); or
(c) a variant of SEQ ID NO: 8 which comprises one or more of (i) to (iv), wherein Faz is introduced at one or more of the specific positions instead of cysteine; or
(d) a variant of any one of SEQ ID NOs: 9 to 23 which comprises a Faz residue at one or more of the position(s) which correspond to those in SEQ ID NO: 8 as defined in (i) to (iv).

73. The apparatus according to claim 71, wherein:
(a) one or more surface negative charges of the plurality of Dda helicases have been neutralised by substituting one or more negatively charged amino acids with one or more positively charged amino acids, uncharged amino acids, non-polar amino acids and/or aromatic amino acids or by introducing one or more positively charged amino acids adjacent to one or more negatively charged amino acids; and/or
(b) the plurality of Dda helicases comprises a variant of SEQ ID NO: 8 and the one or more negatively charged amino acids are one or more of D5, E8, E23, E47, D167, E172, D202 and D212.

74. The apparatus according to claim 71, wherein the plurality of Dda helicase comprises:
(a) a variant of SEQ ID NO: 8 and the one or more native cysteine residues are one or more of C109, C114, C136, C171 and C412; or
(b) a variant of any one of SEQ ID NOs: 9 to 23 and the one or more native cysteine residues correspond to one or more of C109, C114, C136, C171 and C412 in SEQ ID NO: 8.

75. The apparatus according to claim 60, wherein the plurality of Dda helicases are modified to reduce their surface negative charge, wherein:
(a) the plurality of Dda helicases retain their ability to control the movement of a polynucleotide; or
(b) the plurality of Dda helicases retain ability to control the movement of a polynucleotide and one or more surface negative charges of the plurality of Dda helicases have been neutralised by substituting one or more negatively charged amino acids with one or more positively charged amino acids, uncharged amino acids, non-polar amino acids and/or aromatic amino acids or by introducing one or more positively charged amino acids adjacent to one or more negatively charged amino acids in the plurality of Dda helicases.

76. The apparatus according to claim 60, wherein:
(a) two more of the introduced cysteines and/or non-natural amino acids in the plurality of Dda helicases are connected to one another; or
(b) none of the introduced cysteines and/or non-natural amino acids in the plurality of Dda helicases are connected to one another.

77. An apparatus for characterising target polynucleotides in a sample, comprising:
(a) a plurality of pores; and
(b) a plurality of DNA-dependent ATPAase (Dda) helicases comprising a variant of SEQ ID NO: 8 comprising
(i) A360K;
(ii) Y92L and/or A360Y;
(iii) Y92L, Y350N and Y363N;
(iv) Y92L and/or Y363N; or
(v) Y92L;
wherein the plurality of Dda helicases are attached to additional polynucleotide binding moieties; and
wherein the plurality of Dda helicases retain their ability to control the movement of a polynucleotide.

78. The apparatus according to claim 77, wherein the apparatus comprises:
a sensor device capable of supporting the plurality of pores and being operable to perform polynucleotide characterisation using the plurality of pores and the plurality of Dda helicases; and
at least one port for delivery of material for performing the characterisation.

79. The apparatus according to claim 77, wherein the apparatus comprises:
a sensor device capable of supporting the plurality of pores and being operable to perform polynucleotide characterisation using the plurality of pores and the plurality of Dda helicases; and
at least one reservoir for holding material for performing the characterisation.

80. The apparatus according to claim 77, wherein the apparatus comprises:
a sensor device capable of supporting the plurality of pores and being operable to perform polynucleotide characterisation using the plurality of pores and the plurality of Dda helicases;
at least one reservoir for holding material for performing the characterisation;
a fluidics system configured to controllably supply material from the at least one reservoir to the sensor device; and
one or more containers for receiving respective samples, the fluidics system being configured to supply the samples selectively from the one or more containers to the sensor device.


SEQUENCE LISTING (80 sequences)

SEQ ID NO: 1 - DNA, 558 bp, unidentified
  misc_feature 1..558: note = Mycobacterium smegmatis porin A mutant(D90N/D91N/D93N/D118R/D134R/E139K)
  source 1..558: mol_type = other DNA
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, unidentified
  REGION 1..184: note = Mycobacterium smegmatis porin A mutant(D90N/D91N/D93N/D118R/D134R/E139K)
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 885 bp, unidentified
  misc_feature 1..885: note = alpha-hemolysin mutant E111N/K147N
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, unidentified
  REGION 1..293: note = alpha-hemolysin mutant E111N/K147N
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, Mycobacterium smegmatis
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - protein, 439 aa, unidentified
  source 1..439: note = Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - protein, 678 aa, Rhodothermus marinus
MEELSNEQQRVLDHVLAWLERNDAPPIFILTGSAGTGKTLLIRHLVRALQDRRIHYALAAPTGRAARILSERTGDHARTLHSLIYIFDRYQLVEEADRQTDEPLSLQLHFALRSAEHDARLIIVDEASMVSDTAGEEELYRFGSGRLLNDLLTFARLIPKRDRPPTTRLLFVGDPAQLPPVGQSVSPALSAQYLRDTFGLSAETAHLRSVYRQRKGHPILETATALRNALEKGHYHTFRLPEQPPDLRPVGLEEAIETTATDFRRQNPSVLLCRTNALARKLNAAVRARLWGREGLPPQPGDLLLVNRNAPLHGLFNGDLVLVETVGPLEHRRVGRRGRPPVDLYFRDVELLYPHEKPRNRIRCKLLENLLESPDGQLSPDIIQALLIDFYRRHPSLKHGSSEFRLMLANDAYFNALHVRYGYAMTVHKAQGGEWKRATVVFNDWRHFRHAEFFRWAYTAITRAREELLTIGAPSFEALSDMRWQPAPSVPAPEQAAENATRFPLKALETYHQRLSEALTAAGIETTGVELLQYAVRYHLARADRTTRIQYYYRGDGQISRIVTLGGADDPELTQQAYALFERILSEPPADSGELPENPLLREFLERAHLRLEGSGIRIVHWKEMPYALRLYFSADGENVTIDFYYNRRGVWTHAQEVGRSSSGALFARIQSLLQADS

SEQ ID NO: 10 - protein, 496 aa, unidentified
  source 1..496: note = Cyanothece ATCC51142
MSQSVVVPDELGEIITAVIEFYQDAVDKIEPKIVFLELRKNVVDWVSRTQLKIEEKEIQATGLTRQQQTAYKEMINFIENSSEQYFRLSGYAGTGKSFLMAKVIEWLKQEDYKYSVAAPTNKAAKNLTQIARSQGIKIEATTVAKLLKLQPTIDVDTGQQSFEFNSEKELELKDYDVIIIDEYSMLNKDNFRDLQQAVKGGESKFIFVGDSSQLPPVKEKEPIVANHPDIRKSANLTQIVRYDGEIVKVAESIRRNPRWNHQTYPFETVADGTIIKLNTEDWLQQALSHFEKEDWLSNPDYVRMITWRNKTADKYNQAIREALYGENVEQLVVGDRLIAKKPVFRSLPGGKKKEKKIILNNSEECKVIETPKINYNEKYKWEFYQVKVRTDEGGMIELRILTSESEEKRQKKLKELAKRAREEENYSEKKKQWAIYYELDELFDNMAYAYALTCHKAQGSSIDNVFLLVSDMHYCRDKTKMIYTGLTRAKKCCYVG

SEQ ID NO: 11 - protein, 421 aa, Salinibacter ruber
MSTFADAPFTEDQEEAYDHVYDRLAQGERFTGLRGYAGTGKTYLVSRLVEQLLDEDCTVTVCAPTHKAVQVLSDELGDAPVQMQTLHSFLGLRLQPKQDGEYELVAEEERNFAEGVVIVDEASMIGREEWSHIQDAPFWVQWLFVGDPAQLPPVNEDPSPALDVPGPTLETIHRQAADNPILELATKIRTGADGRFGSTFEDGKGVAVTRNREEFLDSILRAFDADAFAEDATHARVLAYRNKTVRRYNREIRAERYGADADRFVEGEWLVGTETWYYDGVQRLTNSEEVRVKKAQVETFEADDQSEWTVWELKIRTPGRGLTRTIHVLHEEERERYENALERRRGKAEDDPSKWDRFFELRERFARVDYAYATTVHRAQGSTYDTVFVDHRDLRVCRGEERGALLYVAVTRPSRRLALLV

SEQ ID NO: 12 - protein, 500 aa, unidentified
  source 1..500: note = Sulfurimonas gotlandica GD1
MKILNKETYKLSLHQEEVFTQIVSQLDTKVSSILKSTNIEDYLLSLTGPAGTGKTFLTTQIAKYLVEKRKESEYPMSSDFDFTITAPTHKAVGVLSKLLRENNIQSSCKTIHSFLGIKPFIDYTTGEEKFVVDKTNKRKDRTSILIVDESSMIGNTLYEYILEAIEDKRVNVVLFIGDPYQLLPIENSKNEIYDLPNRFFLSEVVRQAENSYIIRVATKLRERIKNQDFISLQQFFQENMEDEITFFHNKEAFLEDFYKEEEWYKENKILATYKNKDVDAFNKIIRNKFWEQKGNTTPSTLLAGDMIRFKDAYTVGDITIYHNGQELQLGSTEVKYHDSLHIEYWECKSIYALEQQVFRVVNPDSEAVFNQKLQSLATKAKQAKFPDNKKLWKLYYETRNMFANVQYIHASTIHKLQGSTYDVSYIDIFSLVHNHYMSDEEKYRLLYVAITRASKDIKIFMSAFDRTSDEKVIINNQNSETMNTLKQLHDIDIILKDLDL

SEQ ID NO: 13 - protein, 450 aa, unidentified
  source 1..450: note = Vibrio phage henriette 12B8
MADFELTLGQKTVLGEVISTILKPVNLNDTSRFHTMHGPAGSGKTTVLQRIISQIPAYKTIGFCSPTHKSVKVIRRMAREAGISHRVDIRTIHSALGLVMKPVRGDEVLVKEPFAEERIYDVLIIDEAGMLNDELIMYILESQSSKVIFVGDMCQIGPIQSNLPEEDGYTPTSTDDVSKVFTEVEMMSALTEVVRQAEGSPIIQLATEFRLAQDDIYADLPRIVTNTTPDGNGIITMPNGNWVDSAVARFQSDQFKEDPDHCRIVCYTNAMVDLCNDLVRKRLFGADVPEWLEDEILVAQEMGSTWNNADELRIVSIDDHFDQQYEVPCWRMQLESVEDHKLHNALVVKGDYIEDFKFRLNAIAERANTDKNMSGMHWKEFWGMRKKFNTFKNVYAGTAHKSQGSTFDYTYVFTPDFYKFGATMTIKRLLYTAITRSRYTTYFAMNTGAQ

SEQ ID NO: 14 - protein, 421 aa, unidentified
  source 1..421: note = Vibrio phage phi-pp2
MGLTNCQQGAMDAFLESDGHMTISGPAGSGKTFLMKSILEALESKGKNVTMVTPTHQAKNVLHKATGQEVSTIHSLLKIHPDTYEDQKHFTQSGEVEGLDEIDVLVVEEASMVDEELFQITGRTMPRKCRILAVGDKYQLQPVKHDPGVISPFFTKFTTFEMNEVVRQAKDNPLIQVATEVRNGQWLRTNWSKERRQGVLHVPNVNKMLDTYLSKVNSPEDLLDYRILAYTNDCVDTFNGIIREHVYNTSEPFIPGEYLVTQMPVMVSNGKYPVCVIENGEVVKILDVRQKTIDGMLPKVDNEAFDVAVLTVEKEDGNVYEFTVLWDDLQKERFARYLSVAAGTYKSMRGNTKRYWRAFWGLKEQMIETKSLGASTVHKSQGTTVKGVCLYTQDMGYAEPEILQQLVYVGLTRPTDWALYN

SEQ ID NO: 15 - protein, 434 aa, unidentified
  source 1..434: note = Aeromonas phage 65
MSESEITLTPSQNMAVNEVKNGTGHITISGPPGSGKTFLVKYLIKMLGDELGTVLAAPTHQAKIVLTEMSGIEACTIHSLMKIHPETLEDIQIFDQSKLPDLSNIRYLIVEEASMHSKTLFKITMKSIPPTCRIIAIGDKDQIQPEEHAQGELSPYFTDPRFSQIRLTDIMRQSLDNPIIQVATKIREGGWIEPNWNRDTKTGVYKVSGITDLVNSYLRAVKTPEDLTKYRFLAYTNKVVNKVNSIVREHVYKTKLPFIEGEKIVLQEPVMVEHEDDTIETIFTNGEVVTINEIEVFDRTIRIDGSPEFKVNAAKLSVSSDYSGIEHDFCVLYGSESRLEFEYQLSESAGNIKQMGKGGNQRSAWKSFWAAKKMFIETKSLGASTIHKSQGSTVKGVWLALHDIHYADEELKQQLVYVGVTRPTDFCLYFDGTK

SEQ ID NO: 16 - protein, 420 aa, unidentified
  source 1..420: note = Aeromonas phage CC2
MAVDAVQSGTGHITISGPPGSGKTFLVKYIIKMLGDELGTVLAAPTHQAKIVLTEMSGIEACTIHSLMKIHPETLEDIQIFDQSKMPDLSTVRYLIIEEASMHSKALFNITMKSIPPTCRIIAIGDKDQIQPVDHAPGELSPYFTDSRFTQIRMTDIMRQSLDNPIIQVATTIREGGWIYQNWNKEKKSGVYKVKSITDLINSYLRVVKTPEDLTKYRFLAFTNKVVDKVNSIVRKHVYKTDLPFIEGEKLVLQEPVMVEYDDDTIETIFTNGEVVTVDEIEVSDMNIRIDGSPAFSISVAKLKVTSDFSGVTHDIMSVYGEDSKAEFNYQLSEAAAVIKQMQRGQTKAAWASFWDAKKTFTETKSLGACTIHKSQGSTVKGVWLGLHDISYADTDLQQQLVYVGVTRPTDFCLYFDGSK

SEQ ID NO: 17 - protein, 443 aa, unidentified
  source 1..443: note = Cronobacter phage vB CsaM GAP161
MSELTFDDLSDDQKSAHDRVIHNIQNAIHTTITGGPGVGKTTLVKFVFNTLKGLGISGIWLTAPTHQAKNVLAAATGMDATTIHSALKISPVTNEELRVFEQQKGKKAPDLSTCRVFVVEEVSMVDMDLFRIIRRSIPSNAVILGLGDKDQIRPVNADGRVELSPFFDEEIFDVIRMDKIMRQAEGNPIIQVSRAVRDGKMLKPMSVGDLGVFQHANAVDFLRQYFRRVKTPDDLIENRMFAYTNDNVDKLNATIRKHLYKTTEPFILDEVIVMQEPLVQEMRLNGQIFTEIVYNNNEKIRVLEIIPRREVIKAEKCDEKIEIEFYLLKTVSLEEETEAQIQVVVDPVMKDRLGNYLAYVASTYKRIKQQTGYKAPWHSFWAIKNKFQDVKPLPVCTYHKSQGSTYDHAYMYTRDAYAFADYDLCKQLIYVGVTRARYTVDYV

SEQ ID NO: 18 - protein, 442 aa, unidentified
  source 1..442: note = Klebsiella phage KP15
MSELTFDDLSEDQKNAHDRVIKNIRNKIHTTITGGPGVGKTTLVKFVFETLKKLGISGIWLTAPTHQAKNVLSEAVGMDATTIHSALKISPVTNEELRVFEQQKGKKAADLSECRVFVVEEVSMVDKELFRIIKRTIPSCAVILGLGDKDQIRPVNTEGITELSPFFDEEIFDVIRMDKIMRQAEGNPIIQVSRAIRDGKPLMPLMNGELGVMKHENASDFLRRYFSRVKTPDDLNNNRMFAYTNANVDKLNAVIRKHLYKTDQPFIVGEVVVMQEPLVTEGRVNGVSFVEVIYNNNEQIKILEIIPRSDTIKADRCDPVQIDYFLMKTESMFEDTKADIQVIADPVMQERLGDYLNYVAFQYKKMKQETGYKAPWYSFWQIKNKFQTVKALPVCTYHKGQGSTYDHSYMYTRDAYAYADYELCKQLLYVGTTRARFTVDYV

SEQ ID NO: 19 - protein, 438 aa, unidentified
  source 1..438: note = Stenotrophomonas phage IME13
MVTYDDLTVGQKDAIEKALQAMRTKRHITIRGPAGSGKTTMTRFLLERLFQTGQQGIVLTAPTHQAKKELSKHALRKSYTIQSVLKINPSTLEENQIFEQKGTPDFSKTRVLICDEVSFYTRKLFDILMRNVPSHCVVIGIGDKAQIRGVSEDDTHELSPFFTDNRFEQVELTEVKRHQGPIIEVATDIRNGKWIYEKLDDSGNGVKQFHTVKDFLSKYFERTKTPNDLLENRIMAYTNNSVDKLNSVIRKQLYGANAAPFLPDEILVMQEPLMFDIDIGGQTLKEVIFNNGQNVRVINVKPSRKTLKAKGVGEIEVECTMLECESYEEDEDDYRRAWFTVVHDQNTQYAINEFLSIIAEKYRSREVFPNWKDFWAIRNTFVKVRPLGAMTFHKSQGSTFDNAYLFTPCLHQYCRDPDVAQELIYVGNTRARKNVCFV

SEQ ID NO: 20 - protein, 442 aa, unidentified
  source 1..442: note = Acinetobacter phage Ac42
MNFEDLTEGQKNAYTAAIKAIETVPSSSAEKRHLTINGPAGTGKTTLTKFLIAELIRRGERGVYLAAPTHQAKKVLSQHAGMEASTIHSLLKINPTTYEDSTTFEQKDVPDMSECRVLICDEASMYDLKLFQILMSSIPLCCTVIALGDIAQIRPVEPGAFEGQVSPFFTYEKFEQVSLTEVMRSNAPIIDVATSIRTGNWIYENVIDGAGVHNLTSERSVKSFMEKYFSIVKTPEDLFENRLLAFTNKSVDDLNKIVRKKIYNTLEPFIDGEVLVMQEPLIKSYTYEGKKVSEIVFNNGEMVKVLCCSQTSDEISVRGCSTKYMVRYWQLDLQSLDDPDLTGSINVIVDEAEINKLNLVLGKSAEQFKSGAVKAAWADWWKLKRNFHKVKALPCSTIHKSQGTSVDNVFLYTPCIHKADSQLAQQLLYVGATRARHNVYYI

SEQ ID NO: 21 - protein, 442 aa, unidentified
  source 1..442: note = Shigella phage SP18
MIKFEDLNTGQKEAFDYITEAIQRRSGECITLNGPAGTGKTTLTKFVIDHLVRNGVMGIVLAAPTHQAKKVLSKLSGQTANTIHSILKINPTTYEDQNIFEQREMPDMSKCNVLVCDEASMYDGSLFKIICNSVPEWCTILGIGDMHQLQPVDPGSTQQKISPFFTHPKFKQIHLTEVMRSNAPIIEVATEIRNGGWFRDCMYDGHGVQGFTSQTALKDFMVNYFGIVKDADMLMENRMYAYTNKSVEKLNNIIRRKLYETDKAFLPYEVLVMQEPHMKELEFEGKKFSETIFNNGQLVRIKDCKYTSTILRCKGESHQLVINYWDLEVESIDEDEEYQVDRIKVLPEDQQPKFQAYLAKVADTYKQMKAAGKRPEWKDFWKARRTFLKVRALPVSTIHKAQGVSVDKAFIYTPCIHMAEASLASQLAYVGITRARYDAYYV

SEQ ID NO: 22 - protein, 439 aa, unidentified
  source 1..439: note = Yersinia phage phiR1-RT
MITYDDLTDGQKSAFDNTMEAIKNKKGHITINGPAGTGKTTLTKFIIDHLIKTGEAGIILCAPTHQAKKVLSKLSGMDASTIHSVLKINPTTYEENQIFEQREVPDLAACRVLICDEASFYDRKLFGIILATVPSWCTVIALGDKDQLRPVTPGESEQQLSPFFSHAKFKQVHLTEIKRSNGPIIQVATDIRNGGWLSENIVDGEGVHAFNSNTALKDFMIRYFDVVKTADDLIESRMLAYTNKSVDKLNGIIRRKLYETDKPFINGEVLVMQEPLMKELEFDGKKFHEIVFNNGQLVKILYASETSTFISARNVPGEYMIRYWNLEVETADSDDDYATSQIQVICDPAEMTKFQMFLAKTADTYKNSGVKAYWKDFWSVKNKFKKVKALPVSTIHKSQGCTVNNTFLYTPCIHMADAQLAKQLLYVGATRARTNLYYI

SEQ ID NO: 23 - protein, 441 aa, unidentified
  source 1..441: note = Salmonella phage S16
MITFEQLTSGQKLAFDETIRAIKEKKNHVTINGPAGTGKTTLTKFIMEHLVSTGETGIILTAPTHAAKKVLTKLSGMEANTIHKILKINPTTYEESMLFEQKEVPDLASCRVLICDEASMWDRKLFKILMASIPKWCTIVAIGDVAQIRPVDPGETEAHISPFFIHKDFKQLNLTEVMRSNAPIIDVATDIRNGSWIYEKTVDGHGVHGFTSTTALKDFMMQYFSIVKSPEDLFENRMLAFTNKSVDKLNSIIRRRLYQTEEAFVVGEVIVMQEPLMRELVFEGKKFHETLFTNGQYVRILSADYTSSFLGAKGVSGEHLIRHWVLDVETYDDEEYAREKINVISDEQEMNKFQFFLAKTADTYKNWNKGGKAPWSEFWDAKRKFHKVKALPCSTFHKAQGISVDSSFIYTPCIHVSSDNKFKLELLYVGATRGRHDVFFV

SEQ ID NO: 24 - protein, 65 aa, unidentified
  REGION 1..65: note = preferred HhH domain
GTGSGAWKEWLERKVGEGRARRLIEYFGSAGEVGKLVENAEVSKLLEVPGIGDEAVARLVPGGSS

SEQ ID NO: 25 - protein, 299 aa, unidentified
  source 1..299: note = Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 26 - protein, 232 aa, unidentified
  source 1..232: note = Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 27 - protein, 324 aa, unidentified
  source 1..324: note = Herpes virus 1
MDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKHHHHHH

SEQ ID NO: 28 - protein, 251 aa, unidentified
  REGION 1..251: note = subunit 1 of PCNA
MFKIVYPNAKDFFSFINSITNVTDSIILNFTEDGIFSRHLTEDKVLMAIMRIPKDVLSEYSIDSPTSVKLDVSSVKKILSKASSKKATIELTETDSGLKIIIRDEKSGAKSTIYIKAEKGQVEQLTEPKVNLAVNFTTDESVLNVIAADVTLVGEEMRISTEEDKIKIEAGEEGKRYVAFLMKDKPLKELSIDTSASSSYSAEMFKDAVKGLRGFSAPTMVSFGENLPMKIDVEAVSGGHMIFWIAPRLLE

SEQ ID NO: 29 - protein, 245 aa, unidentified
  REGION 1..245: note = subunit 2 of PCNA
MKAKVIDAVSFSYILRTVGDFLSEANFIVTKEGIRVSGIDPSRVVFLDIFLPSSYFEGFEVSQEKEIIGFKLEDVNDILKRVLKDDTLILSSNESKLTLTFDGEFTRSFELPLIQVESTQPPSVNLEFPFKAQLLTITFADIIDELSDLGEVLNIHSKENKLYFEVIGDLSTAKVELSTDNGTLLEASGADVSSSYGMEYVANTTKMRRASDSMELYFGSQIPLKLRFKLPQEGYGDFYIAPRAD

SEQ ID NO: 30 - protein, 246 aa, unidentified
  REGION 1..246: note = sununit 3 of PCNA
MKVVYDDVRVLKDIIQALARLVDEAVLKFKQDSVELVALDRAHISLISVNLPREMFKEYDVNDEFKFGFNTQYLMKILKVAKRKEAIEIASESPDSVIINIIGSTNREFNVRNLEVSEQEIPEINLQFDISATISSDGFKSAISEVSTVTDNVVVEGHEDRILIKAEGESEVEVEFSKDTGGLQDLEFSKESKNSYSAEYLDDVLSLTKLSDYVKISFGNQKPLQLFFNMEGGGKVTYLLAPKVLE

SEQ ID NO: 31 - protein, 608 aa, unidentified
  source 1..608: note = Bacillus subtilis phage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 32 - protein, 318 aa, unidentified
  source 1..318: note = Herpes virus 1
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 33 - protein, 233 aa, unidentified
  source 1..233: note = Bacteriophage RB69
KGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAAS

SEQ ID NO: 34 - protein, 210 aa, unidentified
  source 1..210: note = Bacteriophage T7
AKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPR

SEQ ID NO: 35 - protein, 99 aa, unidentified
  source 1..99: note = Halorubrum lacusprofundi
SGEELLDLAGVRNVGRKRARRLFEAGIETRADLREADKAVVLGALRGRERTAERILEHAGREDPSMDDVRPDKSASAAATAGSASDEDGEGQASLGDFR

SEQ ID NO: 36 - protein, 102 aa, unidentified
  source 1..102: note = Haloferax volcanii
SGEELLDLAGVRGVGRKRARRLFEAGVETRADLREADKPRVLAALRGRRKTAENILEAAGRKDPSMDAVDEDDAPDDAVPDDAGFETAKERADQQASLGDFE

SEQ ID NO: 37 - protein, 55 aa, unidentified
  REGION 1..55: note = (HhH)2 domain
WKEWLERKVGEGRARRLIEYFGSAGEVGKLVENAEVSKLLEVPGIGDEAVARLVP

SEQ ID NO: 38 - protein, 107 aa, unidentified
  REGION 1..107: note = (HhH)2-(HhH)2 domain
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 39 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 40 - protein, 123 aa, unidentified
  source 1..123: note = Bacillus subtilis phage phi29
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 41 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 42 - protein, 301 aa, unidentified
  source 1..301: note = Enterobacteria phage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 43 - protein, 177 aa, unidentified
  REGION 1..177: note = EcoSSB-CterAla
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMAFAAAIPF

SEQ ID NO: 44 - protein, 177 aa, unidentified
  REGION 1..177: note = EcoSSB-CterNGGN
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMNFGGNIPF

SEQ ID NO: 45 - protein, 152 aa, unidentified
  REGION 1..152: note = EcoSSB-Q152del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQ

SEQ ID NO: 46 - protein, 117 aa, unidentified
  REGION 1..117: note = EcoSSB-G117del
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQG

SEQ ID NO: 47 - protein, 984 aa, Methanopyrus kandleri
MALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 48 - protein, 299 aa, Methanopyrus kandleri
SGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 49 - protein, 853 aa, Escherichia coli
MSAIENFDAHTPMMQQYLRLKAQHPEILLFYRMGDFYELFYDDAKRASQLLDISLTKRGASAGEPIPMAGIPYHAVENYLAKLVNQGESVAICEQIGDPATSKGPVERKVVRIVTPGTISDEALLQERQDNLLAAIWQDSKGFGYATLDISSGRFRLSEPADRETMAAELQRTNPAELLYAEDFAEMSLIEGRRGLRRRPLWEFEIDTARQQLNLQFGTRDLVGFGVENAPRGLCAAGCLLQYAKDTQRTTLPHIRSITMEREQDSIIMDAATRRNLEITQNLAGGAENTLASVLDCTVTPMGSRMLKRWLHMPVRDTRVLLERQQTIGALQDFTAGLQPVLRQVGDLERILARLALRTARPRDLARMRHAFQQLPELRAQLETVDSAPVQALREKMGEFAELRDLLERAIIDTPPVLVRDGGVIASGYNEELDEWRALADGATDYLERLEVRERERTGLDTLKVGFNAVHGYYIQISRGQSHLAPINYMRRQTLKNAERYIIPELKEYEDKVLTSKGKALALEKQLYEELFDLLLPHLEALQQSASALAELDVLVNLAERAYTLNYTCPTFIDKPGIRITEGRHPVVEQVLNEPFIANPLNLSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWACAENLANKIKALTLFATHYFELTQLPEKMEGVANVHLDALEHGDTIAFMHSVQDGAASKSYGLAVAALAGVPKEVIKRARQKLRELESISPNAAATQVDGTQMSLLSVPEETSPAVEALENLDPDSLTPRQALEWIYRLKSLV

SEQ ID NO: 50 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 51 - protein, 99 aa, unidentified
  source 1..99: note = Sulfolobus solfataricus P2
EKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 52 - protein, 88 aa, unidentified
  source 1..88: note = Sulfolobus solfataricus P2
TEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 53 - protein, 107 aa, Escherichia coli
AQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 54 - protein, 237 aa, unidentified
  source 1..237: note = Enterobacteria phage lambda
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 55 - protein, 60 aa, unidentified
  source 1..60: note = Crenarchaea
MSSGKKPVKVKTPAGKEAELVPEKVWALAPKGRKGVKIGLFKDPETGKYFRHKLPDDYPI

SEQ ID NO: 56 - protein, 136 aa, Homo sapiens [sequence omitted]

SEQ ID NO: 57 - protein, 89 aa, unidentified
  source 1..89: note = Enterobacteria phage T4
MAKKEMVEFDEAIHGEDLAKFIKEASDHKLKISGYNELIKDIRIRAKDELGVDGKMFNRLLALYHKDNRDVFEAETEEVVELYDTVFSK

SEQ ID NO: 58 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 59 - protein, 375 aa, unidentified
  source 1..375: note = Citromicrobium bathyomarinum JL354
MKATIERATLLRCLSHVQSVVERRNTIPILSNVLIDADAGGGVKVMATDLDLQVVETMTAASVESAGAITVSAHLLFDIARKLPDGSQVSLETADNRMVVKAGRSRFQLPTLPRDDFPVIVEGELPTSFELPARELAEMIDRTRFAISTEETRYYLNGIFLHVSDEARPVLKAAATDGHRLARYTLDRPEGAEGMPDVIVPRKAVGELRKLLEEALDSNVQIDLSASKIRFALGGEGGVVLTSKLIDGTFPDYSRVIPTGNDKLLRLDPKAFFQGVDRVATIATEKTRAVKMGLDEDKVTLSVTSPDNGTAAEEIAAEYKAEGFEIGFNANYLKDILGQIDSDTVELHLADAGAPTLIRRDENSPALYVLMPMRV

SEQ ID NO: 60 - DNA, 50 bp, synthetic construct
  misc_feature 1..50: note = Synthetic Polynucleotide
  source 1..50: mol_type = other DNA
tttttttttttttttttttttttttttttttttttttttttttttttttt

SEQ ID NO: 61 - DNA, 27 bp, synthetic construct
  misc_feature 1..27: note = Synthetic Polynucleotide
  source 1..27: mol_type = other DNA
ggttgtttctgttggtgctgatattgc

SEQ ID NO: 62 - DNA, 97138 bp, synthetic construct
  misc_feature 1..97138: note = Synthetic Polynucleotide
  source 1..97138: mol_type = other DNA
gctccactaaagggccgattgacgggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcaacaaaaagcagctggctgacattttcggtgcgagtatccgtaccattcagaactggcaggaacagggaatgcccgttctgcgaggcggtggcaagggtaatgaggtgctttatgactctgccgccgtcataaaatggtatgccgaaagggatgctgaaattgagaacgaaaagctgcgccgggaggttgaagaactgcggcaggccagcgaggcagatctccagccaggaactattgagtacgaacgccatcgacttacgcgtgcgcaggccgacgcacaggaactgaagaatgccagagactccgctgaagtggtggaaaccgcattctgtactttcgtgctgtcgcggatcgcaggtgaaattgccagtattctcgacgggctccccctgtcggtgcagcggcgttttccggaactggaaaaccgacatgttgatttcctgaaacgggatatcatcaaagccatgaacaaagcagccgcgctggatgaactgataccggggttgctgagtgaatatatcgaacagtcaggttaacaggctgcggcattttgtccgcgccgggcttcgctcactgttcaggccggagccacagaccgccgttgaatgggcggatgctaattactatctcccgaaagaatccgcataccaggaagggcgctgggaaacactgccctttcagcgggccatcatgaatgcgatgggcagcgactacatccgtgaggtgaatgtggtgaagtctgcccgtgtcggttattccaaaatgctgctgggtgtttatgcctactttatagagcataagcagcgcaacacccttatctggttgccgacggatggtgatgccgagaactttatgaaaacccacgttgagccgactattcgtgatattccgtcgctgctggcgctggccccgtggtatggcaaaaagcaccgggataacacgctcaccatgaagcgtttcactaatgggcgtggcttctggtgcctgggcggtaaagcggcaaaaaactaccgtgaaaagtcggtggatgtggcgggttatgatgaacttgctgcttttgatgatgatattgaacaggaaggctctccgacgttcctgggtgacaagcgtattgaaggctcggtctggccaaagtccatccgtggctccacgccaaaagtgagaggcacctgtcagattgagcgtgcagccagtgaatccccgcattttatgcgttttcatgttgcctgcccgcattgcggggaggagcagtatcttaaatttggcgacaaagagacgccgtttggcctcaaatggacgccggatgacccctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggcgggattgacccgaccattgtgtatgaacgctcgaaaaaacatgggctgttccgggtgatccccattaaaggggcatccgtctacggaaagccggtggccagcatgccacgtaagcgaaacaaaaacggggtttaccttaccgaaatcggtacggataccgcgaaagagcagatttataaccgcttcacactgacgccggaaggggatgaaccgcttcccggtgccgttcacttcccgaataacccggatatttttgatctgaccgaagcgcagcagctgactgctgaagagcaggtcgaaaaatgggtggatggcaggaaaaaaatactgtgggacagcaaaaagcgacgcaatgaggcactcgactgcttcgtttatgcgctggcggcgctgcgcatcagtatttcccgctggcagctggatctcagtgcgctgctggcgagcctgcaggaagaggatggtgcagcaaccaacaagaaaacactggcagattacgcccgtgccttatccggagaggatgaatgacgcgacaggaagaacttgccgctgcccgtgcggcactgcatgacctgatgacaggtaaacgggtggcaacagtacagaaagacggacgaagggtggagtttacggccacttccgtgtctgacctgaaaaaatatattgcagagctggaagtgcagaccggcatgacacagcgacgcaggggacctgcaggattttatgtatgaaaacgcccaccattcccacccttctggggccggacggcatgacatcgctgcgcgaatatgccggttatcacggcggtggcagcggatttggagggcagttgcggtcgtggaacccaccgagtgaaagtgtggatgcagccctgttgcccaactttacccgtggcaatgcccgcgcagacgatctggtacgcaataacggctatgccgccaacgccatccagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcctcgttcattcacgtttttgaacccgtggaggacgggcagactcgcggtgcaaatgtgttttacagcgtgatggagcagatgaagatgctcgacacgctgcagaacacgcagctgcagagcgccattgtgaaggcgatgtatgccgccaccattgagagtgagctggatacgcagtcagcgatggattttattctgggcgcgaacagtcaggagcagcgggaaaggctgaccggctggattggtgaaattgccgcgtattacgccgcagcgccggtccggctgggaggcgcaaaagtaccgcacctgatgccgggtgactcactgaacctgcagacggctcaggatacggataacggctactccgtgtttgagcagtcactgctgcggtatatcgctgccgggctgggtgtctcgtatgagcagctttcccggaattacgcccagatgagctactccacggcacgggccagtgcgaacgagtcgtgggcgtactttatggggcggcgaaaattcgtcgcatcccgtcaggcgagccagatgtttctgtgctggctggaagaggccatcgttcgccgcgtggtgacgttaccttcaaaagcgcgcttcagttttcaggaagcccgcagtgcctgggggaactgcgactggataggctccggtcgtatggccatcgatggtctgaaagaagttcaggaagcggtgatgctgatagaagccggactgagtacctacgagaaagagtgcgcaaaacgcggtgacgactatcaggaaatttttgcccagcaggtccgtgaaacgatggagcgccgtgcagccggtcttaaaccgcccgcctgggcggctgcagcatttgaatccgggctgcgacaatcaacagaggaggagaagagtgacagcagagctgcgtaatctcccgcatattgccagcatggcctttaatgagccgctgatgcttgaacccgcctatgcgcgggttttcttttgtgcgcttgcaggccagcttgggatcagcagcctgacggatgcggtgtccggcgacagcctgactgcccaggaggcactcgcgacgctggcattatccggtgatgatgacggaccacgacaggcccgcagttatcaggtcatgaacggcatcgccgtgctgccggtgtccggcacgctggtcagccggacgcgggcgctgcagccgtactcggggatgaccggttacaacggcattatcgcccgtctgcaacaggctgccagcgatccgatggtggacggcattctgctcgatatggacacgcccggcgggatggtggcgggggcatttgactgcgctgacatcatcgcccgtgtgcgtgacataaaaccggtatgggcgcttgccaacgacatgaactgcagtgcaggtcagttgcttgccagtgccgcctcccggcgtctggtcacgcagaccgcccggacaggctccatcggcgtcatgatggctcacagtaattacggtgctgcgctggagaaacagggtgtggaaatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtgaatccgcagatgaccctgcgtcgcctgccggatgaagatccgcagaatctggcggacccggcttaccgccgccgtcgcatcatcatgcagaacatgcgtgacgaagagctggccattgctcaggtcgaagagatgcaggcagtttctgccgtgcttaagggcaaatacaccatgaccggtgaagccttcgatccggttgaggtggatatgggccgcagtgaggagaataacatcacgcagtccggcggcacggagtggagcaagcgtgacaagtccacgtatgacccgaccgacgatatcgaagcctacgcgctgaacgccagcggtgtggtgaatatcatcgtgttcgatccgaaaggctgggcgctgttccgttccttcaaagccgtcaaggagaagctggatacccgtcgtggctctaattccgagctggagacagcggtgaaagacctgggcaaagcggtgtcctataaggggatgtatggcgatgtggccatcgtcgtgtattccggacagtacgtggaaaacggcgtcaaaaagaacttcctgccggacaacacgatggtgctggggaacactcaggcacgcggtctgcgcacctatggctgcattcaggatgcggacgcacagcgcgaaggcattaacgcctctgcccgttacccgaaaaactgggtgaccaccggcgatccggcgcgtgagttcaccatgattcagtcagcaccgctgatgctgctggctgaccctgatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggacggggcgtaccgcctgccgttaaccgtcgccgctgaaagggggatgtatggccataaaaggtcttgagcaggccgttgaaaacctcagccgtatcagcaaaacggcggtgcctggtgccgccgcaatggccattaaccgcgttgcttcatccgcgatatcgcagtcggcgtcacaggttgcccgtgagacaaaggtacgccggaaactggtaaaggaaagggccaggctgaaaagggccacggtcaaaaatccgcaggccagaatcaaagttaaccggggggatttgcccgtaatcaagctgggtaatgcgcgggttgtcctttcgcgccgcaggcgtcgtaaaaaggggcagcgttcatccctgaaaggtggcggcagcgtgcttgtggtgggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaatccggagagtcagcgatgttcctgaaaaccgaatcatttgaacataacggtgtgaccgtcacgctttctgaactgtcagccctgcagcgcattgagcatctcgccctgatgaaacggcaggcagaacaggcggagtcagacagcaaccggaagtttactgtggaagacgccatcagaaccggcgcgtttctggtggcgatgtccctgtggcataaccatccgcagaagacgcagatgccgtccatgaatgaagccgttaaacagattgagcaggaagtgcttaccacctggcccacggaggcaatttctcatgctgaaaacgtggtgtaccggctgtctggtatgtatgagtttgtggtgaataatgcccctgaacagacagaggacgccgggcccgcagagcctgtttctgcgggaaagtgttcgacggtgagctgagttttgccctgaaactggcgcgtgagatggggcgacccgactggcgtgccatgcttgccgggatgtcatccacggagtatgccgactggcaccgcttttacagtacccattattttcatgatgttctgctggatatgcacttttccgggctgacgtacaccgtgctcagcctgtttttcagcgatccggatatgcatccgctggatttcagtctgctgaaccggcgcgaggctgacgaagagcctgaagatgatgtgctgatgcagaaagcggcagggcttgccggaggtgtccgctttggcccggacgggaatgaagttatccccgcttccccggatgtggcggacatgacggaggatgacgtaatgctgatgacagtatcagaagggatcgcaggaggagtccggtatggctgaaccggtaggcgatctggtcgttgatttgagtctggatgcggccagatttgacgagcagatggccagagtcaggcgtcatttttctggtacggaaagtgatgcgaaaaaaacagcggcagtcgttgaacagtcgctgagccgacaggcgctggctgcacagaaagcggggatttccgtcgggcagtataaagccgccatgcgtatgctgcctgcacagttcaccgacgtggccacgcagcttgcaggcgggcaaagtccgtggctgatcctgctgcaacagggggggcaggtgaaggactccttcggcgggatgatccccatgttcagggggcttgccggtgcgatcaccctgccgatggtgggggccacctcgctggcggtggcgaccggtgcgctggcgtatgcctggtatcagggcaactcaaccctgtccgatttcaacaaaacgctggtcctttccggcaatcaggcgggactgacggcagatcgtatgctggtcctgtccagagccgggcaggcggcagggctgacgtttaaccagaccagcgagtcactcagcgcactggttaaggcgggggtaagcggtgaggctcagattgcgtccatcagccagagtgtggcgcgtttctcctctgcatccggcgtggaggtggacaaggtcgctgaagccttcgggaagctgaccacagacccgacgtcggggctgacggcgatggctcgccagttccataacgtgtcggcggagcagattgcgtatgttgctcagttgcagcgttccggcgatgaagccggggcattgcaggcggcgaacgaggccgcaacgaaagggtttgatgaccagacccgccgcctgaaagagaacatgggcacgctggagacctgggcagacaggactgcgcgggcattcaaatccatgtgggatgcggtgctggatattggtcgtcctgataccgcgcaggagatgctgattaaggcagaggctgcgtataagaaagcagacgacatctggaatctgcgcaaggatgattattttgttaacgatgaagcgcgggcgcgttactgggatgatcgtgaaaaggcccgtcttgcgcttgaagccgcccgaaagaaggctgagcagcagactcaacaggacaaaaatgcgcagcagcagagcgataccgaagcgtcacggctgaaatataccgaagaggcgcagaaggcttacgaacggctgcagacgccgctggagaaatataccgcccgtcaggaagaactgaacaaggcactgaaagacgggaaaatcctgcaggcggattacaacacgctgatggcggcggcgaaaaaggattatgaagcgacgctgaaaaagccgaaacagtccagcgtgaaggtgtctgcgggcgatcgtcaggaagacagtgctcatgctgccctgctgacgcttcaggcagaactccggacgctggagaagcatgccggagcaaatgagaaaatcagccagcagcgccgggatttgtggaaggcggagagtcagttcgcggtactggaggaggcggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaacgggcaaaacgggccgccattgatgcgaaaagccgggggctgactgaccggcaggcagaacgggaagccacggaacagcgcctgaaggaacagtatggcgataatccgctggcgctgaataacgtcatgtcagagcagaaaaagacctgggcggctgaagaccagcttcgcgggaactggatggcaggcctgaagtccggctggagtgagtgggaagagagcgccacggacagtatgtcgcaggtaaaaagtgcagccacgcagacctttgatggtattgcacagaatatggcggcgatgctgaccggcagtgagcagaactggcgcagcttcacccgttccgtgctgtccatgatgacagaaattctgcttaagcaggcaatggtggggattgtcgggagtatcggcagcgccattggcggggctgttggtggcggcgcatccgcgtcaggcggtacagccattcaggccgctgcggcgaaattccattttgcaaccggaggatttacgggaaccggcggcaaatatgagccagcggggattgttcaccgtggtgagtttgtcttcacgaaggaggcaaccagccggattggcgtggggaatctttaccggctgatgcgcggctatgccaccggcggttatgtcggtacaccgggcagcatggcagacagccggtcgcaggcgtccgggacgtttgagcagaataaccatgtggtgattaacaacgacggcacgaacgggcagataggtccggctgctctgaaggcggtgtatgacatggcccgcaagggtgcccgtgatgaaattcagacacagatgcgtgatggtggcctgttctccggaggtggacgatgaagaccttccgctggaaagtgaaacccggtatggatgtggcttcggtcccttctgtaagaaaggtgcgctttggtgatggctattctcagcgagcgcctgccgggctgaatgccaacctgaaaacgtacagcgtgacgctttctgtcccccgtgaggaggccacggtactggagtcgtttctggaagagcacgggggctggaaatcctttctgtggacgccgccttatgagtggcggcagataaaggtgacctgcgcaaaatggtcgtcgcgggtcagtatgctgcgtgttgagttcagcgcagagtttgaacaggtggtgaactgatgcaggatatccggcaggaaacactgaatgaatgcacccgtgcggagcagtcggccagcgtggtgctctgggaaatcgacctgacagaggtcggtggagaacgttattttttctgtaatgagcagaacgaaaaaggtgagccggtcacctggcaggggcgacagtatcagccgtatcccattcaggggagcggttttgaactgaatggcaaaggcaccagtacgcgccccacgctgacggtttctaacctgtacggtatggtcaccgggatggcggaagatatgcagagtctggtcggcggaacggtggtccggcgtaaggtttacgcccgttttctggatgcggtgaacttcgtcaacggaaacagttacgccgatccggagcaggaggtgatcagccgctggcgcattgagcagtgcagcgaactgagcgcggtgagtgcctcctttgtactgtccacgccgacggaaacggatggcgctgtttttccgggacgtatcatgctggccaacacctgcacctggacctatcgcggtgacgagtgcggttatagcggtccggctgtcgcggatgaatatgaccagccaacgtccgatatcacgaaggataaatgcagcaaatgcctgagcggttgtaagttccgcaataacgtcggcaactttggcggcttcctttccattaacaaactttcgcagtaaatcccatgacacagacagaatcagcgattctggcgcacgcccggcgatgtgcgccagcggagtcgtgcggcttcgtggtaagcacgccggagggggaaagatatttcccctgcgtgaatatctccggtgagccggaggctatttccgtatgtcgccggaagactggctgcaggcagaaatgcagggtgagattgtggcgctggtccacagccaccccggtggtctgccctggctgagtgaggccgaccggcggctgcaggtgcagagtgatttgccgtggtggctggtctgccgggggacgattcataagttccgctgtgtgccgcatctcaccgggcggcgctttgagcacggtgtgacggactgttacacactgttccgggatgcttatcatctggcggggattgagatgccggactttcatcgtgaggatgactggtggcgtaacggccagaatctctatctggataatctggaggcgacggggctgtatcaggtgccgttgtcagcggcacagccgggcgatgtgctgctgtgctgttttggttcatcagtgccgaatcacgccgcaatttactgcggcgacggcgagctgctgcaccatattcctgaacaactgagcaaacgagagaggtacaccgacaaatggcagcgacgcacacactccctctggcgtcaccgggcatggcgcgcatctgcctttacggggatttacaacgatttggtcgccgcatcgaccttcgtgtgaaaacgggggctgaagccatccgggcactggccacacagctcccggcgtttcgtcagaaactgagcgacggctggtatcaggtacggattgccgggcgggacgtcagcacgtccgggttaacggcgcagttacatgagactctgcctgatggcgctgtaattcatattgttcccagagtcgccggggccaagtcaggtggcgtattccagattgtcctgggggctgccgccattgccggatcattctttaccgccggagccacccttgcagcatggggggcagccattggggccggtggtatgaccggcatcctgttttctctcggtgccagtatggtgctcggtggtgtggcgcagatgctggcaccgaaagccagaactccccgtatacagacaacggataacggtaagcagaacacctatttctcctcactggataacatggttgcccagggcaatgttctgcctgttctgtacggggaaatgcgcgtggggtcacgcgtggtttctcaggagatcagcacggcagacgaaggggacggtggtcaggttgtggtgattggtcgctgatgcaaaatgttttatgtgaaaccgcctgcgggcggttttgtcatttatggagcgtgaggaatgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagtggcagtgtgaatgcgaactccgggacgctcagtaatgtgacgatagctgaaaactgtacgataaacggtacgctgagggcggaaaaaatcgtcggggacattgtaaaggcggcgagcgcggcttttccgcgccagcgtgaaagcagtgtggactggccgtcaggtacccgtactgtcaccgtgaccgatgaccatccttttgatcgccagatagtggtgcttccgctgacgtttcgcggaagtaagcgtactgtcagcggcaggacaacgtattcgatgtgttatctgaaagtactgatgaacggtgcggtgatttatgatggcgcggcgaacgaggcggtacaggtgttctcccgtattgttgacatgccagcgggtcggggaaacgtgatcctgacgttcacgcttacgtccacacggcattcggcagatattccgccgtatacgtttgccagcgatgtgcaggttatggtgattaagaaacaggcgctgggcatcagcgtggtctgagtgtgttacagaggttcgtccgggaacgggcgttttattataaaacagtgagaggtgaacgatgcgtaatgtgtgtattgccgttgctgtctttgccgcacttgcggtgacagtcactccggcccgtgcggaaggtggacatggtacgtttacggtgggctattttcaagtgaaaccgggtacattgccgtcgttgtcgggcggggataccggtgtgagtcatctgaaagggattaacgtgaagtaccgttatgagctgacggacagtgtgggggtgatggcttccctggggttcgccgcgtcgaaaaagagcagcacagtgatgaccggggaggatacgtttcactatgagagcctgcgtggacgttatgtgagcgtgatggccggaccggttttacaaatcagtaagcaggtcagtgcgtacgccatggccggagtggctcacagtcggtggtccggcagtacaatggattaccgtaagacggaaatcactcccgggtatatgaaagagacgaccactgccagggacgaaagtgcaatgcggcatacctcagtggcgtggagtgcaggtatacagattaatccggcagcgtccgtcgttgttgatattgcttatgaaggctccggcagtggcgactggcgtactgacggattcatcgttggggtcggttataaattctgattagccaggtaacacagtgttatgacagcccgccggaaccggtgggcttttttgtggggtgaatatggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgaaacgaacagaaaagcccactggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcctttccggcaggtgcgccgatcccgtggccatcagatatcgttccgtctggctacgtcctgatgcaggggcaggcgtttgacaaatcagcctacccaaaacttgctgtcgcgtatccatcgggtgtgcttcctgatatgcgaggctggacaatcaaggggaaacccgccagcggtcgtgctgtattgtctcaggaacaggatggaattaagtcgcacacccacagtgccagtgcatccggtacggatttggggacgaaaaccacatcgtcgtttgattacgggacgaaaacaacaggcagtttcgattacggcaccaaatcgacgaataacacgggggctcatgctcacagtctgagcggttcaacaggggccgcgggtgctcatgcccacacaagtggtttaaggatgaacagttctggctggagtcagtatggaacagcaaccattacaggaagtttatccacagttaaaggaaccagcacacagggtattgcttatttatcgaaaacggacagtcagggcagccacagtcactcattgtccggtacagccgtgagtgccggtgcacatgcgcatacagttggtattggtgcgcaccagcatccggttgttatcggtgctcatgcccattctttcagtattggttcacacggacacaccatcaccgttaacgctgcgggtaacgcggaaaacaccgtcaaaaacattgcatttaactatattgtgaggcttgcataatggcattcagaatgagtgaacaaccacggaccataaaaatttataatctgctggccggaactaatgaatttattggtgaaggtgacgcatatattccgcctcataccggtctgcctgcaaacagtaccgatattgcaccgccagatattccggctggctttgtggctgttttcaacagtgatgaggcatcgtggcatctcgttgaagaccatcggggtaaaaccgtctatgacgtggcttccggcgacgcgttatttatttctgaactcggtccgttaccggaaaattttacctggttatcgccgggaggggaatatcagaagtggaacggcacagcctgggtgaaggatacggaagcagaaaaactgttccggatccgggaggcggaagaaacaaaaaaaagcctgatgcaggtagccagtgagcatattgcgccgcttcaggatgctgcagatctggaaattgcaacgaaggaagaaacctcgttgctggaagcctggaagaagtatcgggtgttgctgaaccgtgttgatacatcaactgcacctgatattgagtggcctgctgtccctgttatggagtaatcgttttgtgatatgccgcagaaacgttgtatgaaataacgttctgcggttagttagtatattgtaaagctgagtattggtttatttggcgattattatcttcaggagaataatggaagttctatgactcaattgttcatagtgtttacatcaccgccaattgcttttaagactgaacgcatgaaatatggtttttcgtcatgttttgagtctgctgttgatatttctaaagtcggttttttttcttcgttttctctaactattttccatgaaatacatttttgattattatttgaatcaattccaattacctgaagtctttcatctataattggcattgtatgtattggtttattggagtagatgcttgcttttctgagccatagctctgatatccaaatgaagccataggcatttgttattttggctctgtcagctgcataacgccaaaaaatatatttatctgcttgatcttcaaatgttgtattgattaaatcaattggatggaattgtttatcataaaaaattaatgtttgaatgtgataaccgtcctttaaaaaagtcgtttctgcaagcttggctgtatagtcaactaactcttctgtcgaagtgatatttttaggcttatctaccagttttagacgctctttaatatcttcaggaattattttattgtcatattgtatcatgctaaatgacaatttgcttatggagtaatcttttaattttaaataagttattctcctggcttcatcaaataaagagtcgaatgatgttggcgaaatcacatcgtcacccattggattgtttatttgtatgccaagagagttacagcagttatacattctgccatagattatagctaaggcatgtaataattcgtaatcttttagcgtattagcgacccatcgtctttctgatttaataatagatgattcagttaaatatgaaggtaatttcttttgtgcaagtctgactaacttttttataccaatgtttaacatactttcatttgtaataaactcaatgtcattttcttcaatgtaagatgaaataagagtagcctttgcctcgctatacatttctaaatcgccttgtttttctatcgtattgcgagaatttttagcccaagccattaatggatcatttttccatttttcaataacattattgttataccaaatgtcatatcctataatctggtttttgtttttttgaataataaatgttactgttcttgcggtttggaggaattgattcaaattcaagcgaaataattcagggtcaaaatatgtatcaatgcagcatttgagcaagtgcgataaatctttaagtcttctttcccatggttttttagtcataaaactctccattttgataggttgcatgctagatgctgatatattttagaggtgataaaattaactgcttaactgtcaatgtaatacaagttgtttgatctttgcaatgattcttatcagaaaccatatagtaaattagttacacaggaaatttttaatattattattatcattcattatgtattaaaattagagttgtggcttggctctgctaacacgttgctcataggagatatggtagagccgcagacacgtcgtatgcaggaacgtgctgcggctggctggtgaacttccgatagtgcgggtgttgaatgatttccagttgctaccgattttacatattttttgcatgagagaatttgtaccacctcccaccgaccatctatgactgtacgccactgtccctaggactgctatgtgccggagcggacattacaaacgtccttctcggtgcatgccactgttgccaatgacctgcctaggaattggttagcaagttactaccggattttgtaaaaacagccctcctcatataaaaagtattcgttcacttccgataagcgtcgtaattttctatctttcatcatattctagatccctctgaaaaaatcttccgagtttgctaggcactgatacataactcttttccaataattggggaagtcattcaaatctataataggtttcagatttgcttcaataaattctgactgtagctgctgaaacgttgcggttgaactatatttccttataacttttacgaaagagtttctttgagtaatcacttcactcaagtgcttccctgcctccaaacgatacctgttagcaatatttaatagcttgaaatgatgaagagctctgtgtttgtcttcctgcctccagttcgccgggcattcaacataaaaactgatagcacccggagttccggaaacgaaatttgcatatacccattgctcacgaaaaaaaatgtccttgtcgatatagggatgaatcgcttggtgtacctcatctactgcgaaaacttgacctttctctcccatattgcagtcgcggcacgatggaactaaattaataggcatcaccgaaaattcaggataatgtgcaataggaagaaaatgatctatattttttgtctgtcctatatcaccacaaaatggacatttttcacctgatgaaacaagcatgtcatcgtaatatgttctagcgggtttgtttttatctcggagattattttcataaagcttttctaatttaacctttgtcaggttaccaactactaaggttgtaggctcaagagggtgtgtcctgtcgtaggtaaataactgacctgtcgagcttaatattctatattgttgttctttctgcaaaaaagtggggaagtgagtaatgaaattatttctaacatttatctgcatcataccttccgagcatttattaagcatttcgctataagttctcgctggaagaggtagttttttcattgtactttaccttcatctctgttcattatcatcgcttttaaaacggttcgaccttctaatcctatctgaccattataattttttagaatggtttcataagaaagctctgaatcaacggactgcgataataagtggtggtatccagaatttgtcacttcaagtaaaaacacctcacgagttaaaacacctaagttctcaccgaatgtctcaatatccggacggataatatttattgcttctcttgaccgtaggactttccacatgcaggattttggaacctcttgcagtactactggggaatgagttgcaattattgctacaccattgcgtgcatcgagtaagtcgcttaatgttcgtaaaaaagcagagagcaaaggtggatgcagatgaacctctggttcatcgaataaaactaatgacttttcgccaacgacatctactaatcttgtgatagtaaataaaacaattgcatgtccagagctcattcgaagcagatatttctggatattgtcataaaacaatttagtgaatttatcatcgtccacttgaatctgtggttcattacgtcttaactcttcatatttagaaatgaggctgatgagttccatatttgaaaagttttcatcactacttagttttttgatagcttcaagccagagttgtctttttctatctactctcatacaaccaataaatgctgaaatgaattctaagcggagatcgcctagtgattttaaactattgctggcagcattcttgagtccaatataaaagtattgtgtaccttttgctgggtcaggttgttctttaggaggagtaaaaggatcaaatgcactaaacgaaactgaaacaagcgatcgaaaatatccctttgggattcttgactcgataagtctattattttcagagaaaaaatattcattgttttctgggttggtgattgcaccaatcattccattcaaaattgttgttttaccacacccattccgcccgataaaagcatgaatgttcgtgctgggcatagaattaaccgtcacctcaaaaggtatagttaaatcactgaatccgggagcactttttctattaaatgaaaagtggaaatctgacaattctggcaaaccatttaacacacgtgcgaactgtccatgaatttctgaaagagttacccctctaagtaatgaggtgttaaggacgctttcattttcaatgtcggctaatcgatttggccatactactaaatcctgaatagctttaagaaggttatgtttaaaaccatcgcttaatttgctgagattaacatagtagtcaatgctttcacctaaggaaaaaaacatttcagggagttgactgaattttttatctattaatgaataagtgcttacttcttctttttgacctacaaaaccaattttaacatttccgatatcgcatttttcaccatgctcatcaaagacagtaagataaaacattgtaacaaaggaatagtcattccaaccatctgctcgtaggaatgccttatttttttctactgcaggaatatacccgcctctttcaataacactaaactccaacatatagtaacccttaattttattaaaataaccgcaatttatttggcggcaacacaggatctctcttttaagttactctctattacatacgttttccatctaaaaattagtagtattgaacttaacggggcatcgtattgtagttttccatatttagctttctgcttccttttggataacccactgttattcatgttgcatggtgcactgtttataccaacgatatagtctattaatgcatatatagtatcgccgaacgattagctcttcaggcttctgaagaagcgtttcaagtactaataagccgatagatagccacggacttcgtagccatttttcataagtgttaacttccgctcctcgctcataacagacattcactacagttatggcggaaaggtatgcatgctgggtgtggggaagtcgtgaaagaaaagaagtcagctgcgtcgtttgacatcactgctatcttcttactggttatgcaggtcgtagtgggtggcacacaaagctttgcactggattgcgaggctttgtgcttctctggagtgcgacaggtttgatgacaaaaaattagcgcaagaagacaaaaatcaccttgcgctaatgctctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaagttggcattataaaaaagcattgcttatcaatttgttgcaacgaacaggtcactatcagtcaaaataaaatcattatttgatttcaattttgtcccactccctgcctctgtcatcacgatactgtgatgccatggtgtccgacttatgcccgagaagatgttgagcaaacttatcgcttatctgcttctcatagagtcttgcagacaaactgcgcaactcgtgaaaggtaggcggatccccttcgaaggaaagacctgatgcttttcgtgcgcgcataaaataccttgatactgtgccggatgaaagcggttcgcgacgagtagatgcaattatggtttctccgccaagaatctctttgcatttatcaagtgtttccttcattgatattccgagagcatcaatatgcaatgctgttgggatggcaatttttacgcctgttttgctttgctcgacataaagatatccatctacgatatcagaccacttcatttcgcataaatcaccaactcgttgcccggtaacaacagccagttccattgcaagtctgagccaacatggtgatgattctgctgcttgataaattttcaggtattcgtcagccgtaagtcttgatctccttacctctgattttgctgcgcgagtggcagcgacatggtttgttgttatatggccttcagctattgcctctcggaatgcatcgctcagtgttgatctgattaacttggctgacgccgccttgccctcgtctatgtatccattgagcattgccgcaatttcttttgtggtgatgtcttcaagtggagcatcaggcagacccctccttattgctttaattttgctcatgtaatttatgagtgtcttctgcttgattcctctgctggccaggattttttcgtagcgatcaagccatgaatgtaacgtaacggaattatcactgttgattctcgctgtcagaggcttgtgtttgtgtcctgaaaataactcaatgttggcctgtatagcttcagtgattgcgattcgcctgtctctgcctaatccaaactctttacccgtccttgggtccctgtagcagtaatatccattgtttcttatataaaggttagggggtaaatcccggcgctcatgacttcgccttcttcccatttctgatcctcttcaaaaggccacctgttactggtcgatttaagtcaacctttaccgctgattcgtggaacagatactctcttccatccttaaccggaggtgggaatatcctgcattcccgaacccatcgacgaactgtttcaaggcttcttggacgtcgctggcgtgcgttccactcctgaagtgtcaagtacatcgcaaagtctccgcaattacacgcaagaaaaaaccgccatcaggcggcttggtgttctttcagttcttcaattcgaatattggttacgtctgcatgtgctatctgcgcccatatcatccagtggtcgtagcagtcgttgatgttctccgcttcgataactctgttgaatggctctccattccattctcctgtgactcggaagtgcatttatcatctccataaaacaaaacccgccgtagcgagttcagataaaataaatccccgcgagtgcgaggattgttatgtaatattgggtttaatcatctatatgttttgtacagagagggcaagtatcgtttccaccgtactcgtgataataattttgcacggtatcagtcatttctcgcacattgcagaatggggatttgtcttcattagacttataaaccttcatggaatatttgtatgccgactctatatctataccttcatctacataaacaccttcgtgatgtctgcatggagacaagacaccggatctgcacaacattgataacgcccaatctttttgctcagactctaactcattgatactcatttataaactccttgcaatgtatgtcgtttcagctaaacggtatcagcaatgtttatgtaaagaaacagtaagataatactcaacccgatgtttgagtacggtcatcatctgacactacagactctggcatcgctgtgaagacgacgcgaaattcagcattttcacaagcgttatcttttacaaaaccgatctcactctcctttgatgcgaatgccagcgtcagacatcatatgcagatactcacctgcatcctgaacccattgacctccaaccccgtaatagcgatgcgtaatgatgtcgatagttactaacgggtcttgttcgattaactgccgcagaaactcttccaggtcaccagtgcagtgcttgataacaggagtcttcccaggatggcgaacaacaagaaactggtttccgtcttcacggacttcgttgctttccagtttagcaatacgcttactcccatccgagataacaccttcgtaatactcacgctgctcgttgagttttgattttgctgtttcaagctcaacacgcagtttccctactgttagcgcaatatcctcgttctcctggtcgcggcgtttgatgtattgctggtttctttcccgttcatccagcagttccagcacaatcgatggtgttaccaattcatggaaaaggtctgcgtcaaatccccagtcgtcatgcattgcctgctctgccgcttcacgcagtgcctgagagttaatttcgctcacttcgaacctctctgtttactgataagttccagatcctcctggcaacttgcacaagtccgacaaccctgaacgaccaggcgtcttcgttcatctatcggatcgccacactcacaacaatgagtggcagatatagcctggtggttcaggcggcgcatttttattgctgtgttgcgctgtaattcttctatttctgatgctgaatcaatgatgtctgccatctttcattaatccctgaactgttggttaatacgcttgagggtgaatgcgaataataaaaaaggagcctgtagctccctgatgattttgcttttcatgttcatcgttccttaaagacgccgtttaacatgccgattgccaggcttaaatgagtcggtgtgaatcccatcagcgttaccgtttcgcggtgcttcttcagtacgctacggcaaatgtcatcgacgtttttatccggaaactgctgtctggctttttttgatttcagaattagcctgacgggcaatgctgcgaagggcgttttcctgctgaggtgtcattgaacaagtcccatgtcggcaagcataagcacacagaatatgaagcccgctgccagaaaaatgcattccgtggttgtcatacctggtttctctcatctgcttctgctttcgccaccatcatttccagcttttgtgaaagggatgcggctaacgtatgaaattcttcgtctgtttctactggtattggcacaaacctgattccaatttgagcaaggctatgtgccatctcgatactcgttcttaactcaacagaagatgctttgtgcatacagcccctcgtttattatttatctcctcagccagccgctgtgctttcagtggatttcggataacagaaaggccgggaaatacccagcctcgctttgtaacggagtagacgaaagtgattgcgcctacccggatattatcgtgaggatgcgtcatcgccattgctccccaaatacaaaaccaatttcagccagtgcctcgtccattttttcgatgaactccggcacgatctcgtcaaaactcgccatgtacttttcatcccgctcaatcacgacataatgcaggccttcacgcttcatacgcgggtcatagttggcaaagtaccaggcattttttcgcgtcacccacatgctgtactgcacctgggccatgtaagctgactttatggcctcgaaaccaccgagccggaacttcatgaaatcccgggaggtaaacgggcatttcagttcaaggccgttgccgtcactgcataaaccatcgggagagcaggcggtacgcatactttcgtcgcgatagatgatcggggattcagtaacattcacgccggaagtgaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgacagaatccatgccgacacgttcagccagcttcccagccagcgttgcgagtgcagtactcattcgttttatacctctgaatcaatatcaacctggtggtgagcaatggtttcaaccatgtaccggatgtgttctgccatgcgctcctgaaactcaacatcgtcatcaaacgcacgggtaatggattttttgctggccccgtggcgttgcaaatgatcgatgcatagcgattcaaacaggtgctggggcaggcctttttccatgtcgtctgccagttctgcctctttctcttcacgggcgagctgctggtagtgacgcgcccagctctgagcctcaagacgatcctgaatgtaataagcgttcatggctgaactcctgaaatagctgtgaaaatatcgcccgcgaaatgccgggctgattaggaaaacaggaaagggggttagtgaatgcttttgcttgatctcagtttcagtattaatatccattttttataagcgtcgacggcttcacgaaacatcttttcatcgccaataaaagtggcgatagtgaatttagtctggatagccataagtgtttgatccattctttgggactcctggctgattaagtatgtcgataaggcgtttccatccgtcacgtaatttacgggtgattcgttcaagtaaagattcggaagggcagccagcaacaggccaccctgcaatggcatattgcatggtgtgctccttatttatacataacgaaaaacgcctcgagtgaagcgttattggtatgcggtaaaaccgcactcaggcggccttgatagtcatatcatctgaatcaaatattcctgatgtatcgatatcggtaattcttattccttcgctaccatccattggaggccatccttcctgaccatttccatcattccagtcgaactcacacacaacaccatatgcatttaagtcgcttgaaattgctataagcagagcatgttgcgccagcatgattaatacagcatttaatacagagccgtgtttattgagtcggtattcagagtctgaccagaaattattaatctggtgaagtttttcctctgtcattacgtcatggtcgatttcaatttctattgatgctttccagtcgtaatcaatgatgtattttttgatgtttgacatctgttcatatcctcacagataaaaaatcgccctcacactggagggcaaagaagatttccaataatcagaacaagtcggctcctgtttagttacgagcgacattgctccgtgtattcactcgttggaatgaatacacagtgcagtgtttattctgttatttatgccaaaaataaaggccactatcaggcagctttgttgttctgtttaccaagttctctggcaatcattgccgtcgttcgtattgcccatttatcgacatatttcccatcttccattacaggaaacatttcttcaggcttaaccatgcattccgattgcagcttgcatccattgcatcgcttgaattgtccacaccattgatttttatcaatagtcgtagtcatacggatagtcctggtattgttccatcacatcctgaggatgctcttcgaactcttcaaattcttcttccatatatcaccttaaatagtggattgcggtagtaaagattgtgcctgtcttttaaccacatcaggctcggtggttctcgtgtacccctacagcgagaaatcggataaactattacaacccctacagtttgatgagtatagaaatggatccactcgttattctcggacgagtgttcagtaatgaacctctggagagaaccatgtatatgatcgttatctgggttggacttctgcttttaagcccagataactggcctgaatatgttaatgagagaatcggtattcctcatgtgtggcatgttttcgtctttgctcttgcattttcgctagcaattaatgtgcatcgattatcagctattgccagcgccagatataagcgatttaagctaagaaaacgcattaagatgcaaaacgataaagtgcgatcagtaattcaaaaccttacagaagagcaatctatggttttgtgcgcagcccttaatgaaggcaggaagtatgtggttacatcaaaacaattcccatacattagtgagttgattgagcttggtgtgttgaacaaaactttttcccgatggaatggaaagcatatattattccctattgaggatatttactggactgaattagttgccagctatgatccatataatattgagataaagccaaggccaatatctaagtaactagataagaggaatcgattttcccttaattttctggcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcgctttctactcgtgatttcggtttgcgattcagcgagagaatagggcggttaactggttttgcgcttaccccaaccaacaggggatttgctgctttccattgagcctgtttctctgcgcgacgttcgcggcggcgtgtttgtgcatccatctggattctcctgtcagttagctttggtggtgtgtggcagttgtagtcctgaacgaaaaccccccgcgattggcacattggcagctaatccggaatcgcacttacggccaatgcttcgtttcgtatcacacaccccaaagccttctgctttgaatgctgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatctgaattgctatgtttagtgagttgtatctatttatttttcaataaatacaattggttatgtgttttgggggcgatcgtgaggcaaagaaaacccggcgctgaggccgggttattcttgttctctggtcaaattatatagttggaaaacaaggatgcatatatgaatgaacgatgcagaggcaatgccgatggcgatagtgggtatcatgtagccgcttatgctggaaagaagcaataacccgcagaaaaacaaagctccaagctcaacaaaactaagggcatagacaataactaccgatgtcatatacccatactctctaatcttggccagtcggcgcgttctgcttccgattagaaacgtcaaggcagcaatcaggattgcaatcatggttcctgcatatgatgacaatgtcgccccaagaccatctctatgagctgaaaaagaaacaccaggaatgtagtggcggaaaaggagatagcaaatgcttacgataacgtaaggaattattactatgtaaacaccaggcatgattctgttccgcataattactcctgataattaatccttaactttgcccacctgccttttaaaacattccagtatatcacttttcattcttgcgtagcaatatgccatctcttcagctatctcagcattggtgaccttgttcagaggcgctgagagatggcctttttctgatagataatgttctgttaaaatatctccggcctcatcttttgcccgcaggctaatgtctgaaaattgaggtgacgggttaaaaataatatccttggcaaccttttttatatcccttttaaattttggcttaatgactatatccaatgagtcaaaaagctccccttcaatatctgttgcccctaagacctttaatatatcgccaaatacaggtagcttggcttctaccttcaccgttgttcggccgatgaaatgcatatgcataacatcgtctttggtggttcccctcatcagtggctctatctgaacgcgctctccactgcttaatgacattcctttcccgattaaaaaatctgtcagatcggatgtggtcggcccgaaaacagttctggcaaaaccaatggtgtcgccttcaacaaacaaaaaagatgggaatcccaatgattcgtcatctgcgaggctgttcttaatatcttcaactgaagctttagagcgatttatcttctgaaccagactcttgtcatttgttttggtaaagagaaaagtttttccatcgattttatgaatatacaaataattggagccaacctgcaggtgatgattatcagccagcagagaattaaggaaaacagacaggtttattgagcgcttatctttccctttatttttgctgcggtaagtcgcataaaaaccattcttcataattcaatccatttactatgttatgttctgaggggagtgaaaattcccctaattcgatgaagattcttgctcaattgttatcagctatgcgccgaccagaacaccttgccgatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcatgtactaaggaggttgtatggaacaacgcataaccctgaaagattatgcaatgcgctttgggcaaaccaagacagctaaagatctcggcgtatatcaaagcgcgatcaacaaggccattcatgcaggccgaaagatttttttaactataaacgctgatggaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatctaaggaaatacttacatatggttcgtgcaaacaaacgcaacgaggctctacgaatcgagagtgcgttgcttaacaaaatcgcaatgcttggaactgagaagacagcggaagctgtgggcgttgataagtcgcagatcagcaggtggaagagggactggattccaaagttctcaatgctgcttgctgttcttgaatggggggtcgttgacgacgacatggctcgattggcgcgacaagttgctgcgattctcaccaataaaaaacgcccggcggcaaccgagcgttctgaacaaatccagatggagttctgaggtcattactggatctatcaacaggagtcattatgacaaatacagcaaaaatactcaacttcggcagaggtaactttgccggacaggagcgtaatgtggcagatctcgatgatggttacgccagactatcaaatatgctgcttgaggcttattcgggcgcagatctgaccaagcgacagtttaaagtgctgcttgccattctgcgtaaaacctatgggtggaataaaccaatggacagaatcaccgattctcaacttagcgagattacaaagttacctgtcaaacggtgcaatgaagccaagttagaactcgtcagaatgaatattatcaagcagcaaggcggcatgtttggaccaaataaaaacatctcagaatggtgcatccctcaaaacgagggaaaatcccctaaaacgagggataaaacatccctcaaattgggggattgctatccctcaaaacagggggacacaaaagacactattacaaaagaaaaaagaaaagattattcgtcagagaattctggcgaatcctctgaccagccagaaaacgacctttctgtggtgaaaccggatgctgcaattcagagcggcagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgccaggacaacttctggtccggtaacgtgctgagcccggccaaactccgcgataagtggacccaactcgaaatcaaccgtaacaagcaacaggcaggcgtgacagccagcaaaccaaaactcgacctgacaaacacagactggatttacggggtggatctatgaaaaacatcgccgcacagatggttaactttgaccgtgagcagatgcgtcggatcgccaacaacatgccggaacagtacgacgaaaagccgcaggtacagcaggtagcgcagatcatcaacggtgtgttcagccagttactggcaactttcccggcgagcctggctaaccgtgaccagaacgaagtgaacgaaatccgtcgccagtgggttctggcttttcgggaaaacgggatcaccacgatggaacaggttaacgcaggaatgcgcgtagcccgtcggcagaatcgaccatttctgccatcacccgggcagtttgttgcatggtgccgggaagaagcatccgttaccgccggactgccaaacgtcagcgagctggttgatatggtttacgagtattgccggaagcgaggcctgtatccggatgcggagtcttatccgtggaaatcaaacgcgcactactggctggttaccaacctgtatcagaacatgcgggccaatgcgcttactgatgcggaattacgccgtaaggccgcagatgagcttgtccatatgactgcgagaattaaccgtggtgaggcgatccctgaaccagtaaaacaacttcctgtcatgggcggtagacctctaaatcgtgcacaggctctggcgaagatcgcagaaatcaaagctaagttcggactgaaaggagcaagtgtatgacgggcaaagaggcaattattcattacctggggacgcataatagcttctgtgcgccggacgttgccgcgctaacaggcgcaacagtaaccagcataaatcaggccgcggctaaaatggcacgggcaggtcttctggttatcgaaggtaaggtctggcgaacggtgtattaccggtttgctaccagggaagaacgggaaggaaagatgagcacgaacctggtttttaaggagtgtcgccagagtgccgcgatgaaacgggtattggcggtatatggagttaaaagatgaccatctacattactgagctaataacaggcctgctggtaatcgcaggcctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaaacatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgttctgtttatgtttcttgtttgttagccttttggctaacaaacaagaaacataaacagaacgcgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctgcttccggtattcgcttaattcagcacaacggaaagagcactggctaaccaggctcgccgactcttcacgattatcgactcaatgctcttacctgttgtgcagatataaaaaatcccgaaaccgttatgcaggctctaactattacctgcgaactgtttcgggattgcattttgcagacctctctgcctgcgatggttggagttccagacgatacgtcgaagtgaccaactaggcggaatcggtagtaagcgccgcctcttttcatctcactaccacaacgagcgaattaacccatcgttgagtcaaatttacccaattttattcaataagtcaatatcatgccgttaatatgttgccatccgtggcaatcatgctgctaacgtgtgaccgcattcaaaatgttgtctgcgattgactcttctttgtggcattgcaccaccagagcgtcatacagcggcttaacagtgcgtgaccaggtgggttgggtaaggtttgggattagcatcgtcacagcgcgatatgctgcgcttgctggcatccttgaatagccgacgcctttgcatcttccgcactctttctcgacaactctcccccacagctctgttttggcaatatcaaccgcacggcctgtaccatggcaatctctgcatcttgcccccggcgtcgcggcactacggcaataatccgcataagcgaatgttgcgagcacttgcagtacctttgccttagtatttccttcaagctttgccacaccacggtatttccccgataccttgtgtgcaaattgcatcagatagttgatagccttttgtttgtcgttctggctgagttcgtgcttaccgcagaatgcagccataccgaatccggcttgtgattgcgccatccccatagcagccatcacatcagtaccggaaagagagtcagaagccgtggcccgtggtgagtcgctcatcatcgggctttttggcgaatgaaatttagctacgctttcgagtctcatgcgccttctccctgtacctgaatcaatgttaggtttccgcagaacactgcgccggtatcgatatacatttggttggcaaacttgagtggtttcactgctggcgtatgaccaaagatgaacgtgtccgcgcctttgatttctttcacgatcccgttttgtgagttgctgattcgttcgcggttccagattacctgctgatgatcaactggctttccaaactcgtattcgtcaaagggataatcggcgtggcagataacatattttttatctttgctcaccagttcgatgattaacggaagttcatctgctttatgggcaagagctttagccagaatttctttgtcgtaatcgagattaaagaaccagccaccgccattaagcagccagtgattaacgtttccacgctctgataagccatcaatcatcatttgctcatggtttccacgtacagctctgaaccaggggaatgtgattaattccaggcattcaacgttctctgcaccacgatcaaccaaatcgcccaccgagataagcaggtcttttttgttgtcgaatccaatcgtatccagtttgttcatcaggttcgtgtagcatccgtgcagatcgccaactacccaaatatttcggtatttgctgccatcaattttttcgtaatagcgcatctctttcactccatccgcgatgaaccatgagaacgtcgttgacgatggcgtgcattttcccgtctttatcatcaacgtattttctgaccgtaccgcgactacatttcagtctgcgtgctacttctgtctgatttccgtatgtttcaacgagcatgtctggaatggtttttactgagaacgtcatgcggcctcacttctgctatttcgcaggtctttgagtttctgttggtactctgccttgatcgccttgcactcttcgatagtccagcgatggcggttatggtttgattcgatttcgtctactgcttcctgcccgatgcggctaatcagttcgacgcgatacggaacgagatttccgcttttgtgctggttgcacaccacgcattgcttgtgaatattgcgttcattaaatcggagttgaggtgccgcagcagttgtccggtaatgtccggcatcccactgagcagacgtgagcgttccgcacgagatacatggtaagtcgcggtctctttctctgatgaaggcgtttacggcttgttgggcttgtttaatccagtaactgcggggctttaaggcgagttttcgaatcttaagtttatctttctgtttctgctcctctcgtcgtcgtttcttctctgctgctttttccgctttttcgcgttctttacttcgtcgttcgagtgctatcttggttccacactctggagagcaccaccactgattagcgaatgcagggtgaaaccattcccggcattcatcgtttttacatcgtcttcgcgctggtttagccatcatcttcttcctcgtgcatcgagctattcggatcgctcatcagttctgcgcagcagtgctcacacacgtgaacttccagcacatgcagcttctgaccgcagttagcgcacgttaaagctcgctcgacgctttcttgttcgtaacttcgattttggtcaatcaccttgttttcctcgcacgacgtcttagccaccggatatcccacaggtgagccgtgtagttgaaggtttttacgtcagattcttttgggattggcttgggtttatttctggtgcgtttcgttggaaggtatttgcagttttcgcagattatgtcggtgatacttcgtcgctgtctcgccacacgtcctccttttcctgcggtagtggtaacacccctgttggtgttctttcacaccggagacaccatcgattccagtaaggttgatttggtcggaagcggttatcttctttgcattcaccgcaccgataacatcgcatcatgcagcttccctcccgaagtcgaaatcaagctgccctccaaatatttcgcatgactcagaacaagagccggtatcgaatcttttagctcgtaccatgtcctgatacagggcttgataatcattttctgaatacattttcgcgataccgtccagcgacattcttcctcggtacataatctcctttggcgtttcccgatgtccgtcacgcacatgggatcccgtgatgacctcattaaaaacacgctgcaatccctcctcatctttgcaggcaagtccgattttttgcgttgattttttaatgcagaatatgcagttaccgagatgttccggtatttgcaaatcgaatggttgttgcttccaccatgcgaggatatcttccttctcaaagtctgacagttcagcaagatatctgattccaggctttggctttagccgcttcggttcatcagctctgatgccaatccacgtggtgtaattccctcgcccgaaatggtcatcacagtatttggtgaagggaacgagttttaatctgtcagtgcagaacgcgccgccgacgtatggagtgccatatttctttaccatatcgataaatggcttcagaacaggcattcgcgtctgaatatcctttggttcccataccgtataaccatttggctgtccaagctccgggttgatatcaacctgcaatacggtgagcggtatatcccagaacttcacaacttccctgacaaaccgatatgtcattggatgttcacaacctgtatccatgaaaacgtaatgcacgtctttacctgcccgtcgcttttgctccattagccagagcaaatatgctgacgtcctgccaccggagaaactaacgacatttatcatgcagccctgtctccccatctcgctttccactccagagccagtctcgcttcgtctgaccacttaacgccacgctctgtaccgaatgcctgtataagctctaatagctccgcaaattcgcctacacgcatcctgctggttgactggcctattaccacaaagccattcccggcaaggttaggaacaacatcctgctgctttaatgctgcggtaaacacacacttccagctttctgcatccagccagcgaccatgccattcaacctgacgagagacgtcacctaagcaggcccatagcttcctgttttggtctaagctgcggttgcgttcctgaatggttactacgattggtttggttgggtctggaaggatttgctgtactgcgtgaatagcgttttgctgatgtgctggagatcgaatttcaaaggttagttttttcatgacttccctctcccccaaataaaaaggcctgcgattaccagcaggcctgttattagctcagtaatgtagatggtcatcttttaactccatataccgccaatacccgtttcatcgcggcactctggcgacactccttaaaaaccaggttcgtgctcatctttccttcccgttcttccctggtagcaaaccggtaatacaccgttcgccagaccttaccttcgataaccagaagacctgcccgtgccattttagccgcggcctgatttatgctggttactgttgcgcctgttagcgcggcaacgtccggcgcacagaagctattatgcgtccccaggtaatgaataattgcctctttgcccgtcatacacttgctcctttcagtccgaacttagctttgatttctgcgatcttcgccagagcctgtgcacgatttagaggtctaccgcccatgacaggaagttgttttactggttcagggatcgcctcaccacggttaattctcgcagtcatatggacaagctcatctgcggccttacggcgtaattccgcatcagtaagcgcattggcccgcatgttctgatacaggttggtaaccagccagtagtgcgcgtttgatttccacggataagactccgcatccggatacaggcctcgcttccggcaatactcgtaaaccatatcaaccagctcgctgacgtttggcagtccggcggtaacggatgcttcttcccggcaccatgcaacaaactgcccgggtgatggcagaaatggtcgattctgccgacgggctacgcgcattcctgcgttaacctgttccatcgtggtgatcccgttttcccgaaaagccagaacccactggcgacggatttcgttcacttcgttctggtcacggttagccaggctcgccgggaaagttgccagtaactggctgaacacaccgttgatgatctgcgctacctgctgtacctgcggcttttcgtcgtactgttccggcatgttgttggcgatccgacgcatctgctcacggtcaaagttaaccatctgtgcggcgatgtttttcatagatccaccccgtaaatccagtctgtgtttgtcaggtcgagttttggtttgctggctgtcacgcctgcctgttgcttgttacggttgatttcgagttgggtccacttatcgcggagtttggccgggctcagcacgttaccggaccagaagttgtcctggcatgcccagcggaacagcacacacatgtcgcggtggttacgtccgtcacgttcacgcatcaggcggatatcgttagcccacccagcaaaattcggttttctggctgatggtgcgatagtcttcaccatgtcaaacatccactctgcggcggtcaggtcttctgctgtcccccacttgctgccgctctgaattgcagcatccggtttcaccacagaaaggtcgttttctggctggtcagaggattcgccagaattctctgacgaataatcttttcttttttcttttgtaatagtgtcttttgtgtccccctgttttgagggatagcaatcccccaatttgagggatgttttatccctcgttttaggggattttccctcgttttgagggatgcaccattctgagatgtttttatttggtccaaacatgccgccttgctgcttgataatattcattctgacgagttctaacttggcttcattgcaccgtttgacaggtaactttgtaatctcgctaagttgagaatcggtgattctgtccattggtttattccacccataggttttacgcagaatggcaagcagcactttaaactgtcgcttggtcagatctgcgcccgaataagcctcaagcagcatatttgatagtctggcgtaaccatcatcgagatctgccacattacgctcctgtccggcaaagttacctctgccgaagttgagtatttttgctgtatttgtcataatgactcctgttgatagatccagtaatgacctcagaactccatctggatttgttcagaacgctcggttgccgccgggcgttttttattggtgagaatcgcagcaacttgtcgcgccaatcgagccatgtcgtcgtcaacgaccccccattcaagaacagcaagcagcattgagaactttggaatccagtccctcttccacctgctgatctgcgacttatcaacgcccacagcttccgctgtcttctcagttccaagcattgcgattttgttaagcaacgcactctcgattcgtagagcctcgttgcgtttgtttgcacgaaccatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggcctgcatgaatggccttgttgatcgcgctttgatatacgccgagatctttagctgtcttggtttgcccaaagcgcattgcataatctttcagggttatgcgttgttccatacaacctccttagtacatgcaaccattatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcaggttggctccaattatttgtatattcataaaatcgatggaaaaacttttctctttaccaaaacaaatgacaagagtctggttcagaagataaatcgctctaaagcttcagttgaagatattaagaacagcctcgcagatgacgaatcattgggattcccatcttttttgtttgttgaaggcgacaccattggttttgccagaactgttttcgggccgaccacatccgatctgacagattttttaatcgggaaaggaatgtcattaagcagtggagagcgcgttcagatagagccactgatgaggggaaccaccaaagacgatgttatgcatatgcatttcatcggccgaacaacggtgaaggtagaagccaagctacctgtatttggcgatatattaaaggtcttaggggcaacagatattgaaggggagctttttgactcattggatatagtcattaagccaaaatttaaaagggatataaaaaaggttgccaaggatattatttttaacccgtcacctcaattttcagacattagcctgcgggcaaaagatgaggccggagatattttaacagaacattatctatcagaaaaaggccatctctcagcgcctctgaacaaggtcaccaatgctgagatagctgaagagatggcatattgctacgcaagaatgaaaagtgatatactggaatgttttaaaaggcaggtgggcaaagttaaggattaattatcaggagtaattatgcggaacagaatcatgcctggtgtttacatagtaataattccttacgttatcgtaagcatttgctatctccttttccgccactacattcctggtgtttctttttcagctcatagagatggtcttggggcgacattgtcatcatatgcaggaaccatgattgcaatcctgattgctgccttgacgtttctaatcggaagcagaacgcgccgactggccaagattagagagtatgggtatatgacatcggtagttattgtctatgcccttagttttgttgagcttggagctttgtttttctgcgggttattgcttctttccagcataagcggctacatgatacccactatcgccatcggcattgcctctgcatcgttcattcatatatgcatccttgttttccaactatataatttgaccagagaacaagaataacccggcctcagcgccgggttttctttgcctcacgatcgcccccaaaacacataaccaattgtatttattgaaaaataaatagatacaactcactaaacatagcaattcagatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtaagtgcgattccggattagctgccaatgtgccaatcgcggggggttttcgttcaggactacaactgccacacaccaccaaagctaactgacaggagaatccagatggatgcacaaacacgccgccgcgaacgtcgcgcagagaaacaggctcaatggaaagcagcaaatcccctgttggttggggtaagcgcaaaaccagttaaccgccctattctctcgctgaatcgcaaaccgaaatcacgagtagaaagcgcactaaatccgatagaccttacagtgctggctgaataccacaaacagattgaaagcaacctgcaacgtattgagcgcaagaatcagcgcacatggtacagcaagcctggcgaacgcggcataacatgcagtggacgccagaaaattaagggaaaatcgattcctcttatctagttacttagatattggccttggctttatctcaatattatatggatcatagctggcaactaattcagtccagtaaatatcctcaatagggaataatatatgctttccattccatcgggaaaaagttttgttcaacacaccaagctcaatcaactcactaatgtatgggaattgttttgatgtaaccacatacttcctgccttcattaagggctgcgcacaaaaccatagattgctcttctgtaaggttttgaattactgatcgcactttatcgttttgcatcttaatgcgttttcttagcttaaatcgcttatatctggcgctggcaatagctgataatcgatgcacattaattgctagcgaaaatgcaagagcaaagacgaaaacatgccacacatgaggaataccgattctctcattaacatattcaggccagttatctgggcttaaaagcagaagtccaacccagataacgatcatatacatggttctctccagaggttcattactgaacactcgtccgagaataacgagtggatccatttctatactcatcaaactgtaggggttgtaatagtttatccgatttctcgctgtaggggtacacgagaaccaccgagcctgatgtggttaaaagacaggcacaatctttactaccgcaatccactatttaaggtgatatatggaagaagaatttgaagagttcgaagagcatcctcaggatgtgatggaacaataccaggactatccgtatgactacgactattgataaaaatcaatggtgtggacaattcaagcgatgcaatggatgcaagctgcaatcggaatgcatggttaagcctgaagaaatgtttcctgtaatggaagatgggaaatatgtcgataaatgggcaatacgaacgacggcaatgattgccagagaacttggtaaacagaacaacaaagctgcctgatagtggcctttatttttggcataaataacagaataaacactgcactgtgtattcattccaacgagtgaatacacggagcaatgtcgctcgtaactaaacaggagccgacttgttctgattattggaaatcttctttgccctccagtgtgagggcgattttttatctgtgaggatatgaacagatgtcaaacatcaaaaaatacatcattgattacgactggaaagcatcaatagaaattgaaatcgaccatgacgtaatgacagaggaaaaacttcaccagattaataatttctggtcagactctgaataccgactcaataaacacggctctgtattaaatgctgtattaatcatgctggcgcaacatgctctgcttatagcaatttcaagcgacttaaatgcatatggtgttgtgtgtgagttcgactggaatgatggaaatggtcaggaaggatggcctccaatggatggtagcgaaggaataagaattaccgatatcgatacatcaggaatatttgattcagatgatatgactatcaaggccgcctgagtgcggttttaccgcataccaataacgcttcactcgaggcgtttttcgttatgtataaataaggagcacaccatgcaatatgccattgcagggtggcctgttgctggctgcccttccgaatctttacttgaacgaatcacccgtaaattacgtgacggatggaaacgccttatcgacatacttaatcagccaggagtcccaaagaatggatcaaacacttatggctatccagactaaattcactatcgccacttttattggcgatgaaaagatgtttcgtgaagccgtcgacgcttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatttcatacgttagccgcatccctttcacaaaagctggaaatgatggtggcgaaagcagaagcagatgagagaaaccaggtatgacaaccacggaatgcatttttctggcagcgggcttcatattctgtgtgcttatgcttgccgacatgggacttgttcaatgacacctcagcaggaaaacgcccttcgcagcattgcccgtcaggctaattctgaaatcaaaaaaagccagacagcagtttccggataaaaacgtcgatgacatttgccgtagcgtactgaagaagcaccgcgaaacggtaacgctgatgggattcacaccgactcatttaagcctggcaatcggcatgttaaacggcgtctttaaggaacgatgaacatgaaaagcaaaatcatcagggagctacaggctccttttttattattcgcattcaccctcaagcgtattaaccaacagttcagggattaatgaaagatggcagacatcattgattcagcatcagaaatagaagaattacagcgcaacacagcaataaaaatgcgccgcctgaaccaccaggctatatctgccactcattgttgtgagtgtggcgatccgatagatgaacgaagacgcctggtcgttcagggttgtcggacttgtgcaagttgccaggaggatctggaacttatcagtaaacagagaggttcgaagtgagcgaaattaactctcaggcactgcgtgaagcggcagagcaggcaatgcatgacgactggggatttgacgcagaccttttccatgaattggtaacaccatcgattgtgctggaactgctggatgaacgggaaagaaaccagcaatacatcaaacgccgcgaccaggagaacgaggatattgcgctaacagtagggaaactgcgtgttgagcttgaaacagcaaaatcaaaactcaacgagcagcgtgagtattacgaaggtgttatctcggatgggagtaagcgtattgctaaactggaaagcaacgaagtccgtgaagacggaaaccagtttcttgttgttcgccatcctgggaagactcctgttatcaagcactgcactggtgacctggaagagtttctgcggcagttaatcgaacaagacccgttagtaactatcgacatcattacgcatcgctattacggggttggaggtcaatgggttcaggatgcaggtgagtatctgcatatgatgtctgacgctggcattcgcatcaaaggagagtgagatcggttttgtaaaagataacgcttgtgaaaatgctgaatttcgcgtcgtcttcacagcgatgccagagtctgtagtgtcagatgatgaccgtactcaaacatcgggttgagtattatcttactgtttctttacataaacattgctgataccgtttagctgaaacgacatacattgcaaggagtttataaatgagtatcaatgagttagagtctgagcaaaaagattgggcgttatcaatgttgtgcagatccggtgtcttgtctccatgcagacatcacgaaggtgtttatgtagatgaaggtatagatatagagtcggcatacaaatattccatgaaggtttataagtctaatgaagacaaatccccattctgcaatgtgcgagaaatgactgataccgtgcaaaattattatcacgagtacggtggaaacgatacttgccctctctgtacaaaacatatagatgattaaacccaatattacataacaatcctcgcactcgcggggatttattttatctgaactcgctacggcgggttttgttttatggagatgataaatgcacttccgagtcacaggagaatggaatggagagccattcaacagagttatcgaagcggagaacatcaacgactgctacgaccactggatgatatgggcgcagatagcacatgcagacgtaaccaatattcgaattgaagaactgaaagaacaccaagccgcctgatggcggttttttcttgcgtgtaattgcggagactttgcgatgtacttgacacttcaggagtggaacgcacgccagcgacgtccaagaagccttgaaacagttcgtcgatgggttcgggaatgcaggatattcccacctccggttaaggatggaagagagtatctgttccacgaatcagcggtaaaggttgacttaaatcgaccagtaacaggtggccttttgaagaggatcagaaatgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcagaggtaaggagatcaagacttacggctgacgaatacctgaaaatttatcaagcagcagaatcatcaccatgttggctcagacttgcaatggaactggctgttgttaccgggcaacgagttggtgatttatgcgaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataatgattttattttgactgatagtgacctgttcgttgcaacaaattgataagcaatgcttttttataatgccaacttagtataaaaaagctgaacgagaaacgtaaaatgatataaatatcaatatattaaattagattttgcataaaaaacagactacataatactgtaaaacacaacatatgcagtcactatgaatcaactacttagatggtattagtgacctgtaacagagcattagcgcaaggtgatttttgtcttcttgcgctaattttttgtcatcaaacctgtcgcactccagagaagcacaaagcctcgcaatccagtgcaaagctttgtgtgccacccactacgacctgcataaccagtaagaagatagcagtgatgtcaaacgacgcagctgacttcttttctttcacgacttccccacacccagcatgcatacctttccgccataactgtagtgaatgtctgttatgagcgaggagcggaagttaacacttatgaaaaatggctacgaagtccgtggctatctatcggcttattagtacttgaaacgcttcttcagaagcctgaagagctaatcgttcggcgatactatatatgcattaatagactatatcgttggtataaacagtgcaccatgcaacatgaataacagtgggttatccaaaaggaagcagaaagctaaatatggaaaactacaatacgatgccccgttaagttcaatactactaatttttagatggaaaacgtatgtaatagagagtaacttaaaagagagatcctgtgttgccgccaaataaattgcggttattttaataaaattaagggttactatatgttggagtttagtgttattgaaagaggcgggtatattcctgcagtagaaaaaaataaggcattcctacgagcagatggttggaatgactattcctttgttacaatgttttatcttactgtctttgatgagcatggtgaaaaatgcgatatcggaaatgttaaaattggttttgtaggtcaaaaagaagaagtaagcacttattcattaatagataaaaaattcagtcaactccctgaaatgtttttttccttaggtgaaagcattgactactatgttaatctcagcaaattaagcgatggttttaaacataaccttcttaaagctattcaggatttagtagtatggccaaatcgattagccgacattgaaaatgaaagcgtccttaacacctcattacttagaggggtaactctttcagaaattcatggacagttcgcacgtgtgttaaatggtttgccagaattgtcagatttccacttttcatttaatagaaaaagtgctcccggattcagtgatttaactataccttttgaggtgacggttaattctatgcccagcacgaacattcatgcttttatcgggcggaatgggtgtggtaaaacaacaattttgaatggaatgattggtgcaatcaccaacccagaaaacaatgaatattttttctctgaaaataatagacttatcgagtcaagaatcccaaagggatattttcgatcgcttgtttcagtttcgtttagtgcatttgatccttttactcctcctaaagaacaacctgacccagcaaaaggtacacaatacttttatattggactcaagaatgctgccagcaatagtttaaaatcactaggcgatctccgcttagaattcatttcagcatttattggttgtatgagagtagatagaaaaagacaactctggcttgaagctatcaaaaaactaagtagtgatgaaaacttttcaaatatggaactcatcagcctcatttctaaatatgaagagttaagacgtaatgaaccacagattcaagtggacgatgataaattcactaaattgttttatgacaatatccagaaatatctgcttcgaatgagctctggacatgcaattgttttatttactatcacaagattagtagatgtcgttggcgaaaagtcattagttttattcgatgaaccagaggttcatctgcatccacctttgctctctgcttttttacgaacattaagcgacttactcgatgcacgcaatggtgtagcaataattgcaactcattccccagtagtactgcaagaggttccaaaatcctgcatgtggaaagtcctacggtcaagagaagcaataaatattatccgtccggatattgagacattcggtgagaacttaggtgttttaactcgtgaggtgtttttacttgaagtgacaaattctggataccaccacttattatcgcagtccgttgattcagagctttcttatgaaaccattctaaaaaattataatggtcagataggattagaaggtcgaaccgttttaaaagcgatgataatgaacagagatgaaggtaaagtacaatgaaaaaactacctcttccagcgagaacttatagcgaaatgcttaataaatgctcggaaggtatgatgcagataaatgttagaaataatttcattactcacttccccacttttttgcagaaagaacaacaatatagaatattaagctcgacaggtcagttatttacctacgacaggacacaccctcttgagcctacaaccttagtagttggtaacctgacaaaggttaaattagaaaagctttatgaaaataatctccgagataaaaacaaacccgctagaacatattacgatgacatgcttgtttcatcaggtgaaaaatgtccattttgtggtgatataggacagacaaaaaatatagatcattttcttcctattgcacattatcctgaattttcggtgatgcctattaatttagttccatcgtgccgcgactgcaatatgggagagaaaggtcaagttttcgcagtagatgaggtacaccaagcgattcatccctatatcgacaaggacattttttttcgtgagcaatgggtatatgcaaatttcgtttccggaactccgggtgctatcagtttttatgttgaatgcccggcgaactggaggcaggaagacaaacacagagctcttcatcatttcaagctattaaatattgctaacaggtatcgtttggaggcagggaagcacttgagtgaagtgattactcaaagaaactctttcgtaaaagttataaggaaatatagttcaaccgcaacgtttcagcagctacagtcagaatttattgaagcaaatctgaaacctattatagatttgaatgacttccccaattattggaaaagagttatgtatcagtgcctagcaaactcggaagattttttcagagggatctagaatatgatgaaagatagaaaattacgacgcttatcggaagtgaacgaatactttttatatgaggagggctgtttttacaaaatccggtagtaacttgctaaccaattcctaggcaggtcattggcaacagtggcatgcaccgagaaggacgtttgtaatgtccgctccggcacatagcagtcctagggacagtggcgtacagtcatagatggtcggtgggaggtggtacaaattctctcatgcaaaaaatatgtaaaatcggtagcaactggaaatcattcaacacccgcactatcggaagttcaccagccagccgcagcacgttcctgcatacgacgtgtctgcggctctaccatatctcctatgagcaacgtgttagcagagccaagccacaactctaattttaatacataatgaatgataataataatattaaaaatttcctgtgtaactaatttactatatggtttctgataagaatcattgcaaagatcaaacaacttgtattacattgacagttaagcagttaattttatcacctctaaaatatatcagcatctagcatgcaacctatcaaaatggagagttttatgactaaaaaaccatgggaaagaagacttaaagatttatcgcacttgctcaaatgctgcattgatacatattttgaccctgaattatttcgcttgaatttgaatcaattcctccaaaccgcaagaacagtaacatttattattcaaaaaaacaaaaaccagattataggatatgacatttggtataacaataatgttattgaaaaatggaaaaatgatccattaatggcttgggctaaaaattctcgcaatacgatagaaaaacaaggcgatttagaaatgtatagcgaggcaaaggctactcttatttcatcttacattgaagaaaatgacattgagtttattacaaatgaaagtatgttaaacattggtataaaaaagttagtcagacttgcacaaaagaaattaccttcatatttaactgaatcatctattattaaatcagaaagacgatgggtcgctaatacgctaaaagattacgaattattacatgccttagctataatctatggcagaatgtataactgctgtaactctcttggcatacaaataaacaatccaatgggtgacgatgtgatttcgccaacatcattcgactctttatttgatgaagccaggagaataacttatttaaaattaaaagattactccataagcaaattgtcatttagcatgatacaatatgacaataaaataattcctgaagatattaaagagcgtctaaaactggtagataagcctaaaaatatcacttcgacagaagagttagttgactatacagccaagcttgcagaaacgacttttttaaaggacggttatcacattcaaacattaattttttatgataaacaattccatccaattgatttaatcaatacaacatttgaagatcaagcagataaatatattttttggcgttatgcagctgacagagccaaaataacaaatgcctatggcttcatttggatatcagagctatggctcagaaaagcaagcatctactccaataaaccaatacatacaatgccaattatagatgaaagacttcaggtaattggaattgattcaaataataatcaaaaatgtatttcatggaaaatagttagagaaaacgaagaaaaaaaaccgactttagaaatatcaacagcagactcaaaacatgacgaaaaaccatatttcatgcgttcagtcttaaaagcaattggcggtgatgtaaacactatgaacaattgagtcatagaacttccattattctcctgaagataataatcgccaaataaaccaatactcagctttacaatatactaactaaccgcagaacgttatttcatacaacgtttctgcggcatatcacaaaacgattactccataacagggacagcaggccactcaatatcaggtgcagttgatgtatcaacacggttcagcaacacccgatacttcttccaggcttccagcaacgaggtttcttccttcgttgcaatttccagatctgcagcatcctgaagcggcgcaatatgctcactggctacctgcatcaggcttttttttgtttcttccgcctcccggatccggaacagtttttctgcttccgtatccttcacccaggctgtgccgttccacttctgatattcccctcccggcgataaccaggtaaaattttccggtaacggaccgagttcagaaataaataacgcgtcgccggaagccacgtcatagacggttttaccccgatggtcttcaacgagatgccacgatgcctcatcactgttgaaaacagccacaaagccagccggaatatctggcggtgcaatatcggtactgtttgcaggcagaccggtatgaggcggaatatatgcgtcaccttcaccaataaattcattagttccggccagcagattataaatttttatggtccgtggttgttcactcattctgaatgccattatgcaagcctcacaatatagttaaatgcaatgtttttgacggtgttttccgcgttacccgcagcgttaacggtgatggtgtgtccgtgtgaaccaatactgaaagaatgggcatgagcaccgataacaaccggatgctggtgcgcaccaataccaactgtatgcgcatgtgcaccggcactcacggctgtaccggacaatgagtgactgtggctgccctgactgtccgttttcgataaataagcaataccctgtgtgctggttcctttaactgtggataaacttcctgtaatggttgctgttccatactgactccagccagaactgttcatccttaaaccacttgtgtgggcatgagcacccgcggcccctgttgaaccgctcagactgtgagcatgagcccccgtgttattcgtcgatttggtgccgtaatcgaaactgcctgttgttttcgtcccgtaatcaaacgacgatgtggttttcgtccccaaatccgtaccggatgcactggcactgtgggtgtgcgacttaattccatcctgttcctgagacaatacagcacgaccgctggcgggtttccccttgattgtccagcctcgcatatcaggaagcacacccgatggatacgcgacagcaagttttgggtaggctgatttgtcaaacgcctgcccctgcatcaggacgtagccagacggaacgatatctgatggccacgggatcggcgcacctgccggaaaggccgaattctcaccggccccaaggtattcaagaacatctgcaacggaattttttgccagaatatccctgccaacctgagtcagttcagtcaggctggcggcatcattttccgcaaaatacggtaatttatttttcgccgtggaaagccctgccagcgccgtcagtgtcgcattcttcggttgtttacccgcaagcgcgttagtcatggtggtagcaaaatctggatcattcccgagcgctgcggccagttcattcagcgtattcagtgcgtcaggtgacgcgtcgataacatctgcaatcgcggccagtacaaaagcggtgttcgcaatctgggtattgtttgttcccctgagcgcggttggtgctgttggcgttccggtcagtgccggactgtccagtgggcttttctgttcgtttcatccattaccaccttaaccgcctttggcgttgcagcaagcgtttcagacgtgctgttggttgcactgctgagctgcactatcccctttctcgttgtgtccgcatcctcaagcgcgacagctgaagctatatcttctgcacgttttgccgaattttttgcacgtattgccgccgcttctgccgcacttttgctctgcgatgctgataccgcacttcccgcagcctctgtcgccttcgtggatgccgttgacgcactccccgccgccgctgtttttgcgtctgccgcggcagaggcgctccgttccgctgctgtttcagatgacctggcattcgtctcggacgtttttgccgccctggcagaattttctgccgccgttgccgaggaagctgcacgaccggcacttgatgatgcgttcgtttctgatgattttgctgcctcttttgaggccaccgcatctcgtgctgaagtggcggcctctgacgctttcgtggccgcggtggaggcagacgtggcggctgattgttgtgacgctgcagcattcgtttctgacgttttcgccgcaccggcactggtggccgccgcgttttttgaggactctgcggctgcggcacttttttccgcttcagtggcctttgctgatgccgcttctgcgccggaggacgcttcctgagctgacgatgcagcctgtccggcggacgtgctggcggcgcgtgctgagtcagttgcatcagtcacaagggccgcgacctgagcagctgatgcactggcatcgccggctgatttcttcgcgtctgccgtactctgtgccaccacggacgcgttacgcgccacctcttccaccatcagttcaagacgacgcagcacctccggccgggcatcatcctccgtcatggcacagagaaaatcattcagcgtccccggttgtgaatcttcatacacggtgatggtcccggcgtgcgatggtggaaaaccgtcaacctgcaggatgacactgtactgaccgtactccacatccatgctgtaacgcccggcttcatccggattctctgagcccaccgtgttcaccaccaccgtggtgctgttacgtctggctttcagctgaatggtgcagttctgtaccggttttcctgtgccgtctttcaggactcctgaaatctttactgccatattcaccccacaaaaaagcccaccggttccggcgggctgtcataacactgtgttacctggctaatcagaatttataaccgaccccaacgatgaatccgtcagtacgccagtcgccactgccggagccttcataagcaatatcaacaacgacggacgctgccggattaatctgtatacctgcactccacgccactgaggtatgccgcattgcactttcgtccctggcagtggtcgtctctttcatatacccgggagtgatttccgtcttacggtaatccattgtactgccggaccaccgactgtgagccactccggccatggcgtacgcactgacctgcttactgatttgtaaaaccggtccggccatcacgctcacataacgtccacgcaggctctcatagtgaaacgtatcctccccggtcatcactgtgctgctctttttcgacgcggcgaaccccagggaagccatcacccccacactgtccgtcagctcataacggtacttcacgttaatccctttcagatgactcacaccggtatccccgcccgacaacgacggcaatgtacccggtttcacttgaaaatagcccaccgtaaacgtaccatgtccaccttccgcacgggccggagtgactgtcaccgcaagtgcggcaaagacagcaacggcaatacacacattacgcatcgttcacctctcactgttttataataaaacgcccgttcccggacgaacctctgtaacacactcagaccacgctgatgcccagcgcctgtttcttaatcaccataacctgcacatcgctggcaaacgtatacggcggaatatctgccgaatgccgtgtggacgtaagcgtgaacgtcaggatcacgtttccccgacccgctggcatgtcaacaatacgggagaacacctgtaccgcctcgttcgccgcgccatcataaatcaccgcaccgttcatcagtactttcagataacacatcgaatacgttgtcctgccgctgacagtacgcttacttccgcgaaacgtcagcggaagcaccactatctggcgatcaaaaggatggtcatcggtcacggtgacagtacgggtacctgacggccagtccacactgctttcacgctggcgcggaaaagccgcgctcgccgcctttacaatgtccccgacgattttttccgccctcagcgtaccgtttatcgtacagttttcagctatcgtcacattactgagcgtcccggagttcgcattcacactgccactgatatccgcatttttagcggtcagctttccgtccggtgtcagggaaaaggccggaggattgccgccgctggtaatggtgggggccgtcaggcgcttcaggaacacgtcgttcatgaatatctggttgccctgcgccacaaacatcggcgtttcattcccgtttgccgggtcaataaatgcgatacgattggcggcaaccagaaactggctcagtttgccttcctccgtgtcctccatgctgaggccaatacccgcgacataatgtttgccgtctttggtctgctcaattttgacagcccacatggcattccacttatcactggcatccttccactctttcgaaaactcctccagtctgctggcgttatcctccgtcagctcgactttttccagcagctccttgccgagatgggattcggttatcttgcctttgaaaaaatccaggtaaccttccgcatcatcgctcgcccgaccgacggcctccacgaatgccgatttgccaacggtgttcacactgcggatataaaagtaataatcatggcccggtttgatattgatactggcggctatccagtacagcgccgtaccaagataacgcgtgctggtttcaacctgtctgatatccgcaatctgcttttccgagaaccagaactcaaactgtaccgtcgggtcataaacggcaagatgcggcgtggcggttatctgaaaatagcccggcgtcagctcaatcctcgacggtgctgccggtgcggcaatccggaacgataccgacgccggatcgccctgctgcccccacgcatttaccgcccggactgtcagcctgtagttccccagcgccagttgcgtgaagcggtatgtggtttccgtcgtccgggccgtgctgaccagccgctcactgccgtcgtccgctgttacggtcagacggagcaggaaactcacgcccttcaccaccttcggtgtgtcccatcgcgccagcacctgatattccccgctgtctgcagtgacttctgcggtcaggtgctgcaccgctggcggcgtgacaccattcaccgtgccactctgttcgccgtcaaagtgcgccccgttatccacgatggcctctttttccggcacatgctgcacggcggtgatggcatacgtgccgtcgtcgttctcacggatactcacgcagcggaacagtcgctggcgcagcgtcggcagcttcagctcccatacgctgtattcagcaacaccgtcaggaacacggctcacttttaccttcacgccgtcggtgacggactgaacctccacgctgaccggattgccacttccgtcaaccaggcttatcagcgcggtaccggaggatggcagcgtgatttcacggtcgagcgtcagcgtccgggtctggctgttcaccgccagcacacgaccaccggtgctgataccggcatagtcatcatcgcagatttcaataacatcgcccggtacatggcgaagcccttctgcgccgacgctgaaatccacggtctgcgtttccagcagttctgttttaatcagccacagcccggcgcggtgtgcctgcccccggctggtacagccaaaggcatccatcttcgtaacattacgaccgtaacgggcaatggcctgcgtatcttcaacaagctctgtcgccgtctcccagccgttgttcgggtcaatccagttcacctcaacggcattatggcggtccttcagggcgctgaagctgtagcggaacggcgcgccatcatccggcatcaccacattactgcggttataggtccacgtcttatccgacggtcggtcctgcacgaacgtcagcgtctgcccgttccataccggcatacagcgcatcgccgagcagaaatcgctgagcacatcccacgccttacgctgtgtggtcaggtacgcattacaggtgatgcgcggctccgtgccgccaaagccgtccggcactgactggtcgcagtactggccgatgacatacagcgcccatttatccacatccgccgcaccaagacgtttccccatgccgtagcgcggatgggtcagcatatcccacagacaccaggccatgttgttgctgtatgccggtttaaacgttccgtcccagataccgctgtattgccgcgtctgcgggttatagttcgacggcacctgcagaatacgcccgcgcagatgataattacggctcacctgctggctgccgaactgctccgagtccacctgcacgccgaccagtgccgtgttcgggtagcactgtttcacatcgatgatttcagtgtatgacgaccagagcgttttgttctgcagctggtctgtggtgctgtccggcgtcatcctgcgcatccggatattaaacgggcgcggcggcaggttacccatcaccaccgaggccagatactgcgaggtggttttgcccttaatggtgatgtctttttccgtcacccagccaccgttacgttgtatctgaaccagcaggcggacttccgacggattcctgtcaccctttgaggtggtttccaccagtgcctgtacaccgaaggtaaagcgcagacggtcgatgtttgcagacgtaatggtgcgggtgatcggcgtgtcatatttcacttccgtacccagcaccgtctcggagccggaggattcaaatccctccggcggagtctgctcctgctcaccagcccggaacaccaccgtgacaccggatatgttggtattcccctcagtgtccagcaccggcgtactgttcagcagcacgctttttaagccatccaccggaccttcaatcggcccttcgctgatggcatcgatcacactcagcaactgcgtggacttcaggttgtccttcgcttcgcgcggggtatgccccttactgcttcctttacccattcctcacgctccataaatgacaaaaccgcccgcaggcggtttcacataaaacattttgcatcagcgaccaatcaccacaacctgaccaccgtccccttcgtctgccgtgctgatctcctgagaaaccacgcgtgaccccacgcgcatttccccgtacagaacaggcagaacattgccctgggcaaccatgttatccagtgaggagaaataggtgttctgcttaccgttatccgttgtctgtatacggggagttctggctttcggtgccagcatctgcgccacaccaccgagcaccatactggcaccgagagaaaacaggatgccggtcataccaccggccccaatggctgccccccatgctgcaagggtggctccggcggtaaagaatgatccggcaatggcggcagcccccaggacaatctggaatacgccacctgacttggccccggcgactctgggaacaatatgaattacagcgccatcaggcagagtctcatgtaactgcgccgttaacccggacgtgctgacgtcccgcccggcaatccgtacctgataccagccgtcgctcagtttctgacgaaacgccgggagctgtgtggccagtgcccggatggcttcagcccccgttttcacacgaaggtcgatgcggcgaccaaatcgttgtaaatccccgtaaaggcagatgcgcgccatgcccggtgacgccagagggagtgtgtgcgtcgctgccatttgtcggtgtacctctctcgtttgctcagttgttcaggaatatggtgcagcagctcgccgtcgccgcagtaaattgcggcgtgattcggcactgatgaaccaaaacagcacagcagcacatcgcccggctgtgccgctgacaacggcacctgatacagccccgtcgcctccagattatccagatagagattctggccgttacgccaccagtcatcctcacgatgaaagtccggcatctcaatccccgccagatgataagcatcccggaacagtgtgtaacagtccgtcacaccgtgctcaaagcgccgcccggtgagatgcggcacacagcggaacttatgaatcgtcccccggcagaccagccaccacggcaaatcactctgcacctgcagccgccggtcggcctcactcagccagggcagaccaccggggtggctgtggaccagcgccacaatctcaccctgcatttctgcctgcagccagtcttccggcgacatacggaaatagcctccggctcaccggagatattcacgcaggggaaatatctttccccctccggcgtgcttaccacgaagccgcacgactccgctggcgcacatcgccgggcgtgcgccagaatcgctgattctgtctgtgtcatgggatttactgcgaaagtttgttaatggaaaggaagccgccaaagttgccgacgttattgcggaacttacaaccgctcaggcatttgctgcatttatccttcgtgatatcggacgttggctggtcatattcatccgcgacagccggaccgctataaccgcactcgtcaccgcgataggtccaggtgcaggtgttggccagcatgatacgtcccggaaaaacagcgccatccgtttccgtcggcgtggacagtacaaaggaggcactcaccgcgctcagttcgctgcactgctcaatgcgccagcggctgatcacctcctgctccggatcggcgtaactgtttccgttgacgaagttcaccgcatccagaaaacgggcgtaaaccttacgccggaccaccgttccgccgaccagactctgcatatcttccgccatcccggtgaccataccgtacaggttagaaaccgtcagcgtggggcgcgtactggtgcctttgccattcagttcaaaaccgctcccctgaatgggatacggctgatactgtcgcccctgccaggtgaccggctcacctttttcgttctgctcattacagaaaaaataacgttctccaccgacctctgtcaggtcgatttcccagagcaccacgctggccgactgctccgcacgggtgcattcattcagtgtttcctgccggatatcctgcatcagttcaccacctgttcaaactctgcgctgaactcaacacgcagcatactgacccgcgacgaccattttgcgcaggtcacctttatctgccgccactcataaggcggcgtccacagaaaggatttccagcccccgtgctcttccagaaacgactccagtaccgtggcctcctcacgggggacagaaagcgtcacgctgtacgttttcaggttggcattcagcccggcaggcgctcgctgagaatagccatcaccaaagcgcacctttcttacagaagggaccgaagccacatccataccgggtttcactttccagcggaaggtcttcatcgtccacctccggagaacaggccaccatcacgcatctgtgtctgaatttcatcacgggcacccttgcgggccatgtcatacaccgccttcagagcagccggacctatctgcccgttcgtgccgtcgttgttaatcaccacatggttattctgctcaaacgtcccggacgcctgcgaccggctgtctgccatgctgcccggtgtaccgacataaccgccggtggcatagccgcgcatcagccggtaaagattccccacgccaatccggctggttgcctccttcgtgaagacaaactcaccacggtgaacaatccccgctggctcatatttgccgccggttcccgtaaatcctccggttgcaaaatggaatttcgccgcagcggcctgaatggctgtaccgcctgacgcggatgcgccgccaccaacagccccgccaatggcgctgccgatactcccgacaatccccaccattgcctgcttaagcagaatttctgtcatcatggacagcacggaacgggtgaagctgcgccagttctgctcactgccggtcagcatcgccgccatattctgtgcaataccatcaaaggtctgcgtggctgcactttttacctgcgacatactgtccgtggcgctctcttcccactcactccagccggacttcaggcctgccatccagttcccgcgaagctggtcttcagccgcccaggtctttttctgctctgacatgacgttattcagcgccagcggattatcgccatactgttccttcaggcgctgttccgtggcttcccgttctgcctgccggtcagtcagcccccggcttttcgcatcaatggcggcccgttttgcccgttgctgctgtgcgaatttatccgcctgctgcgccagcgcgttcaggcgctcctgatacgtaaccttgtcgccaagtgcagccagctggcgtttgtactccagcgtctcatctttatgcgccagcagggatttctcctgtgcagacagctggcgacgttgcgccgcctcctccagtaccgcgaactgactctccgccttccacaaatcccggcgctgctggctgattttctcatttgctccggcatgcttctccagcgtccggagttctgcctgaagcgtcagcagggcagcatgagcactgtcttcctgacgatcgcccgcagacaccttcacgctggactgtttcggctttttcagcgtcgcttcataatcctttttcgccgccgccatcagcgtgttgtaatccgcctgcaggattttcccgtctttcagtgccttgttcagttcttcctgacgggcggtatatttctccagcggcgtctgcagccgttcgtaagccttctgcgcctcttcggtatatttcagccgtgacgcttcggtatcgctctgctgctgcgcatttttgtcctgttgagtctgctgctcagccttctttcgggcggcttcaagcgcaagacgggccttttcacgatcatcccagtaacgcgcccgcgcttcatcgttaacaaaataatcatccttgcgcagattccagatgtcgtctgctttcttatacgcagcctctgccttaatcagcatctcctgcgcggtatcaggacgaccaatatccagcaccgcatcccacatggatttgaatgcccgcgcagtcctgtctgcccaggtctccagcgtgcccatgttctctttcaggcggcgggtctggtcatcaaaccctttcgttgcggcctcgttcgccgcctgcaatgccccggcttcatcgccggaacgctgcaactgagcaacatacgcaatctgctccgccgacacgttatggaactggcgagccatcgccgtcagccccgacgtcgggtctgtggtcagcttcccgaaggcttcagcgaccttgtccacctccacgccggatgcagaggagaaacgcgccacactctggctgatggacgcaatctgagcctcaccgcttacccccgccttaaccagtgcgctgagtgactcgctggtctggttaaacgtcagccctgccgcctgcccggctctggacaggaccagcatacgatctgccgtcagtcccgcctgattgccggaaaggaccagcgttttgttgaaatcggacagggttgagttgccctgataccaggcatacgccagcgcaccggtcgccaccgccagcgaggtggcccccaccatcggcagggtgatcgcaccggcaagccccctgaacatggggatcatcccgccgaaggagtccttcacctgccccccctgttgcagcaggatcagccacggactttgcccgcctgcaagctgcgtggccacgtcggtgaactgtgcaggcagcatacgcatggcggctttatactgcccgacggaaatccccgctttctgtgcagccagcgcctgtcggctcagcgactgttcaacgactgccgctgtttttttcgcatcactttccgtaccagaaaaatgacgcctgactctggccatctgctcgtcaaatctggccgcatccagactcaaatcaacgaccagatcgcctaccggttcagccataccggactcctcctgcgatcccttctgatactgtcatcagcattacgtcatcctccgtcatgtccgccacatccggggaagcggggataacttcattcccgtccgggccaaagcggacacctccggcaagccctgccgctttctgcatcagcacatcatcttcaggctcttcgtcagcctcgcgccggttcagcagactgaaatccagcggatgcatatccggatcgctgaaaaacaggctgagcacggtgtacgtcagcccggaaaagtgcatatccagcagaacatcatgaaaataatgggtactgtaaaagcggtgccagtcggcatactccgtggatgacatcccggcaagcatggcacgccagtcgggtcgccccatctcacgcgccagtttcagggcaaaactcagctcaccgtcgaacactttcccgcagaaacaggctctgcgggcccggcgtcctctgtctgttcaggggcattattcaccacaaactcatacataccagacagccggtacaccacgttttcagcatgagaaattgcctccgtgggccaggtggtaagcacttcctgctcaatctgtttaacggcttcattcatggacggcatctgcgtcttctgcggatggttatgccacagggacatcgccaccagaaacgcgccggttctgatggcgtcttccacagtaaacttccggttgctgtctgactccgcctgttctgcctgccgtttcatcagggcgagatgctcaatgcgctgcagggctgacagttcagaaagcgtgacggtcacaccgttatgttcaaatgattcggttttcaggaacatcgctgactctccggattaactggcggtgacggtaatttctgcaaccgcagcaaactcaccattaccggatacaaccggaatgttgaccttgcctgcagcaacgccgttcacggtgatggtcataccactgaccgacacggtggcttttgttttatccgcagacaccgcacgaaagctcttgtcggttacgccctccggctggaaggccacggtcagcgtggtgctctgccctttcaccaccgaggtgctggcaggcgtcacggtcatgccggttgccgctgttaccgtgctgcgatcttctgccatcgacggacgtcccacattggtgactttcaccgtgcgggtgatcacttccttcgccgtcaccgccttaccgatactgctgacccagccacggaacacatcgaccgtgccgttcgggaagcggattttataggcacgggtatcgccttcattaaaccacgccagcagcgcctgctgcccctgctctccgggcatccacgccagcgtgaagctggtatctccggcagatttctgcccctgcccggtcgcagtccagtctgcatcttcatcatcgagatagctgtcgtcataggactcagcggtcagttcgccgggcgtcaggtctttaacttttgccagacgcgaccagtcaacgtctgaaagcggattcgcgtaagggtcaccgctccccttataaacccacagggtggtcccggcacctttcaccggcattgtaggatttggtacaggcatagcgtcctcacatttcataggtaatgacataagtcagatcggctgaactccacaagcccgcatcatcgtcgcgccggtagtcatagccgctggccaccatactggtgatcaaatctgacagtgccgggatatcgctcatcaccggataaatccgggactccatccacgcatccagctctgaatccggcacctgagcaggcaggaaaacttcgatatgcagctccgcctgccaggtatcgctgtccagctcttcgcccgtgtattcagcgccggtgagataaacggcaactgccggaaaatccgcctcatcaaaaacagcggggcgaccatcaaaaaacgtcgccccggtgtcatgcttctccagtgcatccagtacggctgcacggagttcagtatgtttcatcgctttattaccatcctcagttgatgctgcagcgcatagcccagctctttcggaagacgttcacgccgtatccgctcaatattttgtttaaacgccgtggtcagcggcaccgccatcgggattttcaccacatcaatggggtaacggtttttcccagccacacgctgcatgacatgccaccggccatttttcagttgctgaataaacgcgccgggaatacgacggttacccaccacaagcacgctgccgccacctttcagggatgaacgctgcccctttttacgacgcctgcggcgcgaaaggacaacccgcgcattacccagcttgattacgggcaaatccccccggttaactttgattctggcctgcggatttttgaccgtggcccttttcagcctggccctttcctttaccagtttccggcgtacctttgtctcacgggcaacctgtgacgccgactgcgatatcgcggatgaagcaacgcggttaatggccattgcggcggcaccaggcaccgccgttttgctgatacggctgaggttttcaacggcctgctcaagaccttttatggccatacatccccctttcagcggcgacggttaacggcaggcggtacgccccgtccaagccagagatgacaacttccgccatcatccggcgaaacccgatctacccagaaattttcctcaccgatggtcagcgtgtctccacgccgcagctgccgcacctcatcagtccggacaaacagggacgggctggagccttcaacgcgcacgccctgtccggcatagctgatattttcagggtcatcaaaaacaccacgtatcaccgcacctgactgctcaccggatgtaatggtggctgacgttcccatgtacccgcgtatcgtttcatcggcgcgggcaatggcagcatcgaacaggttatcgaaatcagccacagcgcctcccgttattgcattctggccaggccgcgctctgtcatttcggctgccacaccggcagagacacgaaacgccgttcccggcagcacaaatgccacaggttcatcccgcgtggcgtgaagtgcatcagtatgcagcttcaccagtgccacgaccgtgaccagttcagacgtatccagaatcacggtatccggctgcgctgatcccacctcattttcatgtccggtcagcacattttcccggctgagaggggtgtcctgaccggcagtttcatccgtgtcatcaagctcctctttcagctctgccacacggagcgccagttcttctttcgtccccgtcaggctgacatcacggttcagttgttcacccagcgagcggagacgggcaatcagttcatctttcgtcatggactcctccacagagaaacaatggccccgaagggccatgattacgccagttgtacggacacgaactcatcagggtcagccagcagcatcagcggtgctgactgaatcatggtgaactcacgcgccggatcgccggtggtcacccagtttttcgggtaacgggcagaggcgttaatgccttcgcgctgtgcgtccgcatcctgaatgcagccataggtgcgcagaccgcgtgcctgagtgttccccagcaccatcgtgttgtccggcaggaagttctttttgacgccgttttccacgtactgtccggaatacacgacgatggccacatcgccatacatccccttataggacaccgctttgcccaggtctttcaccgctgtctccagctcggaattagagccacgacgggtatccagcttctccttgacggctttgaaggaacggaacagcgcccagcctttcggatcgaacacgatgatattcaccacaccgctggcgttcagcgcgtaggcttcgatatcgtcggtcgggtcatacgtggacttgtcacgcttgctccactccgtgccgccggactgcgtgatgttattctcctcactgcggcccatatccacctcaaccggatcgaaggcttcaccggtcatggtgtatttgcccttaagcacggcagaaactgcctgcatctcttcgacctgagcaatggccagctcttcgtcacgcatgttctgcatgatgatgcgacggcggcggtaagccgggtccgccagattctgcggatcttcatccggcaggcgacgcagggtcatctgcggattcacttcatgcttcggcttgacatatcccggcgtaaattcagaggtggagccgccacgggaacggataacctcaccggaaacaatcggcgaaacgtacagcgccatgtttaccagtcccggaatttgtgagagatagactttctccgtggtgaagggatagctctcacggaaaaagagacgcagaaacagcggatcaaacttaaatttctgctcatttgccgccagcagttgggcggttgtgtacatcgacataaaaaaatcccgtaaaaaaagccgcacaggcggcctttagtgatgaagggtaaagttaaacgatgctgattgccgttccggcaaacgcggtccgttttttcgtctcgtcgctggcagcctccggccagagcacatcctcataacggaacgtgccggacttgtagaacgtcagcgtggtgctggtctggtcagcagcaaccgcaagaatgccaacggcagcaccgtcggtggtgccatcccacgcaaccagcttacggctggaggtgtccagcatcagcggggtcattgcaggcgctttcgcactcaatccgccgggcgcggttgcggtatgagccgggtcactgttgccctgcggctggtaatgggtaaaggtttctttgctcgtcataaacatcccttacactggtgtgttcagcaaatcgttaacggcatcagatgccgggttacctgcagccagcggtgccggtgccccctgcatcagacgatccagcgcagtgtcactgcgcgcctgtgcactctgtggtgctgcggccagaatgcggcgggccgttttcacggtcataccgggggtttctgccagcacgcgtgcctgttcttcgcgtccgtgagcctcctcacagttgaggatccccataatgcggctgttttctgccgcaaccgctgcggtgatctgcgcgttcacgtccggctgcgccgcgctggcgttctcgccctccgtcgctggcaccacgtcagtaacgtcagcctgcgaagcagtggctgaaacagttgttgattgagtctctttggtcattcgccctcctgagagacgggatttacgtgcatccagtgcatcacgcatgacggtgatcgcatcggtgctgttaacaagttcatcagccagtccggcatcaatggcctcctgaccgctgtacactgcagcctcggtatccagcacaacctgcacggacaggccggtatatgccgacaccttctgcgcaaacatctggcgggttgcgtccatccgggactgcagtgtctcccggacgtcatccggaagatggctgtaggggttgccatccaccttatggctgccgctgtaaatcagcgtgatttccacaccctgtttctccagcgcagcaccgtaattactgtgagccatcatgacgccgatggagcctgtccgggcggtctgcgtgaccagacgccgggaggcggcactggcaagcaactgacctgcactgcagttcatgtcgttggcaagcgcccataccggttttatgtcacgcacacgggcgatgatgtcagcgcagtcaaatgcccccgccaccatcccgccgggcgtgtccatatcgagcagaatgccgtccaccatcggatcgctggcagcctgttgcagacgggcgataatgccgttgtaaccggtcatccccgagtacggctgcagcgcccgcgtccggctgaccagcgtgccggacaccggcagcacggcgatgccgttcatgacctgataactgcgggcctgtcgtggtccgtcatcatcaccggataatgccagcgtcgcgagtgcctcctgggcagtcaggctgtcgccggacaccgcatccgtcaggctgctgatcccaagctggcctgcaagcgcacaaaagaaaacccgcgcataggcgggttcaagcatcagcggctcattaaaggccatgctggcaatatgcgggagattacgcagctctgctgtcactcttctcctcctctgttgattgtcgcagcccggattcaaatgctgcagccgcccaggcgggcggtttaagaccggctgcacggcgctccatcgtttcacggacctgctgggcaaaaatttcctgatagtcgtcaccgcgttttgcgcactctttctcgtaggtactcagtccggcttctatcagcatcaccgcttcctgaacttctttcagaccatcgatggccatacgaccggagcctatccagtcgcagttcccccaggcactgcgggcttcctgaaaactgaagcgcgcttttgaaggtaacgtcaccacgcggcgaacgatggcctcttccagccagcacagaaacatctggctcgcctgacgggatgcgacgaattttcgccgccccataaagtacgcccacgactcgttcgcactggcccgtgccgtggagtagctcatctgggcgtaattccgggaaagctgctcatacgagacacccagcccggcagcgatataccgcagcagtgactgctcaaacacggagtagccgttatccgtatcctgagccgtctgcaggttcagtgagtcacccggcatcaggtgcggtacttttgcgcctcccagccggaccggcgctgcggcgtaatacgcggcaatttcaccaatccagccggtcagcctttcccgctgctcctgactgttcgcgcccagaataaaatccatcgctgactgcgtatccagctcactctcaatggtggcggcatacatcgccttcacaatggcgctctgcagctgcgtgttctgcagcgtgtcgagcatcttcatctgctccatcacgctgtaaaacacatttgcaccgcgagtctgcccgtcctccacgggttcaaaaacgtgaatgaacgaggcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctggatggcgttggcggcatagccgttattgcgtaccagatcgtctgcgcgggcattgccacgggtaaagttgggcaacagggctgcatccacactttcactcggtgggttccacgaccgcaactgccctccaaatccgctgccaccgccgtgataaccggcatattcgcgcagcgatgtcatgccgtccggccccagaagggtgggaatggtgggcgttttcatacataaaatcctgcaggtcccctgcgtcgctgtgtcatgccggtctgcacttccagctctgcaatatattttttcaggtcagacacggaagtggccgtaaactccacccttcgtccgtctttctgtactgttgccacccgtttacctgtcatcaggtcatgcagtgccgcacgggcagcggcaagttcttcctgtcgcgtcattcatcctctccggataaggcacgggcgtaatctgccagtgttttcttgttggttgctgcaccatcctcttcctgcaggctcgccagcagcgcactgagatccagctgccagcgggaaatactgatgcgcagcgccgccagcgcataaacgaagcagtcgagtgcctcattgcgtcgctttttgctgtcccacagtatttttttcctgccatccacccatttttcgacctgctcttcagcagtcagctgctgcgcttcggtcagatcaaaaatatccgggttattcgggaagtgaacggcaccgggaagcggttcatccccttccggcgtcagtgtgaagcggttataaatctgctctttcgcggtatccgtaccgatttcggtaaggtaaaccccgtttttgtttcgcttacgtggcatgctggccaccggctttccgtagacggatgcccctttaatggggatcacccggaacagcccatgttttttcgagcgttcatacacaatggtcgggtcaatcccgccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggaggggtcatccggcgtccatttgaggccaaacggcgtctctttgtcgccaaatttaagatactgctcctccccgcaatgcgggcaggcaacatgaaaacgcataaaatgcggggattcactggctgcacgctcaatctgacaggtgcctctcacttttggcgtggagccacggatggactttggccagaccgagccttcaatacgcttgtcacccaggaacgtcggagagccttcctgttcaatatcatcatcaaaagcagcaagttcatcataacccgccacatccaccgacttttcacggtagttttttgccgctttaccgcccaggcaccagaagccacgcccattagtgaaacgcttcatggtgagcgtgttatcccggtgctttttgccataccacggggccagcgccagcagcgacggaatatcacgaatagtcggctcaacgtgggttttcataaagttctcggcatcaccatccgtcggcaaccagataagggtgttgcgctgcttatgctctataaagtaggcataaacacccagcagcattttggaataaccgacacgggcagacttcaccacattcacctcacggatgtagtcgctgcccatcgcattcatgatggcccgctgaaagggcagtgtttcccagcgcccttcctggtatgcggattctttcgggagatagtaattagcatccgcccattcaacggcggtctgtggctccggcctgaacagtgagcgaagcccggcgcggacaaaatgccgcagcctgttaacctgactgttcgatatattcactcagcaaccccggtatcagttcatccagcgcggctgctttgttcatggctttgatgatatcccgtttcaggaaatcaacatgtcggttttccagttccggaaaacgccgctgcaccgacagggggagcccgtcgagaatactggcaatttcacctgcgatccgcgacagcacgaaagtacagaatgcggtttccaccacttcagcggagtctctggcattcttcagttcctgtgcgtcggcctgcgcacgcgtaagtcgatggcgttcgtactcaatagttcctggctggagatctgcctcgctggcctgccgcagttcttcaacctcccggcgcagcttttcgttctcaatttcagcatccctttcggcataccattttatgacggcggcagagtcataaagcacctcattacccttgccaccgcctcgcagaacgggcattccctgttcctgccagttctgaatggtacggatactcgcaccgaaaatgtcagccagctgctttttgttgacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgcccaggtcgccgcccgtcaatcggccctttagtggagc

SEQ ID NO: 63 - DNA, 28 bp, synthetic construct
  misc_feature 1..28: note = Synthetic Polynucleotide
  source 1..28: mol_type = other DNA
gcaatatcagcaccaacagaaacaacct

SEQ ID NO: 64 - DNA, 44 bp, synthetic construct
  misc_feature 1..44: note = Synthetic Polynucleotide
  misc_feature 37: note = Carboxyfluorescein (FAM) attached to T
  source 1..44: mol_type = other DNA
tttttttttttttttttttttttttttttttttttttttttttt

SEQ ID NO: 65 - DNA, 75 bp, synthetic construct
  misc_feature 1..75: note = Synthetic Polynucleotide
  misc_feature 1..75: note = Carboxyfluorescein (FAM) attached to one T in sequence
  source 1..75: mol_type = other DNA
ttttttttttttttttttttttttttttttttttttttttttttttttttttttttttttttttttttttttttt

SEQ ID NO: 66 - protein, 970 aa, Clostridium botulinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 67 - DNA, 46 bp, synthetic construct
  misc_feature 1..46: note = Synthetic Polynucleotide
  source 1..46: mol_type = other DNA
tttttttttttttttttttttttttttttttttttttttttttttt

SEQ ID NO: 68 - DNA, 1292 bp, synthetic construct
  misc_feature 1..1292: note = Synthetic Polynucleotide
  source 1..1292: mol_type = other DNA
gccatcagattgtgtttgttagtcgctttttttttttggaattttttttttggaattttttttttgacgctcagtaatgtgacgatagctgaaaactgtacgataaacggtacgctgagggcggaaaaaatcgtcggggacattgtaaaggcggcgagcgcggcttttccgcgccagcgtgaaagcagtgtggactggccgtcaggtacccgtactgtcaccgtgaccgatgaccatccttttgatcgccagatagtggtgcttccgctgacgtttcgcggaagtaagcgtactgtcagcggcaggacaacgtattcgatgtgttatctgaaagtactgatgaacggtgcggtgatttatgatggcgcggcgaacgaggcggtacaggtgttctcccgtattgttgacatgccagcgggtcggggaaacgtgatcctgacgttcacgcttacgtccacacggcattcggcagatattccgccgtatacgtttgccagcgatgtgcaggttatggtgattaagaaacaggcgctgggcatcagcgtggtctgagtgtgttttttttttggaattttttttttggaattttttttttcatcgtcgtgagtagtgaaccgtaagctgcgttctgtttcggatgtatgaaaacatacatccgaaacagaacgcagcttacggttcactactcacgacgatgaaaaaaaaaattccaaaaaaaaaattccaaaaaaaaaacacactcagaccacgctgatgcccagcgcctgtttcttaatcaccataacctgcacatcgctggcaaacgtatacggcggaatatctgccgaatgccgtgtggacgtaagcgtgaacgtcaggatcacgtttccccgacccgctggcatgtcaacaatacgggagaacacctgtaccgcctcgttcgccgcgccatcataaatcaccgcaccgttcatcagtactttcagataacacatcgaatacgttgtcctgccgctgacagtacgcttacttccgcgaaacgtcagcggaagcaccactatctggcgatcaaaaggatggtcatcggtcacggtgacagtacgggtacctgacggccagtccacactgctttcacgctggcgcggaaaagccgcgctcgccgcctttacaatgtccccgacgattttttccgccctcagcgtaccgtttatcgtacagttttcagctatcgtcacattactgagcgtcaaaaaaaaaattccaaaaaaaaaattccaaaaaaaaaaagcgactaacaaacacaatctgatggc

SEQ ID NO: 69 - DNA, 7240 bp, synthetic construct
  misc_feature 1..7240: note = Synthetic Polynucleotide
  source 1..7240: mol_type = other DNA
gccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcacgttctgtttatgtttcttgtttgttagccttttggctaacaaacaagaaacataaacagaacgtgcttacggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggcagcgactaacaaacacaatctgatggc

SEQ ID NO: 70 - DNA, 20 bp, synthetic construct
  misc_feature 1..20: note = Synthetic Polynucleotide
  source 1..20: mol_type = other DNA
tttttttttttttttttttt

SEQ ID NO: 71 - DNA, 7240 bp, synthetic construct; identical to SEQ ID NO: 69
  misc_feature 1..7240: note = Synthetic Polynucleotide
  source 1..7240: mol_type = other DNA

SEQ ID NO: 72 - DNA, 3653 bp, synthetic construct
  misc_feature 1..3653: note = Synthetic Polynucleotide
  misc_feature 3648: note = n is uridine
  source 1..3653: mol_type = other DNA
gccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcagcgacggctgagaagttccactcaagcctctgacactgattgacacggtttagtagaacnttttt

SEQ ID NO: 73 - DNA, 3643 bp, synthetic construct
  misc_feature 1..3643: note = Synthetic Polynucleotide
  source 1..3643: mol_type = other DNA
cttctcaatgtgtacgtgtcctctagaggcttgagtggaacttctcagccgtcgctgcttacggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggcagcgactaacaaacacaatctgatggc

SEQ ID NO: 74 - DNA, 45 bp, synthetic construct
  misc_feature 1..45: note = Synthetic Polynucleotide
  source 1..45: mol_type = other DNA
gcaatatcagcaccaacagaaacaacctttgaggcgagcggtcaa

SEQ ID NO: 75 - DNA, 15 bp, synthetic construct
  misc_feature 1..15: note = Synthetic Polynucleotide
  source 1..15: mol_type = other DNA
ttgaccgctcgcctc

SEQ ID NO: 76 - DNA, 10 bp, synthetic construct
  misc_feature 1..10: note = Synthetic Polynucleotide
  source 1..10: mol_type = other DNA
tttttttttt

SEQ ID NO: 77 - DNA, 59 bp, synthetic construct
  misc_feature 1..59: note = Synthetic Polynucleotide
  source 1..59: mol_type = other DNA
ggttgtttctgttggtgctgatattgcactgagtgaccaatcagctacgtttttttttt

SEQ ID NO: 78 - DNA, 3636 bp, synthetic construct
  misc_feature 1..3636: note = Synthetic Polynucleotide
  source 1..3636: mol_type = other DNA
ggttgtttctgttggtgctgatattgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagccgtcctgtcgctgtgtctcggacactgattgacacggtttagtagagc

SEQ ID NO: 79 - DNA, 52 bp, synthetic construct
  misc_feature 1..52: note = Synthetic Polynucleotide
  source 1..52: mol_type = other DNA
ttttttttttttttttttttttttttttcgagacacagcgacaggacgtcct

SEQ ID NO: 80 - DNA, 83 bp, synthetic construct
  misc_feature 1..83: note = Synthetic Polynucleotide
  source 1..83: mol_type = other DNA
cgtagctgattgaggtcactcagtgcaatatcagcaccaacagaaacaacctttgaggcgagcggtcaagcgacgaggtgtcc